(19) United States
(12) Patent Application Publication
(10) Pub. No.: US 2011/0052534 A1
(43) Pub. Date: Mar. 3, 2011

(54) Imidazopyridazine Compounds for Treating Viral Infections

(76) Inventors: Martin Leivers, Durham, NC (US); John Miller, Durham, NC (US)

(21) Appl. No.: 12/872,443

(22) Filed: Aug. 31, 2010

Related U.S. Application Data
(60) Provisional application No. 61/238,318, filed on Aug. 31, 2009.
(60) Provisional application No. 61/238,328, filed on Aug. 31, 2009.
(60) Provisional application No. 61/238,336, filed on Aug. 31, 2009.

(51) Int. Cl. A61K 31/5025; C07D 487/00; A61K 38/21; A61K 31/7056; C07F 9/6561; A61K 31/675; A61P 31/14

(52) U.S. Cl. 424/85.4; 544/236; 514/248; 514/43; 514/81

(57) ABSTRACT

Disclosed are compounds and compositions of Formula (I), pharmaceutically acceptable salts and solvates thereof, and their preparation and uses for treating viral infections mediated at least in part by a virus in the Flaviviridae family of viruses:

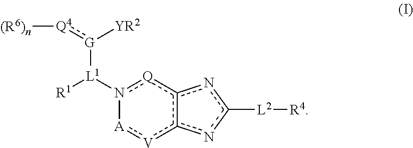

### CROSS REFERENCE TO RELATED PATENTS AND PATENT APPLICATIONS

This application is a U.S. Nonprovisional application and claims the priority benefit of U.S. Provisional Patent Application No. 61/238,318, filed Aug. 31, 2009; U.S. Provisional Patent Application No. 61/238,328, filed Aug. 31, 2009; and U.S. Provisional Patent Application No. 61/238,336, filed Aug. 31, 2009; all three of which are incorporated herein by reference in their entireties.

### FIELD OF THE INVENTION

The present invention relates to the field of pharmaceuticals. Provided herein are compounds and compositions, methods for their preparation, and methods for their use in treating viral infections in patients mediated, at least in part, by a virus in the Flaviviridae family of viruses.

### BACKGROUND OF THE INVENTION

Chronic infection with HCV is a major health problem associated with liver cirrhosis, hepatocellular carcinoma, and liver failure. HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-N52-N53-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory, and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load and there is a clear need for more effective antiviral therapy of HCV infection.

A number of approaches are being pursued to combat the virus. These include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4a protease/helicase and the NS5b RNA-dependent RNA polymerase are considered the most promising viral targets for new drugs.

Besides targeting viral genes and their transcription and translation products, antiviral activity can also be achieved by targeting host cell proteins that are necessary for viral replication. For example, antiviral activity can be achieved by inhibiting host cell cyclophilins. Alternatively, a potent TLR7 agonist has been shown to reduce HCV plasma levels in humans.

In view of the worldwide epidemic level of HCV and other members of the Flaviviridae family of viruses, and further in view of the limited treatment options, there is a strong need for new effective drugs for treating infections cause by these viruses.

### SUMMARY OF THE INVENTION

In accordance with some embodiments of the present invention, a compound is disclosed having the Formula (I):

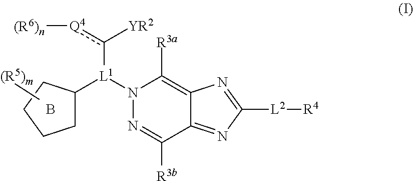

or a pharmaceutically acceptable salt or solvate thereof, wherein:

[symbol] represents a single or double bond;

ring B is a 5-membered aromatic ring wherein 1 to 3 ring carbon atoms are optionally replaced by nitrogen or oxygen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 8- or 9-membered bicyclic ring;

L¹ is independently C₃₋₆ cycloalkylene or C₁₋₅ alkylene, where one or two CH₂ groups of said C₁₋₅ alkylene are optionally replaced with NR^{a}, S, (C═O), or O and optionally two adjacent carbon atoms form a double bond;

L² is a bond or independently C₃₋₆ cycloalkylene or is C₁₋₅ alkylene where one or two CH₂ groups of said C₁₋₅ alkylene are optionally replaced with NR^{b}, S, (C═O), or O and optionally two adjacent carbon atoms form a double bond or triple bond, and wherein said C₁₋₅ alkylene is optionally substituted with one to three groups independently selected from halo, alkyl, and spirocycloalkyl;

Y is a bond, O, S, or NR^{c};

Q⁴ is O, S, or NR⁷;

R² is selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl;

R^{3a} and R^{3b} are independently selected from the group consisting of hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl;

R⁴ is independently selected from the group consisting of aryl, substituted aryl, heteroaryl, substituted heteroaryl, heterocyclyl, substituted heterocyclyl, cycloalkyl, and substituted cycloalkyl;

R⁵ is independently selected from the group consisting of hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, substituted sulfonyl, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

R⁶ is selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, and substituted sulfonyl;

R⁷ is selected from the group consisting of hydrogen, halo, aminocarbonyl, imino, amidino, aminocarbonylamino, amidinocarbonylamino, carboxy, carboxy ester, hydroxy, alkoxy, substituted alkoxy, acyl, acyloxy, cyano, thiol, alkylthio, substituted alkylthio, sulfonyl, and substituted sulfonyl;

R^{a}, R^{b}, and R^{c} are independently selected from the group consisting of hydrogen, alkyl, and substituted alkyl; and

m is from 0 to 4;

n is from 0 to 1, provided that n is 0 when [symbol] represents a double bond.

Also provided is a pharmaceutical composition comprising a pharmaceutically acceptable carrier and a therapeutically effective amount of a compound of Formula (I), or a pharmaceutically acceptable salt or solvate thereof.

Also provided are methods for preparing the compounds of Formula (I), or a pharmaceutically acceptable salt or solvate thereof, and compositions thereof and for their therapeutic uses. In some embodiments, provided is a method for treating a viral infection in a patient mediated at least in part by a virus in the Flaviviridae family of viruses, comprising administering to the patient a composition comprising a compound Formula (I), or a pharmaceutically acceptable salt or solvate thereof. In some embodiments, the viral infection is mediated by hepatitis C virus.

More generally, the present invention is directed to a method of forming a prodrug of an anti-viral compound. Rather than being bound to a heteroatom of a heterocyclic ring structure, the prodrug moiety is instead bonded to a C₃₋₆ cycloalkylene or C₁₋₅ alkylene, where one or two —CH₂— groups of said C₁₋₅ alkylene are optionally replaced with —NR^{a}—, —S—, —(C═O)—, or —O— and optionally two adjacent carbon atoms form a double bond. The prodrug moiety may be released when administered to a biological system or patient to generate the drug substance, i.e. active ingredient, as a result of spontaneous chemical reaction(s), enzyme catalyzed chemical reaction(s) (e.g., hydrolysis, oxidation, etc.), photolysis, and/or metabolic chemical reaction(s). For example, the prodrug compounds may be administered to a patient in vivo and undergo modification to generate a carboxylated compound, which may then undergo spontaneous decarboxylation to generate a physiologically active target compound.

These and other embodiments are further described in the text that follows.

### DETAILED DESCRIPTION OF REPRESENTATIVE EMBODIMENTS

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow,

reference will be made to a number of terms that shall be defined to have the following meanings.

“Alkyl” refers to monovalent saturated aliphatic hydrocarbyl groups having from 1 to 10 carbon atoms and, in some embodiments, from 1 to 6 carbon atoms. “C_{x-y}alkyl” refers to alkyl groups having from x to y carbon atoms. This term includes, by way of example, linear and branched hydrocarbyl groups such as methyl (CH₃—, often referred to as “Me”), ethyl (CH₃CH₂—, often referred to as “Et”), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 and, in some embodiments, 1 to 3 or 1 to 2 substituents selected from alkenyl, substituted alkenyl, alkynyl, substituted alkynyl, alkoxy, substituted alkoxy, acyl, acylamino, acyloxy, amino, substituted amino, quaternary amino, aminocarbonyl, imino, amidino, aminocarbonylamino, amidinocarbonylamino, aminothiocarbonyl, aminocarbonylamino, aminothiocarbonylamino, aminocarbonyloxy, aminosulfonyl, aminosulfonyloxy, aminosulfonylamino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, oxy, thione, spirocycloalkyl, phosphate, phosphonate, phosphinate, phosphonamidate, phosphorodiamidate, phosphoramidate monoester, cyclic phosphoramidate, cyclic phosphorodiamidate, phosphoramidate diester, sulfate, sulfonate, sulfonyl, substituted sulfonyl, sulfonyloxy, thioacyl, thiocyanate, thiol, alkylthio, and substituted alkylthio, wherein said substituents are as defined herein.

“Alkylidene” or “alkylene” refers to divalent saturated aliphatic hydrocarbyl groups having from 1 to 10 carbon atoms and, in some embodiments, from 1 to 6 carbon atoms. “(Cᵤ₋ᵥ)alkylene” refers to alkylene groups having from u to v carbon atoms. The alkylidene and alkylene groups include branched and straight chain hydrocarbyl groups. For example “(C₁₋₆)alkylene” is meant to include methylene, ethylene, propylene, 2-methypropylene, pentylene, and so forth.

“Substituted alkylidene” or “substituted alkylene” refers to an alkylidene group having from 1 to 5 and, in some embodiments, 1 to 3 or 1 to 2 substituents selected from alkoxy, substituted alkoxy, acyl, acylamino, acyloxy, amino, substituted amino, quaternary amino, aminocarbonyl, imino, amidino, aminocarbonylamino, amidinocarbonylamino, aminothiocarbonyl, aminocarbonylamino, aminothiocarbonylamino, aminocarbonyloxy, aminosulfonyl, aminosulfonyloxy, aminosulfonylamino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, phosphate, phosphonate, phosphinate, phosphonamidate, phosphorodiamidate, phosphoramidate monoester, cyclic phosphoramidate, cyclic phosphorodiamidate, phosphoramidate diester, sulfate, sulfonate, sulfonyl, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, 1,3-butadienyl, and so forth.

“Substituted alkenyl” refers to alkenyl groups having from 1 to 3 substituents and, in some embodiments, 1 to 2 substituents selected from alkoxy, substituted alkoxy, acyl, acylamino, acyloxy, amino, substituted amino, quaternary amino, aminocarbonyl, imino, amidino, aminocarbonylamino, amidinocarbonylamino, aminothiocarbonyl, aminocarbonylamino, aminothiocarbonylamino, aminocarbonyloxy, aminosulfonyl, aminosulfonyloxy, aminosulfonylamino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, phosphate, phosphonate, phosphinate, phosphonamidate, phosphorodiamidate, phosphoramidate monoester, cyclic phosphoramidate, cyclic phosphorodiamidate, phosphoramidate diester, sulfate, sulfonate, sulfonyl, substituted sulfonyl, sulfonyloxy, thioacyl, thiocyanate, thiol, alkylthio, and substituted alkylthio, wherein said substituents are defined herein and with the proviso that any hydroxy or thiol substitution is not attached to a vinyl (unsaturated) carbon atom.

“Stabilized alkenyloxyaryl” refers to groups (stabilized alkenyl)-O-(aryl), where stabilized alkenyl is alkenyl having 1 to 3 electron withdrawing substituents, independently selected from the group —F, —Cl, —CF₃, —CH₂F, —CHF₂, and —NO₂, directly attached to the vinyl carbons (>C═C<). Examples of stabilized alkenyloxyaryl are:

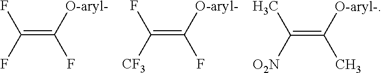

“Stabilized alkenyloxyheteroaryl” refers to groups (stabilized alkenyl)-O-(heteroaryl), where stabilized alkenyl is alkenyl having 1 to 3 electron withdrawing substituents, independently selected from the group —F, —Cl, —CF₃, —CH₂F, —CHF₂, and —NO₂, directly attached to the vinyl carbons (>C═C<). Examples of stabilized alkenyloxyheteroaryl are:

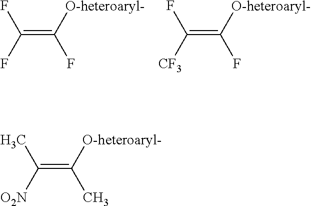

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and so forth.

“Substituted alkynyl” refers to alkynyl groups having from 1 to 3 substituents and, in some embodiments, from 1 to 2 substituents selected from alkoxy, substituted alkoxy, acyl, acylamino, acyloxy, amino, substituted amino, quaternary amino, aminocarbonyl, imino, amidino, aminocarbonylamino, amidinocarbonylamino, aminothiocarbonyl, aminocarbonylamino, aminothiocarbonylamino, aminocarbonyloxy, aminosulfonyl, aminosulfonyloxy, aminosulfonylamino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, phosphate, phosphonate, phosphinate, phosphonamidate, phosphorodiamidate, phosphoramidate monoester, cyclic phosphoramidate, cyclic phosphorodiamidate, phosphoramidate diester, sulfate, sulfonate, sulfonyl, substituted sulfonyl, sulfonyloxy, thioacyl, thiocyanate, thiol, alkylthio, and substituted alkylthio and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, substituted hydrazino, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Oxyacyl” refers to the groups alkyl-OC(O)—, substituted alkyl-OC(O)—, alkenyl-OC(O)—, substituted alkenyl-OC(O)—, alkynyl-OC(O)—, substituted alkynyl-OC(O)—, aryl-OC(O)—, substituted aryl-OC(O), cycloalkyl-OC(O)—, substituted cycloalkyl-OC(O)—, heteroaryl-OC(O)—, substituted heteroaryl-OC(O)—, heterocyclic-OC(O)—, and substituted heterocyclic-OC(O)—.

“Amino” refers to the group —NH₂.

“Substituted amino” refers to the group —NR²¹R²² where R²¹ and R²² are independently selected from the group consisting of hydrogen, amino, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Quaternary amino” refers to the group —NR²³R²⁴R²⁵ where R²³, R²⁴, and R²⁵ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²³, R²⁴, and/or R²⁵ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. In Formula (I), for example, Y may be O and R² may be an alkyl substituted with quaternary amino. The quaternary amino may be neutralized with an acid to form the corresponding salt where —YR² is —O-alkyl-N⁺R²³R²⁴R²⁵M⁻ where M is a pharmaceutically acceptable counterion, such as chlorine, fluorine, bromine, etc.

“Hydroxyamino” refers to the group —NHOH.

“Alkoxyamino” refers to the group —NHO-alkyl wherein alkyl is defined herein.

“Aminocarbonyl” refers to the group —C(O)NR²⁶R²⁷ where R²⁶ and R²⁷ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R²⁶ and R²⁷ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonyl” refers to the group —C(S)NR²⁸R²⁹ where R²⁸ and R²⁹ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²⁸ and R²⁹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonylamino” refers to the group —NR³⁰C(O)NR³¹R³² where R³⁰ is hydrogen or alkyl and R³¹ and R³² are independently selected from hydrogen, amino, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R³¹ and R³² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. One example of an aminocarbonylamino group is a semicarbazide group (where R³⁰ and R³¹ are hydrogen, and R³² is amino).

“Amidinocarbonylamino” refers to the group —CR⁶⁷(═N)NR⁶⁸C(O)NR⁶⁹R⁷⁰ where R⁶⁷, R⁶⁸, R⁶⁹, and R⁷⁰ are independently selected from hydrogen, amino, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R³¹ and R³² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. One example of an amidinocarbonylamino group is a semicarbazone group.

“Aminothiocarbonylamino” refers to the group —NR³³C(S)NR³⁴R³⁵ where R³³ is hydrogen or alkyl and R³⁴ and R³⁵ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R³⁴ and R³⁵ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonyloxy” refers to the group —O—C(O)NR³⁶R³⁷ where R³⁶ and R³⁷ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R³⁶ and R³⁷ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyl” refers to the group —SO₂NR³⁸R³⁹ where R³⁸ and R³⁹ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R³⁸ and R³⁹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyloxy” refers to the group —O—SO₂NR⁴⁰R⁴¹ where R⁴⁰ and R⁴¹ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R⁴⁰ and R⁴¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonylamino” refers to the group —NR⁴²—SO₂NR⁴³R⁴⁴ where R⁴² is hydrogen or alkyl and R⁴³ and R⁴⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R⁴³ and R⁴⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amidino” refers to the group —CR⁴⁵(═N)NR⁴⁶R⁴⁷ where R⁴⁵, R⁴⁶, and R⁴⁷ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R⁴⁶ and R⁴⁷ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. One example of an amidino group is a hydrazone group (where R⁴⁶ and R⁴⁷ are hydrogen).

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“Substituted aryl” refers to aryl groups which are substituted with 1 to 8 and, in some embodiments, 1 to 5, 1 to 3, or 1 to 2 substituents selected from alkyl, substituted alkyl, alkenyl, substituted alkenyl, alkynyl, substituted alkynyl, alkoxy, substituted alkoxy, acyl, acylamino, acyloxy, amino, substituted amino, quaternary amino, aminocarbonyl, imino, amidino, aminocarbonylamino, amidinocarbonylamino, aminothiocarbonyl, aminocarbonylamino, aminothiocarbonylamino, aminocarbonyloxy, aminosulfonyl, aminosulfonyloxy, aminosulfonylamino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, phosphate, phosphonate, phosphinate, phosphonamidate, phosphorodiamidate, phosphoramidate monoester, cyclic phosphoramidate, cyclic phosphorodiamidate, phosphoramidate diester, sulfate, sulfonate, sulfonyl, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthyloxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Arylalkyl” refers to the group -alkyl-aryl, where aryl is as defined herein, that includes, by way of example, phenylmethyl.

“Azido” refers to the group —N₃.

“Hydrazino” refers to the group —NHNH₂.

“Substituted hydrazino” refers to the group —NR⁴⁸NR⁴⁹R⁵⁰ where R⁴⁸, R⁴⁹, and R⁵⁰ are independently selected from hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R⁴⁹ and R⁵⁰ are optionally joined, together with the nitrogen bound thereto to form a heterocyclic or substituted heterocyclic group, provided that R⁴⁹ and R⁵⁰ are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COON or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR⁵¹—C(O)O-alkyl, —NR⁵¹—O(O)O-substituted alkyl, —NR⁵¹—C(O)O-alkenyl, —NR⁵¹—O(O)O-substituted alkenyl, —NR⁵¹—C(O)O-alkynyl, —NR⁵¹—O(O)O-substituted alkynyl, —NR⁵¹—C(O)O-aryl, —NR⁵¹—O(O)O-substituted aryl, —NR⁵¹—O(O)O-cycloalkyl, —NR⁵¹—O(O)O-substituted cycloalkyl, —NR⁵¹—C(O)O-heteroaryl, —NR⁵¹—C(O)O-substituted heteroaryl, —NR⁵¹—C(O)O-heterocyclic, and —NR⁵¹—C(O)O-substituted heterocyclic wherein R⁵¹ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms.

“Cycloalkenyl” refers to a partially saturated cycloalkyl ring having at least one site of >C═C< ring unsaturation.

“Cycloalkylene” refer to divalent cycloalkyl groups as defined herein. Examples of cycloalkyl groups include those having three to six carbon ring atoms such as cyclopropylene, cyclobutylene, cyclopentylene, and cyclohexylene.

“Substituted cycloalkyl” refers to a cycloalkyl group, as defined herein, having from 1 to 8, or 1 to 5, or in some embodiments 1 to 3 substituents selected from alkyl, substituted alkyl, alkenyl, substituted alkenyl, alkynyl, substituted alkynyl, alkoxy, substituted alkoxy, acyl, acylamino, acyloxy, amino, substituted amino, quaternary amino, aminocarbonyl, imino, amidino, aminocarbonylamino, amidinocarbonylamino, aminothiocarbonyl, aminocarbonylamino, aminothiocarbonylamino, aminocarbonyloxy, aminosulfonyl, aminosulfonyloxy, aminosulfonylamino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, phosphate, phosphonate, phosphinate, phosphonamidate, phosphorodiamidate, phosphoramidate monoester, cyclic phosphoramidate, cyclic phosphorodiamidate, phosphoramidate diester, sulfate, sulfonate, sulfonyl, substituted sulfonyl, sulfonyloxy, thioacyl, thiocyanate, thiol, alkylthio, and substituted alkylthio. The term “substituted cycloalkyl” includes substituted cycloalkenyl groups.

“Cycloalkyloxy” refers to —O-cycloalkyl wherein cycloalkyl is as defined herein.

“Substituted cycloalkyloxy refers to —O-(substituted cycloalkyl) wherein substituted cycloalkyl is as defined herein.

“Cycloalkylthio” refers to —S-cycloalkyl wherein cycloalkyl is as defined herein.

“Substituted cycloalkylthio” refers to —S-(substituted cycloalkyl).

“Guanidino” refers to the group —NHC(═NH)NH₂.

“Substituted guanidino” refers to —NR⁵²C(═NR⁵²)N(R⁵²)₂ where each R⁵² is independently selected from hydrogen, alkyl, substituted alkyl, aryl, substituted aryl, heteroaryl, substituted heteroaryl, heterocyclyl, and substituted heterocyclyl and two R⁵² groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo, and iodo.

“Haloalkyl” refers to substitution of alkyl groups with 1 to 5 or in some embodiments 1 to 3 halo groups.

“Haloalkoxy” refers to substitution of alkoxy groups with 1 to 5 or in some embodiments 1 to 3 halo groups.

“Hydroxy” or “hydroxyl”, used interchangeably herein, refers to the group —OH.

“Heteroaryl” refers to an aromatic group of from 1 to 14 carbon atoms and 1 to 6 heteroatoms selected from oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In some embodiments, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, imidazolinyl, isoxazolyl, pyrrolyl, pyrazolyl, pyridazinyl, pyrimidinyl, purinyl, phthalazyl, naphthylpryidyl, benzofuranyl, tetrahydrobenzofuranyl, isobenzofuranyl, benzothiazolyl, benzoisothiazolyl, benzotriazolyl, indolyl, isoindolyl, indolizinyl, dihydroindolyl, indazolyl, indolinyl, benzoxazolyl, quinolyl, isoquinolyl, quinolizyl, quianazolyl, quinoxalyl, tetrahydroquinolinyl, isoquinolyl, quinazolinonyl, benzimidazolyl, benzisoxazolyl, benzothienyl, benzopyridazinyl, pteridinyl, carbazolyl, carbolinyl, phenanthridinyl, acridinyl, phenanthrolinyl, phenazinyl, phenoxazinyl, phenothiazinyl, and phthalimidyl.

“Substituted heteroaryl” refers to heteroaryl groups that are substituted with from 1 to 8 or in some embodiments 1 to 5, or 1 to 3, or 1 to 2 substituents selected from the substituents defined for substituted aryl.

“Heteroaryloxy” refers to —O-heteroaryl wherein heteroaryl is as defined herein.

“Substituted heteroaryloxy” refers to the group —O-(substituted heteroaryl) wherein substituted heteroaryl is as defined herein.

“Heteroarylthio” refers to the group —S-heteroaryl wherein heteroaryl is as defined herein.

“Substituted heteroarylthio” refers to the group —S-(substituted heteroaryl) wherein substituted heteroaryl is as defined herein.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from nitrogen, sulfur, or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. decahydroquinolin-6-yl). In some embodiments, the nitrogen and/or sulfur atom(s) of the heterocyclic group are optionally oxidized to provide for the N-oxide, sulfinyl, sulfonyl moieties. More specifically the heterocyclyl includes, but is not limited to, azetidinyl, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, thiomorpholinyl, imidazolidinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocyclic” or “substituted heterocycle” or “substituted heterocycloalkyl” or “substituted heterocyclyl” refers to heterocyclic groups, as defined herein, that are substituted with from 1 to 5 or in some embodiments 1 to 3 of the substituents as defined for substituted cycloalkyl.

“Heterocyclyloxy” refers to the group —O-heterocycyl wherein heterocyclyl is as defined herein.

“Substituted heterocyclyloxy” refers to the group —O-(substituted heterocycyl) wherein substituted heterocyclyl is as defined herein.

“Heterocyclylthio” refers to the group —S-heterocycyl wherein heterocyclyl is as defined herein.

“Substituted heterocyclylthio” refers to the group —S-(substituted heterocycyl) wherein substituted heterocyclyl is as defined herein.

“Imino” refers to the group —CR⁷¹═NR⁷², where R⁷¹ is hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic, and wherein R⁷² is hydrogen, amino, substituted amino, alkyl, substituted alkyl, aryl, substituted aryl, or hydroxyl.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Phosphate” refers to the groups —OP(O)(OR⁶⁰)₂ (monophosphate or phospho), —OP(O)(OR⁶⁰)OP(O)(OR⁶⁰)₂ (diphosphate or diphospho), —OP(O)(OR⁶⁰)OP(O)(OR⁶⁰)OP(O)(OR⁶⁰)₂ (triphosphate or triphospho), and salts thereof (including partial salts), wherein R⁶⁰ is independently selected from hydrogen, alkyl, substituted alkyl, carboxylic acid, and carboxyl ester. In some embodiments, for example, Y may be O and R² may be an alkyl substituted with phosphate. The phosphate may be neutralized with a base to form the corresponding salt where —YR² is —O-alkyl-OP(O)OR⁶⁰O⁻M⁺, where M is a pharmaceutically acceptable counterion, such as sodium, potassium, etc. It should also be understood, of course, that the initial oxygen of the phosphate may be “Y” in Formula (I) herein. For instance, —YR² may be —OP(O)(OR⁶⁰)₂ or a salt thereof.

“Phosphonate” refers to the group —OP(O)(R⁵³)(OR⁵⁴) (monophosphonate), —OP(O)(OR⁵⁴)R⁵³P(O)(OR⁵⁴)₂ (diphosphonate), —OP(O)(OR⁵⁴)R⁵³P(O)(OR⁵⁴)R⁵³P(O)(OR⁵⁴)₂ (triphosphonate), and salts thereof (including partial salts), wherein R⁵³ is independently selected from hydrogen, alkyl, and substituted alkyl, and R⁵⁴ is independently selected from hydrogen, alkyl, substituted alkyl, carboxylic acid, and carboxyl ester. It is understood, of course, that the initial oxygen of the phosphonate may be “Y” in Formula (I) herein.

“Phosphinate” refers to the group —OP(O)(R⁶³)₂ (monophosphinate), —OP(O)(R⁶³)R⁶⁴P(O)(R⁶³)(OR⁶³) (diphosphinate), —OP(O)(R⁶³)R⁶⁴P(O)(R⁶³)R⁶⁴P(O)(R⁶³)(OR⁶³) (triphosphinate), and salts thereof (including partial salts), wherein R⁶⁴ is independently selected from hydrogen, alkyl, and substituted alkyl, and R⁶³ is independently selected from hydrogen, alkyl, substituted alkyl, carboxylic acid, and carboxyl ester. It is understood, of course, that the initial oxygen of the phosphonate may be “Y” in Formula (I) herein.

“Phosphorodiamidate” refers to the group:

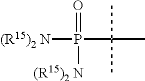

where each R¹⁵ may be the same or different and each is hydrogen, alkyl, substituted alkyl, cycloalkyl, or substituted cycloalkyl. A particularly preferred phosphorodiamidate is the following group:

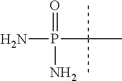

“Phosphoramidate monoester” refers to the group below, where R⁵⁵ is selected from hydrogen, alkyl, substituted alkyl, aryl, substituted aryl, cycloalkyl, substituted cycloalkyl, heteroaryl, substituted heteroaryl, heterocyclic and substituted heterocyclic and a side-chain of an amino acid; and R⁵⁶ is hydrogen or alkyl. In a preferred embodiment R⁵⁵ is derived from an L-amino acid.

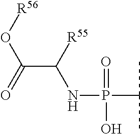

“Phosphoramidate diester” refers to the group below, where R⁵⁷ is selected from alkyl, substituted alkyl, aryl, substituted aryl, cycloalkyl, substituted cycloalkyl, heteroaryl, substituted heteroaryl, heterocyclic and substituted heterocyclic, and R⁵⁵ and R⁵⁶ are as defined herein. In a preferred embodiment, R⁵⁵ is derived from an L-amino acid.

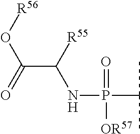

“Cyclic phosphoramidate” refers to the group below, where q is 1 to 3, more preferably q is 1 to 2.

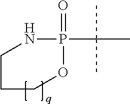

“Cyclic phosphorodiamidate” refers to the group below, where q is 1 to 3, more preferably q is 1 to 2.

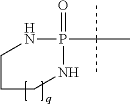

“Phosphonamidate” refers to the group below, where R₁₄ is hydrogen, alkyl, substituted alkyl, cycloalkyl, or substituted cycloalkyl.

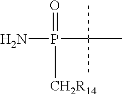

“Spirocycloalkyl” refers to a 3 to 10 member cyclic substituent formed by replacement of two hydrogen atoms at a common carbon atom with an alkylene group having 2 to 9 carbon atoms, as exemplified by the following structure wherein the methylene group shown here attached to bonds marked with wavy lines is substituted with a spirocycloalkyl group:

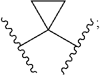

“Sulfate” refers to the groups —OS(O)₂(OR⁶⁵) and salts thereof (including partial salts), wherein R⁶⁵ is independently selected from hydrogen, alkyl, substituted alkyl, carboxylic acid, and carboxyl ester. In some embodiments, Y may be O and R² may an alkyl substituted with sulfate. The sulfate may be neutralized with a base to form the corresponding salt where —YR² is —O-alkyl-OS(O)₂O⁻M⁺, where M is a pharmaceutically acceptable counterion, such as sodium, potassium, etc. It should also be understood, of course, that the initial oxygen of the sulfate may be “Y” in Formula (I) herein. For instance, —YR² may be —OS(O)₂(OR⁶⁵) or a salt thereof.

“Sulfonate” refers to the group —S(O)₂(OR⁶⁶) and salts thereof (including partial salts), wherein R⁶⁶ is independently selected from hydrogen, alkyl, substituted alkyl, carboxylic acid, and carboxyl ester.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Racemates” refers to a mixture of enantiomers.

“Solvate” or “solvates” of a compound refer to those compounds, where compounds is as defined herein, that are bound to a stoichiometric or non-stoichiometric amount of a solvent. Solvates of a compound includes solvates of all forms of the compound. In some embodiments, solvents are volatile, non-toxic, and/or acceptable for administration to humans in trace amounts. Suitable solvents include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable counterion” refers to pharmaceutically acceptable organic or inorganic, monatomic or polyatomic ions well known in the art. Such pharmaceutically acceptable counterions typically have a valency of 1 or 2. Examples of positively charged, pharmaceutically acceptable counterions may include, for instance, calcium, magnesium, potassium, sodium, ammonium, tetralkylammonium, etc. Examples of negatively charged, pharmaceutically acceptable counterions may include, for instance, chloride, fluoride, bromide, phosphate, sulfate, acetate, formate, oxalate, tartarate, mesylate, maleate, etc.

“Pharmaceutically acceptable salt” refers to pharmaceutically acceptable salts derived from pharmaceutically acceptable counterions. Suitable salts include those described in P. Heinrich Stahl, Camille G. Wermuth (Eds.), Handbook of Pharmaceutical Salts Properties, Selection, and Use; 2002.

“Patient” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of “substituents”, “groups”, “functionality”, or “moieties”, used interchangeably herein, are arrived at by naming the terminal portion of the functionality followed by the adjacent functionality toward the point of attachment. For example, the substituent “arylalkyloxycarbonyl” refers to the group (aryl)-(alkyl)-O—C(O)—, with (aryl) being furthest away from the point of attachment and carbonyl “(CO)—” being directly adjacent to the point of attachment to the parent molecule. Furthermore, in some instances, the substitutents may contain an indication showing the point of their attachment (i.e., their “bond”) to the parent compound. Such indications showing the substituent point of attachment to the parent compound may include, for example: (1) a hyphen mark at the point of attachment such as “—” shown in this substituent: —OH; (2) a wavy line at the point of attachment such as shown in this substituent:

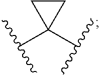

(3) a wavy line crossing the point of attachment such as shown in this substituent:

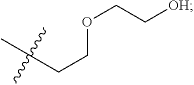

or a straight dashed line crossing the point of attachment such as shown in this substituent:

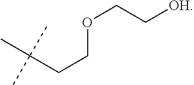

It is understood that in all substituted groups defined herein, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the herein definitions are not intended to include impermissible substitution patterns (e.g., methyl substituted with 5 fluoro groups). Such impermissible substitution patterns are well known to the skilled artisan.

Accordingly, in some embodiments, provided is a compound that is Formula (I):

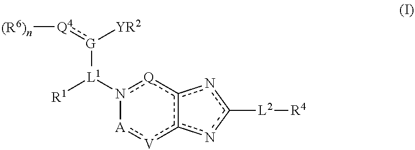

or a pharmaceutically acceptable salt or solvate thereof, wherein:

the dotted lines represent an optional double bond, provided that no two double bonds are adjacent to one another, and that the dotted lines represent at least 3 double bonds, optionally 4 double bonds, and optionally 5 double bonds;

G is C when the bond between G and Q⁴ is a double bond and G is C-Q¹ when the bond between G and Q⁴ is a single bond;

A, Q, and V are independently selected from N and CR³;

L¹ is independently O₃₋₆ cycloalkylene or C₁₋₅ alkylene, where one or two CH₂ groups of said C₁₋₅ alkylene are optionally replaced with NR^{a}, S, (C═O), or O and optionally two adjacent carbon atoms form a double bond;

L² is a bond or independently C₃₋₆ cycloalkylene or is C₁₋₅ alkylene where one or two CH₂ groups of said C₁₋₅ alkylene are optionally replaced with NR^{b}, S, (C═O), or O and optionally two adjacent carbon atoms form a double bond or triple bond, and wherein said C₁₋₅ alkylene is optionally substituted with one to three groups independently selected from halo, alkyl, and spirocycloalkyl;

Y is a bond, O, S, or NR^{c};

Q¹ is selected from hydrogen, halo, amino, acylamino, alkyl, substituted alkyl, alkenyl, substituted alkenyl, alkynyl, substituted alkynyl, carboxy, carboxy ester, azido, hydroxy, alkoxy, substituted alkoxy, acyl, acyloxy, cyano, thiol, alkylthio, substituted alkylthio, and substituted sulfonyl;

Q⁴ is O, S, or NR⁷;

R¹ is selected from aryl, substituted aryl, heteroaryl, substituted heteroaryl, cycloalkyl, substituted cycloalkyl, heterocyclyl, and substituted heterocyclyl;

R² is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl, and a pharmaceutically acceptable counterion;

R³ is selected from hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl;

R⁴ is independently selected from aryl, substituted aryl, heteroaryl, substituted heteroaryl, heterocyclyl, substituted heterocyclyl, cycloalkyl, and substituted cycloalkyl;

R⁶ is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl, and a pharmaceutically acceptable counterion;

R⁷ is selected from hydrogen, halo, aminocarbonyl, imino, amidino, aminocarbonylamino, amidinocarbonylamino, carboxy, carboxy ester, hydroxy, alkoxy, substituted alkoxy, acyl, acyloxy, cyano, thiol, alkylthio, substituted alkylthio, sulfonyl, and substituted sulfonyl;

R^{a}, R^{b}, and R^{c} are independently selected from hydrogen, alkyl, and substituted alkyl; and

n is from 0 to 1, provided that n is 0 when the bond between G and Q⁴ is a double bond.

In some embodiments, a compound is provided that is a pharmaceutically acceptable salt of Formula (I).

In some embodiments, a compound is provided that is a solvate of Formula (I). In some embodiments, the solvate is a solvate of a pharmaceutically acceptable salt of Formula (I).

In some embodiments, A is N, and Q and V are CR³, where R³ is independently selected from hydrogen and a lower alkyl. In some embodiments, R³ is hydrogen.

In some embodiments, V is N, and A and Q are CR³, where R³ is independently selected from hydrogen and a lower alkyl. In some embodiments, R³ is hydrogen.

In some embodiments, A, Q, and V are CR³, where R³ is independently selected from hydrogen and a lower alkyl. In some embodiments, R³ is hydrogen.

In some embodiments, Y is a bond, NH, or O. In some embodiments, Y is O. In some embodiments, Y is NH. In some embodiments, Y is a bond.

In some embodiments, [symbol] represents a single or double bond. In some embodiments, [symbol] represents a double bond. In some embodiments, [symbol] represents a single bond.

In some embodiments, R² is C₁-C₆ alkyl, such as methyl, ethyl, n-propyl, i-propyl, t-butyl, i-butyl, n-butyl, or n-hexyl. In some embodiments, R² is methyl or ethyl. In some embodiments, R² is C₁-C₆ alkyl optionally substituted with 1 to 5 substituents independently selected from hydroxyl, oxy, alkoxy, substituted alkoxy, carboxyl, phosphate, sulfate, amino, substituted amino, quaternary amino, acylamino, aminocarbonyl, aminocarbonylamino, or a combination thereof. In some embodiments, R² is C₁-C₆ alkyl substituted with hydroxyl (e.g., 3-hydroxypropyl and 2,3-dihydroxylpropyl). In some embodiments, R² is C₁-C₆ alkyl substituted with alkoxy (—O-alkyl) or substituted alkoxy in which the alkyl portion is substituted with alkoxy and/or hydroxyl (e.g., 2-(hydroxylethyl)oxyethyl, 2-(methyloxy)ethyloxyethyl, 2-[(2-hydroxylethyl)oxy]ethyloxyethyl, or 2-[2-(2-hydroxylethoxy)ethoxy]ethoxyethyl). In some embodiments, R² is C₁-C₆ alkyl substituted with carboxyl (e.g., butanoic acid). In some embodiments, R² is C₁-C₆ alkyl substituted with phosphate and/or sulfate (e.g., 3-(phosphonoxy)propyl or 3-(sulfonyloxy)propyl). In some embodiments, R² is C₁-C₆ alkyl substituted with amino (e.g., butan-1-amino), substituted amino, and/or quaternary amino (e.g., propyl(trimethyl ammonium chloride)). In some embodiments, R² is C₁-C₆ alkyl substituted with acylamino, aminocarbonyl, and/or aminocarbonylamino (e.g., 2-[[2-formamidopropanoyl]amino]-3-phenyl-propanoic acid and 2-formamidopropanoic acid).

In some embodiments, R² is selected from the group consisting of alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, and substituted sulfonyl.

In some embodiments, R² is C₁₋₆ alkyl, wherein said C₁₋₆ alkyl is substituted with 1 to 5 substituents independently selected from the group consisting of hydroxyl, alkoxy, substituted alkoxy, oxy, carboxyl, phosphate, sulfate, amino, substituted amino, quaternary amino, acylamino, aminocarbonyl, and aminocarbonylamino, or a combination thereof.

In some embodiments, R² is C₁₋₆ alkyl, wherein said C₁₋₆ alkyl is substituted with 1 to 5 substituents independently selected from the group consisting of hydroxyl, alkoxy, substituted alkoxy, oxy, carboxyl, phosphate, sulfate, amino, substituted amino, quaternary amino, acylamino, aminocarbonyl, and aminocarbonylamino.

In some embodiments, R² is aryl and/or substituted aryl (e.g., benzyl or methylbenzyl).

In some embodiments, R² is selected from the group consisting of hydrogen, C₁₋₆alkyl, phenyl, -A¹, -A¹-(X¹)_{w}R⁸R⁹, -A¹R¹⁰, -A¹R¹¹, A¹-N(R⁹)_{z}, -A¹-NHA²-R¹¹, -A¹NHA²-NHA³R¹¹, and -A¹-R⁸R⁹; wherein:

A¹, A², A³, and A⁴ are each independently selected from C₁₋₆ alkylene, wherein one to four independent CH₂ groups of each of said A¹, A², A³, and A⁴ are optionally substituted with one to two R¹² groups;

each X¹ is independently selected from the group consisting of —(R⁸-A¹), —(R⁸-A²), —(R⁸-A³), and —(R⁸-A⁴);

R⁸ is O;

each R⁹ is independently selected from the group consisting of hydrogen and C₁₋₆ alkyl;

R¹⁹ is selected from the group consisting of phosphate, phosphonate, and sulfate;

R¹¹ is carboxyl;

each R¹² is independently selected from the group consisting of C₁₋₆ alkyl, oxo, aryl, arylalkyl, and hydroxyl;

w is an integer from 1 to 3; and

z is an integer from 2 to 3.

In some embodiments, R² is hydroxylalkoxyalkyl.

In some embodiments, R² is hydroxylethoxyethyl.

In some embodiments, R² is a group having the structure:

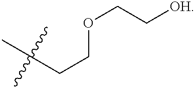

In some embodiments, R² is a pharmaceutically acceptable counterion, such as sodium. In some embodiments, R² is hydrogen.

In some embodiments, the bond between G and Q⁴ is a double bond. In some embodiments, the bond between G and Q⁴ is a single bond.

In some embodiments, G is C. In some embodiments, G is CH.

In some embodiments, Q⁴ is —O—. In some embodiments, Q⁴ and R⁶ is hydrogen, alkyl, or substituted alkyl.

In some embodiments, Q⁴ is NR⁷ and n is 0. In some embodiments, Q⁴ is NR⁷ and R⁶ is hydrogen, alkyl, or substituted alkyl.

In some embodiments, L¹ is C₁₋₃ alkylene. In some embodiments, L¹ is CH₂.

In some embodiments, L² is a bond.

In some embodiments, R⁴ is substituted phenyl or substituted heteroaryl. In some embodiments, R⁴ is substituted with at least one halo group, such as with at least one fluoro group. In some embodiments, R⁴ is phenyl substituted with at least one fluoro group. In some embodiments, R⁴ is 2,3-difluorophenyl.

In some embodiments R¹ is:

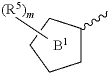

wherein,

the wavy line represents the point of attachment to the remainder of the molecule;

ring B¹ is a 5-membered aromatic ring wherein 1 to 3 ring carbon atoms are optionally replaced by nitrogen or oxygen, wherein each nitrogen is optionally oxidized, and wherein ring B¹ may be optionally fused to a 5- or 6-membered aryl, substituted aryl, heteroaryl, substituted heteroaryl, heterocycle or substituted heterocycle to form a 8- or 9-membered bicyclic ring;

R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, substituted sulfonyl, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl; and

m is from 0 to 4, in some embodiments m is 1, 2, 3, or 4, in some embodiments, m is 1, 2, or 3, in some embodiments m is 1 or 2, and in some embodiments, m is 1.

In some embodiments the ring B¹ is selected from:

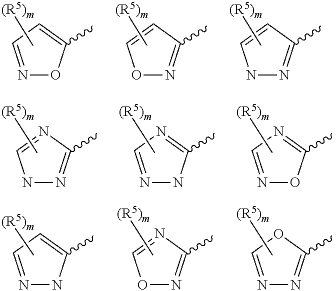

wherein,

the wavy line represents the point of attachment to the remainder of the molecule;

m is 1, 2, 3 or 4, in some embodiments m is 1, 2, or 3, in some embodiments m is 1 or 2, and in some embodiments, m is 1; and

R⁵ is independently selected from halo, haloalkyl, haloalkoxy, aryl, substituted aryl, heteroaryl, substituted heteroaryl, heterocyclyl, substituted heterocyclyl, cycloalkyl, and substituted cycloalkyl.

In some embodiments ring B¹ is selected from:

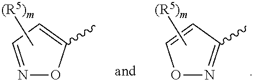

In some embodiments, ring B¹ is:

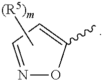

In some embodiments, R¹ is

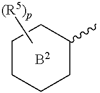

wherein,

the wavy line represents the point of attachment to the remainder of the molecule;

ring B² is a 6-membered aromatic ring wherein 1 to 3 ring carbon atoms are optionally replaced by nitrogen or oxygen, wherein each nitrogen is optionally oxidized, and wherein ring B² may be optionally fused to a 5- or 6-membered aryl, substituted aryl, heteroaryl, substituted heteroaryl, heterocycle or substituted heterocycle to form a 9- or 10-membered bicyclic ring;

R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, substituted sulfonyl, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl; and

p is from 0 to 5, in some embodiments p is 1, 2, 3, or 4, in some embodiments, p is 1, 2, or 3, in some embodiments p is 1 or 2, and in some embodiments, p is 1.

In some embodiments ring B² is selected from:

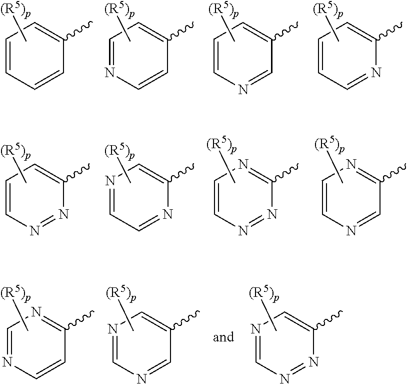

wherein,

the wavy line represents the point of attachment to the remainder of the molecule;

p is 1, 2, 3, or 4, in some embodiments p is 1, 2, or 3, in some embodiments p is 1 or 2, and in some embodiments, p is 1; and

R⁵ is independently selected from halo, haloalkyl, haloalkoxy, aryl, substituted aryl, heteroaryl, substituted heteroaryl, heterocyclyl, substituted heterocyclyl, cycloalkyl, and substituted cycloalkyl.

In some embodiments the ring B² is selected from:

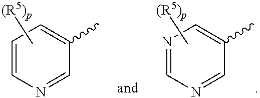

In some embodiments, R⁵ in ring B¹ and/or B² is substituted phenyl or substituted heteroaryl. In some embodiments, R⁵ is phenyl or heteroaryl, each of which is substituted with at least one group selected from alkyl, haloalkyl, and optionally substituted alkoxy. In some embodiments, R⁵ is phenyl or heteroaryl, each of which is substituted with at least one group selected from lower alkyl, CF₃, and optionally substituted methoxy. In some embodiments, R⁵ is phenyl substituted with at least one group selected from lower alkyl, CF₃, and optionally substituted methoxy. In some embodiments, R⁵ is phenyl substituted with at least one group selected from lower alkyl, CF₃, and R¹¹—CH₂O— wherein R¹¹ is optionally substituted heteroaryl. In some embodiments, R⁵ is phenyl substituted with at least one group selected from lower alkyl, CF₃, and R¹¹—CH₂O— wherein R¹¹ is optionally substituted pyridinyl. In some embodiments, R⁵ is phenyl substituted with at least one group selected from lower alkyl, CF₃, and R¹¹—CH₂O— wherein R¹¹ is pyridinyl.

In some embodiments, R⁵ is selected from:

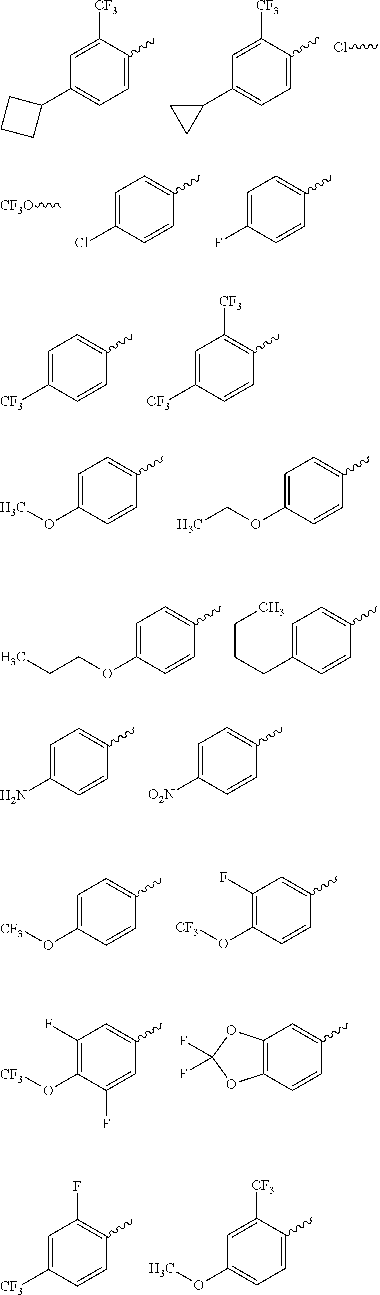

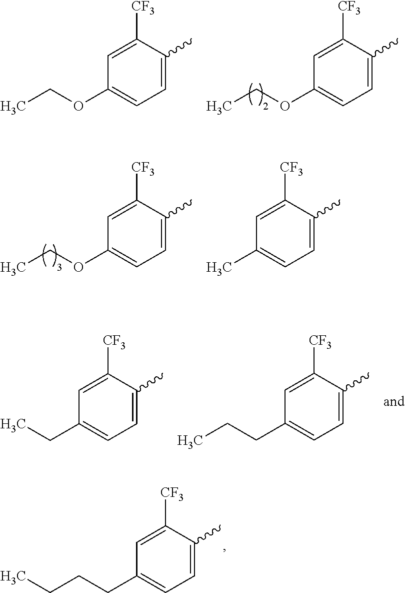

wherein the wavy line represents the point of attachment to the remainder of the molecule.

In some embodiments, R⁵ is selected from:

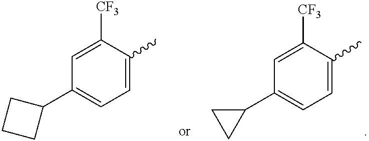

In some embodiments, R⁵ is:

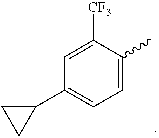

In some embodiments, provided is a compound that is Formula (II)

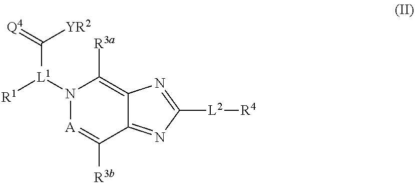

or a pharmaceutically acceptable salt or solvate thereof, wherein,

A is N or CR³;

R^{3a} and R^{3b} are independently R³; and

R¹, R², R³, R⁴, Y, L¹, L², and Q⁴ are as defined herein.

In some embodiments, provided is a compound that is Formula (III)

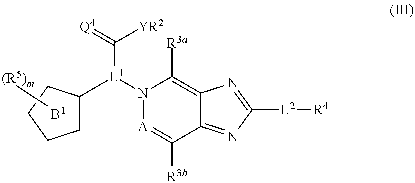

or a pharmaceutically acceptable salt or solvate thereof, wherein,

A is N or CR³;

R^{3a} and R^{3b} are independently R³; and

B¹, R², R³, R⁴, R⁵, Y, L¹, L², Q⁴, and m are as defined herein.

In some embodiments, provided is a compound that is Formula (IV)

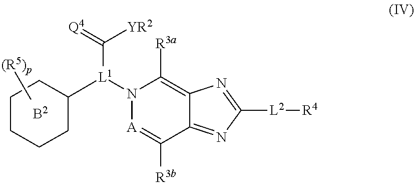

or a pharmaceutically acceptable salt or solvate thereof, wherein,

A is N or CR³;

R^{3a} and R^{3b} are independently R³; and

B², R², R³, R⁴, R⁵, Y, L¹, L², Q⁴, and p are as defined herein.

In some embodiments, provided is a compound that is Formula (V)

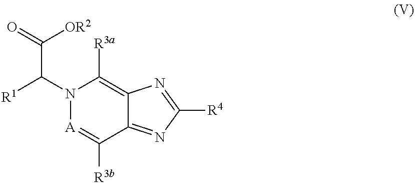

or a pharmaceutically acceptable salt or solvate thereof, wherein,

A is N or CR³;

R^{3a} and R^{3b} are independently R³; and

R¹, R², R³, and R⁴ are as defined herein.

In some embodiments, provided is a compound that is Formula (V)

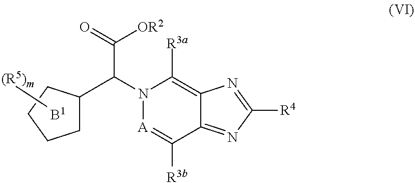

or a pharmaceutically acceptable salt or solvate thereof, wherein,

A is N or CR³;

R^{3a} and R^{3b} are independently R³; and

B¹, R², R³, R⁴, and m are as defined herein.

In some embodiments, provided is a compound that is Formula (VII)

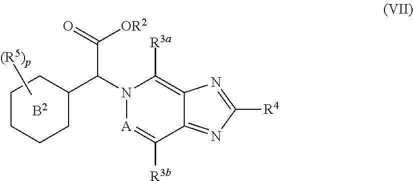

or a pharmaceutically acceptable salt or solvate thereof, wherein,

A is N or CR³;

R^{3a} and R^{3b} are independently R³; and

B², R², R³, R⁴, and p are as defined herein.

In some embodiments, provided is a compound that is Formula (I):

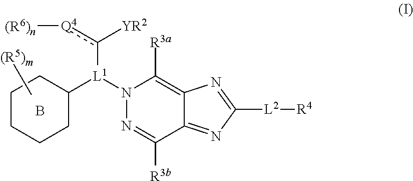

or a pharmaceutically acceptable salt or solvate thereof, wherein:

[symbol] represents a single or double bond;

ring B is a 6-membered aromatic ring wherein 1 to 3 ring carbon atoms are optionally replaced by nitrogen or oxygen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;

L¹ is independently C₃₋₆ cycloalkylene or C₁₋₅ alkylene, where one or two CH₂ groups of said C₁₋₅ alkylene are optionally replaced with NR^{a}, S, (C═O), or O and optionally two adjacent carbon atoms form a double bond;

L² is a bond or independently C₃₋₆ cycloalkylene or is C₁₋₅ alkylene where one or two CH₂ groups of said C₁₋₅ alkylene are optionally replaced with NR^{b}, S, (C═O), or O and optionally two adjacent carbon atoms form a double bond or triple bond, and wherein said C₁₋₅ alkylene is optionally substituted with one to three groups independently selected from halo, alkyl, and spirocycloalkyl;

Y is a bond, O, S, or NR^{c};

Q⁴ is O, S, or NR⁷;

R² is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl, and a pharmaceutically acceptable counterion;

R^{3a} and R^{3b} are independently selected from hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl;

R⁴ is independently selected from aryl, substituted aryl, heteroaryl, substituted heteroaryl, heterocyclyl, substituted heterocyclyl, cycloalkyl, and substituted cycloalkyl;

R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, substituted sulfonyl, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

R⁶ is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl, and a pharmaceutically acceptable counterion;

R⁷ is selected from hydrogen, halo, aminocarbonyl, imino, amidino, aminocarbonylamino, amidinocarbonylamino, carboxy, carboxy ester, hydroxy, alkoxy, substituted alkoxy, acyl, acyloxy, cyano, thiol, alkylthio, substituted alkylthio, sulfonyl, and substituted sulfonyl;

R^{a}, R^{b}, and R^{c} are independently selected from hydrogen, alkyl, and substituted alkyl; and

m is from 0 to 5;

n is from 0 to 1, provided that n is 0 when [symbol] represents a double bond.

In some embodiments, a compound is provided that is a pharmaceutically acceptable salt of Formula (I).

In some embodiments, a compound is provided that is a solvate of Formula (I). In some embodiments, the solvate is a solvate of a pharmaceutically acceptable salt of Formula (I).

In some embodiments, Y is O. In some embodiments, Y is NH. In some embodiments, Y is a bond.

In some embodiments, R² is C₁-C₆ alkyl, such as methyl, ethyl, n-propyl, i-propyl, t-butyl, i-butyl, n-butyl, or n-hexyl. In some embodiments, R² is methyl or ethyl. In some embodiments, R² is C₁-C₆ alkyl optionally substituted with 1 to 5 substituents independently selected from hydroxyl, alkoxy, substituted alkoxy, carboxyl, phosphate, sulfate, amino, substituted amino, quaternary amino, acylamino, aminocarbonyl, aminocarbonylamino, or a combination thereof. In some embodiments, R² is C₁-C₆ alkyl substituted with hydroxyl (e.g., 3-hydroxypropyl and 2,3-dihydroxypropyl). In some embodiments, R² is C₁-C₆ alkyl substituted with alkoxy (—O-alkyl) or substituted alkoxy in which the alkyl portion is substituted with alkoxy and/or hydroxy (e.g., 2-(hydroxyethyl)oxyethyl, 2-(methyloxy)ethyloxyethyl, 2-[(2-hydroxyethyl)oxy]ethyloxyethyl, or 2-[2-(2-hydroxyethoxy)ethoxy]ethoxyethyl). In some embodiments, R² is C₁-C₆ alkyl substituted with carboxyl (e.g., butanoic acid). In some embodiments, R² is C₁-C₆ alkyl substituted with phosphate and/or sulfate (e.g., 3-(phosphonoxy)propyl or 3-(sulfonyloxy)propyl). In some embodiments, R² is C₁-C₆ alkyl substituted with amino (e.g., butan-1-amino), substituted amino, and/or quaternary amino (e.g., propyl(trimethyl ammonium chloride)). In some embodiments, R² is C₁-C₆ alkyl substituted with acylamino, aminocarbonyl, and/or aminocarbonylamino (e.g., 2-[[2-formamidopropanoyl]amino]-3-phenyl-propanoic acid and 2-formamidopropanoic acid).

In some embodiments, R² is aryl and/or substituted aryl (e.g., benzyl or methylbenzyl). In some embodiments, R² is a pharmaceutically acceptable counterion, such as sodium. In some embodiments, R² is hydrogen.

In some embodiments, a compound is provided that is a pharmaceutically acceptable salt of Formula (I).

In some embodiments, a compound is provided that is a solvate of Formula (I). In some embodiments, the solvate is a solvate of a pharmaceutically acceptable salt of Formula (I).

In some embodiments, Y is a bond, NH, or O. In some embodiments, Y is O. In some embodiments, Y is NH. In some embodiments, Y is a bond.

In some embodiments, [symbol] represents a single or double bond. In some embodiments, [symbol] represents a double bond. In some embodiments, [symbol] represents a single bond.

In some embodiments, R² is selected from the group consisting of alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, and substituted sulfonyl.

In some embodiments, R² is C₁₋₆ alkyl, wherein said C₁₋₆ alkyl is substituted with 1 to 5 substituents independently selected from the group consisting of hydroxyl, alkoxy, substituted alkoxy, oxy, carboxyl, phosphate, sulfate, amino, substituted amino, quaternary amino, acylamino, aminocarbonyl, and aminocarbonylamino, or a combination thereof.

In some embodiments, R² is C₁₋₆ alkyl, wherein said C₁₋₆ alkyl is substituted with 1 to 5 substituents independently selected from the group consisting of hydroxyl, alkoxy, substituted alkoxy, oxy, carboxyl, phosphate, sulfate, amino, substituted amino, quaternary amino, acylamino, aminocarbonyl, and aminocarbonylamino.

In some embodiments, R² is C₁-C₆ alkyl, such as methyl, ethyl, n-propyl, i-propyl, t-butyl, i-butyl, n-butyl, or n-hexyl. In some embodiments, R² is methyl or ethyl. In some embodiments, R² is C₁-C₆ alkyl optionally substituted with 1 to 5 substituents independently selected from hydroxyl, oxy, alkoxy, substituted alkoxy, carboxyl, phosphate, sulfate, amino, substituted amino, quaternary amino, acylamino, aminocarbonyl, aminocarbonylamino, or a combination thereof. In some embodiments, R² is C₁-C₆ alkyl substituted with hydroxyl (e.g., 3-hydroxypropyl and 2,3-dihydroxylpropyl). In some embodiments, R² is C₁-C₆ alkyl substituted with alkoxy (—O-alkyl) or substituted alkoxy in which the alkyl portion is substituted with alkoxy and/or hydroxyl (e.g., 2-(hydroxylethyl)oxyethyl, 2-(methyloxy)ethyloxyethyl, 2-[(2-hydroxylethyl)oxy]ethyloxyethyl, or 2-[2-(2-hydroxylethoxy)ethoxy]ethoxyethyl). In some embodiments, R² is C₁-C₆ alkyl substituted with carboxyl (e.g., butanoic acid). In some embodiments, R² is C₁-C₆ alkyl substituted with phosphate and/or sulfate (e.g., 3-(phosphonoxy)propyl or 3-(sulfonyloxy)propyl). In some embodiments, R² is C₁-C₆ alkyl substituted with amino (e.g., butan-1-amino), substituted amino, and/or quaternary amino (e.g., propyl(trimethyl ammonium chloride)). In some embodiments, R² is C₁-C₆ alkyl substituted with acylamino, aminocarbonyl, and/or aminocarbonylamino (e.g., 2-[[2-formamidopropanoyl]amino]-3-phenyl-propanoic acid and 2-formamidopropanoic acid).

In some embodiments, R² is aryl and/or substituted aryl (e.g., benzyl or methylbenzyl).

In some embodiments, R² is selected from the group consisting of hydrogen, C₁₋₆ alkyl, phenyl, -A¹, -A¹-(X¹)_{w}R⁸R⁹, -A¹R¹⁰, -A¹R¹¹, -A¹-N(R⁹)_{z}, -A¹-NHA²-R¹¹, -A¹-NHA²-NHA³R¹¹, and -A¹-R⁸R⁹; wherein:

A¹, A², A³, and A⁴ are each independently selected from C₁₋₆ alkylene, wherein one to four independent CH₂ groups of each of said A¹, A², A³, and A⁴ are optionally substituted with one to two R¹² groups;

each X¹ is independently selected from the group consisting of —(R⁸-A¹), —(R⁸-A²), —(R⁸-A³), and —(R⁸-A⁴);

R⁸ is O;

each R⁹ is independently selected from the group consisting of hydrogen and C₁₋₆ alkyl;

R¹⁰ is selected from the group consisting of phosphate, phosphonate, and sulfate;

R¹¹ is carboxyl;

each R¹² is independently selected from the group consisting of C₁₋₆ alkyl, oxo, aryl, arylalkyl, and hydroxyl;

w is an integer from 1 to 3; and

z is an integer from 2 to 3.

In some embodiments, R² is hydroxylalkoxyalkyl.

In some embodiments, R² is hydroxylethoxyethyl.

In some embodiments, R² is a group having the structure:

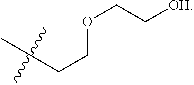

In some embodiments, Q⁴ is O and [symbol] represents a double bond. In some embodiments, Q⁴ is O, [symbol] represents a single bond, and R⁶ is hydrogen, alkyl, or substituted alkyl.

In some embodiments, Q⁴ is NR⁷ and [symbol] represents a double bond. In some embodiments, Q⁴ is NR⁷, [symbol] represents a single bond, and R⁶ is hydrogen, alkyl, or substituted alkyl.

In some embodiments, L¹ is C₁₋₃ alkylene. In some embodiments, L¹ is CH₂.

In some embodiments, L² is a bond.

In some embodiments, R^{3a} and R^{3b} are hydrogen.

In some embodiments, R⁴ is substituted phenyl or substituted heteroaryl. In some embodiments, R⁴ is substituted with at least one halo group, such as with at least one fluoro group. In some embodiments, R⁴ is phenyl substituted with at least one fluoro group. In some embodiments, R⁴ is 2,3-difluorophenyl.

In some embodiments, the ring B is selected from:

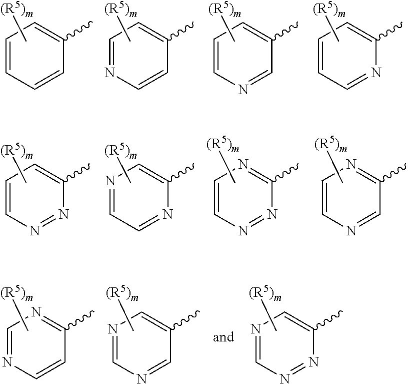

wherein,

the wavy line represents the point of attachment to the remainder of the molecule;

m is 1, 2, 3, or 4, in some embodiments m is 1, 2, or 3, in some embodiments m is 1 or 2, and in some embodiments, m is 1; and

R⁵ is independently selected from halo, haloalkyl, haloalkoxy, aryl, substituted aryl, heteroaryl, substituted heteroaryl, heterocyclyl, substituted heterocyclyl, cycloalkyl, and substituted cycloalkyl.

In some embodiments R¹ is selected from:

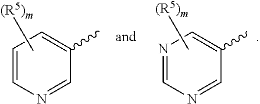

In some embodiments, R⁵ is substituted phenyl or substituted heteroaryl. In some embodiments, R⁵ is phenyl or heteroaryl, each of which is substituted with at least one group selected from alkyl, haloalkyl, and optionally substituted alkoxy. In some embodiments, R⁵ is phenyl or heteroaryl, each of which is substituted with at least one group selected from lower alkyl, CF₃, and optionally substituted methoxy. In some embodiments, R⁵ is phenyl substituted with at least one group selected from lower alkyl, CF₃, and optionally substituted methoxy. In some embodiments, R⁵ is phenyl substituted with at least one group selected from lower alkyl, CF₃, and R¹¹—CH₂O— wherein R¹¹ is optionally substituted heteroaryl. In some embodiments, R⁵ is phenyl substituted with at least one group selected from lower alkyl, CF₃, and R¹¹—CH₂O— wherein R¹¹ is optionally substituted pyridinyl. In some embodiments, R⁵ is phenyl substituted with at least one group selected from lower alkyl, CF₃, and R¹¹—CH₂O— wherein R¹¹ is pyridinyl.

In some embodiments, provided is a compound that is Formula (II)

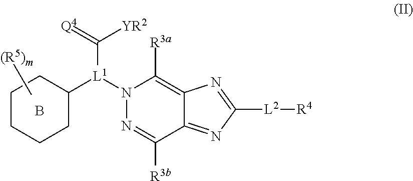

or a pharmaceutically acceptable salt or solvate thereof, wherein,

Y is a bond, O or NR^{c};

Q⁴ is O or NR⁷; and

R², R^{3a}, R^{3b}, R⁴, R⁵, R⁷, R^{c}, L¹, L², and m are as defined herein.

In some embodiments, provided is a compound that is Formula (III)

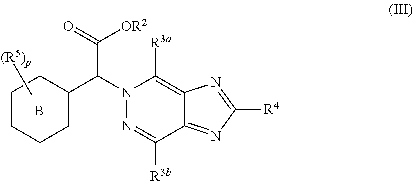

or a pharmaceutically acceptable salt or solvate thereof, wherein, R², R^{3a}, R^{3b}, R⁴, R⁵, and m are as defined herein.

In some embodiments, provided is a compound that is Formula (I):

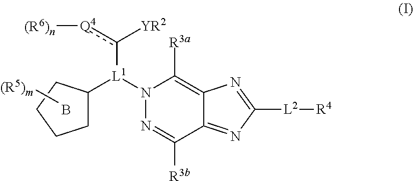

or a pharmaceutically acceptable salt or solvate thereof, wherein:

[symbol] represents a single or double bond;

ring B is a 5-membered aromatic ring wherein 1 to 3 ring carbon atoms are optionally replaced by nitrogen or oxygen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 8- or 9-membered bicyclic ring;

L¹ is independently O₃₋₆ cycloalkylene or C₁₋₅ alkylene, where one or two CH₂ groups of said C₁₋₅ alkylene are optionally replaced with NR^{a}, S, (C═O), or O and optionally two adjacent carbon atoms form a double bond;

L² is a bond or independently C₃₋₆ cycloalkylene or is C₁₋₅ alkylene where one or two CH₂ groups of said C₁₋₅ alkylene are optionally replaced with NR^{b}, S, (C═O), or O and optionally two adjacent carbon atoms form a double bond or triple bond, and wherein said C₁₋₅ alkylene is optionally substituted with one to three groups independently selected from halo, alkyl, and spirocycloalkyl;

Y is a bond, O, S, or NR^{c};

Q⁴ is O, S, or NR⁷;

R² is selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, and substituted sulfonyl;

R^{3a} and R^{3b} are independently selected from the group consisting of hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl;

R⁴ is independently selected from the group consisting of aryl, substituted aryl, heteroaryl, substituted heteroaryl, heterocyclyl, substituted heterocyclyl, cycloalkyl, and substituted cycloalkyl;

R⁵ is independently selected from the group consisting of hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, substituted sulfonyl, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

R⁶ is selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, and substituted sulfonyl;

R⁷ is selected from the group consisting of hydrogen, halo, aminocarbonyl, imino, amidino, aminocarbonylamino, amidinocarbonylamino, carboxy, carboxy ester, hydroxy, alkoxy, substituted alkoxy, acyl, acyloxy, cyano, thiol, alkylthio, substituted alkylthio, sulfonyl, and substituted sulfonyl;

R^{a}, R^{b}, and R^{c} are independently selected from the group consisting of hydrogen, alkyl, and substituted alkyl; and

m is from 0 to 4;

n is from 0 to 1, provided that n is 0 when [symbol] represents a double bond.

In some embodiments, provided is a compound that is Formula (I):

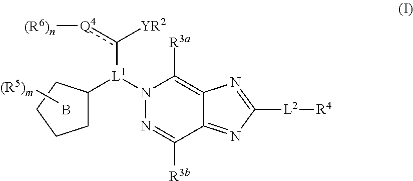

or a pharmaceutically acceptable salt or solvate thereof, wherein:

[symbol] represents a single or double bond;

ring B is a 5-membered aromatic ring wherein 1 to 3 ring carbon atoms are optionally replaced by nitrogen or oxygen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 8- or 9-membered bicyclic ring;

L¹ is independently C₃₋₆ cycloalkylene or C₁₋₅ alkylene, where one or two CH₂ groups of said C₁₋₅ alkylene are optionally replaced with NR^{a}, S, (C═O), or O and optionally two adjacent carbon atoms form a double bond;

L² is a bond or independently C₃₋₆ cycloalkylene or is C₁₋₅ alkylene where one or two CH₂ groups of said C₁₋₅ alkylene are optionally replaced with NR^{b}, S, (C═O), or O and optionally two adjacent carbon atoms form a double bond or triple bond, and wherein said C₁₋₅ alkylene is optionally substituted with one to three groups independently selected from halo, alkyl, and spirocycloalkyl;

Y is a bond, O, S, or NR^{c};

Q⁴ is O, S, or NR⁷;

R² is selected from the group consisting of hydrogen, alkyl, aryl, -A¹, -A¹-(X¹)_{w}-R⁸R⁹, -A¹-R¹⁰, -A¹-R¹¹, -A¹-N(R⁹)_{z}, -A¹-NHA²-R¹¹, -A¹NHA²-NHA³R¹¹, and -A¹-R⁸R⁹;

A¹, A2, A³, and A⁴ are each independently selected from C₁₋₆ alkylene, wherein one to four independent CH₂ groups of each of said A¹, A², A³, and A⁴ are optionally substituted with one to two R¹² groups;

each X¹ is independently selected from the group consisting of —(R⁸-A¹), —(R⁸-A²), —(R⁸-A³), and —(R⁸-A⁴);

R^{3a} and R^{3b} are independently selected from the group consisting of hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl;

R⁴ is independently selected from the group consisting of aryl, substituted aryl, heteroaryl, substituted heteroaryl, heterocyclyl, substituted heterocyclyl, cycloalkyl, and substituted cycloalkyl;

each R⁵ is independently selected from the group consisting of hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, substituted sulfonyl, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

each R⁶ is selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, and substituted sulfonyl;

R⁷ is selected from the group consisting of hydrogen, halo, aminocarbonyl, imino, amidino, aminocarbonylamino, amidinocarbonylamino, carboxy, carboxy ester, hydroxy, alkoxy, substituted alkoxy, acyl, acyloxy, cyano, thiol, alkylthio, substituted alkylthio, sulfonyl, and substituted sulfonyl;

R⁸ is O;

each R⁹ is independently selected from the group consisting of hydrogen and C₁₋₆ alkyl;

R¹⁹ is selected from the group consisting of phosphate, phosphonate, and sulfate;

R¹¹ is carboxyl;

each R¹² is independently selected from the group consisting of C₁₋₆ alkyl, oxo, aryl, arylalkyl, and hydroxyl;

R^{a}, R^{b}, and R^{c} are independently selected from the group consisting of hydrogen, alkyl, and substituted alkyl;

m is 0 or an integer from 1 to 4;

n is 0 or 1, provided that n is 0 when [symbol] represents a double bond;

w is 0 or an integer from 1 to 3; and

z is an integer from 2 to 3.

In some embodiments, a compound is provided that is a pharmaceutically acceptable salt of Formula (I).

In some embodiments, a compound is provided that is a solvate of Formula (I). In some embodiments, the solvate is a solvate of a pharmaceutically acceptable salt of Formula (I).

In some embodiments, Y is a bond, NH, or O. In some embodiments,

Y is O. In some embodiments, Y is NH. In some embodiments, Y is a bond.

In some embodiments, [symbol] represents a single or double bond. In some embodiments, [symbol] represents a double bond. In some embodiments, [symbol] represents a single bond.

In some embodiments, R² is selected from the group consisting of alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, and substituted sulfonyl.

In some embodiments, R² is C₁₋₆ alkyl, wherein said C₁₋₆ alkyl is substituted with 1 to 5 substituents independently selected from the group consisting of hydroxyl, alkoxy, substituted alkoxy, oxy, carboxyl, phosphate, sulfate, amino, substituted amino, quaternary amino, acylamino, aminocarbonyl, and aminocarbonylamino, or a combination thereof.

In some embodiments, R² is C₁₋₆ alkyl, wherein said C₁₋₆ alkyl is substituted with 1 to 5 substituents independently selected from the group consisting of hydroxyl, alkoxy, substituted alkoxy, oxy, carboxyl, phosphate, sulfate, amino, substituted amino, quaternary amino, acylamino, aminocarbonyl, and aminocarbonylamino.

In some embodiments, R² is C₁-C₆ alkyl, such as methyl, ethyl, n-propyl, i-propyl, t-butyl, i-butyl, n-butyl, or n-hexyl. In some embodiments, R² is methyl or ethyl. In some embodiments, R² is C₁-C₆ alkyl optionally substituted with 1 to 5 substituents independently selected from hydroxyl, oxy, alkoxy, substituted alkoxy, carboxyl, phosphate, sulfate, amino, substituted amino, quaternary amino, acylamino, aminocarbonyl, aminocarbonylamino, or a combination thereof. In some embodiments, R² is C₁-C₆ alkyl substituted with hydroxyl (e.g., 3-hydroxypropyl and 2,3-dihydroxylpropyl). In some embodiments, R² is C₁-C₆ alkyl substituted with alkoxy (—O-alkyl) or substituted alkoxy in which the alkyl portion is substituted with alkoxy and/or hydroxyl (e.g., 2-(hydroxylethyl)oxyethyl, 2-(methyloxy)ethyloxyethyl, 2-[(2-hydroxylethyl)oxy]ethyloxyethyl, or 2-[2-(2-hydroxylethoxy)ethoxy]ethoxyethyl). In some embodiments, R² is C₁-C₆ alkyl substituted with carboxyl (e.g., butanoic acid). In some embodiments, R² is C₁-C₆ alkyl substituted with phosphate and/or sulfate (e.g., 3-(phosphonoxy)propyl or 3-(sulfonyloxy)propyl). In some embodiments, R² is C₁-C₆ alkyl substituted with amino (e.g., butan-1-amino), substituted amino, and/or quaternary amino (e.g., propyl(trimethyl ammonium chloride)). In some embodiments, R² is C₁-C₆ alkyl substituted with acylamino, aminocarbonyl, and/or aminocarbonylamino (e.g., 2-[[2-formamidopropanoyl]amino]-3-phenyl-propanoic acid and 2-formamidopropanoic acid).

In some embodiments, R² is aryl and/or substituted aryl (e.g., benzyl or methylbenzyl).

In some embodiments, R² is selected from the group consisting of hydrogen, C₁₋₆ alkyl, phenyl, -A¹, -A¹-(X¹)_{w}-R⁸R⁹, -A¹-R¹⁰, -A¹-R¹¹, -A¹-N (R⁹)_{z}, -A¹-NHA2-R¹¹, -A¹-NHA²-NHA³R¹¹, and -A¹-R⁸R⁹; wherein:

A¹, A², A³, and A⁴ are each independently selected from C₁₋₆ alkylene, wherein one to four independent CH₂ groups of each of said A¹, A², A³, and A⁴ are optionally substituted with one to two R¹² groups;

each X¹ is independently selected from the group consisting of —(R⁸-A¹), —(R⁸-A²), —(R⁸-A³), and —(R⁸-A⁴);

R⁸ is O;

each R⁹ is independently selected from the group consisting of hydrogen and C₁₋₆ alkyl;

R¹⁰ is selected from the group consisting of phosphate, phosphonate, and sulfate;

R¹¹ is carboxyl;

each R¹² is independently selected from the group consisting of C₁₋₆ alkyl, oxo, aryl, arylalkyl, and hydroxyl;

w is an integer from 1 to 3; and

z is an integer from 2 to 3.

In some embodiments, R² is hydroxylalkoxyalkyl.

In some embodiments, R² is hydroxylethoxyethyl.

In some embodiments, R² is a group having the structure:

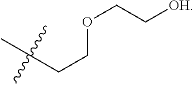

In some embodiments, R² is a pharmaceutically acceptable counterion, such as sodium. In some embodiments, R² is hydrogen.

In some embodiments, Q⁴ is O and [symbol] represents a double bond. In some embodiments, Q⁴ is O, [symbol] represents a single bond, and R⁶ is hydrogen, alkyl, or substituted alkyl.

In some embodiments, Q⁴ is NR⁷ and [symbol] represents a double bond. In some embodiments, Q⁴ is NR⁷, [symbol] represents a single bond, and R⁶ is hydrogen, alkyl, or substituted alkyl.

In some embodiments, L¹ is C₁₋₃ alkylene. In some embodiments, L¹ is CH₂.

In some embodiments, L² is a bond.

In some embodiments, R^{3a} and R^{3b} are hydrogen.

In some embodiments, R⁴ is substituted phenyl or substituted heteroaryl. In some embodiments, R⁴ is substituted with at least one halo group, such as with at least one fluoro group. In some embodiments, R⁴ is phenyl substituted with at least one fluoro group. In some embodiments, R⁴ is selected from the group consisting of aryl, substituted aryl, heteroaryl, and substituted heteroaryl. In some embodiments, R⁴ is aryl or substituted aryl. In some embodiments, R⁴ is phenyl or substituted phenyl. In some embodiments, R⁴ is aryl. In some embodiments, R⁴ is phenyl. In some embodiments, R⁴ is substituted with at least one halo group. In some embodiments, R⁴ is substituted with one to two fluoro groups. In some embodiments, R⁴ is fluorophenyl. In some embodiments, R⁴ is difluorophenyl. In some embodiments, R⁴ is 2-fluorophenyl. In some embodiments, R⁴ is 2,3-difluorophenyl.

In some embodiments the ring B is selected from:

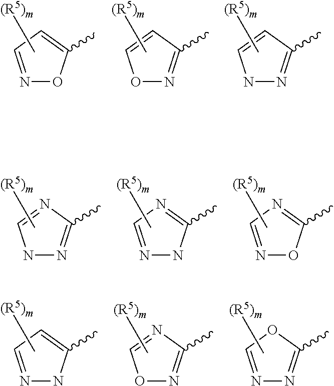

wherein,

the wavy line represents the point of attachment to the remainder of the molecule;

m is 1, 2, 3 or 4, in some embodiments m is 1, 2, or 3, in some embodiments m is 1 or 2, and in some embodiments, m is 1; and

In some embodiments, ring B is selected from:

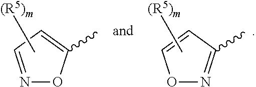

In some embodiments, ring B is:

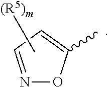

R⁵ is independently selected from halo, haloalkyl, haloalkoxy, aryl, substituted aryl, heteroaryl, substituted heteroaryl, heterocyclyl, substituted heterocyclyl, cycloalkyl, and substituted cycloalkyl. In some embodiments, R⁵ is substituted phenyl or substituted heteroaryl. In some embodiments, R⁵ is phenyl or heteroaryl, each of which is optionally substituted with at least one group selected from alkyl, cycloalkyl, haloalkyl, and optionally substituted alkoxy. In some embodiments, R⁵ is phenyl which is substituted with at least one group selected from alkyl, cycloalkyl, haloalkyl, and optionally substituted alkoxy. In some embodiments, R⁵ is phenyl or heteroaryl, each of which is substituted with at least one group selected from lower alkyl, CF₃, and optionally substituted methoxy. In some embodiments, R⁵ is phenyl substituted with at least one group selected from lower alkyl, CF₃, and optionally substituted methoxy. In some embodiments, R⁵ is phenyl substituted with at least one group selected from lower alkyl, CF₃, and R¹³—CH₂O— wherein R¹³ is optionally substituted heteroaryl. In some embodiments, R⁵ is phenyl substituted with at least one group selected from lower alkyl, CF₃, and R¹³—CH₂O— wherein R¹³ is optionally substituted pyridinyl. In some embodiments, R⁵ is phenyl substituted with at least one group selected from lower alkyl, CF₃, and R¹³—CH₂O— wherein R¹³ is pyridinyl. In some embodiments, R⁵ is phenyl which is substituted with at least one group selected from cycloalkyl and haloalkyl. In some embodiments, R⁵ is phenyl which is substituted with two groups selected from cyclopropyl, cyclobutyl, and trifluoromethyl. In some embodiments, R⁵ is phenyl which is substituted with a cyclopropyl group and a trifluoromethyl group. In some embodiments, R⁵ is phenyl or heteroaryl, each of which is substituted with at least one group selected from the group consisting of alkyl, haloalkyl, cycloalkyl, and optionally substituted alkoxy.

In some embodiments, R⁵ is selected from:

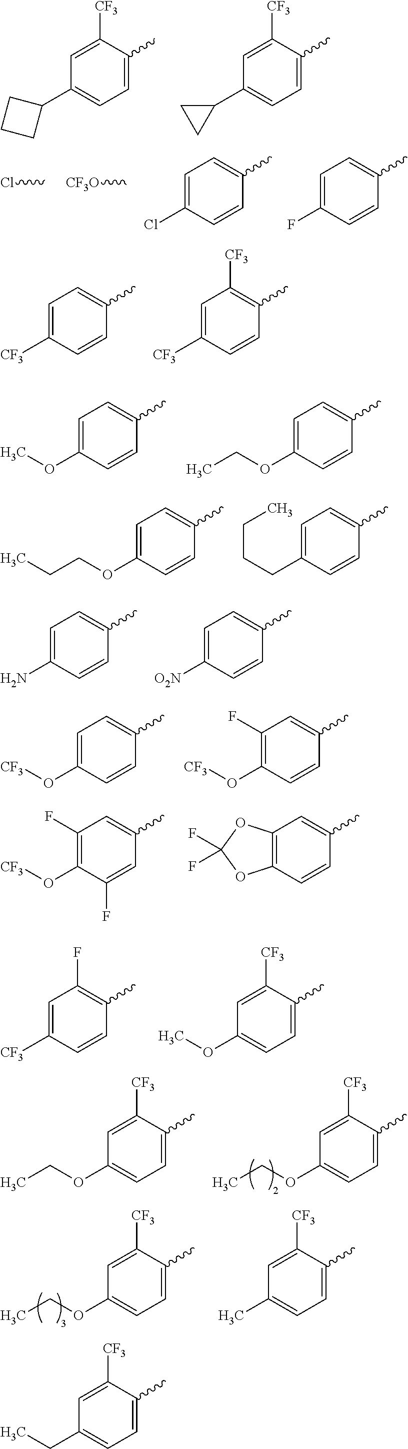

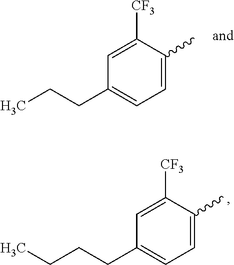

wherein the wavy line represents the point of attachment to the remainder of the molecule.

In some embodiments, R⁵ is selected from:

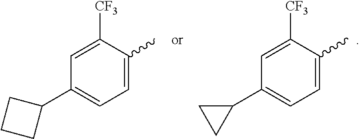

In some embodiments, R⁵ is:

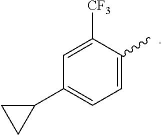

In some embodiments, R⁶ is selected from the group consisting of hydrogen, alkyl, and substituted alkyl. In some embodiments, n is 0. In some embodiments, n is 1.

In some embodiments, provided is a compound that is Formula (II)

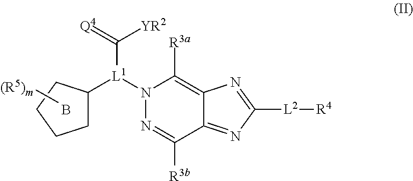

or a pharmaceutically acceptable salt or solvate thereof, wherein,

Y is a bond, O or NR^{c};

Q⁴ is O or NR⁷; and

R², R^{3a}, R^{3b}, R⁴, R⁵, R⁷, R^{c}, L¹, L², and m are as defined herein.

In some embodiments, provided is a compound that is Formula (III)

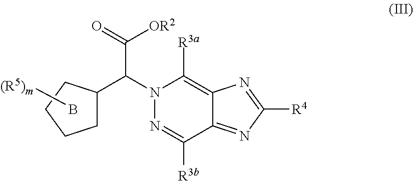

or a pharmaceutically acceptable salt or solvate thereof, wherein, R², R^{3a}, R^{3b}, R⁴, R⁵, and m are as defined herein.

In some embodiments, the present invention provides one or more compound(s) selected from the group consisting of:
Methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
Ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
Propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
1-Methylethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
3-Hydroxypropyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
1,1-Dimethylethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
Butyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
2-[(2-Hydroxyethyl)oxy]ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
2-{[2-(Methyloxy)ethyl]oxy}ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
Hexyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
2-({2-[(2-Hydroxyethyl)oxy]ethyl}oxy)ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
2-{[2-({2-[(2-Hydroxyethyl)oxy]ethyl}oxy)ethyl]oxy}ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
2,3-Dihydroxypropyl 2-[2-(2,3-difluorophenyl)-5H-imidazo[4,5-d]pyridazin-5-yl]-2-[3-[4-propyloxy-2-(trifluoromethyl)phenyl]isoxazol-5-yl]acetate,
3-(Phosphonooxy)propyl[2-(2,3-difluorophenyl)-5H-imidazo[4,5-d]pyridazin-5-yl]{3-[4-(propyloxy)-2-(trifluoromethyl)phenyl]-5-isoxazolyl}acetate mono sodium salt,
3-(Phosphonooxy)propyl[2-(2,3-difluorophenyl)-5H-imidazo[4,5-d]pyridazin-5-yl]{3-[4-(propyloxy)-2-(trifluoromethyl)phenyl]-5-isoxazolyl}acetate,
3-(Sulfooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate sodium salt,
3-(Sulfooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoic acid sodium salt,
3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoic acid,
3-[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]oxypropyl-trimethyl-ammonium chloride,
N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanine,
N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanyl-L-phenylalanine,
2-[2-(2,3-Difluorophenyl)-5H-imidazo[4,5-d]pyridazin-5-yl]-N-methyl-2-{3-[4-(propyloxy)-2-(trifluoromethyl)phenyl]-5-isoxazolyl}acetamide,
2-[2-(2,3-Difluorophenyl)-5H-imidazo[4,5-d]pyridazin-5-yl]-N-ethyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-propyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-isopropyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-(3-hydroxypropyl)-2-[3-[4-propyloxy-2-trifluoromethyl)phenyl]isoxazol-5-yl]acetamide,
2-[2-(2,3-difluorophenyl)-5H-imidazo[4,5-d]pyridazin-5-yl]-N-tert-butyl-2-[3-[4-propyloxy-2-(trifluoromethyl)phenyl]isoxazol-5-yl]acetamide,
2-[2-(2,3-difluorophenyl)-5H-imidazo[4,5-d]pyridazin-5-yl]-N-butyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-[2-(2-hydroxyethoxy)ethyl]-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-[2-(2-methoxyethoxy)ethyl]-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-hexyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-[2-[2-(2-hydroxyethoxy)ethoxy]ethyl]-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-[2-[2-[2-(2-hydroxyethoxy)ethoxy]ethoxy]ethyl]-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-(2,3-dihydroxypropyl)-2-[3-[4-propyloxy-2-(trifluoromethyl)phenyl]isoxazol-5-yl]acetamide,
N-3-(phosphonooxy)propyl-2-[2-(2,3-difluorophenyl)-5H-imidazo[4,5-d]pyridazin-5-yl]-2-[3-[4-propyloxy-2-(trifluoromethyl)phenyl]isoxazol-5-yl]acetamide mono sodium salt,
N-3-(phosphonooxy)propyl-2-[2-(2,3-difluorophenyl)-5H-imidazo[4,5-d]pyridazin-5-yl]-2-[3-[4-propyloxy-2-(trifluoromethyl)phenyl]isoxazol-5-yl]acetamide,
N-3-(Sulfonyloxy)propyl-2-[2-(2,3-difluorophenyl)-5H-imidazo[4,5-d]pyridazin-5-yl]-2-[3-[4-propyloxy-2-(trifluoromethyl)phenyl]isoxazol-5-yl]acetamide mono sodium salt,
N-3-(Sulfonyloxy)propyl-2-[2-(2,3-difluorophenyl)-5H-imidazo[4,5-d]pyridazin-5-yl]-2-[3-[4-propyloxy-2-(trifluoromethyl)phenyl]isoxazol-5-yl]acetamide,
3-[[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]amino]propanoic acid sodium salt,
3-[[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]amino]propanoic acid,
3-[[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]amino]propyl-trimethyl-ammonium chloride,
2-[3-[[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]amino]propanoylamino]propanoic acid,
2-[2-[3-[[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]amino]propanoylamino]propanoylamino]propanoic acid,
Methyl [2-(2,3-difluorophenyl)-5H-imidazo[4,5-d]pyridazin-5-yl]{6-[4-(propyloxy)-2-(trifluoromethyl)phenyl]-3-pyridinyl}acetate,
Ethyl [2-(2,3-difluorophenyl)-5H-imidazo[4,5-d]pyridazin-5-yl]{6-[4-(propyloxy)-2-(trifluoromethyl)phenyl]-3-pyridinyl}acetate,
Propyl [2-(2,3-difluorophenyl)-5H-imidazo[4,5-d]pyridazin-5-yl]{6-[4-(propyloxy)-2-(trifluoromethyl)phenyl]-3-pyridinyl}acetate,
1-Methylethyl [2-(2,3-difluorophenyl)-5H-imidazo[4,5-d]pyridazin-5-yl]{6-[4-(propyloxy)-2-(trifluoromethyl)phenyl]-3-pyridinyl}acetate,
3-Hydroxypropyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
Tert-butyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
Butyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
2-(2-Hydroxyethoxy)ethyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
2-(2-Methoxyethoxy)ethyl 2-[2-(2,3-difluorophenyl)-5H-imidazo[4,5-d]pyridazin-5-yl]-2-[6-[4-propoxy-2-(trifluoromethyl)phenyl]-3-pyridinyl]acetate,
Hexyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
2-[2-(2-Hydroxyethoxy)ethoxy]ethyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
2-[2-[2-(2-Hydroxyethoxy)ethoxy]ethoxy]ethyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
2,3-Dihydroxypropyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
3-(Phosphonooxy)propyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate mono sodium salt,
3-(Phosphonooxy)propyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
3-(Sulfonyloxy)propyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate mono sodium salt,
3-(Sulfonyloxy)propyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
3-[2-[2-(2,3-Difluorophenyl)-5H-imidazo[4,5-d]pyridazin-5-yl]-2-[6-[4-propyloxy-2-(trifluoromethyl)phenyl]-3-pyridinyl]acetyl]oxypropanoic acid sodium salt,
3-[2-[2-(2,3-Difluorophenyl)-5H-imidazo[4,5-d]pyridazin-5-yl]-2-[6-[4-propyloxy-2-(trifluoromethyl)phenyl]-3-pyridinyl]acetyl]oxypropanoic acid,
3-[2-[2-(2,3-Difluorophenyl)-5H-imidazo[4,5-d]pyridazin-5-yl]-2-[6-[4-propyloxy-2-(trifluoromethyl)phenyl]-3-pyridinyl]acetyl]oxypropyl-dimethyl-ammonium chloride,
2-[3-[2-[2-(2,3-Difluorophenyl)-5H-imidazo[4,5-d]pyridazin-5-yl]-2-[6-[4-propyloxy-2-(trifluoromethyl)phenyl]-3-pyridinyl]acetyl]oxypropanoylamino]propanoic acid,
2-[2-[3-[2-[2-(2,3-Difluorophenyl)-5H-imidazo[4,5-d]pyridazin-5-yl]-2-[6-[4-propyloxy-2-(trifluoromethyl)phenyl]-3-pyridinyl]acetyl]oxypropanoylamino]propanoylamino]propanoic acid,
Methyl [2-(2,3-difluorophenyl)-5H-imidazo[4,5-d]pyridazin-5-yl]{2-[4-(propyloxy)-2-(trifluoromethyl)phenyl]-5-pyrimidinyl}acetate,
Ethyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
Propyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
Isopropyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
3-Hydroxypropyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
Tert-butyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
Butyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
2-(2-Hydroxyethoxy)ethyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
2-(2-Methoxyethoxy)ethyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
Hexyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
2-[2-(2-Hydroxyethoxy)ethoxy]ethyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
2-[2-[2-(2-Hydroxyethoxy)ethoxy]ethoxy]ethyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
2,3-Dihydroxypropyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
3-(Phosphonooxy)propyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate mono sodium salt,
3-(Phosphonooxy)propyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
3-(Sulfonyloxy)propyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate mono sodium salt,
3-(Sulfonyloxy)propyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
3-[2-[2-(2,3-Difluorophenyl)-5H-imidazo[4,5-d]pyridazin-5-yl]-2-[2-[4-propyloxy-2-(trifluoromethyl)phenyl]-5-pyrimidinyl]acetyl]oxypropanoic acid sodium salt,
3-[2-[2-(2,3-Difluorophenyl)-5H-imidazo[4,5-d]pyridazin-5-yl]-2-[2-[4-propyloxy-2-(trifluoromethyl)phenyl]-5-pyrimidinyl]acetyl]oxypropanoic acid,
3-[2-[2-(2,3-Difluorophenyl)-5H-imidazo[4,5-d]pyridazin-5-yl]-2-[2-[4-propyloxy-2-(trifluoromethyl)phenyl]-5-pyrimidinyl]acetyl]oxypropyl-trimethyl-ammonium chloride,
2-[3-[2-[2-(2,3-Difluorophenyl)-5H-imidazo[4,5-d]pyridazin-5-yl]-2-[2-[4-propyloxy-2-(trifluoromethyl)phenyl]-5-pyrimidinyl]acetyl]oxypropanoylamino]propanoic acid,
2-[2-[3-[2-[2-(2,3-Difluorophenyl)-5H-imidazo[4,5-d]pyridazin-5-yl]-2-[2-[4-propyloxy-2-(trifluoromethyl)phenyl]-5-pyrimidinyl]acetyl]oxypropanoylamino]propanoylamino]propanoic acid,
Methyl 2-[6-[2,4-bis(trifluoromethyl)phenyl]pyridazin-3-yl]-2-[2-(2-fluorophenyl)-5H-imidazo[4,5-c]pyridin-5-yl]acetate,
Ethyl 2-[6-[2,4-bis(trifluoromethyl)phenyl]pyridazin-3-yl]-2-[2-(2-fluorophenyl)-5H-imidazo[4,5-c]pyridin-5-yl]acetate,
Propyl 2-[6-[2,4-bis(trifluoromethyl)phenyl]pyridazin-3-yl]-2-[2-(2-fluorophenyl)-5H-imidazo[4,5-c]pyridin-5-yl]acetate,
Isopropyl 2-[6-[2,4-bis(trifluoromethyl)phenyl]pyridazin-3-yl]-2-[2-(2-fluorophenyl)-5H-imidazo[4,5-c]pyridin-5-yl]acetate,
3-hydroxypropyl 2-[6-[2,4-bis(trifluoromethyl)phenyl]pyridazin-3-yl]-2-[2-(2-fluorophenyl)-5H-imidazo[4,5-c]pyridin-5-yl]acetate,
Tert-butyl 2-[6-[2,4-bis(trifluoromethyl)phenyl]pyridazin-3-yl]-2-[2-(2-fluorophenyl)-5H-imidazo[4,5-c]pyridin-5-yl]acetate,
Butyl 2-[6-[2,4-bis(trifluoromethyl)phenyl]pyridazin-3-yl]-2-[2-(2-fluorophenyl)-5H-imidazo[4,5-c]pyridin-5-yl]acetate,
2-(2-Hydroxyethoxy)ethyl 2-[6-[2,4-bis(trifluoromethyl)phenyl]pyridazin-3-yl]-2-[2-(2-fluorophenyl)-5H-imidazo[4,5-c]pyridin-5-yl]acetate,
2-(2-Methyloxyethoxy)ethyl 2-[6-[2,4-bis(trifluoromethyl)phenyl]pyridazin-3-yl]-2-[2-(2-fluorophenyl)-5H-imidazo[4,5-c]pyridin-5-yl]acetate,
Hexyl 2-[6-[2,4-bis(trifluoromethyl)phenyl]pyridazin-3-yl]-2-[2-(2-fluorophenyl)-5H-imidazo[4,5-c]pyridin-5-yl]acetate,
2-[2-(2-Hydroxyethoxy)ethoxy]ethyl 2-[6-[2,4-bis(trifluoromethyl)phenyl]pyridazin-3-yl]-2-[2-(2-fluorophenyl)-5H-imidazo[4,5-c]pyridin-5-yl]acetate,
2-[2-[2-(2-Hyd roxyethoxy)ethoxy]ethoxy]ethyl 2-[6-[2,4-bis(trifluoromethyl)phenyl]pyridazin-3-yl]-2-[2-(2-fluorophenyl)-5H-imidazo[4,5-c]pyridin-5-yl]acetate,
2,3-Dihydroxypropyl 2-[6-[2,4-bis(trifluoromethyl)phenyl]pyridazin-3-yl]-2-[2-[(2-fluorophenyl)methyl]-5H-imidazo[4,5-c]pyridin-5-yl]acetate,
3-(Phosphonooxy)propyl 2-[6-[2,4-bis(trifluoromethyl)phenyl]pyridazin-3-yl]-2-[2-(2-fluorophenyl)-5H-imidazo[4,5-c]pyridin-5-yl]acetate mono sodium salt,
3-(Phosphonooxy)propyl 2-[6-[2,4-bis(trifluoromethyl)phenyl]pyridazin-3-yl]-2-[2-(2-fluorophenyl)-5H-imidazo[4,5-c]pyridin-5-yl]acetate,
3-(Sulfonyloxy)propyl 2-[6-[2,4-bis(trifluoromethyl)phenyl]pyridazin-3-yl]-2-[2-(2-fluorophenyl)-5H-imidazo[4,5-c]pyridin-5-yl]acetate mono sodium salt,
3-(Sulfonyloxy)propyl 2-[6-[2,4-bis(trifluoromethyl)phenyl]pyridazin-3-yl]-2-[2-(2-fluorophenyl)-5H-imidazo[4,5-c]pyridin-5-yl]acetate,
3-[2-[6-[2,4-Bis(trifluoromethyl)phenyl]pyridazin-3-yl]-2-[2-(2-fluorophenyl)-5H-imidazo[4,5-c]pyridin-5-yl]acetyl]oxypropanoate sodium salt,
3-[2-[6-[2,4-Bis(trifluoromethyl)phenyl]pyridazin-3-yl]-2-[2-(2-fluorophenyl)-5H-imidazo[4,5-c]pyridin-5-yl]acetyl]oxypropanoic acid,
3-[2-[6-[2,4-Bis(trifluoromethyl)phenyl]pyridazin-3-yl]-2-[2-(2-fluorophenyl)-5H-imidazo[4,5-c]pyridin-5-yl]acetyl]oxypropyl-trimethyl-ammonium chloride,
2-[3-[2-[6-[2,4-Bis(trifluoromethyl)phenyl]pyridazin-3-yl]-2-[2-(2-fluorophenyl)-5H-imidazo[4,5-c]pyridin-5-yl]acetyl]oxypropanoylamino]propanoic acid,
2-[2-[3-[2-[6-[2,4-Bis(trifluoromethyl)phenyl]pyridazin-3-yl]-2-[2-(2-fluorophenyl)-5H-imidazo[4,5-c]pyridin-5-yl]acetyl]oxypropanoylamino]propanoylamino]propanoic acid,
Methyl 2-[6-[2,4-bis(trifluoromethyl)phenyl]pyridazin-3-yl]-2-[8-(2-fluorophenyl)-1H-purin-1-yl]acetate,
Ethyl 2-[6-[2,4-bis(trifluoromethyl)phenyl]pyridazin-3-yl]-2-[8-(2-fluorophenyl)-1H-purin-1-yl]acetate,
Propyl 2-[6-[2,4-bis(trifluoromethyl)phenyl]pyridazin-3-yl]-2-[8-(2-fluorophenyl)-1H-purin-1-yl]acetate,
Isopropyl 2-[6-[2,4-bis(trifluoromethyl)phenyl]pyridazin-3-yl]-2-[8-(2-fluorophenyl)-1H-purin-1-yl]acetate,
3-Hydroxypropyl 2-[6-[2,4-bis(trifluoromethyl)phenyl]pyridazin-3-yl]-2-[8-(2-fluorophenyl)-1H-purin-1-yl]acetate,
Tert-butyl 2-[6-[2,4-bis(trifluoromethyl)phenyl]pyridazin-3-yl]-2-[8-(2-fluorophenyl)-1H-purin-1-yl]acetate,
Butyl 2-[6-[2,4-bis(trifluoromethyl)phenyl]pyridazin-3-yl]-2-[8-(2-fluorophenyl)-1H-purin-1-yl]acetate,
2-(2-Hydroxyethoxy)ethyl 2-[6-[2,4-bis(trifluoromethyl)phenyl]pyridazin-3-yl]-2-[8-(2-fluorophenyl)-1H-purin-1-yl]acetate,
2-(2-Methyloxyethoxy)ethyl 2-[6-[2,4-bis(trifluoromethyl)phenyl]pyridazin-3-yl]-2-[8-(2-fluorophenyl)-1H-purin-1-yl]acetate,
Hexyl 2-[6-[2,4-bis(trifluoromethyl)phenyl]pyridazin-3-yl]-2-[8-(2-fluorophenyl)-1H-purin-1-yl]acetate,
Methyl 2-[2-(2-fluorophenyl)-5H-imidazo[4,5-d]pyridazin-5-yl]-2-[3-[4-propyloxy-2-(trifluoromethyl)phenyl]isoxazol-5-yl]acetate,
Methyl 2-[2-(2,3-difluorophenyl)-5H-imidazo[4,5-d]pyridazin-5-yl]-2-[3-[2,4-bis(trifluoromethyl)phenyl]isoxazol-5-yl]acetate,
Methyl 2-[2-(2-fluorophenyl)-5H-imidazo[4,5-d]pyridazin-5-yl]-2-[3-[2,4-bis(trifluoromethyl)phenyl]isoxazol-5-yl]acetic acid,
Ethyl 2-[2-(2-fluorophenyl)-5H-imidazo[4,5-d]pyridazin-5-yl]-2-[3-[4-propyloxy-2-(trifluoromethyl)phenyl]isoxazol-5-yl]acetate,
Ethyl 2-[2-(2,3-difluorophenyl)-5H-imidazo[4,5-d]pyridazin-5-yl]-2-[3-[2,4-bis(trifluoromethyl)phenyl]isoxazol-5-yl]acetate,
Ethyl 2-[2-(2-fluorophenyl)-5H-imidazo[4,5-d]pyridazin-5-yl]-2-[3-[2,4-bis(trifluoromethyl)phenyl]isoxazol-5-yl]acetate,
Propyl 2-[2-(2-fluorophenyl)-5H-imidazo[4,5-d]pyridazin-5-yl]-2-[3-[4-propyloxy-2-(trifluoromethyl)phenyl]isoxazol-5-yl]acetate,
Propyl 2-[2-(2,3-difluorophenyl)-5H-imidazo[4,5-d]pyridazin-5-yl]-2-[3-[2,4-bis(trifluoromethyl)phenyl]isoxazol-5-yl]acetate,
Propyl 2-[2-(2-fluorophenyl)-5H-imidazo[4,5-d]pyridazin-5-yl]-2-[3-[2,4-bis(trifluoromethyl)phenyl]isoxazol-5-yl]acetate,
2-[2-(2-Fluorophenyl)-5H-imidazo[4,5-d]pyridazin-5-yl]-N-methyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-methyl-2-[3-[2,4-bis(trifluoromethyl)phenyl]isoxazol-5-yl]acetamide,
2-[2-(2-Fluorophenyl)-5H-imidazo[4,5-d]pyridazin-5-yl]-N-methyl-2-[3-[2,4-bis(trifluoromethyl)phenyl]isoxazol-5-yl]acetamide,
N-ethyl-2-[2-(2-Fluorophenyl)-5H-imidazo[4,5-d]pyridazin-5-yl]-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-ethyl-2-[3-[2,4-bis(trifluoromethyl)phenyl]isoxazol-5-yl]acetamide,
N-ethyl-2-[2-(2-fluorophenyl)-5H-imidazo[4,5-d]pyridazin-5-yl]-2-[3-[2,4-bis(trifluoromethyl)phenyl]isoxazol-5-yl]acetamide,
2-[2-(2-Fluorophenyl)-5H-imidazo[4,5-d]pyridazin-5-yl]-2-[3-[4-propyloxy-2-(trifluoromethyl)phenyl]isoxazol-5-yl]-N-propyl-acetamide,
2-[2-(2,3-Difluorophenyl)-5H-imidazo[4,5-d]pyridazin-5-yl]-2-[3-[2,4-bis(trifluoromethyl)phenyl]isoxazol-5-yl]-N-propyl-acetamide,
2-[2-(2-Fluorophenyl)-5H-imidazo[4,5-d]pyridazin-5-yl]-2-[3-[2,4-bis(trifluoromethyl)phenyl]isoxazol-5-yl]-N-propyl-acetamide,
Methyl 2-[2-(2-fluorophenyl)-5H-imidazo[4,5-d]pyridazin-5-yl]-2-[6-[4-propyloxy-2-(trifluoromethyl)phenyl]-3-pyridinyl]acetate,
Methyl 2-[2-(2,3-difluorophenyl)-5H-imidazo[4,5-d]pyridazin-5-yl]-2-[6-[2,4-bis(trifluoromethyl)phenyl]-3-pyridinyl]acetate,
Methyl 2-[2-(2-fluorophenyl)-5H-imidazo[4,5-d]pyridazin-5-yl]-2-[6-[2,4-bis(trifluoromethyl)phenyl]-3-pyridinyl]acetate,
Ethyl 2-[2-(2-fluorophenyl)-5H-imidazo[4,5-d]pyridazin-5-yl]-2-[6-[4-propyloxy-2-(trifluoromethyl)phenyl]-3-pyridinyl]acetate,
Ethyl 2-[2-(2,3-difluorophenyl)-5H-imidazo[4,5-d]pyridazin-5-yl]-2-[6-[2,4-bis(trifluoromethyl)phenyl]-3-pyridinyl]acetate,
Ethyl 2-[2-(2-fluorophenyl)-5H-imidazo[4,5-d]pyridazin-5-yl]-2-[6-[2,4-bis(trifluoromethyl)phenyl]-3-pyridinyl]acetate,
Propyl 2-[2-(2-fluorophenyl)-5H-imidazo[4,5-d]pyridazin-5-yl]-2-[6-[4-propyloxy-2-(trifluoromethyl)phenyl]-3-pyridinyl]acetate,
Propyl 2-[2-(2,3-difluorophenyl)-5H-imidazo[4,5-d]pyridazin-5-yl]-2-[6-[2,4-bis(trifluoromethyl)phenyl]-3-pyridinyl]acetate,
Propyl 2-[2-(2-fluorophenyl)-5H-imidazo[4,5-d]pyridazin-5-yl]-2-[6-[2,4-bis(trifluoromethyl)phenyl]-3-pyridinyl]acetate,
Methyl 2-[2-(2-fluorophenyl)-5H-imidazo[4,5-d]pyridazin-5-yl]-2-[2-[4-propyloxy-2-(trifluoromethyl)phenyl]-5-pyrimidinyl]acetate,
Methyl 2-[2-(2,3-difluorophenyl)-5H-imidazo[4,5-d]pyridazin-5-yl]-2-[2-[2,4-bis(trifluoromethyl)phenyl]-5-pyrimidinyl]acetate,
Methyl 2-[2-(2-fluorophenyl)-5H-imidazo[4,5-d]pyridazin-5-yl]-2-[2-[2,4-bis(trifluoromethyl)phenyl]-5-pyrimidinyl]acetate,
Ethyl 2-[2-(2-fluorophenyl)-5H-imidazo[4,5-d]pyridazin-5-yl]-2-[2-[4-propyloxy-2-(trifluoromethyl)phenyl]-5-pyrimidinyl]acetate,
Ethyl 2-[2-(2,3-difluorophenyl)-5H-imidazo[4,5-d]pyridazin-5-yl]-2-[2-[2,4-bis(trifluoromethyl)phenyl]-5-pyrimidinyl]acetate,
Ethyl 2-[2-(2-fluorophenyl)-5H-imidazo[4,5-d]pyridazin-5-yl]-2-[2-[2,4-bis(trifluoromethyl)phenyl]-5-pyrimidinyl]acetate,
Propyl 2-[2-(2-fluorophenyl)-5H-imidazo[4,5-d]pyridazin-5-yl]-2-[2-[4-propyloxy-2-(trifluoromethyl)phenyl]-5-pyrimidinyl]acetate,
Propyl 2-[2-(2,3-difluorophenyl)-5H-imidazo[4,5-d]pyridazin-5-yl]-2-[2-[2,4-bis(trifluoromethyl)phenyl]-5-pyrimidinyl]acetate,
Propyl 2-[2-(2-fluorophenyl)-5H-imidazo[4,5-d]pyridazin-5-yl]-2-[2-[2,4-bis(trifluoromethyl)phenyl]-5-pyrimidinyl]acetate,
Methyl 2-[2-(2,3-difluorophenyl)-5H-imidazo[4,5-c]pyridin-5-yl]-2-[6-[2,4-bis(trifluoromethyl)phenyl]pyridazin-3-yl]acetate,
Methyl 2-[2-(2-fluorophenyl)-5H-imidazo[4,5-c]pyridin-5-yl]-2-[6-[4-propyloxy-2-(trifluoromethyl)phenyl]pyridazin-3-yl]acetate,
Methyl 2-[2-(2,3-difluorophenyl)-5H-imidazo[4,5-c]pyridin-5-yl]-2-[6-[4-propyloxy-2-(trifluoromethyl)phenyl]pyridazin-3-yl]acetate,
Ethyl 2-[2-(2,3-difluorophenyl)-5H-imidazo[4,5-c]pyridin-5-yl]-2-[6-[2,4-bis(trifluoromethyl)phenyl]pyridazin-3-yl]acetate,
Ethyl 2-[2-(2-fluorophenyl)-5H-imidazo[4,5-c]pyridin-5-yl]-2-[6-[4-propyloxy-2-(trifluoromethyl)phenyl]pyridazin-3-yl]acetate,
Ethyl 2-[2-(2,3-difluorophenyl)-5H-imidazo[4,5-c]pyridin-5-yl]-2-[6-[4-propyloxy-2-(trifluoromethyl)phenyl]pyridazin-3-yl]acetate,
Propyl 2-[2-(2,3-difluorophenyl)-5H-imidazo[4,5-c]pyridin-5-yl]-2-[6-[2,4-bis(trifluoromethyl)phenyl]pyridazin-3-yl]acetate,
Propyl 2-[2-(2-fluorophenyl)-5H-imidazo[4,5-c]pyridin-5-yl]-2-[6-[4-propyloxy-2-(trifluoromethyl)phenyl]pyridazin-3-yl]acetate,
Propyl 2-[2-(2,3-difluorophenyl)-5H-imidazo[4,5-c]pyridin-5-yl]-2-[6-[4-propyloxy-2-(trifluoromethyl)phenyl]pyridazin-3-yl]acetate,
Methyl 2-[8-(2,3-difluorophenyl)-1H-purin-1-yl]-2-[6-[2,4-bis(trifluoromethyl)phenyl]pyridazin-3-yl]acetate,
Methyl 2-[8-(2-fluorophenyl)-1H-purin-1-yl]-2-[6-[4-propyloxy-2-(trifluoromethyl)phenyl]pyridazin-3-yl]acetate,
Methyl 2-[8-(2,3-difluorophenyl)-1H-purin-1-yl]-2-[6-[4-propyloxy-2-(trifluoromethyl)phenyl]pyridazin-3-yl]acetate,
Ethyl 2-[8-(2,3-difluorophenyl)-1H-purin-1-yl]-2-[6-[2,4-bis(trifluoromethyl)phenyl]pyridazin-3-yl]acetate,
Ethyl 2-[8-(2-fluorophenyl)-1H-purin-1-yl]-2-[6-[4-propyloxy-2-(trifluoromethyl)phenyl]pyridazin-3-yl]acetate,
Ethyl 2-[8-(2,3-difluorophenyl)-1H-purin-1-yl]-2-[6-[4-propyloxy-2-(trifluoromethyl)phenyl]pyridazin-3-yl]acetate,
Propyl 2-[8-(2,3-difluorophenyl)-1H-purin-1-yl]-2-[6-[2,4-bis(trifluoromethyl)phenyl]pyridazin-3-yl]acetate,
Propyl 2-[8-(2-fluorophenyl)-1H-purin-1-yl]-2-[6-[4-propyloxy-2-(trifluoromethyl)phenyl]pyridazin-3-yl]acetate,
Propyl 2-[8-[(2,3-difluorophenyl)methyl]-1H-purin-1-yl]-2-[6-[4-propyloxy-2-(trifluoromethyl)phenyl]pyridazin-3-yl]acetate,
2-Methylpropyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
Phenylmethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
4-(Dimethylamino)butyl [2-(2,3-difluorophenyl)-5H-imidazo[4,5-d]pyridazin-5-yl]{3-[4-(propyloxy)-2-(trifluoromethyl)phenyl]-5-isoxazolyl}acetate dihydrochloride,
4-(Dimethylamino)butyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanine sodium salt,
N-{3-[([2-(2,3-Difluorophenyl)-5H-imidazo[4,5-d]pyridazin-5-yl113-[4-(propyloxy)-2-(trifluoromethyl)phenyl]-5-isoxazolyl}acetyl)oxy]propanoyl]-L-alanine,
N-{3-[([2-(2,3-Difluorophenyl)-5H-imidazo[4,5-d]pyridazin-5-yl113-[4-(propyloxy)-2-(trifluoromethyl)phenyl]-5-isoxazolyl}acetyl)oxy]propanoyl]-L-alanyl-L-phenylalanine,
Methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
2-[(2-hydroxyethyl)oxy]ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
3-hydroxy-2-(hydroxymethyl)-2-methylpropyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
Ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
3-Hydroxypropyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
Methyl {3-[4-cyclobutyl-2-(trifluoromethyl)phenyl]-5-isoxazolyl}[2-(2,3-difluorophenyl)-5H-imidazo[4,5-d]pyridazin-5-yl]acetate,
Propyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
4-hydroxybutyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
3-({bis[(1,1-dimethylethyl)oxy]phosphoryl}oxy)propyl {3-[4-cyclopropyl-2-(trifluoromethyl)phenyl]-5-isoxazolyl}[2-(2,3-difluorophenyl)-5H-imidazo[4,5-d]pyridazin-5-yl]acetate, and
Methyl [2-(2-fluorophenyl)-5H-imidazo[4,5-c]pyridin-5-yl]{3-[4-(propyloxy)-2-(trifluoromethyl)phenyl]-5-isoxazolyl}acetate,
   and pharmaceutically acceptable salts and solvates thereof.

In some embodiments, the present invention provides a compound having the structure:

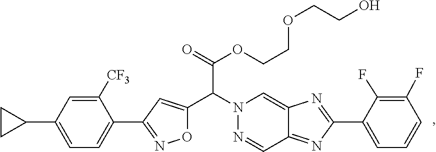

or a pharmaceutically acceptable salt thereof.

In some embodiments, the present invention provides the compound: 2-[(2-hydroxyethyl)oxy]ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate; or a pharmaceutically acceptable salt thereof.

Also provided are compounds selected from Table 1 or a pharmaceutically acceptable salt or solvate thereof.

TABLE 1

| Cmpd | | |
|---|---|---|
| # | Structure | Name |
| 1 | 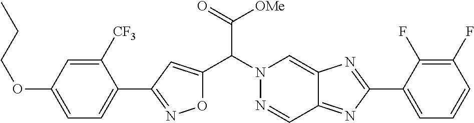 | Methyl [2-(2,3-difluorophenyl)-5H- imidazo[4,5-d]pyridazin-5-yl]{3-[4- (propyloxy)-2- (trifluoromethyl)phenyl]-5- isoxazolyl}acetate |
| 2 | 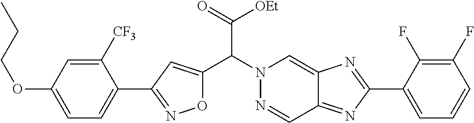 | Ethyl [2-(2,3-difluorophenyl)-5H- imidazo[4,5-d]pyridazin-5-yl]{3-[4- (propyloxy)-2- (trifluoromethyl)phenyl]-5- isoxazolyl}acetate |
| 3 | 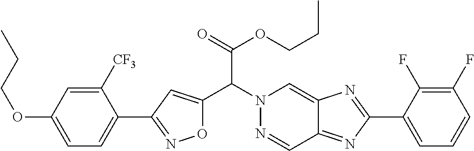 | Propyl [2-(2,3-difluorophenyl)-5H- imidazo[4,5-d]pyridazin-5-yl]{3[4- (propyloxy)-2- (trifluoromethyl)phenyl]-5- isoxazolyl}acetate |
| 4 | 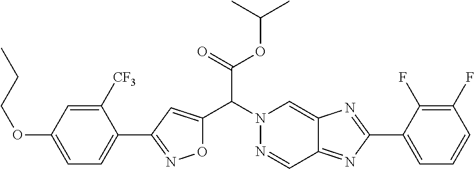 | 1-Methylethyl [2-(2,3- difluorophenyl)-5H-imidazo[4,5- d]pyridazin-5-yl]{3-[4-(propyloxy)- 2-(trifluoromethyl)phenyl]-5- isoxazolyl}acetate |
| 5 | 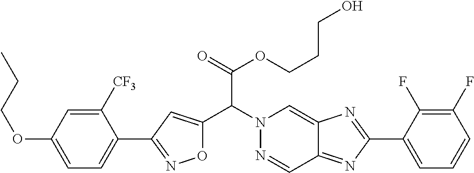 | 3-Hydroxypropyl [2-(2,3- difluorophenyl)-5H-imidazo[4,5- d]pyridazin-5-yl]{3-[4-(propyloxy)- 2-(trifluoromethyl)phenyl]-5- isoxazolyl}acetate |
| 6 | 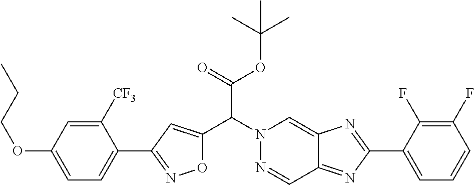 | 1,1-Dimethylethyl [2-(2,3- difluorophenyl)-5H-imidazo[4,5- d]pyridazin-5-yl]{3-[4-(propyloxy)- 2-(trifluoromethyl)phenyl]-5- isoxazolyl}acetate |
| 7 | 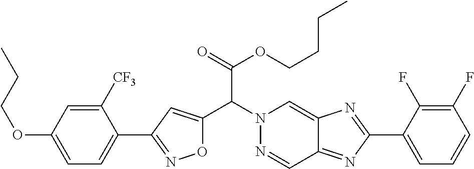 | Butyl [2-(2,3-difluorophenyl)-5H- imidazo[4,5-d]pyridazin-5-yl]{3-[4- (propyloxy)-2- (trifluoromethyl)phenyl]-5- isoxazolyl}acetate |
| 8 | 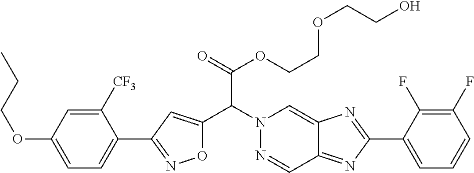 | 2-[(2-Hydroxyethyl)oxy]ethyl [2- (2,3-difluorophenyl)-5H- imidazo[4,5-d]pyridazin-5-yl]{3-[4- (propyloxy)-2- (trifluoromethyl)phenyl]-5- isoxazolyl}acetate |
| 9 | 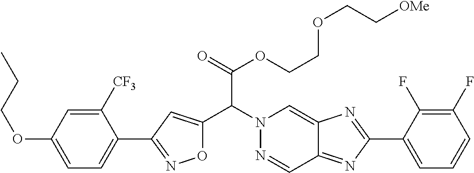 | 2-{[2-(Methyloxy)ethyl]oxy}ethyl [2-(2,3-difluorophenyl)-5H- imidazo[4,5-d]pyridazin-5-yl]{3-[4- (propyloxy)-2- (trifluoromethyl)phenyl]-5- isoxazolyl}acetate |
| 10 | 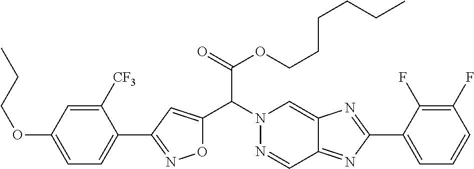 | Hexyl [2-(2,3-difluorophenyl )-5H- imidazo[4,5-d]pyridazin-5-yl]{3-[4- (propyloxy)-2- (trifluoromethyl)phenyl]-5- isoxazolyl}acetate |
| 11 | 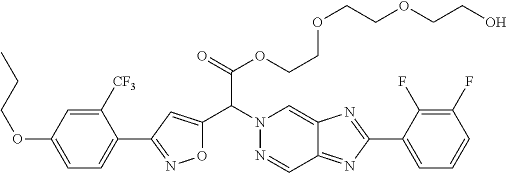 | 2-({2-[(2- Hydroxyethyl)oxy]ethyl}oxy) ethyl [2-(2,3-difluorophenyl)-5H- imidazo[4,5-d]pyridazin-5-yl]{3-[4- (propyloxy)-2- (trifluoromethyl)phenyl]-5- isoxazolyl}acetate |
| 12 | 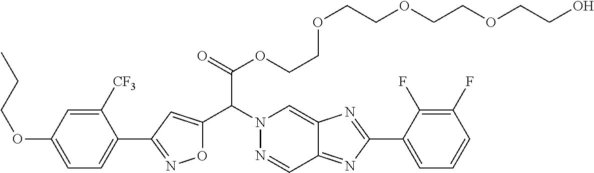 | 2-{[2-({2-[(2- Hydroxyethyl)oxy]ethyl}oxy)ethyl] oxy}ethyl [2-(2,3-difluorophenyl)- 5H-imidazo[4,5-d]pyridazin-5- yl]{3-[4(propyloxy)-2- (trifluoromethyl)phenyl]-5- isoxazolyl}acetate |
| 13 | 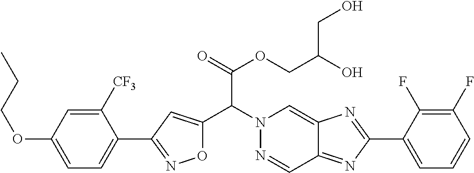 | 2,3-Dihydroxypropyl 2-[2-(2,3- difluorophenyl)-5H-imidazo[4,5- d]pyridazin-5-yl]-2-[3-[4-propyloxy- 2-(trifluoromethyl)phenyl]isoxazol- 5-yl]acetate |
| 14 | 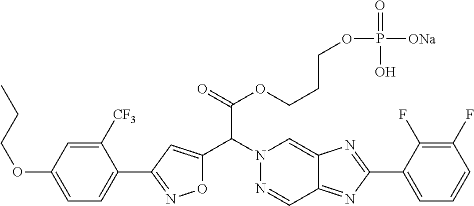 | 3-(Phosphonooxy)propyl[2-(2,3- difluorophenyl)-5H-imidazo[4,5- d]pyridazin-5-yl]{3-[4-(propyloxy)- 2-(trifluoromethyl)phenyl]-5- isoxazolyl}acetate mono sodium salt |
| 14a | 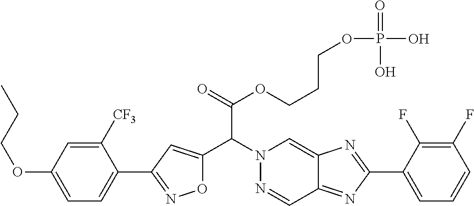 | 3-(Phosphonooxy)propyl[2-(2,3- difluorophenyl)-5H-imidazo[4,5- d]pyridazin-5-yl]{3-[4-(propyloxy)- 2-(trifluoromethyl)phenyl]-5- isoxazolyl]acetate |
| 15 | 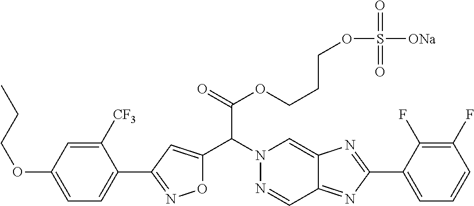 | 3-(Sulfooxy)propyl [2-(2,3- difluorophenyl)-5H-imidazo[4,5- d]pyridazin-5-yl]{3-[4-(propyloxy)- 2-(trifluoromethyl)phenyl]-5- isoxazolyl}acetate sodium salt |
| 15a | 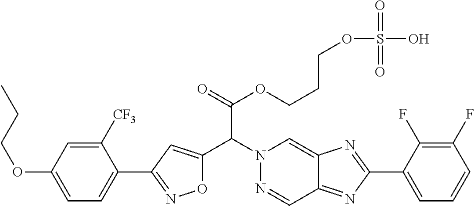 | 3-(Sulfooxy)propyl [2-(2,3- difluorophenyl)-5H-imidazo[4,5- dpyridazin-5-yl]{3-[4-(propyloxy)- 2-(trifluoromethyl)phenyl]-5- isoxazolyl}acetate |
| 16 | 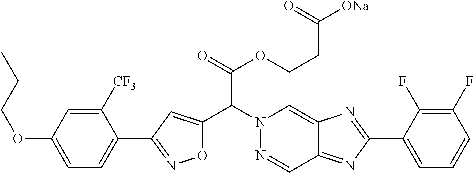 | 3-[([2-(2,3-Difluorophenyl)-5H- imidazo[4,5-d]pyridazin-5-yl]{3-[4- (propyloxy)-2- (trifluoromethyl)phenyl]-5- isoxazolyl}acetyl)oxy] propanoic acid sodium salt |
| 16a | 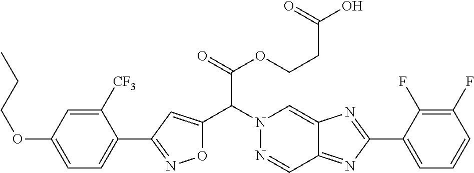 | 3-[([2-(2,3-Difluorophenyl)-5H- imidazo[4,5-d]pyridazin-5-yl]{3-[4- (propyloxy)-2- (trifluoromethyl)phenyl]-5- isoxazolyl}acetyl)oxy] propanoic acid |
| 17 | 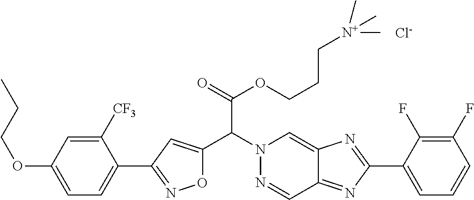 | 3-[2-[2-(2,3-Difluorophenyl)-5H- imidazo[4,5-d]pyridazin-5-yl]-2-[3- [4-propyloxy-2- (trifluoromethyl)phenyl]isoxazol-5- yl]acetyl]oxypropyl-trimethyl- ammonium chloride |
| 18 | 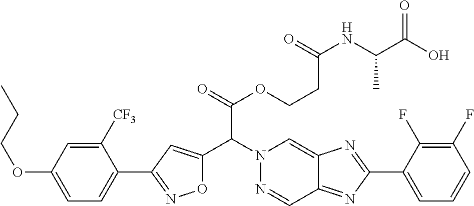 | N-{3-[([2-(2,3-Difluorophenyl)- 5H-imidazo[4,5-d]pyridazin-5- yl]{3-[4-(propyloxy)-2- (trifluoromethyl)phenyl]-5- isoxazolyl}acetyl)oxy]propanoyl}- L-alanine |
| 19 | 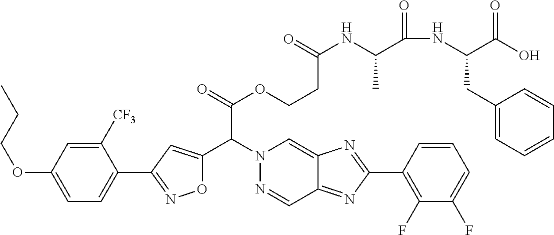 | N-{3-[([2-(2,3-Difluorophenyl)- 5H-imidazo[4,5-d]pyridazin-5- yl]{3-[4-(propyloxy)-2- (trifluoromethyl)phenyl]-5- isoxazolyl}acetyl)oxy]propanoyl}- L-alanyl-L-phenylalanine |
| 20 | 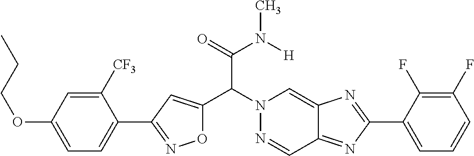 | 2-[2-(2,3-Difluorophenyl)-5H- imidazo[4,5-d]pyridazin-5-yl]-N- methyl-2-{3-[4-(propyloxy)-2- (trifluoromethyl)phenyl]-5- isoxazolyl}acetamide |
| 21 | 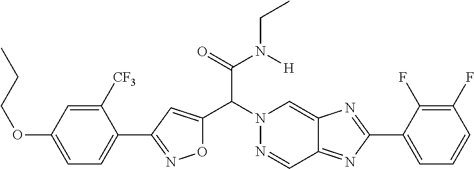 | 2-[2-(2,3-Difluorophenyl)-5H- imidazo[4,5-d]pyridazin-5-yl]-N- ethyl-2-[3-[4-propyloxy-2- (trifluoromethyl)phenyl]isoxazol-5- yl]acetamide |
| 22 | 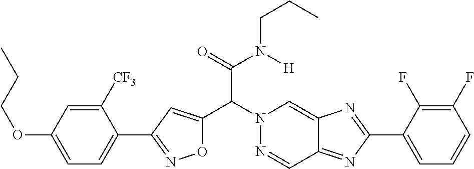 | 2-[2-(2,3-Difluorophenyl)-5H- imidazo[4,5-d]pyridazin-5-yl]-N- propyl-2-[3-[4-propyloxy-2- (trifluoromethyl)phenyl]isoxazol-5- yl]acetamide |
| 23 | 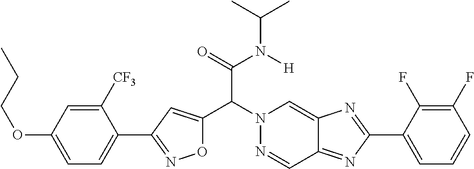 | 2-[2-(2,3-Difluorophenyl)-5H- imidazo[4,5-d]pyridazin-5-yl]-N- isopropyl-2-[3-[4-propyloxy-2- (trifluoromethyl)phenyl] isoxazol-5-yl]acetamide |
| 24 | 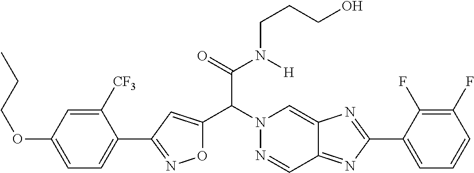 | 2-[2-(2,3-Difluorophenyl)-5H- imidazo[4,5-d]pyridazin-5-yl]-N-(3- hydroxypropyl)-2-[3-[4-propyloxy- 2-trifluoromethyl)phenyl]isoxazol- 5-yl]acetamide |
| 25 | 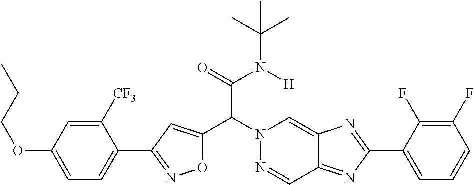 | 2-[2-(2,3-difluorophenyl)-5H- imidazo[4,5-d]pyridazin-5-yl]-N- tert-butyl-2-[3-[4-propyloxy-2- (trifluoromethyl)phenyl] isoxazol-5-yl]acetamide |
| 26 | 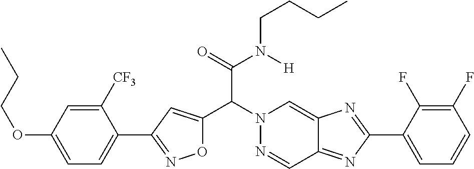 | 2-[2-(2,3-Difluorophenyl)-5H- imidazo[4,5-d]pyridazin-5-yl]-N- butyl-2-[3-[4-propyloxy-2- (trifluoromethyl)phenyl]isoxazol-5- yl]acetamide |
| 27 | 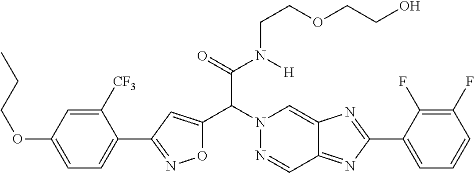 | 2-[2-(2,3-Difluorophenyl)-5H- imidazo[4,5-d]pyridazin-5-yl]-N-[2- (2-hydroxyethoxy)ethyl]-2-[3-[4- propyloxy)-2- (trifluoromethyl)phenyl]isoxazol-5- yl]acetamide |
| 28 | 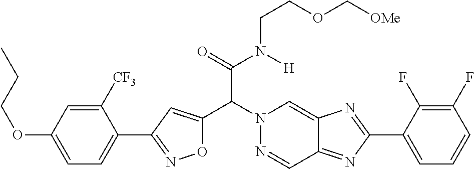 | 2-[2-(2,3-Difluorophenyl)-5H- imidazo[4,5-d]pyridazin-5-yl]-N-[2- (2-methoxyethoxy)ethyl]-2-[3-[4- propyloxy)-2- (trifluoromethyl)phenyl]isoxazol-5- yl]acetamide |
| 29 | 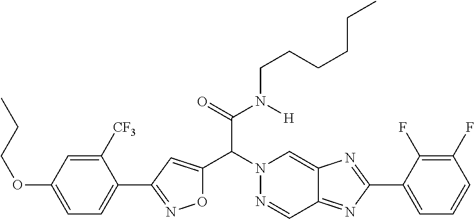 | 2-[2-(2,3-Difluorophenyl)-5H- imidazo[4,5-d]pyridazin-5-yl]-N- hexyl-2-{3-[4-propyloxy-2- (trifluoromethyl)phenyl]isoxazol-5- yl]acetamide |
| 30 | 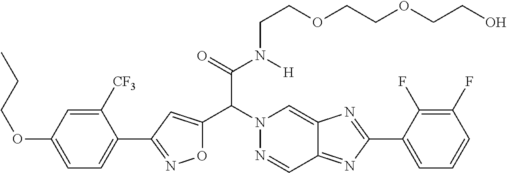 | 2-[2-(2,3-Difluorophenyl)-5H- imidazo[4,5-d]pyridazin-5-yl]-N-[2- [2-(2-hydroxyethoxy)ethoxy]ethyl]- 2-[3-[4- propyloxy-2- (trifluoromethyl)phenyl]isoxazol-5- yl]acetamide |
| 31 | 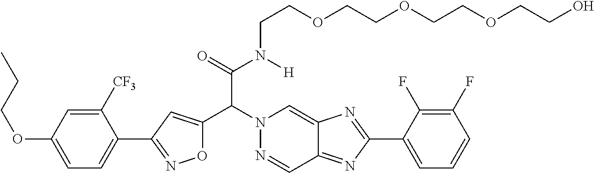 | 2-[2-(2,3-Difluorophenyl)-5H- imidazo[4,5-d]pyridazin-5-yl]-N-[2- [2-[2-(2- hydroxyethoxy)ethoxy]ethoxy]ethyl]- 2-[3-[4- propyloxy-2- (trifluoromethyl)phenyl]isoxazol-5- yl]acetamide |
| 32 | 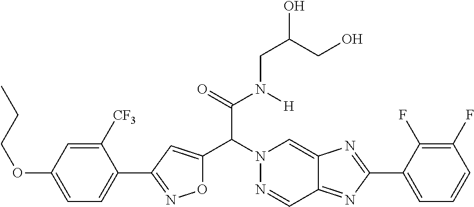 | 2-[2-(2,3-Difluorophenyl)-5H- imidazo[4,5-d]pyridazin-5-yl]-N- (2,3-dihydroxypropyl)-2-[3-[4- propyloxy-2- (trifluoromethyl)phenyl]isoxazol-5- yl]acetamide |
| 33 | 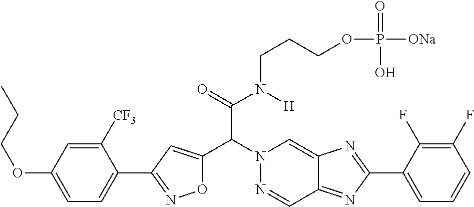 | N-3-(phosphonooxy)propyl-2-[2- (2,3-difluorophenyl)-5H- imidazo[4,5-d]pyridazin-5-yl]-2-[3- [4-propyloxy-2- (trifluoromethyl)phenyl]isoxazol-5- yl]acetamide mono sodium salt |
| 33a | 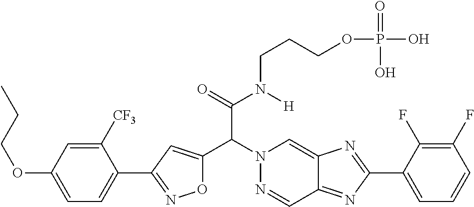 | N-3-(phosphonooxy)propyl-2-[2- (2,3-difluorophenyl)-5H- imidazo[4,5-d]pyridazin-5-yl]-2-[3- [4-propyloxy-2- (trifluoromethyl)phenyl]isoxazol-5- yl]acetamide |
| 34 | 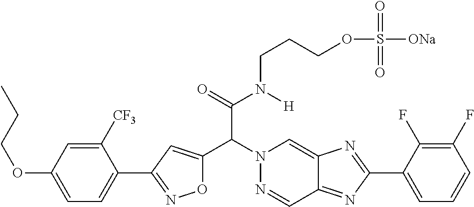 | N-3-(Sulfonyloxy)propyl-2-[2-(2,3- difluorophenyl)-5H-imidazo[4,5- d]pyridazin-5-yl]-2-[3-[4-propyloxy- 2-(trifluoromethyl)phenyl]isoxazol- 5-yl]acetamide mono sodium salt |
| 34a | 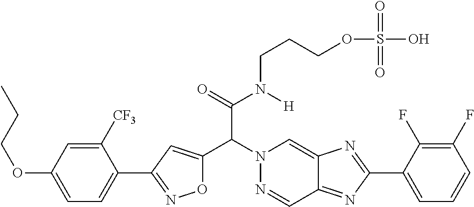 | N-3-(Sulfonyloxy)propyl-2-[2-(2,3- difluorophenyl)-5H-imidazo[4,5- d]pyridazin-5-yl]-2-[3-[4-propyloxy- 2-(trifluoromethyl)phenyl]isoxazol- 5-yl]acetamide |
| 35 | 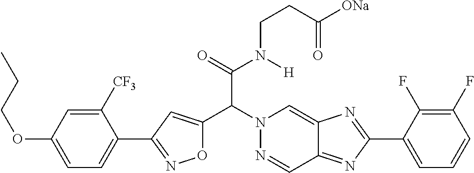 | 3-[[2-[2-(2,3-Difluorophenyl)-5H- imidazo[4,5-d]pyridazin-5-yl]-2-[3- [4-propyloxy-2- (trifluoromethyl)phenyl]isoxazol-5- yl]acetyl]amino]propanoic acid sodium salt |
| 35a | 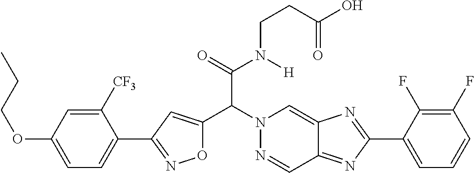 | 3-[[2-[2-(2,3-Difluorophenyl)-5H- imidazo[4,5-d]pyridazin-5-yl]-2-[3- [4-propyloxy-2- (trifluoromethyl)phenyl]isoxazol-5- yl]acetyl]amino]propanoic acid |
| 36 | 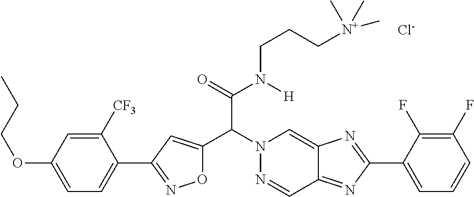 | 3-[[2-[2-(2,3-Difluorophenyl)-5H- imidazo[4,5-d]pyridazin-5-yl]-2-[3- [4-propyloxy-2- (trifluoromethyl)phenyl]isoxazol-5- yl]acetyl]amino]propyl-trimethyl- ammonium chloride |
| 37 | 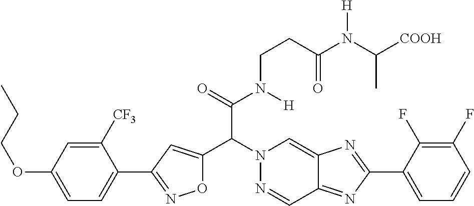 | 2-[3-[[2-[2-(2,3-Difluorophenyl)- 5H-imidazo[4,5-d]pyridazin-5-yl]- 2-[3-[4-propyloxy-2- (trifluoromethyl)phenyl]isoxazol-5- yl]acetyl]amino]propanoylamino] propanoic acid |
| 38 | 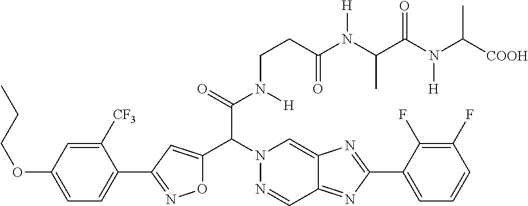 | 2-[3-[[2-[2-(2,3-Difluorophenyl)- 5H-imidazo[4,5-d]pyridazin-5-yl]- 2-[3-[4-propyloxy-2- (trifluoromethyl)phenyl]isoxazol-5- yl]acetyl]amino]propanoyl amino]propanoylamino] propanoic acid |
| 39 | 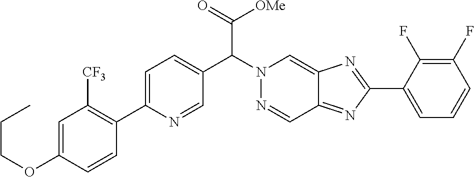 | Methyl [2-(2,3-difluorophenyl)-5H- imidazo[4,5-d]pyridazin-5-yl]{6-[4- (propyloxy)-2- (trifluoromethyl)phenyl]-3- pyridinyl}acetate |
| 40 | 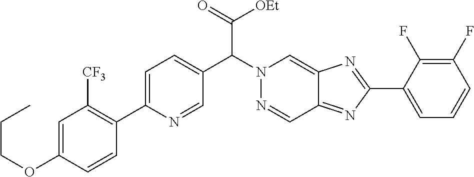 | Ethyl [2-(2,3-difluorophenyl)-5H- imidazo[4,5-d]pyridazin-5-yl]{6-[4- (propyloxy)-2- (trifluoromethyl)phenyl]-3- pyridinyl}acetate |
| 41 | 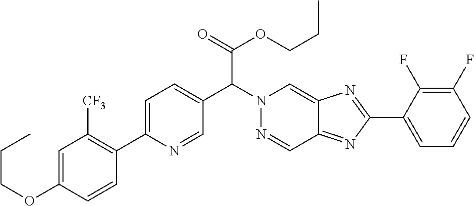 | Propyl [2-(2,3-difluorophenyl)-5H- imidazo[4,5-d]pyridazin-5-yl]{6-[4- (propyloxy)-2- (trifluoromethyl)phenyl]-3- pyridinyl}acetate |
| 42 | 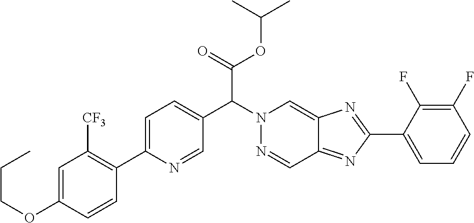 | 1-Methylethyl [2-(2,3- difluorophenyl)-5H-imidazo[4,5- d]pyridazin-5-yl]{6-[4-(propyloxy)- 2-(trifluoromethyl)phenyl]-3- pyridinyl}acetate |
| 43 | 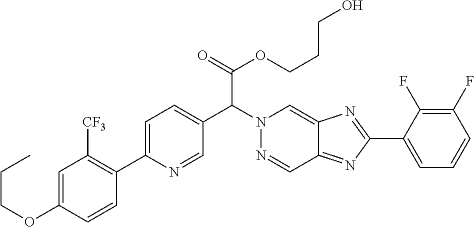 | 3-Hydroxypropyl 2-[2-(2,3- difluorophenyl)-5H-imidazo[4,5- d]pyridazin-5-yl]-2-{6- [4-propyloxy- 2-(trifluoromethyl)phenyl]-3- pyridinyl]acetate |
| 44 | 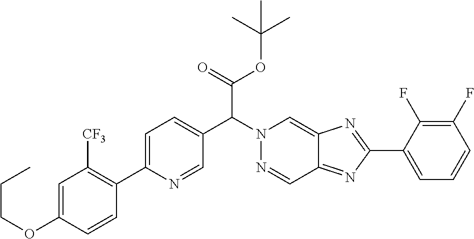 | Tert-butyl 2-[2-(2,3- difluorophenyl)-5H-imidazo[4,5- c]pyridazin-5-yl]-2-[6-[4-propyloxy- 2-(trifluoromethyl)phenyl]-3- pyridinyl]acetate |
| 45 | 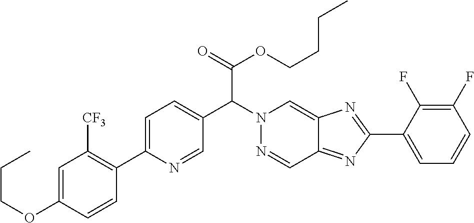 | Butyl 2-[2-(2,3-difluorophenyl)-5H- imidazo[4,5-d]pyridazin-5-yl]-2-[6- [4-propyloxy-2- (trifluoromethyl)phenyl]-3- pyridinyl]acetate |
| 46 | 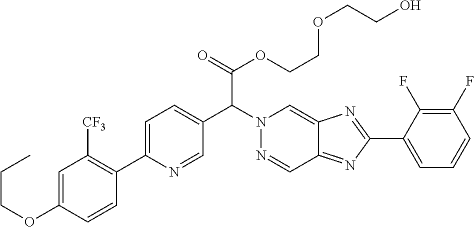 | 2-(2-Hydroxyethoxy)ethyl 2-[2- (2,3-difluorophenyl)-5H- imidazo[4,5-d]pyridazin-5-yl]-2-[6- [4-propyloxy-2- (trifluoromethyl)phenyl]-3- pyridinyl]acetate |
| 47 | 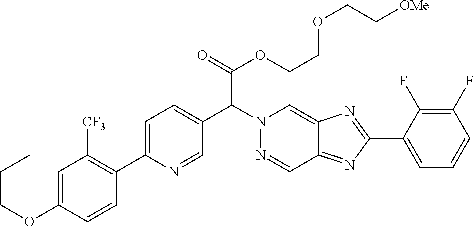 | 2-(2-Methoxyethoxy)ethyl 2-[2- (2,3-difluorophenyl)-5H- imidazo[4,5-d]pyridazin-5-yl]-2-[6- [4-propyloxy-2- (trifluoromethyl)phenyl]-3- pyridinyl]acetate |
| 48 | 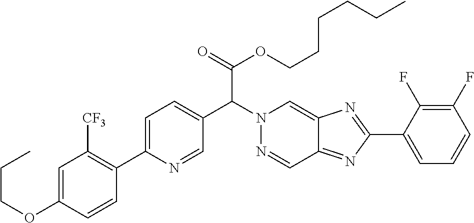 | Hexyl 2-[2-(2,3-difluorophenyl)- 5H-imidazo[4,5-d]pyridazin-5-yl]- 2-[6-[4-propyloxy-2- (trifluoromethyl)phenyl]-3- pyridinyl]acetate |
| 49 | 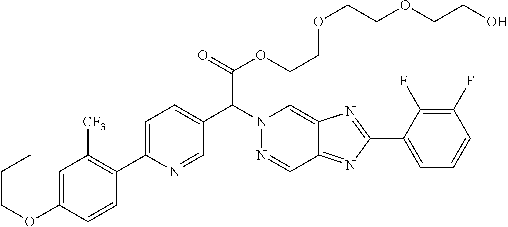 | 2-[2-(2- Hydroxyethoxy)ethoxy]ethyl 2-[2- (2,3-difluorophenyl)-5H- imidazo[4,5-d] pyridazin-5-yl]-2-[6- [4-propyloxy-2- (trifluoromethyl)phenyl]-3- pyridinyl]acetate |
| 50 | 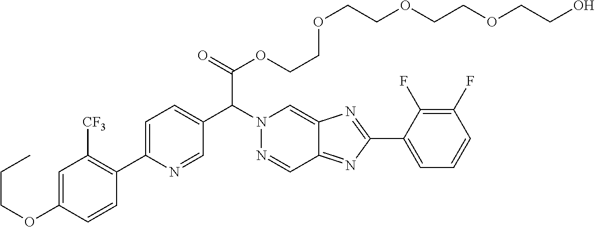 | 2-[2-[2-(2- Hydroxyethoxy)ethoxy]ethyl 2-[2- (2,3-difluorophenyl)-5H- imidazo[4,5-d] pyridazin-5-yl]-2-[6- [4-propyloxy-2- (trifluoromethyl)phenyl]-3- pyridinyl]acetate |
| 51 | 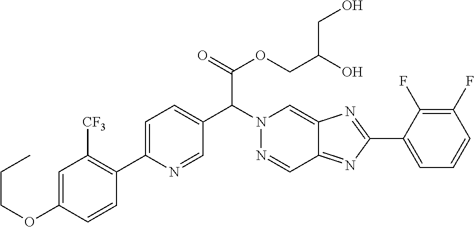 | 2,3-Dihydroxypropyl 2-[2-(2,3- difluorophenyl)-5H-imidazo[4,5- d]pyridazin-5-yl]-2- [6-[4-propyloxy- 2-(trifluoromethyl)phenyl]-3- pyridinyl]acetate |
| 52 | 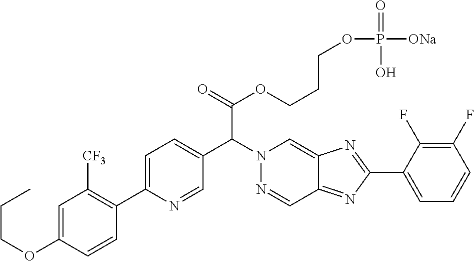 | 3-(Phosphonooxy)propyl 2-[2-(2,3- difluorophenyl)-5H-imidazo[4,5- d]pyridazin-5-yl]-2- [6-[4-propyloxy- 2-(trifluoromethyl)phenyl]-3- pyridinyl]acetate mono sodium salt |
| 52a | 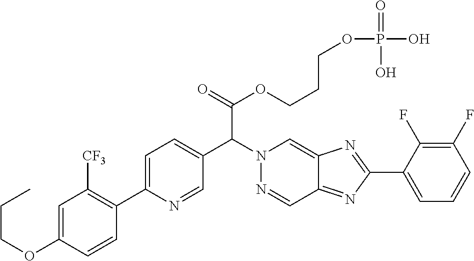 | 3-(Phosphonooxy)propyl 2-[2-(2,3- difluorophenyl)-5H-imidazo[4,5- d]pyridazin-5-yl]-2- [6-[4-propyloxy- 2-(trifluoromethyl)phenyl]-3- pyridinyl]acetate |
| 53 | 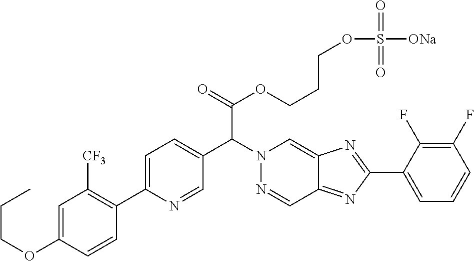 | 3-(Sulfonyloxy)propyl 2-[2-(2,3- difluorophenyl)-5H-imidazo[4,5- d]pyridazin-5-yl]-2- [6-[4-propyloxy- 2-(trifluoromethyl)phenyl]-3- pyridinyl]acetate mono sodium salt |
| 53a | 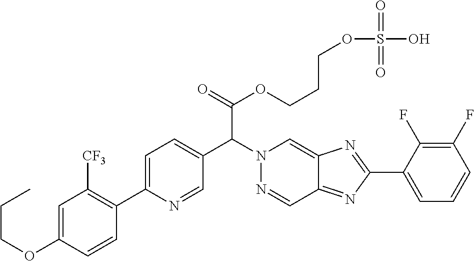 | 3-(Sulfonyloxy)propyl 2-[2-(2,3- difluorophenyl)-5H-imidazo[4,5- d]pyridazin-5-yl]-2- [6-[4-propyloxy- 2-(trifluoromethyl)phenyl]-3- pyridinyl]acetate |
| 54 | 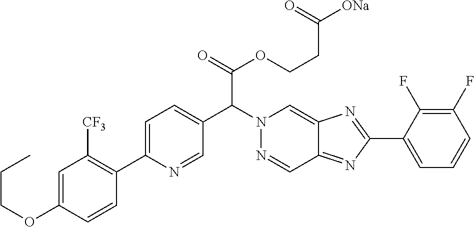 | 3-[2-[2-(2,3-Difluorophenyl)-5H- imidazo[4,5-d]pyridazin-5-yl]-2-[6- [4-propyloxy-2- (trifluoromethyl)phenyl]-3- pyridinyl]acetyl]oxypropanoic acid sodium salt |
| 54a | 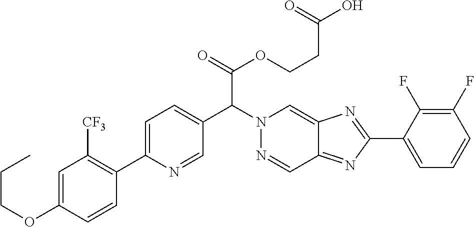 | 3-[2-[2-(2,3-Difluorophenyl)-5H- imidazo[4,5-d]pyridazin-5-yl]-2-[6- [4-propyloxy-2- (trifluoromethyl)phenyl]-3- pyridinyl]acetyl]oxypropanoic acid |
| 55 | 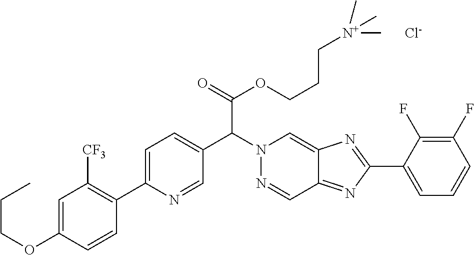 | 3-[2-[2-(2,3-Difluorophenyl)-5H- imidazo[4,5-d]pyridazin-5-yl]-2-[6- [4-propyloxy-2- (trifluoromethyl)phenyl]-3- pyridinyl]acetyl]oxypropyl- dimethyl-ammonium chloride |
| 56 | 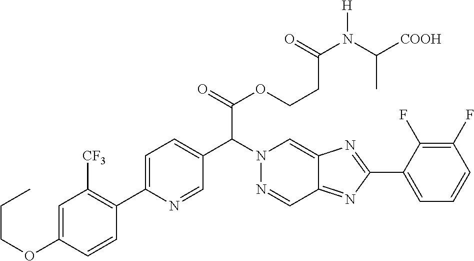 | 2-[3-[2-[2-(2,3-Difluorophenyl)-5H- imidazo[4,5-d]pyridazin-5-yl]-2-[6- [4-propyloxy-2- (trifluoromethyl)phenyl]-3- pyridinyl]acetyl]oxypropanoyl amino]propanoic acid |
| 57 | 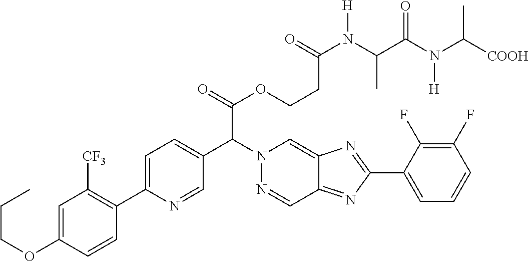 | 2-[2-[3-[2-[2-(2,3-Difluorophenyl)- 5H-imidazo[4,5-d]pyridazin-5-yl]- 2-[6-[4-propyloxy-2- (trifluoromethyl)phenyl]-3- pyridinyl]acetyl]oxypropanoyl amino]propanoylamino] propanoic acid |
| 58 | 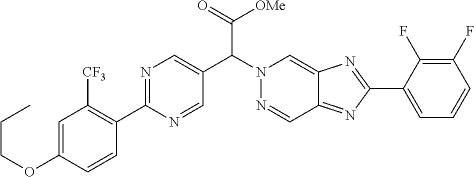 | Methyl [2-(2,3-difluorophenyl)-5H- imidazo[4,5-d]pyridazin-5-yl]{2-[4- (propyloxy)-2- (trifluoromethyl)phenyl]-5- pyrimidinyl}acetate |
| 59 | 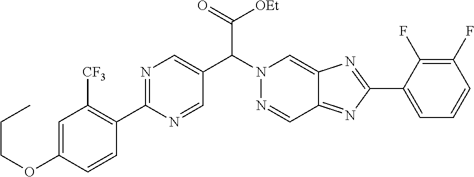 | Ethyl 2-[2-(2,3-difluorophenyl)-5H- imidazo[4,5-d]pyridazin-5-yl]-2-[2- [4-propyloxy-2- (trifluoromethyl)phenyl]-5- pyrimidinyl]acetate |
| 60 | 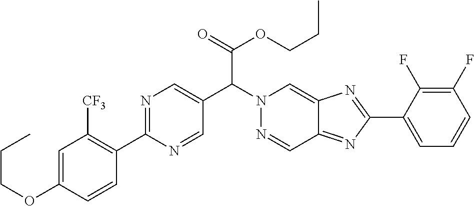 | Propyl 2-[2-(2,3-difluorophenyl)- 5H-imidazo[4,5-d]pyridazin-5-yl]- 2-[2-[4- propyloxy-2- (trifluoromethyl)phenyl]-5- pyrimidinyl]acetate |
| 61 | 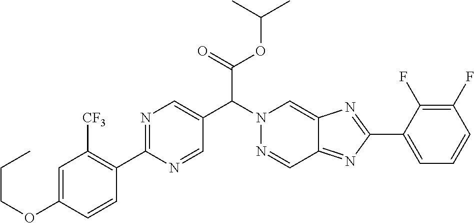 | Isopropyl 2-[2-(2,3- difluorophenyl)-5H-imidazo[4,5- d]pyridazin-5-yl]- 2-[2-[4-propyloxy- 2-(trifluoromethyl)phenyl]-5- pyrimidinyl]acetate |
| 62 | 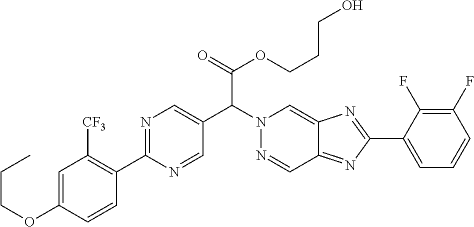 | 3-Hydroxypropyl 2-[2-(2,3- difluorophenyl)-5H-imidazo[4,5- d]pyridazin-5-yl]- 2-[2-[4-propyloxy- 2-(trifluoromethyl)phenyl]-5- pyrimidinyll]acetate |
| 63 | 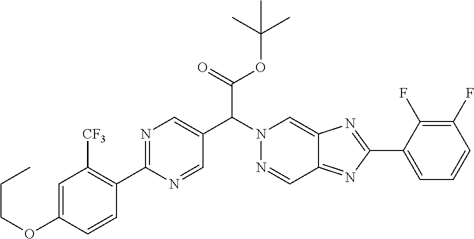 | Tert-butyl 2-[2-(2,3- difluorophenyl)-5H-imidazo[4,5- d]pyridazin-5-yl]- 2-[2-[4-propyloxy- 2-(trifluoromethyl)phenyl]-5- pyrimidinyl]acetate |
| 64 | 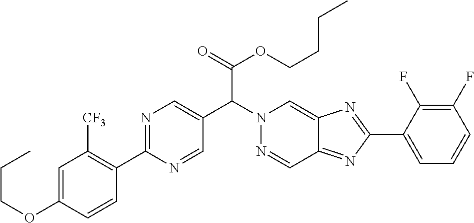 | Butyl 2-[2-(2,3-difluorophenyl)-5H- imidazo[4,5-d]pyridazin-5-yl]-2-[2- [4-propyloxy-2- (trifluoromethyl)phenyl]-5- pyrimidinyl]acetate |
| 65 | 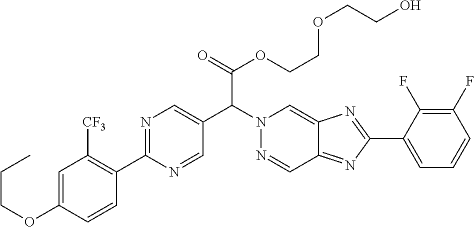 | 2-(2-Hydroxyethoxy)ethyl 2-[2- (2,3-difluorophenyl)-5H- imidazo[4,5-d] pyridazin-5-yl]-2-[2- [4-propyloxy-2- (trifluoromethyl)phenyl]-5- pyrimidinyl]acetate |
| 66 | 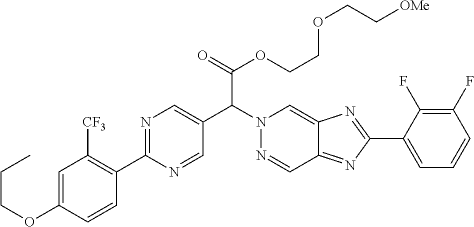 | 2-(2-Methoxyethoxy)ethyl 2-[2- (2,3-difluorophenyl)-5H- imidazo[4,5-d] pyridazin-5-yl]-2-[2- [4-propyloxy-2- (trifluoromethyl)phenyl]-5- pyrimidinyl]acetate |
| 67 | 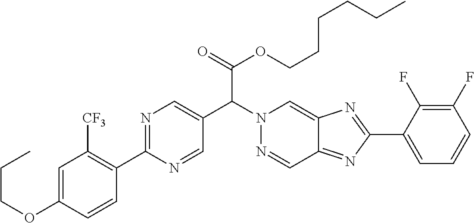 | Hexyl 2-[2-(2,3-difluorophenyl)- 5H-imidazo[4,5-d] pyridazin-5-yl]- 2-[2-[4-propyloxy-2- (trifluoromethyl)phenyl]-5- pyrimidinyl]acetate |
| 68 | 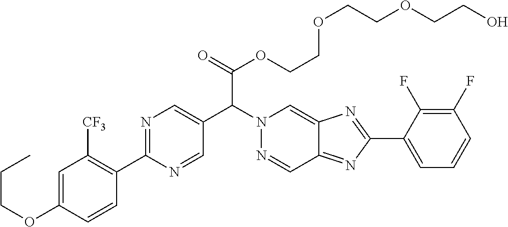 | 2-[2-(2- Hydroxyethoxy)ethoxy]ethyl 2-[2- (2,3-difluorophenyl)-5H- imidazo[4,5-d] pyridazin-5-yl]-2-[2- [4-propyloxy-2- (trifluoromethyl)phenyl]-5- pyrimidinyl]acetate |
| 69 | 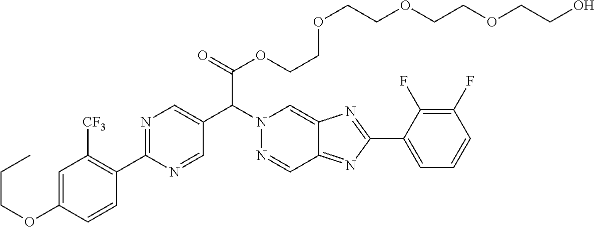 | 2-[2-[2-(2- Hydroxyethoxy)ethoxy]ethoxy]ethyl 2-[2- (2,3-difluorophenyl)-5H- imidazo[4,5-d] pyridazin-5-yl]-2-[2- [4-propyloxy-2- (trifluoromethyl)phenyl]-5- pyrimidinyl]acetate |
| 70 | 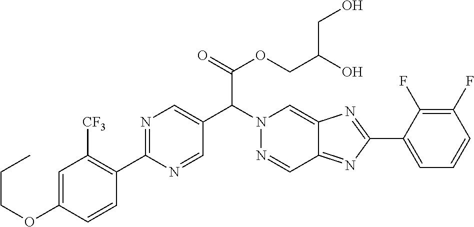 | 2,3-Dihydroxypropyl 2-[2-(2,3- difluorophenyl)-5H-imidazo[4,5- d]pyridazin-5-yl]-2- [2-[4-propyloxy- 2-(trifluoromethyl)phenyl]-5- pyrimidinyl]acetate |
| 71 | 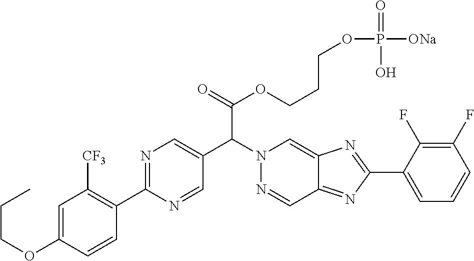 | 3-(Phosphonooxy)propyl 2-[2-(2,3- difluorophenyl)-5H-imidazo[4,5- d]pyridazin-5-yl]-2- [2-[4-propyloxy- 2-(trifluoromethyl)phenyl]-5- pyrimidinyl]acetate mono sodium salt |
| 71a | 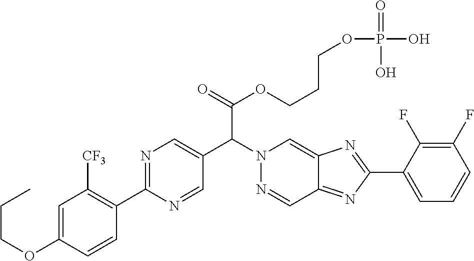 | 3-(Phosphonooxy)propyl 2-[2-(2,3- difluorophenyl)-5H-imidazo[4,5- d]pyridazin-5-yl]-2- [2-[4-propyloxy- 2-(trifluoromethyl)phenyl]-5- pyrimidinyl]acetate |
| 72 | 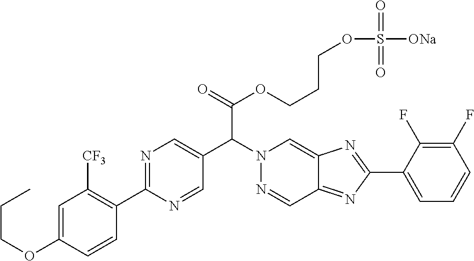 | 3-(Sulfonyloxy)propyl 2-[2-(2,3- difluorophenyl)-5H-imidazo[4,5- d]pyridazin-5-yl]-2- [2-[4-propyloxy- 2-(trifluoromethyl)phenyl]-5- pyrimidinyl]acetate mono sodium salt |
| 72a | 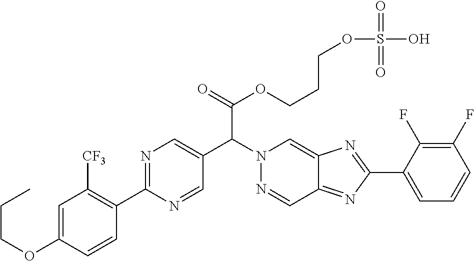 | 3-(Sulfonyloxy)propyl 2-[2-(2,3- difluorophenyl)-5H-imidazo[4,5- d]pyridazin-5-yl]-2- [2-[4-propyloxy- 2-(trifluoromethyl)phenyl]-5- pyrimidinyl]acetate |
| 73 | 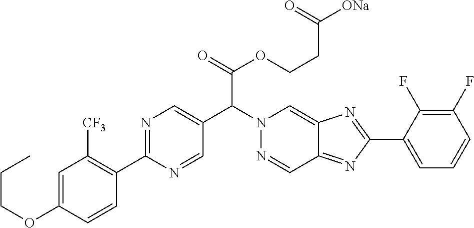 | 3-[2-[2-(2,3-Difluorophenyl)-5H- imidazo[4,5-d]pyridazin-5-yl]-2-[2- [4-propyloxy-2- (trifluoromethyl)phenyl]-5- pyrimidinyl]acetyl]oxy propanoic acid sodium salt |
| 73a | 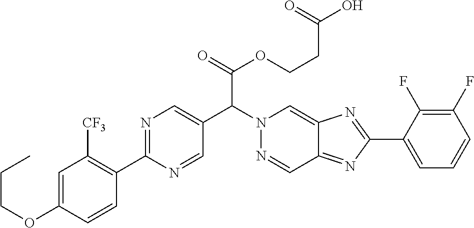 | 3-[2-[2-(2,3-Difluorophenyl)-5H- imidazo[4,5-d]pyridazin-5-yl]-2-[2- [4-propyloxy-2- (trifluoromethyl)phenyl]-5- pyrimidinyl]acetyl]oxy propanoic acid |
| 74 | 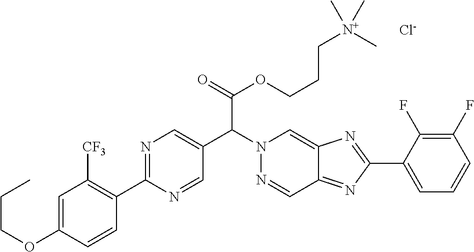 | 3-[2-[2-(2,3-Difluorophenyl)-5H- imidazo[4,5-d]pyridazin-5-yl]-2-[2- [4-propyloxy-2- (trifluoromethyl)phenyl]-5- pyrimidinyl]acetyl]oxypropyl- trimethyl-ammonium chloride |
| 75 | 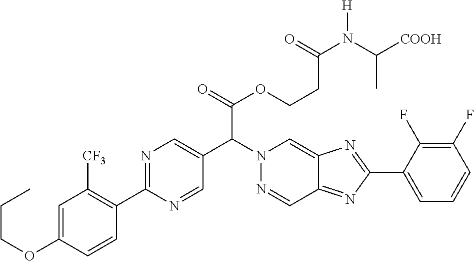 | 2-[3-[2-[2-(2,3-Difluorophenyl)-5H- imidazo[4,5-d]pyridazin-5-yl]-2-[2- [4-propyloxy-2- (trifluoromethyl)phenyl]-5- pyrimidinyl]acetyl] oxypropanoylamino] propanoic acid |
| 76 | 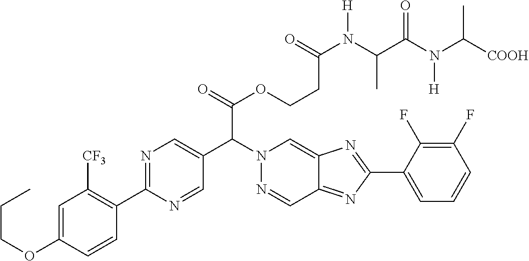 | 2-[3-[2-[2-(2,3-Difluorophenyl)- 5H-imidazo[4,5-d]pyridazin-5-yl]- 2-[2-[4-propyloxy-2- (trifluoromethyl)phenyl]-5- pyrimidinyl]acetyl] oxypropanoylamino] propanoylamino]propanoic acid |
| 77 | 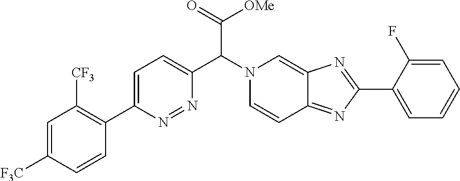 | Methyl 2-[6-[2,4- bis(trifluoromethyl)phenyl] pyridazin-3-yl]-2-[2-(2- fluorophenyl)-5H-imidazo[4,5- c]pyridin-5-yl]acetate |
| 78 | 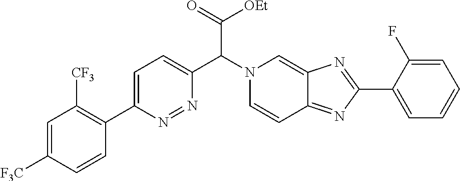 | Ethyl 2-[6-[2,4- bis(trifluoromethyl)phenyl] pyridazin-3-yl]-2-[2-(2- fluorophenyl)-5H-imidazo[4,5- c]pyridin-5-yl]acetate |
| 79 | 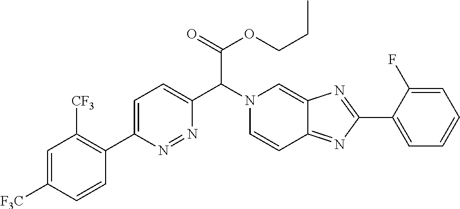 | Propyl 2-[6-[2,4- bis(trifluoromethyl)phenyl] pyridazin-3-yl]-2-[2-(2- fluorophenyl)-5H-imidazo[4,5- c]pyridin-5-yl]acetate |
| 80 | 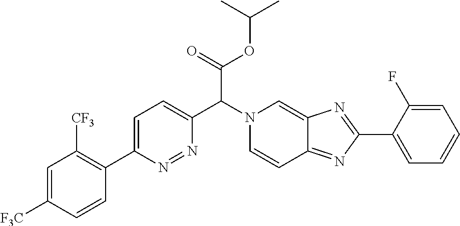 | Isopropyl 2-[6-[2,4- bis(trifluoromethyl)phenyl] pyridazin-3-yl]-2-[2-(2- fluorophenyl)-5H-imidazo[4,5- c]pyridin-5-yl]acetate |
| 81 | 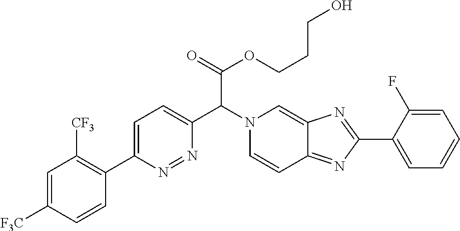 | 3-hydroxypropyl 2-[6-[2,4- bis(trifluoromethyl)phenyl] pyridazin-3-yl]-2-[2-(2- fluorophenyl)-5H-imidazo[4,5- c]pyridin-5-yl]acetate |
| 82 | 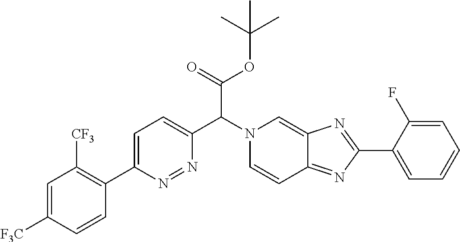 | Tert-butyl 2-[6-[2,4- bis(trifluoromethyl)phenyl] pyridazin-3-yl]-2-[2-(2- fluorophenyl)-5H-imidazo[4,5- c]pyridin-5-yl]acetate |
| 83 | 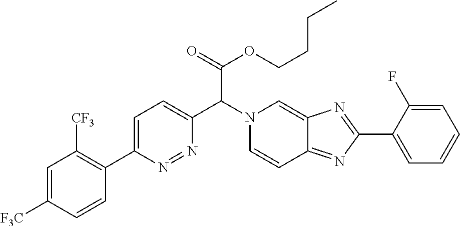 | Butyl 2-[6-[2,4- bis(trifluoromethyl)phenyl] pyridazin-3-yl]-2-[2-(2- fluorophenyl)-5H-imidazo[4,5- c]pyridin-5-yl]acetate |
| 84 | 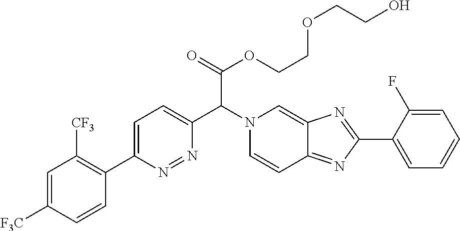 | 2-(2-Hydroxyethoxy)ethyl 2-[6- [2,4-bis(trifluoromethyl)phenyl] pyridazin-3-yl]-2-[2-(2- fluorophenyl)-5H-imidazo[4,5- c]pyridin-5-yl]acetate |
| 85 | 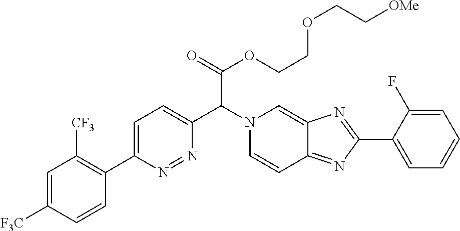 | 2-(2-Methyloxyethoxy)ethyl 2-[6- [2,4-bis(trifluoromethyl)phenyl] pyridazin-3-yl]-2-[2-(2- fluorophenyl)-5H-imidazo[4,5- c]pyridin-5-yl]acetate |
| 86 | 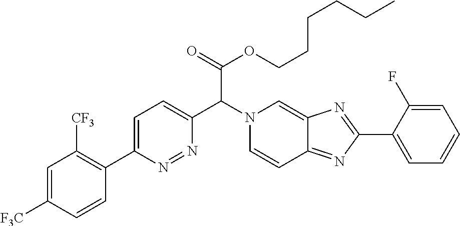 | Hexyl 2-[6-[2,4- bis(trifluoromethyl)phenyl] pyridazin-3-yl]-2-[2-(2- fluorophenyl)-5H-imidazo[4,5- c]pyridin-5-yl]acetate |
| 87 | 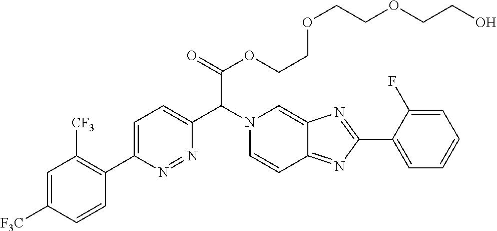 | 2-[2-[2- Hydroxyethoxy)ethoxy]ethyl 2-[6- [2,4-bis(trifluoromethyl)phenyl] pyridazin-3-yl]-2-[2-(2- fluorophenyl)-5H-imidazo[4,5- c]pyridin-5-yl]acetate |
| 88 | 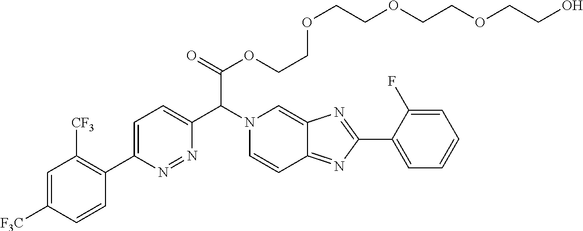 | 2-[2-[2-(2- Hydroxyethoxy)ethoxy] ethoxy]ethyl 2-[6-[2,4- bis(trifluoromethyl)phenyl] pyridazin-3-yl]-2-[2-(2- fluorophenyl)-5H-imidazo[4,5- c]pyridin-5-yl]acetate |
| 89 | 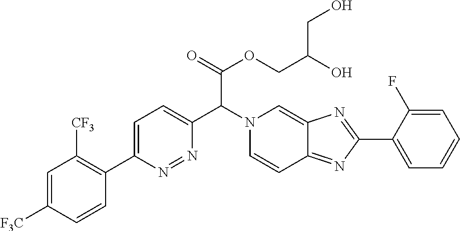 | 2,3-Dihydroxypropyl 2-[6-[2,4- bis(trifluoromethyl)phenyl] pyridazin-3-yl]-2-[2-[(2- fluorophenyl)methyl]-5H- imidazo[4,5-c]pyridin-5-yl]acetate |
| 90 | 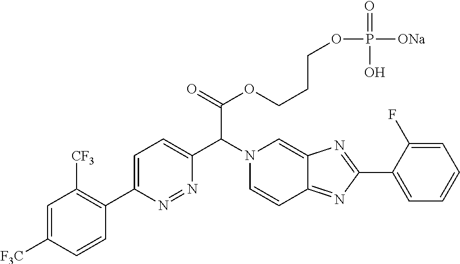 | 3-(Phosphonooxy)propyl 2-[6-[2,4- bis(trifluoromethyl)phenyl] pyridazin-3-yl]-2-[2-(2- fluorophenyl)-5H-imidazo[4,5- c]pyridin-5-yl]acetate mono sodium salt |
| 90a | 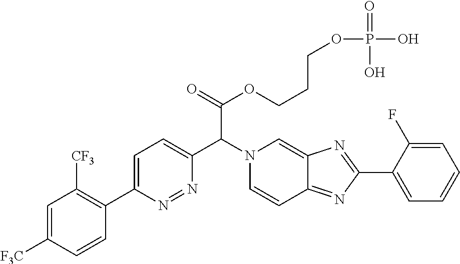 | 3-(Phosphonooxy)propyl 2-[6-[2,4- bis(trifluoromethyl)phenyl] pyridazin-3-yl]-2-[2-(2- fluorophenyl)-5H-imidazo[4,5- c]pyridin-5-yl]acetate |
| 91 | 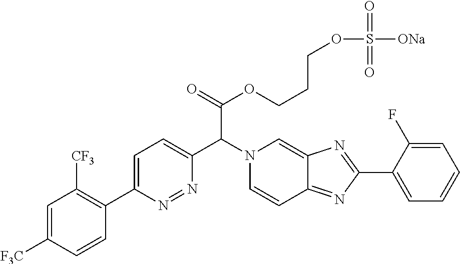 | 3-(Sulfonyloxy)propyl 2-[6-[2,4- bis(trifluoromethyl)phenyl] pyridazin-3-yl]-2-[2-(2- fluorophenyl)-5H-imidazo[4,5- c]pyridin-5-yl]acetate mono sodium salt |
| 91a | 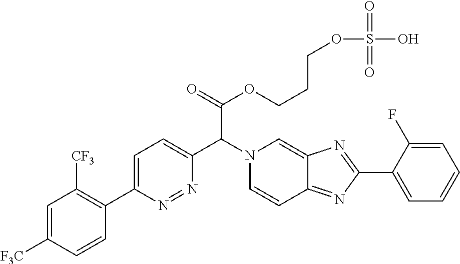 | 3-(Sulfonyloxy)propyl 2-[6-[2,4- bis(trifluoromethyl)phenyl] pyridazin-3-yl]-2-[2-(2- fluorophenyl)-5H-imidazo[4,5- c]pyridin-5-yl]acetate |
| 92 | 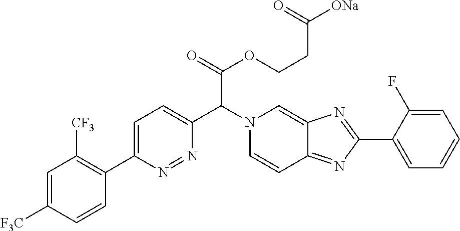 | 3-[2-[6-[2,4- Bis(trifluoromethyl)phenyl] pyridazin-3-yl]-2-[2-(2- fluorophenyl)-5H-imidazo[4,5- c]pyridin-5-yl]acetyl] oxypropanoate sodium salt |
| 92a | 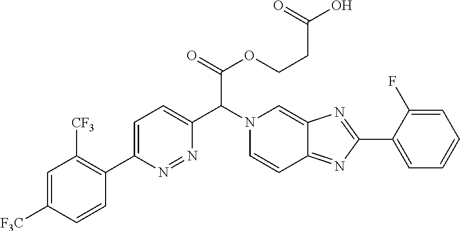 | 3-[2-[6-[2,4- Bis(trifluoromethyl)phenyl] pyridazin-3-yl]-2-[2-(2- fluorophenyl)-5H-imidazo[4,5- c]pyridin-5-yl]acetyl] oxypropanoic acid |
| 93 | 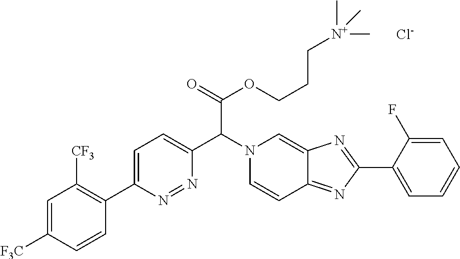 | 3-[2-[6-[2,4- Bis(trifluoromethyl)phenyl] pyridazin-3-yl]-2-[2-(2- fluorophenyl)-5H-imidazo[4,5- c]pyridin-5-yl]acetyl]oxypropyl- trimethyl-ammonium chloride |
| 94 | 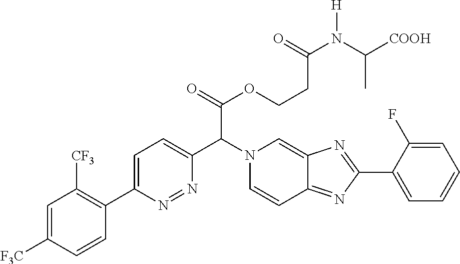 | 2-[3-[2-[6-[2,4- Bis(trifluoromethyl)phenyl] pyridazin-3-yl]-2-[2-(2- fluorophenyl)-5H-imidazo[4,5- c]pyridin-5-yl]acetyl] oxypropanoylamino]propanoic acid |
| 95 | 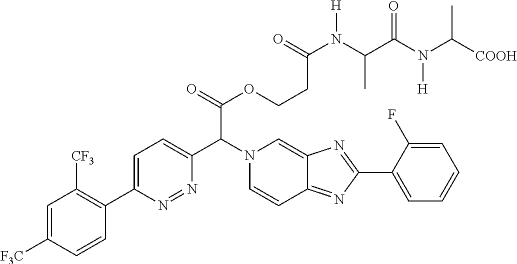 | 2-[2-[3-[2-[6-[2,4- Bis(trifluoromethyl)phenyl] pyridazin-3-yl]-2-[2-(2- fluorophenyl)-5H-imidazo[4,5- c]pyridin-5-yl]acetyl] oxypropanoylamino] propanoylamino] propanoic acid |
| 96 | 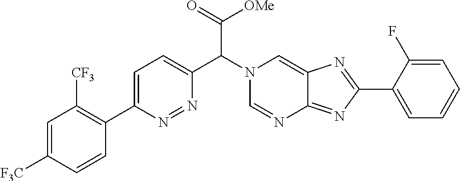 | Methyl 2-[6-[2,4- bis(trifluoromethyl)phenyl] pyridazin-3-yl]-2-[8-(2- fluorophenyl)-1H-purin-1- yl]acetate |
| 97 | 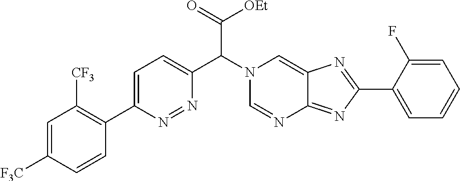 | Ethyl 2-[6-[2,4- bis(trifluoromethyl)phenyl] pyridazin-3-yl]-2-[8-(2- fluorophenyl)-1H-purin-1- yl]acetate |
| 98 | 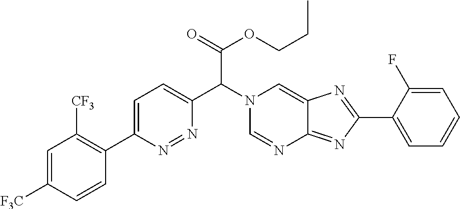 | Propyl 2-[6-[2,4- bis(trifluoromethyl)phenyl] pyridazin-3-yl]-2-[8-(2- fluorophenyl)-1H-purin-1- yl]acetate |
| 99 | 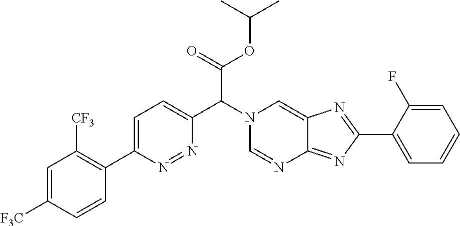 | Isopropyl 2-[6-[2,4- bis(trifluoromethyl)phenyl] pyridazin-3-yl]-2-[8-(2- fluorophenyl)-1H-purin-1- yl]acetate |
| 100 | 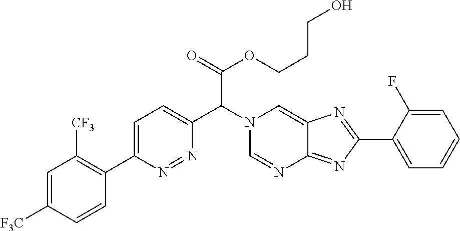 | 3-Hydroxypropyl 2-[6-[2,4- bis(trifluoromethyl)phenyl] pyridazin-3-yl]-2-[8-(2- fluorophenyl)-1H-purin-1- yl]acetate |
| 101 | 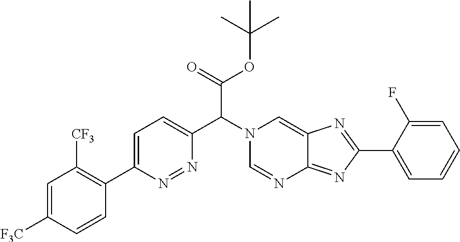 | Tert-butyl 2-[6-[2,4- bis(trifluoromethyl)phenyl] pyridazin-3-yl]-2-[8-(2- fluorophenyl)-1H-purin-1- yl]acetate |
| 102 | 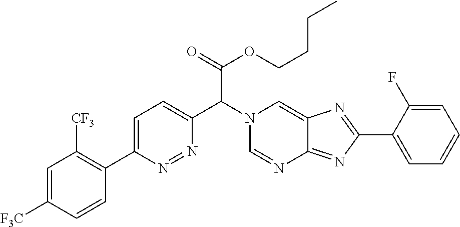 | Butyl 2-[6-[2,4- bis(trifluoromethyl)phenyl] pyridazin-3-yl]-2-[8-(2- fluorophenyl)-1H-purin-1- yl]acetate |
| 103 | 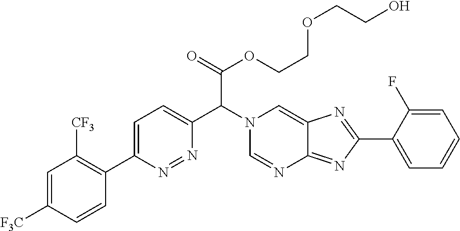 | 2-(2-Hydroxyethoxy)ethyl 2-[6- [2,4-bis(trifluoromethyl)phenyl] pyridazin-3-yl]-2-[8-(2- fluorophenyl)-1H-purin-1- yl]acetate |
| 104 | 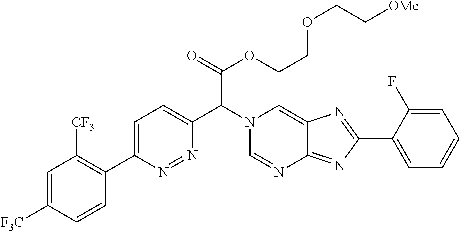 | 2-(2-Methyloxyethoxy)ethyl 2-[6- [2,4-bis(trifluoromethyl)phenyl] pyridazin-3-yl]-2-[8-(2- fluorophenyl)-1H-purin-1- yl]acetate |
| 105 | 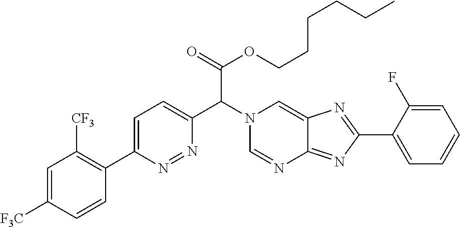 | Hexyl 2-[6-[2,4- bis(trifluoromethyl)phenyl] pyridazin- 3-yl]-2-[8-(2-fluorophenyl)-1H- purin-1-yl]acetate |
| 115 | 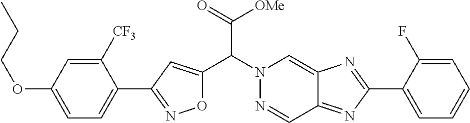 | Methyl 2-[2-(2-fluorophenyl)-5H- imidazo[4,5-d]pyridazin-5-yl]-2-[3- [4-propyloxy-2- (trifluoromethyl)phenyl] isoxazol-5-yl]acetate |
| 116 | 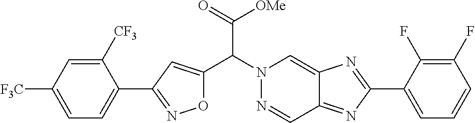 | Methyl 2-[2-(2,3-difluorophenyl)- 5H-imidazo[4,5-d]pyridazin-5-yl]- 2-[3-[2,4- bis(trifluoromethyl)phenyl] isoxazol-5-yl]acetate |
| 117 | 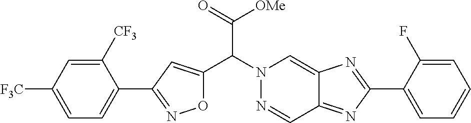 | Methyl 2-[2-(2-fluorophenyl)-5H- imidazo[4,5-d]pyridazin-5-yl]- 2-[3- [2,4-bis(trifluoromethyl)phenyl] isoxazol-5-yl]acetic acid |
| 118 | 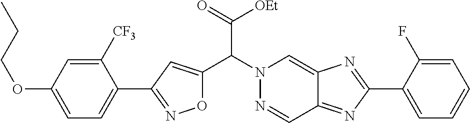 | Ethyl 2-[2-(2-fluorophenyl)-5H- imidazo[4,5-d]pyridazin-5-yl]-2-[3- [4-propyloxy-2- (trifluoromethyl)phenyl]isoxazol-5- yl]acetate |
| 119 | 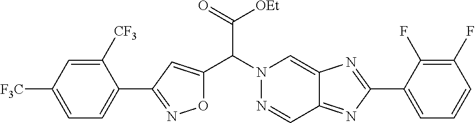 | Ethyl 2-[2-(2,3-difluorophenyl)-5H- imidazo[4,5-d]pyridazin-5-yl]-2-[3- [2,4-bis(trifluoromethyl)phenyl] isoxazol-5-yl]acetate |
| 120 | 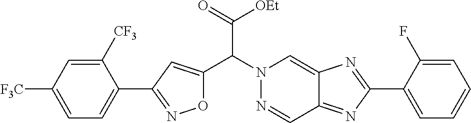 | Ethyl 2-[2-(2-fluorophenyl)-5H- imidazo[4,5-d]pyridazin-5-yl]-2-[3- [2,4-bis(trifluoromethyl)phenyl] isoxazol-5-yl]acetate |
| 121 | 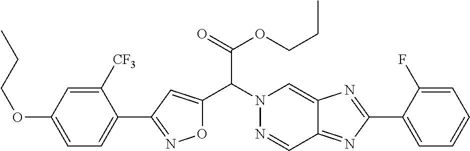 | Propyl 2-[2-(2-fluorophenyl)-5H- imidazo[4,5-d]pyridazin-5-yl]-2-[3- [4-propyloxy-2- (trifluoromethyl)phenyl] isoxazol-5-yl]acetate |
| 122 | 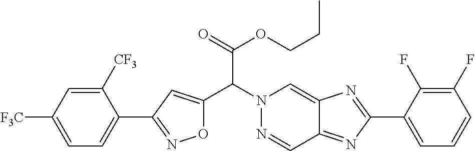 | Propyl 2-[2-(2,3-difluorophenyl)- 5H-imidazo[4,5-d]pyridazin-5-yl]- 2-[3-[2,4- bis(trifluoromethyl)phenyl] isoxazol-5-yl]acetate |
| 123 | 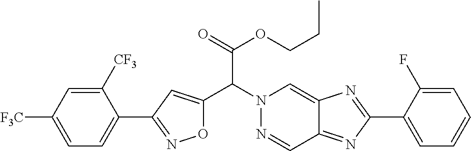 | Propyl 2-[2-(2-fluorophenyl)-5H- imidazo[4,5-d]pyridazin-5-yl]-2-[3- [2,4-bis(trifluoromethyl)phenyl] isoxazol-5-yl]acetate |
| 124 | 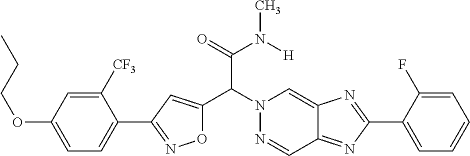 | 2-[2-(2-Fluorophenyl)-5H- imidazo[4,5-d]pyridazin-5-yl]-N- methyl-2-[3-[4-propyloxy-2- (trifluoromethyl)phenyl]isoxazol-5- yl]acetamide |
| 125 | 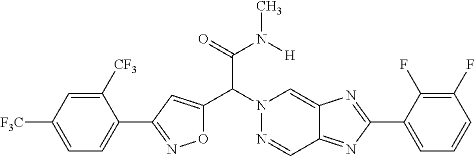 | 2-[2-(2,3-Difluorophenyl)-5H- imidazo[4,5-d]pyridazin-5-yl]-N- methyl-2-[3-[2,4- bis(trifluoromethyl)phenyl] isoxazol-5-yl]acetamide |
| 126 | 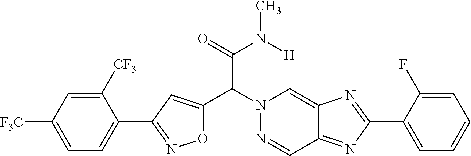 | 2-[2-(2-Fluorophenyl)-5H- imidazo[4,5-d]pyridazin-5-yl]-N- methyl-2-[3-[2,4- bis(trifluoromethyl)phenyl] isoxazol-5-yl]acetamide |
| 127 | 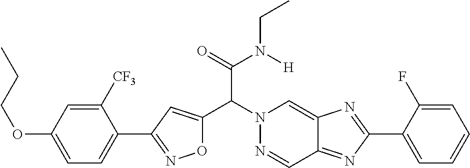 | N-ethyl-2-[2-(2-Fluorophenyl)-5H- imidazo[4,5-d]pyridazin-5-yl]-2-[3- [4-propyloxy-2- (trifluoromethyl)phenyl] isoxazol-5-yl]acetate |
| 128 | 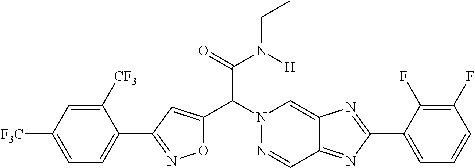 | 2-[2-(2,3-Difluorophenyl)-5H- imidazo[4,5-d]pyridazin-5-yl]-N- ethyl-2-[3-[2,4- bis(trifluoromethyl)phenyl] isoxazol-5-yl]acetamide |
| 129 | 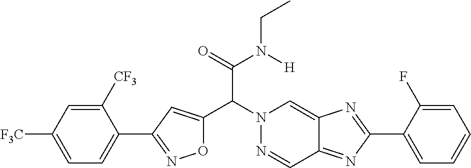 | N-ethyl-2-[2-(2-fluorophenyl)-5H- imidazo[4,5-d]pyridazin-5-yl]-2-[3- [2,4-bis(trifluoromethyl)phenyl] isoxazol-5-yl]acetamide |
| 130 | 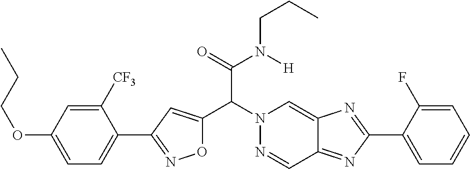 | 2-[2-(2-Fluorophenyl)-5H- imidazo[4,5-d]pyridazin-5-yl]-2-[3- [4-propyloxy-2- (trifluoromethyl)phenyl]isoxazol-5- yl]-N-propyl-acetamide |
| 131 | 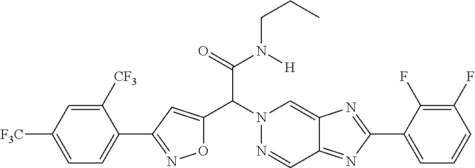 | 2-[2-(2,3-Difluorophenyl)-5H- imidazo[4,5-d]pyridazin-5-yl]-2-[3- [2,4-bis(trifluoromethyl)phenyl] isoxazol-5-yl]-N-propyl-acetamide |
| 132 | 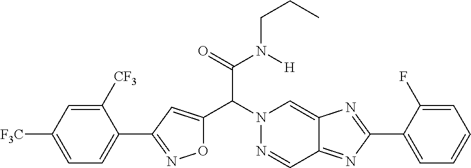 | 2-[2-(2-Fluorophenyl)-5H- imidazo[4,5-d]pyridazin-5-yl]-2-[3- [2,4-bis(trifluoromethyl)phenyl] isoxazol-5-yl]-N-propyl-acetamide |
| 133 | 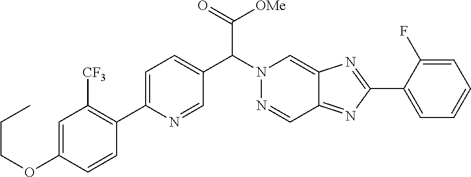 | Methyl 2-[2-(2-fluorophenyl)-5H- imidazo[4,5-d]pyridazin- 5-yl]-2-[6- [4-propyloxy-2- (trifluoromethyl)phenyl]-3- pyridinyl]acetate |
| 134 | 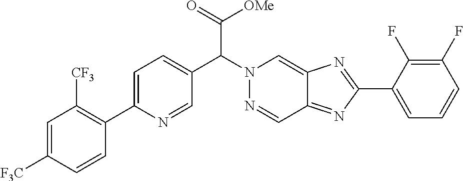 | Methyl 2-[2-(2,3-difluorophenyl)- 5H-imidazo[4,5-d]pyridazin-5-yl]- 2-[6-[2,4- bis(trifluoromethyl)phenyl]-3- pyridinyl]acetate |
| 135 | 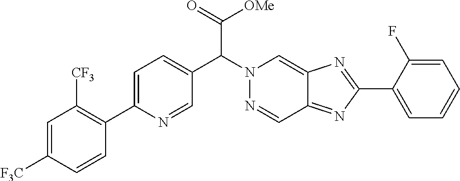 | Methyl 2-[2-(2-fluorophenyl)-5H- imidazo[4,5-d]pyridazin-5-yl]-2-[6- [2,4-bis(trifluoromethyl)phenyl]-3- pyridinyl]acetate |
| 136 | 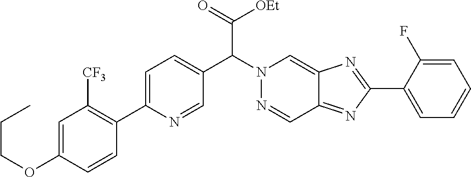 | Ethyl 2-[2-(2-fluorophenyl)-5H- imidazo[4,5-d]pyridazin-5-yl]-2-[6- [4-propyloxy-2- (trifluoromethyl)phenyl]-3- pyridinyl]acetate |
| 137 | 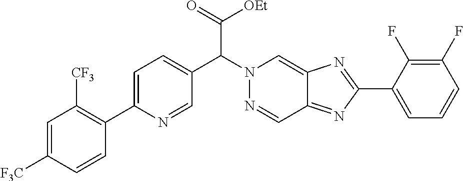 | Ethyl 2-[2-(2,3-difluorophenyl)-5H- imidazo[4,5-d]pyridazin-5-yl]-2-[6- [2,4-bis(trifluoromethyl)phenyl]-3- pyridinyl]acetate |
| 138 | 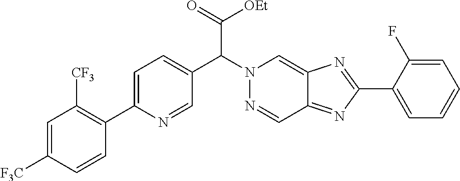 | Ethyl 2-[2-(2-fluorophenyl)-5H- imidazo[4,5-d]pyridazin-5-yl]-2-[6- [2,4-bis(trifluoromethyl)phenyl]-3- pyridinyl]acetate |
| 139 | 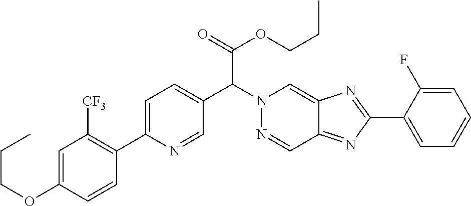 | Propyl 2-[2-(2-fluorophenyl)-5H- imidazo[4,5-d]pyridazin-5-yl]-2-[6- [4-propyloxy-2- (trifluoromethyl)phenyl]-3- pyridinyl]acetate |
| 140 | 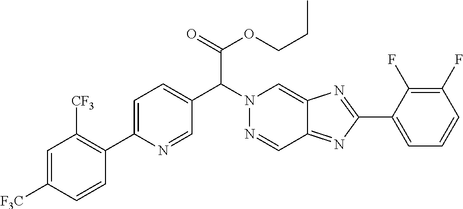 | Propyl 2-[2-(2,3-difluorophenyl)- 5H-imidazo[4,5-d]pyridazin-5-yl]- 2-[6-[2,4- bis(trifluoromethyl)phenyl]-3- pyridinyl]acetate |
| 141 | 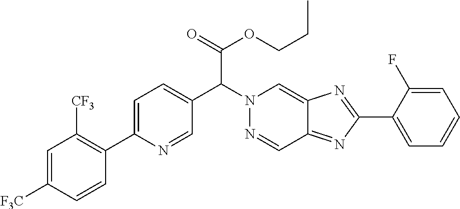 | Propyl 2-[2-(2-fluorophenyl)-5H- imidazo[4,5-d]pyridazin-5-yl]-2-[6- [2,4-bis(trifluoromethyl)phenyl]-3- pyridinyl]acetate |
| 142 | 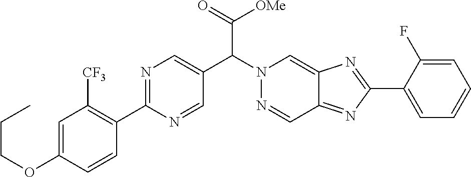 | Methyl 2-[2-(2-fluorophenyl)-5H- imidazo[4,5-d]pyridazin- 5-yl]-2-[2- [4-propyloxy-2- (trifluoromethyl)phenyl]-5- pyrimidinyl]acetate |
| 143 | 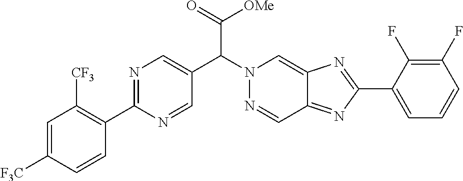 | Methyl 2-[2-(2,3-difluorophenyl)- 5H-imidazo[4,5-d]pyridazin-5-yl]- 2-[2-(2,4- bis(trifluoromethyl)phenyl]-5- pyrimidinyl]acetate |
| 144 | 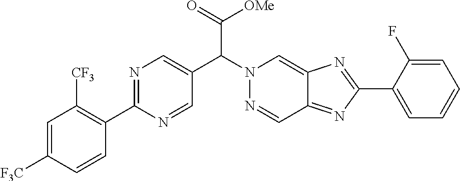 | Methyl 2-[2-(2-fluorophenyl)-5H- imidazo[4,5-d]pyridazin-5-yl]-2-[2- [2,4-bis(trifluoromethyl)phenyl]-5- pyrimidinyl]acetate |
| 145 | 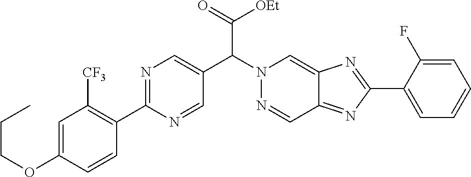 | Ethyl 2-[2-(2-fluorophenyl)-5H- imidazo[4,5-d]pyridazin- 5-yl]-2-[2- [4-propyloxy-2- (trifluoromethyl)phenyl]-5- pyrimidinyl]acetate |
| 146 | 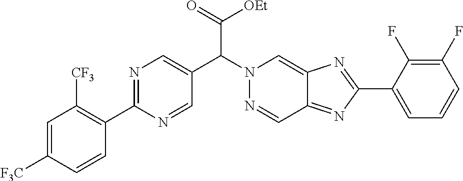 | Ethyl 2-[2-(2,3-difluorophenyl)-5H- imidazo[4,5-d]pyridazin- 5-yl]-2-[2- [2,4-bis(trifluoromethyl)phenyl]-5- pyrimidinyl]acetate |
| 147 | 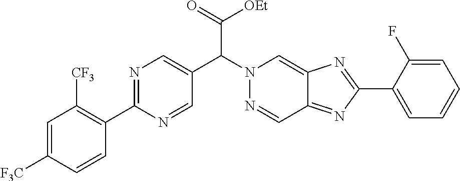 | Ethyl 2-[2-(2-fluorophenyl)-5H- imidazo[4,5-d]pyridazin-5-yl]-2-[2- [2,4-bis(trifluoromethyl)phenyl]-5- pyrimidinyl]acetate |
| 148 | 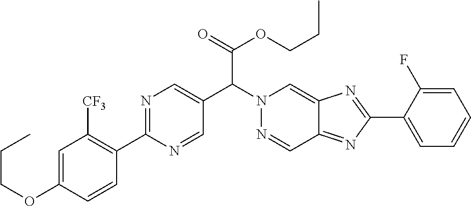 | Propyl 2-[2-(2-fluorophenyl)-5H- imidazo[4,5-d]pyridazin-5-yl]-2-[2- [4-propyloxy-2- (trifluoromethyl)phenyl]-5- pyrimidinyl]acetate |
| 149 | 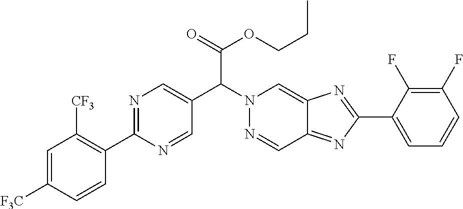 | Propyl 2-[2-(2,3-difluorophenyl)- 5H-imidazo[4,5-d]pyridazin-5-yl]- 2-[2-(2,4- bis(trifluoromethyl)phenyl]-5- pyrimidinyl]acetate |
| 150 | 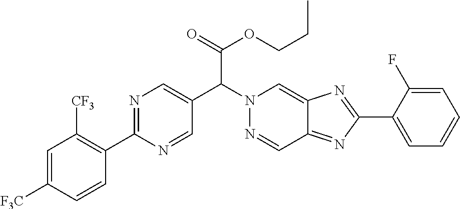 | Propyl 2-[2-(2-fluorophenyl)-5H- imidazo[4,5-d]pyridazin-5-yl]-2-[2- [2,4-bis(trifluoromethyl)phenyl]-5- pyrimidinyl]acetate |
| 151 | 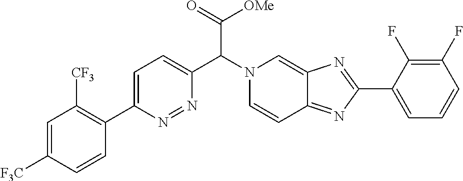 | Methyl 2-[2-(2,3-difluorophenyl)- 5H-imidazo[4,5-c]pyridin-5-yl]-2- [6-[2,4-bis(trifluoromethyl)phenyl] pyridazin-3-yl]acetate |
| 152 | 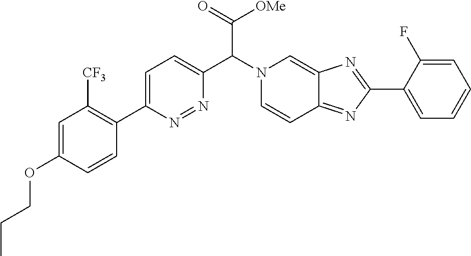 | Methyl 2-[2-(2-fluorophenyl)-5H- imidazo[4,5-c]pyridin-5-yl]-2-[6-[4- propyloxy-2 - (trifluoromethyl)phenyl] pyridazin-3-yl]acetate |
| 153 | 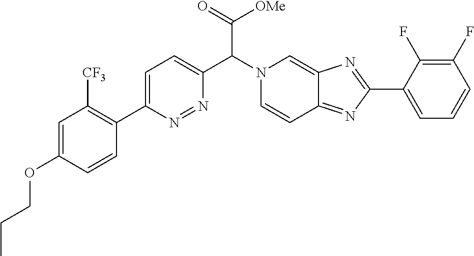 | Methyl 2-[2-(2,3-difluorophenyl)- 5H-imidazo[4,5-c]pyridin-5-yl]-2- [6-[4-propyloxy-2- (trifluoromethyl)phenyl] pyridazin-3-yl]acetate |
| 154 | 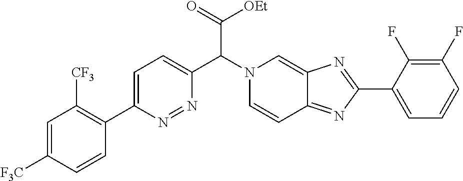 | Ethyl 2-[2-(2,3-difluorophenyl)-5H- imidazo[4,5-c]pyridin-5-yl]-2-[6- [2,4-bis(trifluoromethyl)phenyl] pyridazin-3-yl]acetate |
| 155 | 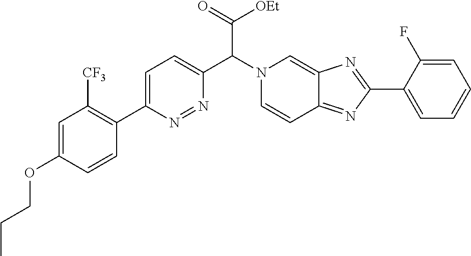 | Ethyl 2-[2-(2-fluorophenyl)-5H- imidazo[4,5-c]pyridin-5-yl]-2-[6-[4- propyloxy-2 - (trifluoromethyl)phenyl] pyridazin-3-yl]acetate |
| 156 | 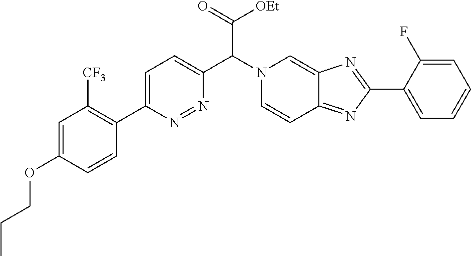 | Ethyl 2-[2-(2,3-difluorophenyl)-5H- imidazo[4,5-c]pyridin-5-yl]-2-[6-[4- propyloxy-2 - (trifluoromethyl)phenyl] pyridazin-3-yl]acetate |
| 157 | 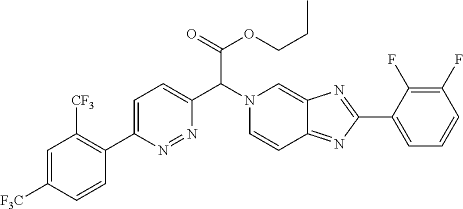 | Propyl 2-[2-(2,3-difluorophenyl)- 5H-imidazo[4,5-c]pyridin-5-yl]-2- [6-[2,4-bis(trifluoromethyl)phenyl] pyridazin-3-yl]acetate |
| 158 | 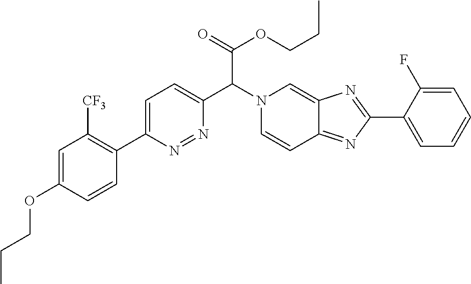 | Propyl 2-[2-(2-fluorophenyl)-5H- imidazo[4,5-c]pyridin-5-yl]-2-[6-[4- propyloxy-2- (trifluoromethyl)phenyl] pyridazin-3-yl]acetate |
| 159 | 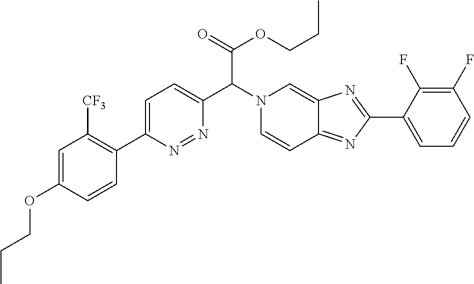 | Propyl 2-[2-(2,3-difluorophenyl)- 5H-imidazo[4,5-c]pyridin-5-yl]-2- [6-[4-propyloxy-2- (trifluoromethyl)phenyl] pyridazin-3-yl]acetate |
| 160 | 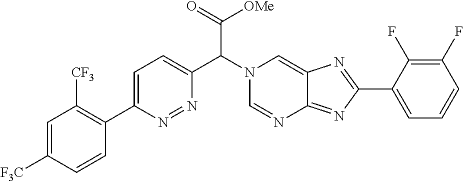 | Methyl 2-[8-(2,3-difluorophenyl)- 1H-purin-1-yl]-2-[6-[2,4- bis(trifluoromethyl)phenyl] pyridazin-3-yl]acetate |
| 161 | 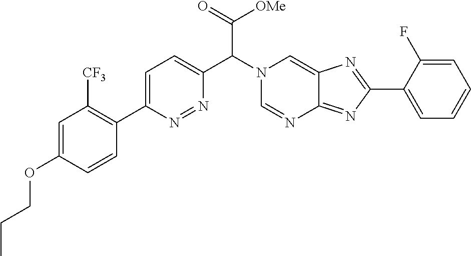 | Methyl 2-[8-(2-fluorophenyl)-1H- purin-1-yl]-2-[6-[4-propyloxy-2- (trifluoromethyl)phenyl] pyridazin-3-yl]acetate |
| 162 | 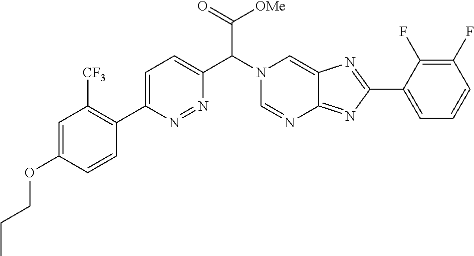 | Methyl 2-[8-(2,3-difluorophenyl)- 1H-purin-1-yl]-2-[6-[4-propyloxy-2- (trifluoromethyl)phenyl] pyridazin-3-yl]acetate |
| 163 | 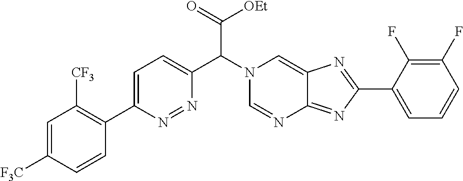 | Ethyl 2-[8-(2,3-difluorophenyl)-1H- purin-1-yl]-2-[6-[2,4- bis(trifluoromethyl)phenyl] pyridazin-3-yl]acetate |
| 164 | 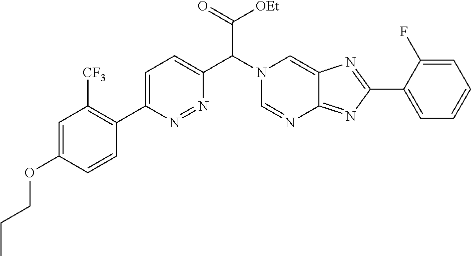 | Ethyl 2-[8-(2-fluorophenyl)-1H- purin-1-yl]-2-[6-[4-propyloxy-2- (trifluoromethyl)phenyl] pyridazin-3-yl]acetate |
| 165 | 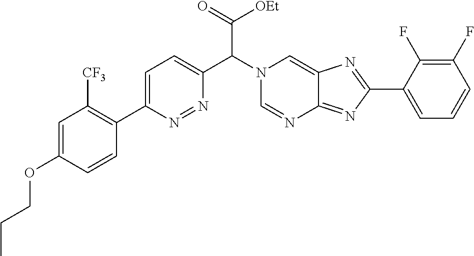 | Ethyl 2-[8-(2,3-difluorophenyl)-1H- purin-1-yl]-2-[6-[4-propyloxy-2- (trifluoromethyl)phenyl] pyridazin-3-yl]acetate |
| 166 | 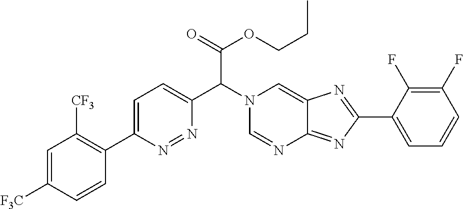 | Propyl 2-[8-(2,3-difluorophenyl)- 1H-purin-1-yl]-2-[6-[2,4- bis(trifluoromethyl)phenyl] pyridazin-3-yl]acetate |
| 167 | 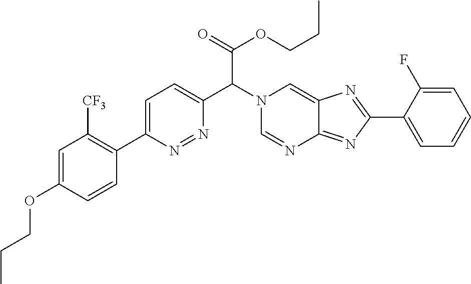 | Propyl 2-[8-(2-fluorophenyl)-1H- purin-1-yl]-2-[6-[4-propyloxy-2- (trifluoromethyl)phenyl] pyridazin-3-yl]acetate |
| 168 | 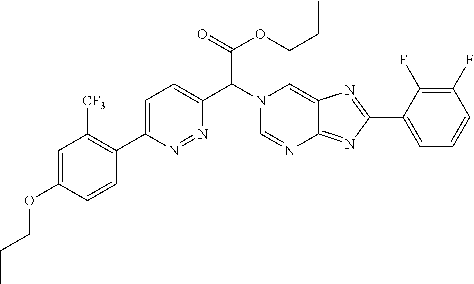 | Propyl 2-[8-[(2,3- difluorophenyl)methyl]-1H-purin-1- yl]-2-[6-[4-propyloxy-2- (trifluoromethyl)phenyl] pyridazin-3-yl]acetate |
| 169 | 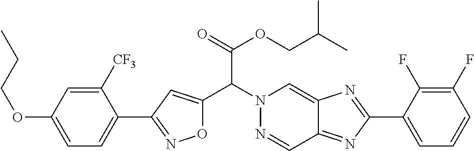 | 2-Methylpropyl [2-(2,3- difluorophenyl)-5H-imidazo[4,5- d]pyridazin-5-yl]{3-[4-(propyloxy)- 2-(trifluoromethyl)phenyl]-5- isoxazolyl}acetate |
| 171 | 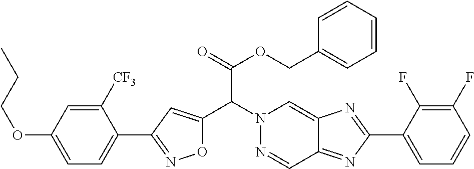 | Phenylmethyl [2-(2,3- difluorophenyl)-5H- imidazo[4,5-d]pyridazin-5-yl]{3- [4-(propyloxy)-2- (trifluoromethyl)phenyl]-5- isoxazolyl}acetate |
| 173 | 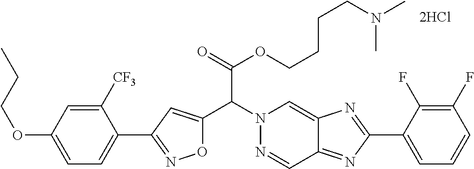 | 4-(Dimethylamino)butyl [2- (2,3-difluorophenyl)-5H- imidazo[4,5-d]pyridazin-5-yl]{3- [4-(propyloxy)-2- (trifluoromethyl)phenyl]-5- isoxazolyl}acetate dihydrochloride |
| 174 | 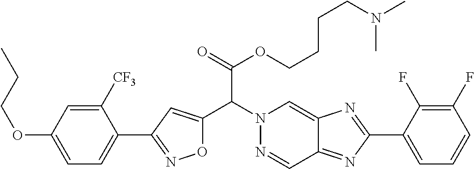 | 4-(Dimethylamino)butyl [2- (2,3-difluorophenyl)-5H- imidazo[4,5-d]pyridazin-5-yl]{3- [4-(propyloxy)-2- (trifluoromethyl)phenyl]-5- isoxazolyl}acetate |
| 175 | 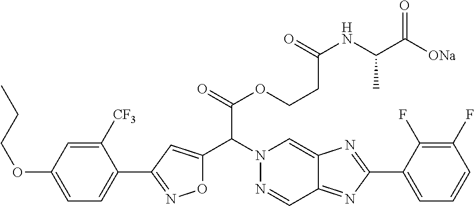 | N-{3-[([2-(2,3-Difluorophenyl)- 5H-imidazo[4,5-d]pyridazin-5- yl]{3-[4-(propyloxy)-2- (trifluoromethyl)phenyl]-5- isoxazolyl}acetyl)oxy]propanoyl}- L-alanine sodium salt |
| 176 | 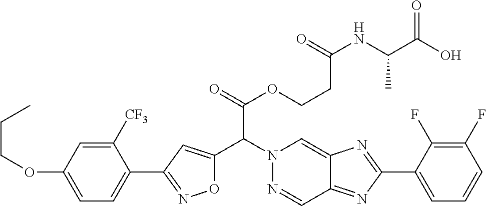 | N-{3-[([2-(2,3-Difluorophenyl)- 5H-imidazo[4,5-d]pyridazin-5- yl]{3-[4-(propyloxy)-2- (trifluoromethyl)phenyl]-5- isoxazolyl}acetyl)oxy]propanoyl}- L-alanine |
| 177 | 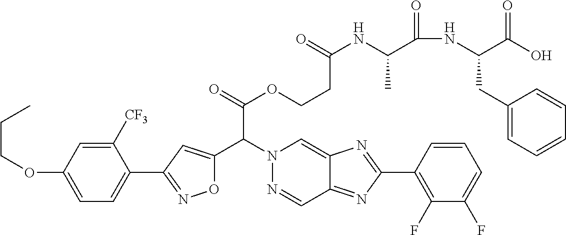 | N-{3-[([2-(2,3-Difluorophenyl)- 5H-imidazo[4,5-d]pyridazin-5- yl]{3-[4-(propyloxy)-2- (trifluoromethyl)phenyl]-5- isoxazolyl}acetyl)oxy]propanoyl}- L-alanyl-L-phenylalanine |
| 178 | 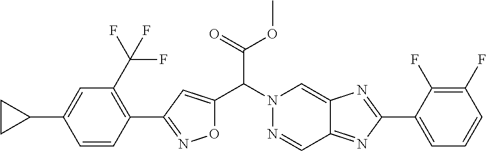 | Methyl {3-[4-cyclopropyl-2- (trifluoromethyl)phenyl]-5- isoxazolyl}[2-(2,3- difluorophenyl)-5H- imidazo[4,5-d]pyridazin-5- yl]acetate |
| 179 | 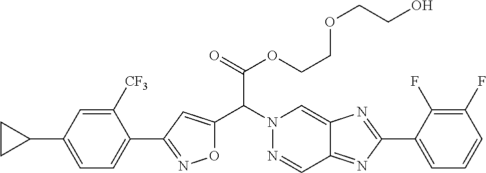 | 2-[(2-hydroxyethyl)oxy]ethyl {3-[4-cyclopropyl-2- (trifluoromethyl)phenyl]-5- isoxazolyl}[2-(2,3- difluorophenyl)-5H- imidazo[4,5-d]pyridazin-5- yl]acetate |
| 180 | 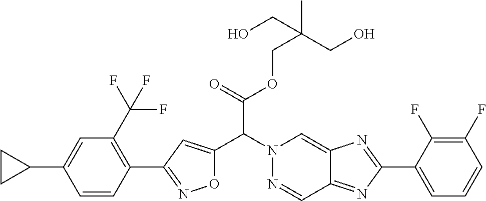 | 3-hydroxy-2-(hydroxymethyl)- 2-methylpropyl {3-[4- cyclopropyl-2- (trifluoromethyl)phenyl]-5- isoxazolyl}[2-(2,3- difluorophenyl)-5H- imidazo[4,5-d]pyridazin-5- yl]acetate |
| 181 | 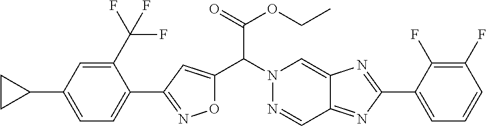 | Ethyl {3-[4-cyclopropyl-2- (trifluoromethyl)phenyl]-5- isoxazolyl}[2-(2,3- difluorophenyl)-5H- imidazo[4,5-d]pyridazin-5- yl]acetate |
| 182 | 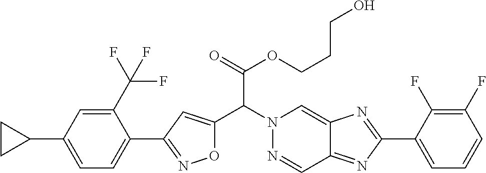 | 3-Hydroxypropyl {3-[4- cyclopropyl-2- (trifluoromethyl)phenyl]-5- isoxazolyl}[2-(2,3- difluorophenyl)-5H- imidazo[4,5-d]pyridazin-5- yl]acetate |
| 183 | 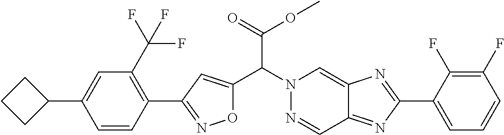 | Methyl {3-[4-cyclobutyl-2- (trifluoromethyl)phenyl]-5- isoxazolyl}[2-(2,3- difluorophenyl)-5H- imidazo[4,5-d]pyridazin-5- yl]acetate |
| 184 | 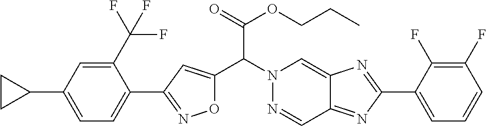 | Propyl {3-[4-cyclopropyl-2- (trifluoromethyl)phenyl]-5- isoxazolyl}[2-(2,3- difluorophenyl)-5H- imidazo[4,5-d]pyridazin-5- yl]acetate |
| 185 | 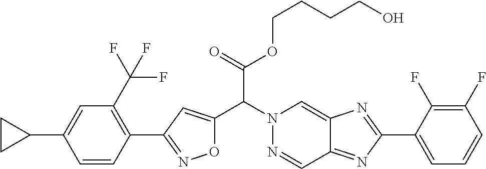 | 4-hydroxybutyl {3-[4- cyclopropyl-2- (trifluoromethyl)phenyl]-5- isoxazolyl}[2-(2,3- difluorophenyl)-5H- imidazo[4,5-d]pyridazin-5- yl]acetate |
| 186 | 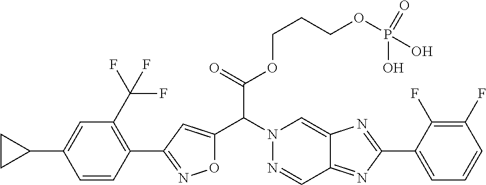 | 3-({bis[(1,1- dimethylethyl)oxy]phosphoryl} oxy)propyl {3-[4-cyclopropyl-2- (trifluoromethyl)phenyl]-5- isoxazolyl}[2-(2,3- difluorophenyl)-5H- imidazo[4,5-d]pyridazin-5- yl]acetate |
| 187 | 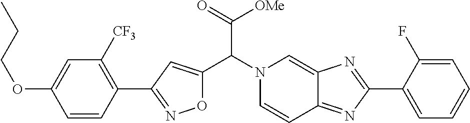 | Methyl [2-(2-fluorophenyl)-5H- imidazo[4,5-c]pyridin-5-yl]{3- [4-(propyloxy)-2- (trifluoromethyl)phenyl]-5- isoxazolyl}acetate |

The compounds of Formula (I) contain a prodrug moiety covalently linked to the L¹ group of the molecule. The prodrug moiety may enhance the intrinsic solubility of the compounds in aqueous solutions. The compounds may also exhibit improved DMPK modulation, active transport, and selective tissue distribution. The prodrug moiety may be released when administered to a biological system or patient to generate the drug substance, i.e. active ingredient, as a result of spontaneous chemical reaction(s), enzyme catalyzed chemical reaction(s), photolysis, and/or metabolic chemical reaction(s).

By way of example, prodrug compounds of Formula (VIII) (wherein, G is C, Q⁴ is O, and Y is O, S, or NR⁷), may be administered to a patient in vivo. Upon administration, modification of the prodrug compound is believed to proceed as follows:

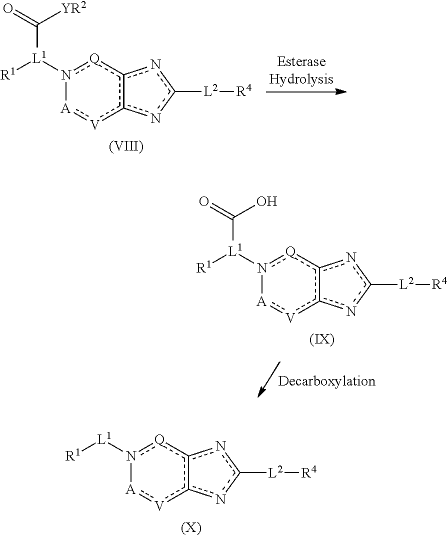

As indicated herein, the moiety “YR²” may be hydrolytically cleaved via an esterase-mediated reaction to generate a carboxylated compound of Formula (IX). This compound may then undergo spontaneous decarboxylation to generate a physiologically active compound of Formula (X). In this manner, the prodrug compound can function as a “double” prodrug that undergoes sequential modification to result in the active compound. It should also be understood that the prodrug compounds themselves may also be therapeutically active in their own right.

Of course, the present invention is by no means limited to a particular metabolic pathway for the prodrug compound. For instance, prodrug compounds of Formula (XI) (wherein, G is C, Q⁴ is NR⁷, and Y is a bond), may be administered to a patient in vivo and undergo modification as follows:

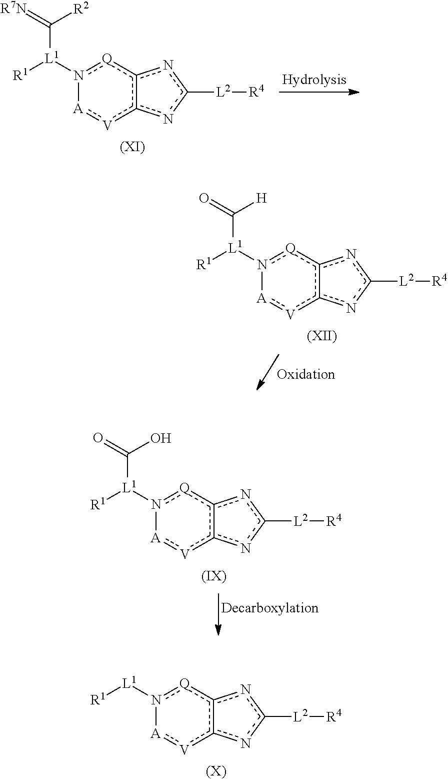

As indicated herein, the moiety “(NR⁷)CHR²” may be hydrolyzed to generate an aldehyde compound of Formula (XII) that may be subsequently oxidized through a variety of oxidase and/or dehydrogenase enzymes (e.g., cytochrome P450 oxidase, NADPH oxidase, etc.) to generate the aforementioned carboxylated compound (IX), which as described herein, can undergo spontaneous decarboxylation to generate a physiologically active compound of Formula (X).

The aldehyde may also be formed through a variety of other metabolic pathways. For example, the aldehyde of Formula (XII) may be formed via an alcohol compound of Formula (XIII), which may undergo oxidation in a manner such as described herein to form the aldehyde of Formula (XII):

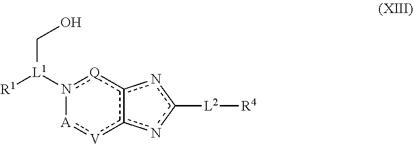

If desired, the aldehyde of Formula (XII) may itself be employed as the prodrug compound of the present invention (e.g., wherein, G is C, Q⁴ is O, Y is a bond, and R² is H). Similarly, the alcohol of Formula (XIII) may be employed as the prodrug compound of the present invention (e.g., wherein, G is C, Q⁴ is O, Y is O, and R² is H).

By way of other examples, prodrug compounds of Formula (IV) (wherein, Q⁴ is O, and Y is O, S, or NR⁷), may be administered to a patient in vivo. Upon administration, modification of the prodrug compound is believed to proceed as follows:

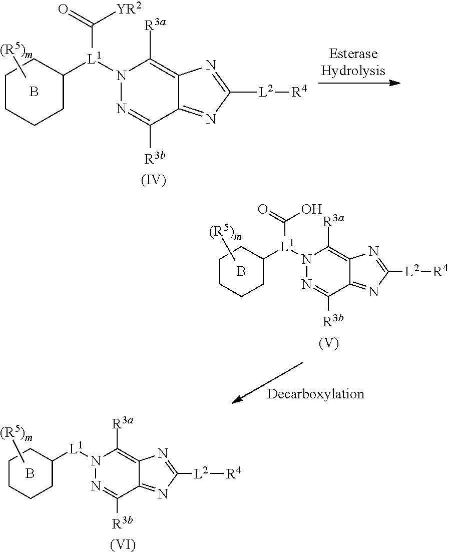

As indicated herein, the moiety “YR²” may be hydrolytically cleaved via an esterase-mediated reaction to generate a carboxylated compound of Formula (V). This compound may then undergo spontaneous decarboxylation to generate a physiologically active compound of Formula (VI). In this manner, the prodrug compound can function as a “double” prodrug that undergoes sequential modification to result in the active compound. It should also be understood that the prodrug compounds themselves may also be therapeutically active in their own right.

Of course, the present invention is by no means limited to a particular metabolic pathway for the prodrug compound. For instance, prodrug compounds of Formula (VII) (wherein, Q⁴ is NR⁷, and Y is a bond), may be administered to a patient in vivo and undergo modification as follows:

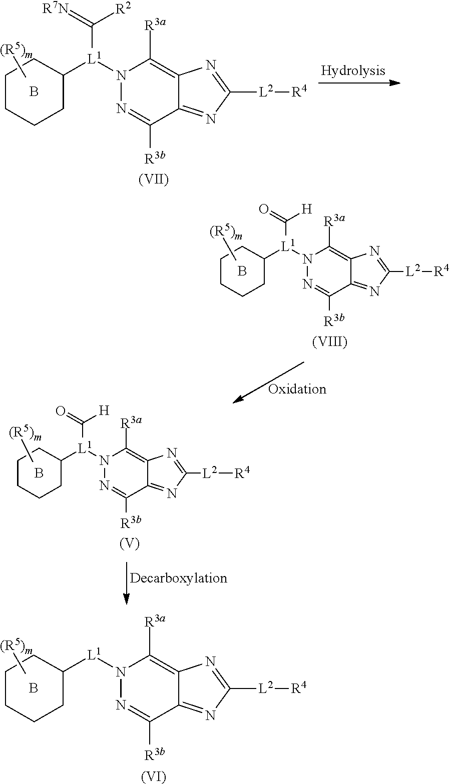

As indicated herein, the moiety “(NR⁷)CHR²” may be hydrolyzed to generate an aldehyde compound of Formula (VIII) that may be subsequently oxidized through a variety of oxidase and/or dehydrogenase enzymes (e.g., cytochrome P450 oxidase, NADPH oxidase, etc.) to generate the aforementioned carboxylated compound (V), which as described herein, can undergo spontaneous decarboxylation to generate a physiologically active compound of Formula (VI).

The aldehyde may also be formed through a variety of other metabolic pathways. For example, the aldehyde of Formula (VIII) may be formed via an alcohol compound of Formula (IX), which may undergo oxidation in a manner such as described herein to form the aldehyde of Formula (VIII):

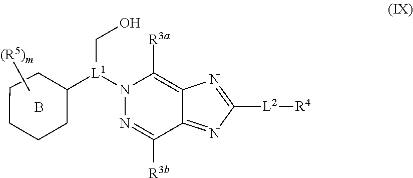

If desired, the aldehyde of Formula (VIII) may itself be employed as the prodrug compound of the present invention (e.g., wherein, Q⁴ is O, Y is a bond, and R² is H). Similarly, the alcohol of Formula (IX) may be employed as the prodrug compound of the present invention (e.g., wherein, Q⁴ is O, Y is O, and R² is H).

By way of still further examples, prodrug compounds of Formula (IV) (wherein, Q⁴ is O, and Y is O, S, or NR⁷), may be administered to a patient in vivo. Upon administration, modification of the prodrug compound is believed to proceed as follows:

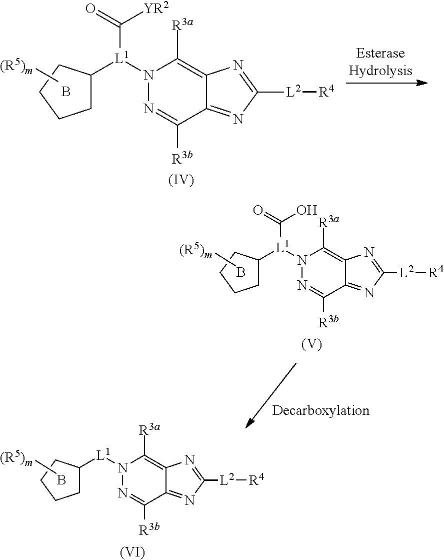

As indicated herein, the moiety “YR²” may be hydrolytically cleaved via an esterase-mediated reaction to generate a carboxylated compound of Formula (V). This compound may then undergo spontaneous decarboxylation to generate a physiologically active compound of Formula (VI). In this manner, the prodrug compound can function as a “double” prodrug that undergoes sequential modification to result in the active compound. It should also be understood that the prodrug compounds themselves may also be therapeutically active in their own right.

Of course, the present invention is by no means limited to a particular metabolic pathway for the prodrug compound. For instance, prodrug compounds of Formula (VII) (wherein, Q⁴ is NR⁷, and Y is a bond), may be administered to a patient in vivo and undergo modification as follows:

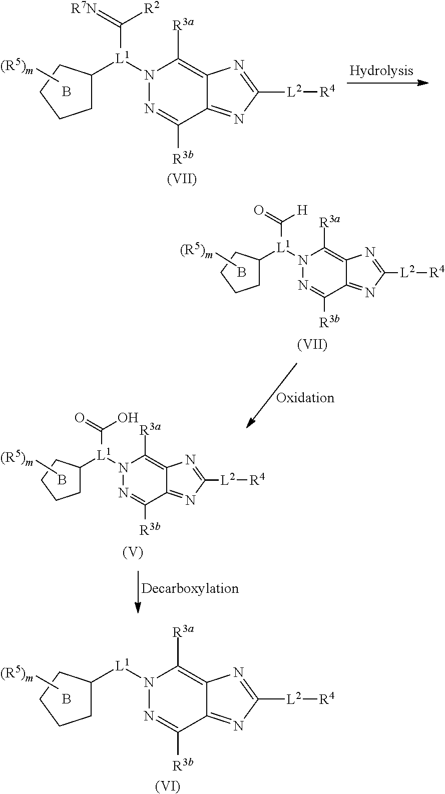

As indicated herein, the moiety “(NR⁷)CHR²” may be hydrolyzed to generate an aldehyde compound of Formula (VIII) that may be subsequently oxidized through a variety of oxidase and/or dehydrogenase enzymes (e.g., cytochrome P450 oxidase, NADPH oxidase, etc.) to generate the aforementioned carboxylated compound (V), which as described herein, can undergo spontaneous decarboxylation to generate a physiologically active compound of Formula (VI).

The aldehyde may also be formed through a variety of other metabolic pathways. For example, the aldehyde of Formula (VIII) may be formed via an alcohol compound of Formula (IX), which may undergo oxidation in a manner such as described herein to form the aldehyde of Formula (VIII):

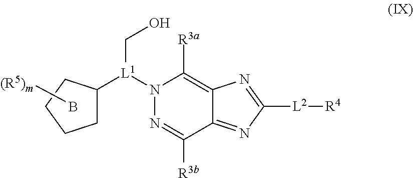

If desired, the aldehyde of Formula (VIII) may itself be employed as the prodrug compound of the present invention (e.g., wherein, Q⁴ is O, Y is a bond, and R² is H). Similarly, the alcohol of Formula (IX) may be employed as the prodrug compound of the present invention (e.g., wherein, Q⁴ is O, Y is O, and R² is H).

In some embodiments, pharmaceutical compositions are provided that contain a pharmaceutically acceptable diluent and a therapeutically effective amount of one of the compounds, or pharmaceutically acceptable salts or solvates, described herein or mixtures of one or more of such compounds, or pharmaceutically acceptable salts or solvates.

In some embodiments, methods are provided for treating patients having a viral infection mediated at least in part by a virus in the Flaviviridae family of viruses, such as HCV, which methods include administering to a patient that has been diagnosed with the viral infection or is at risk of developing the viral infection a pharmaceutical composition comprising a pharmaceutically acceptable diluent and a therapeutically effective amount of one of the compounds, or pharmaceutically acceptable salts or solvates, described herein or mixtures of one or more of such compounds, or pharmaceutically acceptable salts or solvates. In some embodiments, provided are use of the compounds of Formula (I), or pharmaceutically acceptable salts or solvates, for the preparation of a medicament for treating or preventing said infections. In some embodiments, the patient is a human.

In some embodiments, methods are provided for treating or preventing viral infections in patients in combination with the administration of a therapeutically effective amount of one or more agents active against HCV. Active agents against HCV include ribavirin, levovirin, viramidine, thymosin alpha-1, an inhibitor of NS3 serine protease, and inhibitor of inosine monophosphate dehydrogenase, interferon-alpha, pegylated interferon-alpha, alone or in combination with ribavirin or viramidine. In one example, the additional agent active against HCV is interferon-alpha or pegylated interferon-alpha alone or in combination with ribavirin or viramidine. In another example, the active agent is interferon.

### General Synthetic Methods

The compounds disclosed herein can be prepared by following the general procedures and examples set forth below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds, or pharmaceutically acceptable salts or solvates, described herein contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and so forth.

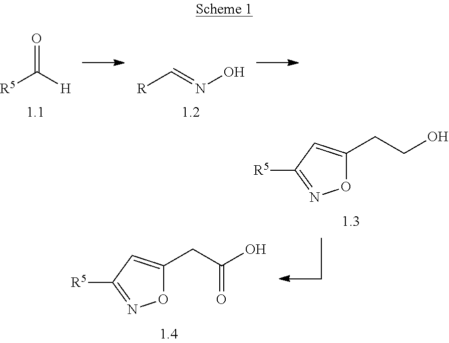

Scheme 1 shows the synthesis of 3-substituted carboxylated isoxazole intermediates wherein R⁵ is as defined for Formula (I). Aldehyde 1.1 is treated with hydroxylamine under oxime reaction conditions to give 1.2 that is then cyclized to isoxazole 1.3 through treatment with a chlorinating agent (e.g., N-chlorosuccinnimide or NaOCl), base (e.g., triethylamine), and an acetylenic alcohol (e.g., 3-butyn-1-ol). Isoxazole 1.3 then reacts with an oxidizing agent (e.g., pyridinium chlorochromate) to form carboxylated isoxazole 1.4.

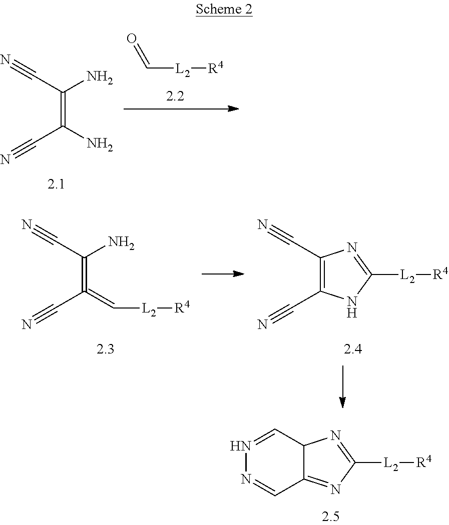

Scheme 2 shows the synthesis of imidazo[4,5-d]pyridazine intermediates wherein L² and R⁴ are as defined for Formula (I). A dinitrile 2.1 (Heterocycles, 29, 1325, 1989) is condensed with aldehyde 2.2 and oxidatively cyclized to the 2-substituted imidazole 4,5 dinitrile 2.3. This is then reduced with reagents such as diisobutylaluminium hydride (DIBAL-H) in a solvent (e.g., THF) to afford 2.4 and then subsequently cyclized with hydrazine or its derivatives to give 2-substituted-5H-imidazo[4,5-c]pyridazine 2.5.

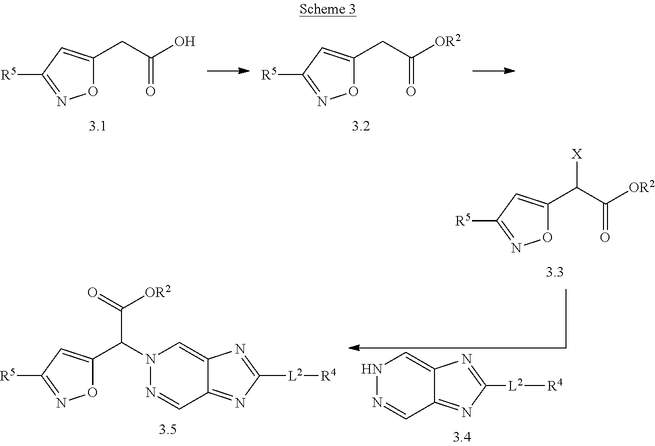

Scheme 3 shows the synthesis of the compounds of Formula (I) where for illustrative purposes G is C; Q⁴ is O; n is 0; Y is O; L¹ is CH; Q and V are CH; A is N; R¹ is isoxazolyl substituted with (R⁵)ₘ; and m is 1; and R², R⁵, L², and R⁴ are previously defined. Isoxazolyl ester 3.2 is formed from carboxylated isoxazole 3.1 through any of a variety of esterification reactions known to those skilled in the art, such as Fischer esterification, Steglich esterification, direct olefinic esterification, etc. Fischer esterification may occur, for instance, in the presence of an alcohol (e.g., methanol, ethanol, n-propanol, isopropanol, etc.) and an acid catalyst (e.g., HCl). Steglich esterification may occur in the presence of an alcohol, coupling reagent (e.g., N-ethyl-N′-(3-dimethylaminopropyl)carbodiimide), and catalyst (4-dimethylaminopyridine). Direct esterification may likewise be accomplished through reaction of 3.1 with an olefin (e.g., isobutylene) in the presence of an acid (e.g., sulfuric acid).

The isoxazolyl ester 3.2 may be halogenated to give 3.3 (X is, for example, Br or I) using known techniques, such as through reaction with a brominating reagent (e.g., N-bromosuccinimide) in the presence of a radical initiator (e.g., azo-bis-isobutyronitrile (AIBN) or benzoyl peroxide). Compound 3.5 may be synthesized from halogenated isoxazolyl ester 3.3 and substituted-5H-imidazo[4,5-d]pyridazine 3.4 through a variety of coupling reactions well known to those skilled in the art. These include, but are not limited to, Heck reactions, Suzuki reactions, Stille reactions, Sonogashira reactions, carbonylation reactions, cyanation reactions, and so forth. A number of catalyst, base, and solvent combinations may be employed to carry out the desired reaction. Compounds of general formula 3.5 may, for example, be synthesized in the presence of a base (e.g., Na₂CO₃, K₂CO₃, KF, CsF, etc.) and solvent (e.g., dimethylformaldehyde or dimethoxyethane) at temperatures between 50 and 250° C., optionally with the assistance of a microwave (e.g., Sm ithSynthesizer).

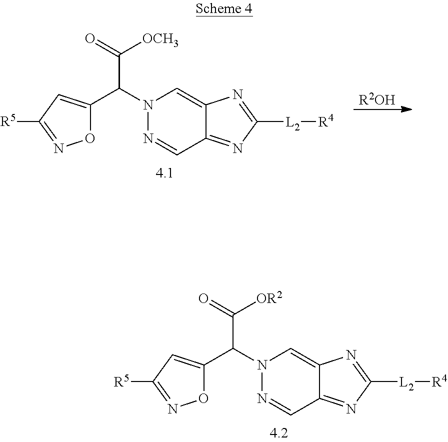

Scheme 4 shows the synthesis of the compounds of Formula (I) where for illustrative purposes G is C; Q⁴ is O; n is 0; Y is O; L¹ is CH; Q and V are CH; A is N; R¹ is isoxazolyl substituted with (R⁵)ₘ; and m is 1; and R², R⁵, L², and R⁴ are previously defined. Methyl ester 4.1 undergoes transesterification with “R²OH” (e.g., ethanol, n-propanol, isopropanol, i-butanol, n-butanol, trimethylene glycol, diethylene glycol, triethylene glycol, tetraethylene glycol, diethylene glycol methyl ether, and benzyl alcohol) in the presence of a base (e.g., Na₂CO₃, K₂CO₃, KF, CsF, etc.) to give a new ester 4.2.

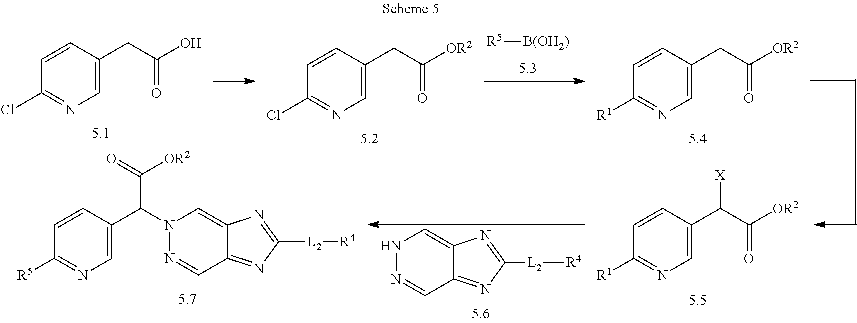

Scheme 5 shows the synthesis of the compounds of Formula (I) where for illustrative purposes G is C; Q⁴ is O; n is 0; Y is O; L¹ is CH; Q and V are CH; A is N; R¹ is pyridine substituted with (R⁵)ₘ; and m is 1; and R², R⁵, L², and R⁴ are previously defined. Pyridine ester 5.2 is synthesized from 2-chloropyridine-5-acetic acid 5.1 (a commercially available compound) using any of a variety of esterification reactions known to those skilled in the art, etc., such as described herein. Pyridine ester 5.2 may then undergo a metal-mediated coupling reaction with boronic acid 5.3 (or boronic ester) as is known in the art to give 2-substituted pyridine ester 5.4. Such boronic acids and boronate esters are commercially available or synthesized by standard methods. The coupling reaction may occur using any of a variety of known catalyst, base, and solvent combinations. Compounds of general formula 5.4 may, for example, may be synthesized in the presence of a base (e.g., Na₂CO₃, K₂CO₃, KF, CsF, etc.), solvent (e.g., dimethylformaldehyde or dimethoxyethane), and palladium catalyst (e.g., Pd(Ph₃P)₄ or PdCl₂(Ph₃P)₂).

2-substituted pyridine ester 5.4 may then be halogenated to give 5.5 (X is, for example, Br or I) using known techniques, such as through reaction with a brominating reagent (e.g., N-bromosuccinimide) in the presence of a radical initiator (e.g., azo-bis-isobutyronitrile (AIBN) or benzoyl peroxide). Compounds of formula 5.7 may be synthesized from halogenated compound 5.5 and substituted-5H-imidazo[4,5-d]pyridazine 5.6 through a variety of coupling reactions well known to those skilled in the art and described herein. Compounds of general formula 5.7 may, for example, be synthesized in the presence of a base (e.g., K₂CO₃) and solvent (e.g., dimethylformaldehyde or dimethoxyethane) at temperatures between 50 and 250° C., optionally with the assistance of a microwave (e.g., SmithSynthesizer).

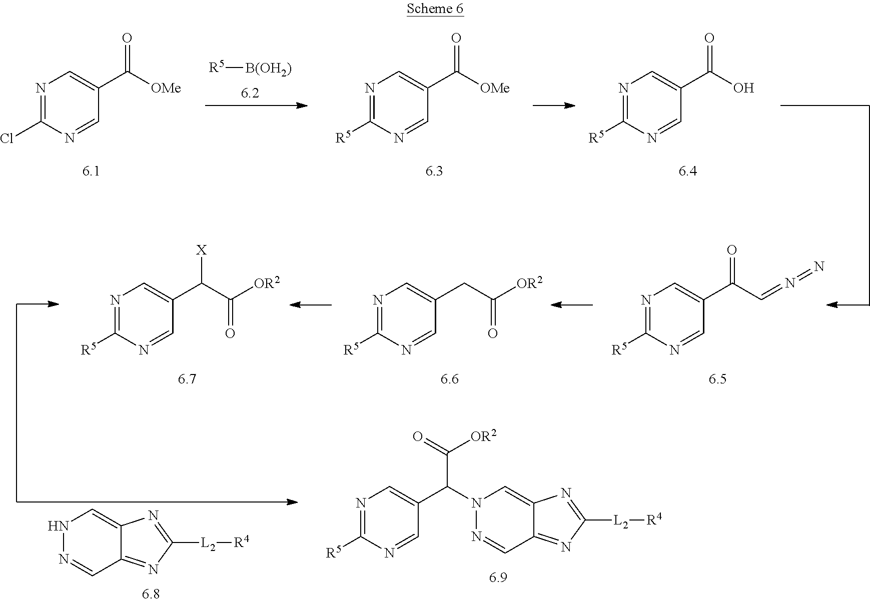

Scheme 6 shows the synthesis of the compounds of Formula (I) where for illustrative purposes G is C; Q⁴ is O; n is 0; Y is O; L¹ is CH; Q and V are CH; A is N; R¹ is pyrimidine substituted with (R⁵)ₘ; and m is 1; and R², R⁵, L², and R⁴ are previously defined. Pyrimidine ester 6.1 (a commercially available compound) may initially undergo a metal-mediated coupling reaction with boronic acid 6.2 (or ester) as is known in the art to give 2-substituted pyrimidine ester 6.3. Compound 6.3 is then hydrolyzed, such as through the use of lithium hydroxide in aqueous tetrahydrofuran (“THF”), to give carboxylated pyridimine 6.4. Compound 6.4 may then undergo a two-step reaction with a thionyl chloride to give an acid chloride, which is then reacted with a diazo (e.g., diazomethane) in the presence of a solvent (e.g., dichloromethane) to form α-diazo ketone 6.5. The α-diazo ketone 6.5 may undergo a Wolff rearrangement in the presence of a transition metal catalyst (e.g., silver benzoate or silver oxide), base (e.g., triethylamine, trimethylamine, etc.), and solvent R²OH (e.g., methanol, ethanol, etc.) to form ester 6.6. Compound 6.6 may be halogenated to give 6.7 (X is, for example, Br or I) using known techniques, such as through reaction with a brominating reagent (e.g., N-bromosuccinimide) in the presence of a radical initiator (e.g., azo-bis-isobutyronitrile (AIBN) or benzoyl peroxide). Compounds of formula 6.9 may be synthesized from halogenated compound 6.7 and substituted-5H-imidazo[4,5-d]pyridazine 6.8 through a variety of coupling reactions well known to those skilled in the art. These include, but are not limited to, Heck reactions, Suzuki reactions, Stille reactions, Sonogashira reactions, carbonylation reactions, cyanation reactions, and so forth. A number of catalyst, base, and solvent combinations may be employed to carry out the desired reaction. Compounds of general formula 6.9 may, for example, be synthesized in the presence of a base (e.g., K₂CO₃) and solvent (e.g., dimethylformaldehyde or dimethoxyethane) at temperatures between 50 and 250° C., optionally with the assistance of a microwave (e.g., SmithSynthesizer).

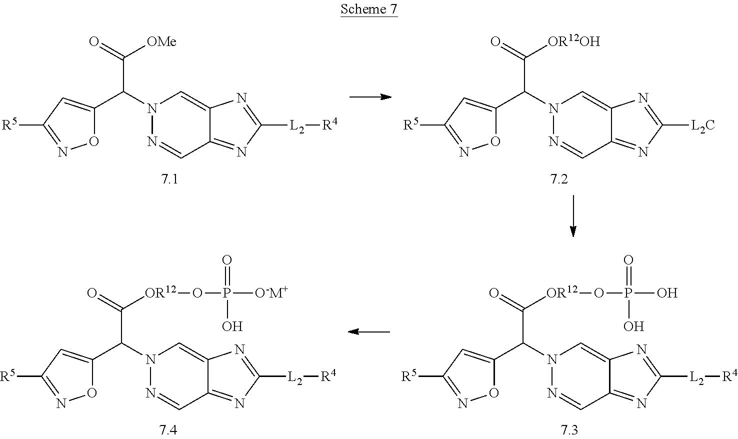

Scheme 7 shows the synthesis of the compounds of Formula (I) where for illustrative purposes G is C; Q⁴ is O; n is 0; Y is O; L¹ is CH; R² is R¹² (e.g., alkyl) substituted with phosphate; Q and V are CH; A is N; R¹ is isoxazolyl substituted with (R⁵)ₘ; and m is 1; and R⁵, L², and R⁴ are previously defined. Methyl ester 7.1 undergoes transesterification with a diol “HOR¹²OH” (e.g., trimethylene glycol, diethylene glycol, triethylene glycol, propylene glycol, and diethylene glycol methyl ether) in the presence of a base (e.g., Na₂CO₃, K₂CO₃, KF, CsF, etc.) to give a hydroxyester 7.2. Hydroxyester 7.2 reacts with phosphoryl chloride and water to give phosphoric acid 7.3. Neutralization may be accomplished through the addition of pharmaceutically acceptable counterions “M” (e.g., sodium) in the presence of a solvent (e.g., acetonitrile and water) to give the monophosphate ester 7.4.

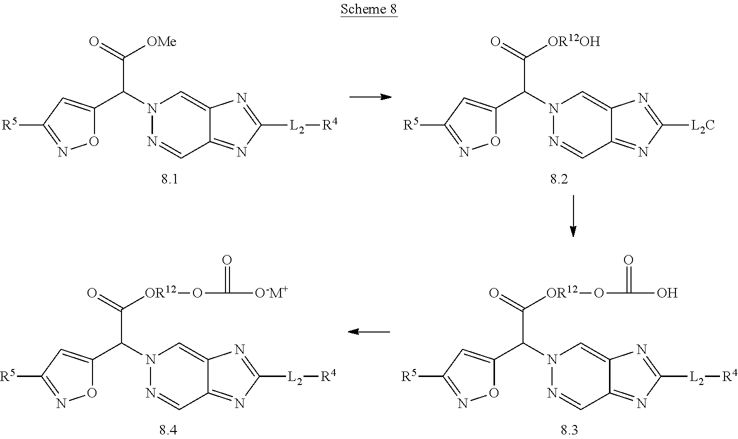

Scheme 8 shows the synthesis of the compounds of Formula (I) where for illustrative purposes G is C; Q⁴ is O; n is 0; Y is O; L¹ is CH; R² is R¹² (e.g., alkyl) substituted with carboxyl; Q and V are CH; A is N; R¹ is isoxazolyl substituted with (R⁵)ₘ; and m is 1; and R⁵, L², and R⁴ are previously defined. Methyl ester 8.1 undergoes transesterification with a diol “HOR¹²OH” as described herein to give a hydroxyester 8.2. Hydroxyester 8.2 then reacts with an oxidizing agent (e.g., orthoperiodic acid) in the presence of a catalyst (e.g., pyridinium chlorochromate) and solvent (e.g., acetonitrile) to form carboxylic acid 8.3. Neutralization may be accomplished through the addition of pharmaceutically acceptable counterions “M” (e.g., sodium) in the presence of a solvent (e.g., acetonitrile and water) to give the carboxylate salt 8.4.

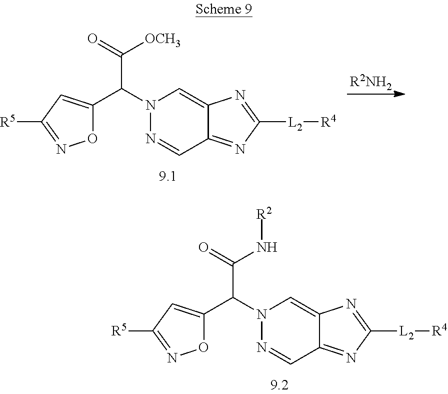

Scheme 9 shows the synthesis of the compounds of Formula (I) where for illustrative purposes G is C; Q⁴ is O; n is 0; Y is NH; L¹ is CH; Q and V are CH; A is N; R¹ is isoxazolyl substituted with (R⁵)ₘ; and m is 1; and R², R⁵, L², and R⁴ are previously defined. Methyl ester 9.1 reacts with a primary amine “R²NH₂” (e.g., methylamine, ethylamine, etc.) in the presence of an alcohol (e.g., methanol) to give a secondary amide 9.2.

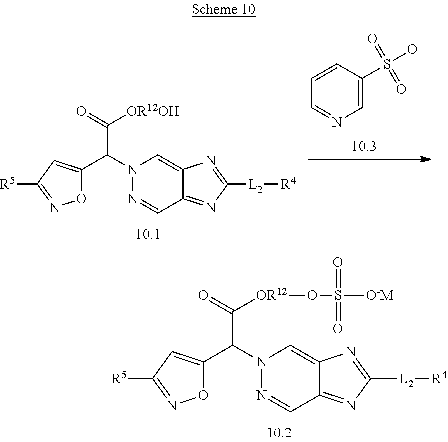

Scheme 10 shows the synthesis of the compounds of Formula (I) where for illustrative purposes G is C; Q⁴ is O; n is 0; Y is O; L¹ is CH; R² is R¹² (e.g., alkyl) substituted with sulfate; Q and V are CH; A is N; R¹ is isoxazolyl substituted with (R⁵)ₘ; and m is 1; and R⁵, L², and R⁴ are previously defined. Hydroxyester 10.1 reacts with pyridine sulfonate 10.3 in the presence of a solvent (e.g., DMF) to afford a monosulfate ester, which may be neutralized through the addition of pharmaceutically acceptable counterions “M” (e.g., sodium) in the presence of a solvent (e.g., acetonitrile and water) to give the salt 10.2.

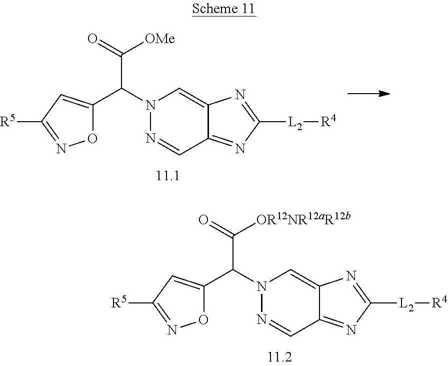

Scheme 11 shows the synthesis of the compounds of Formula (I) where for illustrative purposes G is C; Q⁴ is O; n is 0; Y is O; L¹ is CH; Q and V are CH; A is N; R¹ is isoxazolyl substituted with (R⁵)ₘ; R² is R¹² (e.g., alkyl) substituted with substituted amino (e.g., dimethylamine); and m is 1; and R⁵, L², and R⁴ are previously defined. As shown, methyl ester 11.1 reacts with an amino alcohol “OHR¹²NR^{12a}R^{12b}” (where R^{12a} and R^{12b} are independently hydrogen or alkyl), such as 4-(dimethylamino)-butan-1-ol, in the presence of a base (e.g., Na₂CO₃, K₂CO₃, KF, CsF, etc.) to give the amino ester 11.2. Neutralization may optionally be accomplished through the addition of pharmaceutically acceptable counterions (e.g., hydrogen chloride).

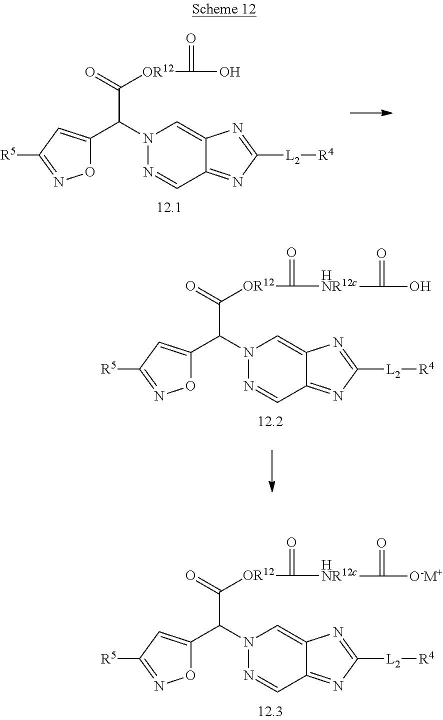

Scheme 12 shows the synthesis of the compounds of Formula (I) where for illustrative purposes G is C; Q⁴ is O; n is 0; Y is O; L¹ is CH; Q and V are CH; A is N; R¹ is isoxazolyl substituted with (R⁵)ₘ; m is 1; and R² is R¹² substituted with C(O)NHR^{12c}C(O)O⁻M⁺, where R¹² and R^{12c} are independently alkyl; and R⁵, L², and R⁴ are previously defined. Hydroxyester 12.1, such as described herein, undergoes a coupling reaction with an amino acid carbonyl “OH(O)CR^{12c}NHC(O)R^{12d}” (e.g., L-alanine-O-t-butyl) in the presence of a base (e.g., N,N-diisopropylethylamine) and solvent (e.g., acetonitrile). Conventional amino acid coupling reagents may be employed to facilitate the reaction, such as 2-(7-aza-1H-benzotriazole-1-yl)-1,1,3,3-tetramethyluronium hexafluorophosphate (“HATU”). The resulting carboxylic acid 12.2 may be neutralized through the addition of pharmaceutically acceptable counterions “M” (e.g., sodium) in the presence of a solvent (e.g., acetonitrile and water) to give the salt 12.3.

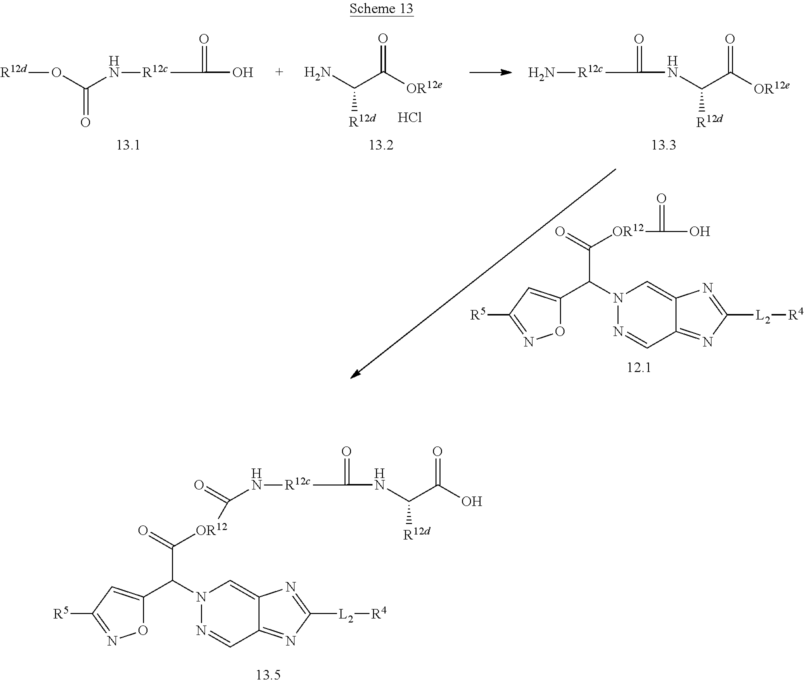

Scheme 13 shows the synthesis of the compounds of Formula (I) where for illustrative purposes G is C; Q⁴ is O; n is 0; Y is O; L¹ is CH; R² is R¹² substituted with C(O)NHR^{12c}C(O)NHCHR^{12d}C(O)OH, where R¹², R^{12c}, and R^{12d} are independently alkyl, substituted alkyl (e.g., methylphenyl), aryl, or substituted aryl; Q and V are CH; A is N; R¹ is isoxazolyl substituted with (R⁵)ₘ; and m is 1; and R⁵, L², and R⁴ are previously defined. Amino acid derivative 13.1 (e.g., (2S)-2-benzyloxycarbonylaminopropanoic acid) initially undergoes a coupling reaction with an amino acid ester 13.2 (where R^{12e} is, for example, an alkyl group (e.g., t-butyl)), such as (3S)-3-amino-4-phenyl-butan-2-one. Conventional amino acid coupling reagents may be employed to facilitate the reaction, such as 2-(7-aza-1H-benzotriazole-1-yl)-1,1,3,3-tetramethyluronium hexafluorophosphate (“HATU”). The resulting amide 13.3 may then be coupled to compound 12.1 (described herein) in the presence of a coupling agent (e.g., HATU), base (e.g., N,N-diisopropylethylamine), and solvent (e.g., acetonitrile) to give the compound 13.5.

The foregoing and other aspects of the present invention may be better understood in connection with the following representative examples.

### EXAMPLES

In the examples below and the synthetic schemes herein, the following abbreviations have the following meanings. If an abbreviation is not defined, it has its generally accepted meaning.
μL=microliters
PM=micromolar
pmol=micromoles
NMR=nuclear magnetic resonance
br=broad
d=doublet
δ=chemical shift
° C.=degrees celsius
AcOH=acetyl alcohol
AIBN=2-2′-azobis(isobutyronitrile)
DCM=dichloromethane
dd=doublet of doublets
DME=1,2-dimethoxyethane
DMEM=Dulbeco's Modified Eagle's Medium
DMF=N,N-dimethylformamide
DMSO=dimethylsulfoxide
EDCl=1-ethyl-3-(3-dimethylaminopropyl) carbodiimide)
EtOAc=ethyl acetate
EtOH=ethanol
g=gram
h or hr=hours
HCV=hepatitus C virus
HPLC=high performance liquid chromatography
Hz=hertz
IC₅₀=inhibitory concentration at 50% inhibition
iPrOH=isopropyl alcohol
J=coupling constant (given in Hz unless otherwise indicated)
LCMS=Liquid chromatography coupled mass spectroscopy
m=multiplet
M=molar
M+H⁺=parent mass spectrum peak plus H⁺
MeCN=acetonitrile
MeOH=methanol
mg=milligram
mL=milliliter
mM=millimolar
mmol=millimole
MS=mass spectrum
nBuOH=n-butanol
NBS=N-bromosuccinimide
nm=nanomolar
ng=nanogram
PCC=pyridinium chlorochromate
Ph=phenyl
ppm=parts per million
R_{f}=retention factor (for TLC)
RT=room temperature
s=Singlet
t=triplet
TFA=trifluoroacetic acid
TLC=thin layer chromatography
wt %=weight percent

### Example 1

### Methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 1

### Step A

### 4-(Propyloxy)-2-(trifluoromethyl)benzaldehyde oxime

A solution of 16.3 g (70.2 mmol) of 4-(propyloxy)-2-(trifluoromethyl)benzaldehyde in 70 mL of EtOH was treated with 10 mL of 50% hydroxylamine/water (140 mmol) and the resulting solution was stirred at RT. After 2 hours TLC (silica gel, 8:2 hexane/EtOAc) indicated complete conversion of the aldehyde starting material to a new, lower R_{f} spot. The solution was concentrated by rotary evaporation to a volume of approximately 25 mL at which point a white solid precipitated. The suspension was diluted with 60 mL of water and stirred for 45 minutes. The solid was collected by vacuum filtration on a medium frit. The filter cake was washed twice with water, suction dried for 20 minutes and then dried in vacuo overnight to afford 16.9 g of a very pale yellow crystalline solid. The crude material was recrystallized from hexane/EtOAc to afford 14.5 (84%) of 4-(propyloxy)-2-(trifluoromethyl)benzaldehyde oxime as a white crystalline solid. ¹H NMR (400 MHz, DMSO-d₆) δ ppm 11.54 (s, 1H) 8.07-8.23 (m, 1H) 7.88 (d, J=8.8 Hz, 1H) 7.11-7.28 (m, 2H) 3.99 (t, J=6.5 Hz, 2H) 1.70 (sxt, J=7.0 Hz, 2H) 0.93 (t, J=7.4 Hz, 3H). LCMS m/z 248 (M+1).

### Step B

### 2-{3-[4-(Propyloxy)-2-(trifluoromethyl)phenyl]-5-isoxazolyl}ethanol

A stirred solution of 3.00 g (12.1 mmol) of 4-(propyloxy)-2-(trifluoromethyl)benzaldehyde oxime in 25 mL of DMF was cooled to 0° C. and was treated with 1.70 g (12.7 mmol) of NCS by slow addition over 1 minute. After 1 hour the solution was diluted with EtOAc, washed with water (2×), brine (1×), dried over sodium sulfate and concentrated to dryness at reduced pressure. The residue was dissolved in 35 mL of 1,2-DCE and the solution treated with 1.30 g (18.2 mmol) of 3-butyn-1-ol followed by 2.60 mL (18.2 mmol) of TEA. A solid immediately precipitated (TEA-HCl salt). The suspension was heated to reflux with stirring at which point the solid had gone into solution. After 30 minutes the solution was cooled to RT and stirred overnight. The solution was diluted 2-fold with DCM, washed with water (3×), brine (1×), dried over sodium sulfate and concentrated to dryness at reduced pressure to afford 4.02 g (95%) of 2-{3-[4-(propyloxy)-2-(trifluoromethyl)phenyl]-5-isoxazolyl}ethanol as a viscous yellow oil. ¹H NMR (400 MHz, CHLOROFORM-d) δ ppm 7.53 (d, J=8.6 Hz, 1H) 7.27 (d, J=2.6 Hz, 1H) 7.07 (dd, J=8.5, 2.6 Hz, 1H) 6.27 (d, J=0.7 Hz, 1H) 3.98 (td, J=6.4, 3.1 Hz, 4H) 2.99-3.11 (m, 2H) 1.73-1.92 (m, 2H) 1.04 (t, J=7.4 Hz, 3H). LCMS m/z 316 (M+1).

### Step C

### {3-[4-(Propyloxy)-2-(trifluoromethyl)phenyl]-5-isoxazolyl}acetic acid

Periodic acid (4.58 g, 20.1 mmol) was added to 70 mL of anhydrous MeCN and the mixture stirred at RT for 15 minutes. The solid reagent slowly dissolved to afford a clear solution. A solution of 2.88 g (9.13 mmol) of 2-{3-[4-(propyloxy)-2-(trifluoromethyl)phenyl]-5-isoxazolyl}ethanol in 15 mL of MeCN was added and the resulting solution was cooled in an ice water bath. The solution was then treated with 40 mg (0.18 mmol) of PCC. A light yellow precipitate was rapidly produced. The ice bath was removed and the reaction mixture was allowed to warm to RT with stirring. After 3 hours LCMS indicated complete conversion to the desired product. The mixture was subjected to rotary evaporation to a volume of approximately 15 mL and was then diluted with 100 mL of 9:1 chloroform/iPrOH. The rapidly stirred suspension was treated with 100 mL of 10% aqueous sodium bisulfite and the mixture stirred vigorously for a short time. The mixture was transferred to a separatory funnel and the phases separated. The organic solution was washed with water (3×), brine (1×), dried over sodium sulfate and concentrated to dryness at reduced pressure to afford 2.85 g (95%) of {3-[4-(propyloxy)-2-(trifluoromethyl)phenyl]-5-isoxazolyl}acetic acid as a light tan solid. ¹H NMR (400 MHz, DMSO-d₆) δ ppm 12.89 (br s, 1H) 7.58 (d, J=8.4 Hz, 1H) 7.27 ˜7.42 (m, 2H) 6.57 (s, 1H) 4.08 (t, J=6.5 Hz, 2H) 3.98 (s, 2H) 1.77 (sxt, J=7.0 Hz, 2H) 1.00 (t, J=7.4 Hz, 3H). LCMS m/z 330 (M+1).

### Step D

### Methyl {3-[4-(propyloxy)-2-(trifluoromethyl)phenyl]-5-isoxazolyl}acetate

Thionyl chloride (4 mL) was slowly added to a stirred 20 mL portion of MeOH. After 10 minutes a solution of 0.530 g (1.61 mmol) of {3-[4-(propyloxy)-2-(trifluoromethyl)phenyl]-5-isoxazolyl}acetic acid in 6 mL of MeOH was added and the resulting solution was stirred at RT. After 2 hours the solution was concentrated to dryness at reduced pressure and the residue dissolved in EtOAc. The solution was washed with saturated aqueous sodium bicarbonate (2×), brine (1×), dried over sodium sulfate and concentrated to dryness at reduced pressure to afford 0.55 g (99%) of methyl {3-[4-(propyloxy)-2-(trifluoromethyl)phenyl]-5-isoxazolyl}acetate as a light yellow oil. ¹H NMR (400 MHz, DMSO-d₆) δ ppm 7.59 (d, J=8.4 Hz, 1H) 7.29-7.41 (m, 2H) 6.60 (s, 1H) 4.12 (s, 2H) 4.08 (t, J=6.5 Hz, 2H) 3.69 (s, 3H) 1.68-1.84 (m, 2H) 1.00 (t, J=7.4 Hz, 3H). LCMS m/z 344 (M+1).

### Step E

### Methyl bromo{3-[4-(propyloxy)-2-(trifluoromethyl)phenyl]-5-isoxazolyl}acetate

A mixture of 0.383 g (1.12 mmol) of methyl {3-[4-(propyloxy)-2-(trifluoromethyl)phenyl]-5-isoxazolyl}acetate, 0.238 g (1.34 mmol) of NBS and 10 mg (0.061 mmol) of AIBN in 20 mL of carbon tetrachloride was heated to reflux with stirring under a nitrogen atmosphere. After 2 hours TLC (silica gel, 1:1 hexane /EtOAc) indicated approximately 50% conversion of the starting material to a slightly higher R_{f} component. The mixture was treated with an additional 200 mg (1.12 mmol) of NBS followed by 10 mg (0.061 mmol) of AIBN and stirring at reflux continued. After 3 more hours of refluxing the reaction mixture was cooled to RT and stirred overnight. The mixture was diluted with DCM and the solids removed by filtration through a medium fritted funnel. The filtrate was concentrated to dryness at reduced pressure and the residue subjected to flash chromatography (silica gel, gradient elution from hexane to 4:6 hexane/EtOAc) to afford 268 mg (57%) of methyl bromo{3-[4-(propyloxy)-2-(trifluoromethyl)phenyl]-5-isoxazolyl}acetate as a clear oil. ¹H NMR (400 MHz, DMSO-d₆) δ ppm 7.63 (d, J=8.5 Hz, 1H) 7.33-7.42 (m, 2H) 6.90 (s, 1H) 6.44 (s, 1H) 4.09 (t, J=6.5 Hz, 2H) 3.76-3.86 (m, 3H) 1.70-1.82 (m, 2H) 1.00 (t, J=7.4 Hz, 3H).

### Step F

### Methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

A mixture of 0.313 g (1.35 mmol) of 2-(2,3-difluorophenyl)-5H-imidazo[4,5-d]pyridazine in 10 mL of DMF was briefly warmed to dissolve the starting material. The resulting solution was treated with 0.622 g (4.50 mmol) of potassium carbonate and cooled in an ice water bath. A solution of 0.380 g (0.900 mmol) of methyl bromo{3-[4-(propyloxy)-2-(trifluoromethyl)phenyl]-5-isoxazolyl}acetate in 4 mL of DMF was added dropwise over 3 minutes. The mixture was allowed to warm to RT. After 1.5 hours the mixture was partitioned between 10% aqueous NaCl and EtOAc and the phases separated. The aqueous phase was back extracted with EtOAc (3×). The combined EtOAc solutions were washed with 10% aqueous NaCl (2×), saturated aqueous brine (1×), dried over sodium sulfate and concentrated to dryness at reduced pressure. The crude product was subjected to flash chromatography (silica gel, gradient elution from DCM to 9:1 DCM/MeOH) followed by reverse phase HPLC purification (C18, gradient elution from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in EtOAc. The solution was washed with saturated aqueous sodium bicarbonate (1×), brine (1×), dried over sodium sulfate and concentrated to dryness at reduced pressure to afford 280 mg (54%) of methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a white foam. ¹H NMR (400 MHz, CHLOROFORM-d) δ ppm 9.40 (s, 1H) 9.29 (d, J=0.9 Hz, 1H) 8.15-8.23 (m, 1H) 7.58 (d, J=8.6 Hz, 1H) 7.17-7.34 (m, 3H) 7.12 (dd, J=8.5, 2.5 Hz, 1H) 6.88 (s, 1H) 6.85 (s, 1H) 4.00 (t, J=6.5 Hz, 2H) 3.94 (s, 3H) 1.84 (sxt, J=7.1 Hz, 2H) 1.05 (t, J=7.4 Hz, 3H). LCMS m/z 574 (M+1).

### Example 2

Ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 2

### Step A

### Ethyl {3-[4-(propyloxy)-2-(trifluoromethyl)phenyl]-5-isoxazolyl}acetate

A solution of 0.250 g (0.759 mmol) of {3-[4-(propyloxy)-2-(trifluoromethyl)phenyl]-5-isoxazolyl}acetic acid in 10 mL of anhydrous DCM was treated with 0.44 mL (7.6 mmol) of absolute EtOH followed by 10 mg (0.082 mmol) of DMAP and then 0.291 g (1.52 mmol) of EDCl. The resulting solution was stirred at RT. After 2 hours LCMS indicated complete reaction. The solution was concentrated to dryness at reduced pressure. The residue was partitioned between EtOAc and 10% aqueous citric acid and the phases separated. The EtOAc solution was washed with 10% citric acid (2×), saturated aqueous sodium bicarbonate (2×), brine (1×), dried over sodium sulfate and concentrated to dryness to afford 0.254 g (94%) of ethyl {3-[4-(propyloxy)-2-(trifluoromethyl)phenyl]-5-isoxazolyl}acetate as a light yellow oil. ¹H NMR (400 MHz, DMSO-d₆) δ ppm 7.59 (d, J=8.4 Hz, 1H) 7.30-7.40 (m, 2H) 6.60 (s, 1H) 4.15 (q, J=7.1 Hz, 2H) 4.05-4.11 (m, 4H) 1.77 (sxt, J=7.0 Hz, 2H) 1.21 (t, J=7.1 Hz, 3H) 1.00 (t, J=7.4 Hz, 3H). LCMS m/z 358 (M+1).

### Step B

### Ethyl bromo{3-[4-(propyloxy)-2-(trifluoromethyl)phenyl]-5-isoxazolyl}acetate

A mixture of 0.250 g (0.700 mmol) of ethyl {3-[4-(propyloxy)-2-(trifluoromethyl)phenyl]-5-isoxazolyl}acetate, 0.187 g (1.05 mmol) of NBS, and 10 mg (0.061 mmol) of AIBN in 25 mL of CCl₄ was heated to reflux with stirring. After 18 hours TLC indicated approximately 30% conversion to a new, higher R_{f} component. The solution was treated with an additional 50 mg (0.28 mmol) of NBS and stirred at reflux for another 7 hours. The solution was cooled to RT and stirred for three days. The reaction mixture was concentrated to dryness and the residue subjected to flash chromatography (silica gel, gradient elution from hexane to EtOAc) to afford 0.141 g (46%) of ethyl bromo{3-[4-(propyloxy)-2-(trifluoromethyl)phenyl]-5-isoxazolyl}acetate as a clear oil. ¹H NMR (400 MHz, CHLOROFORM-d) δ ppm 7.55 (d, J=8.6 Hz, 1H) 7.28 (d, J=2.5 Hz, 1H) 7.09 (dd, J=8.6, 2.5 Hz, 1H) 6.74 (s, 1H) 5.49 (s, 1H) 4.25-4.38 (m, 2H) 3.98 (t, J=6.5 Hz, 2H) 1.83 (sxt, J=7.0 Hz, 2H) 1.32 (t, J=7.1 Hz, 3H) 1.04 (t, J=7.4 Hz, 3H).

### Step C

### Ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

A mixture of 77 mg (0.33 mmol) of 2-(2,3-difluorophenyl)-5H-imidazo[4,5-d]pyridazine in 6 mL of DMF was briefly warmed to dissolve the starting material. The resulting solution was treated with 0.134 g (0.970 mmol) of K₂CO₃ and cooled in an ice water bath. A solution of 0.141 g (0.323 mmol) of ethyl bromo{3-[4-(propyloxy)-2-(trifluoromethyl)phenyl]-5-isoxazolyl}acetate in 2 mL of DMF was added by dropwise addition. The mixture was allowed to warm to RT. After 1 hour the mixture was diluted with EtOAc, washed with half saturated brine (2×), saturated brine (1×), dried over sodium sulfate and concentrated to dryness at reduced pressure. The crude residue was subjected to reverse phase HPLC purification (C18, gradient elution from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in EtOAc. The solution was washed with saturated aqueous sodium bicarbonate (1×), brine (1×), dried over sodium sulfate and concentrated to dryness at reduced pressure to afford 0.107 g (56%) of ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a light yellow foam. ¹H NMR (400 MHz, DMSO-d₆) δ ppm 10.11 (d, J=0.9 Hz, 1H) 9.56 (d, J=1.0 Hz, 1H) 8.10-8.25 (m, 1H) 7.49-7.74 (m, 3H) 7.28-7.45 (m, 3H) 7.11 (s, 1H) 4.21-4.45 (m, 2H) 4.09 (t, J=6.5 Hz, 2H) 1.69-1.86 (m, 2H) 1.13-1.28 (m, 3H) 1.00 (t, J=7.4 Hz, 3H). LCMS m/z 588 (M+1).

### Example 3

### Propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 3

### Step A

### Propyl {3-[4-(propyloxy)-2-(trifluoromethyl)phenyl]-5-isoxazolyl}acetate

This compound was prepared in 90% yield from {3-[4-(propyloxy)-2-(trifluoromethyl)phenyl]-5-isoxazolyl}acetic acid and n-propanol according to the method described herein in Example 2 for the preparation of ethyl {3-[4-(propyloxy)-2-(trifluoromethyl)phenyl]-5-isoxazolyl}acetate. ¹H NMR (400 MHz, DMSO-d₆) δ ppm 7.58 (d, J=8.4 Hz, 1H) 7.28-7.41 (m, 2H) 6.59 (s, 1H) 3.97-4.16 (m, 6H) 1.70-1.86 (m, 2H) 1.60 (sxt, J=7.1 Hz, 2H) 1.00 (t, J=7.4 Hz, 3H) 0.88 (t, 3H). LCMS m/z 372 (M+1).

### Step B

### Propyl bromo{3-[4-(propyloxy)-2-(trifluoromethyl)phenyl]-5-isoxazolyl}acetate

This compound was prepared in 52% yield by NBS bromination of propyl {3-[4-(propyloxy)-2-(trifluoromethyl)phenyl]-5-isoxazolyl}acetate according to the method described herein in Example 2 for the preparation of ethyl bromo{3-[4-(propyloxy)-2-(trifluoromethyl)phenyl]-5-isoxazolyl}acetate. ¹H NMR (400 MHz, CHLOROFORM-d) δ ppm 7.56 (d, J=8.5 Hz, 1H) 7.28 (d, J=2.6 Hz, 1H) 7.09 (dd, J=8.5, 2.6 Hz, 1H) 6.74 (s, 1H) 5.50 (s, 1H) 4.22 (td, J=6.6, 2.6 Hz, 2H) 3.99 (t, J=6.5 Hz, 2H) 1.84 (sxt, J=7.1 Hz, 2H) 1.66-1.77 (m, 2H) 1.05 (t, J=7.4 Hz, 3H) 0.96 (t, J=7.4 Hz, 3H).

### Step C

### Propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

Compound 3 was also prepared in 31% yield from propyl bromo{3-[4-(propyloxy)-2-(trifluoromethyl)phenyl]-5-isoxazolyl}acetate and 2-(2,3-difluorophenyl)-5H-imidazo[4,5-d]pyridazine according to the method described herein in Example 2 for the synthesis of ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate. ¹H NMR (400 MHz, DMSO-d₆) δ ppm 10.11 (d, J=0.9 Hz, 1H) 9.56 (d, J=0.9 Hz, 1H) 8.16-8.23 (m, 1H) 7.70 (s, 1H) 7.54-7.67 (m, 2H) 7.32-7.42 (m, 3H) 7.10 (s, 1H) 4.24 (qt, J=10.9, 6.3 Hz, 2H) 4.09 (t, J=6.5 Hz, 2H) 1.70-1.85 (m, 2H) 1.50-1.67 (m, 2H) 1.00 (t, J=7.4 Hz, 3H) 0.80 (t, J=7.4 Hz, 3H). LCMS m/z 602 (M+1).

### Example 4

### 1,1-Dimethylethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 6

### Step A

### 1,1-Dimethylethyl {3-[4-(propyloxy)-2-(trifluoromethyl)phenyl]-5-isoxazolyl}acetate

Approximately 60 mL of isobutylene was condensed into a 150 mL pressure bottle cooled in an iPrOH/dry ice bath. To this was added 2.00 g (6.07 mmol) of {3-[4-(propyloxy)-2-(trifluoromethyl)phenyl]-5-isoxazolyl}acetic acid dissolved in 25 mL of ethyl ether. The mixture was then treated with 0.5 mL of concentrated sulfuric acid, the vessel sealed, and warmed to RT with vigorous stirring. After 4 days the reaction vessel was cooled in a dry ice acetone bath, the cap removed and the solution poured into 250 mL of rapidly stirred saturated aqueous sodium bicarbonate. The mixture was diluted with 150 mL of EtOAc and stirred for several minutes. The phases were separated and the aqueous solution extracted with an additional portion of EtOAc. The combined EtOAc solutions were washed with saturated sodium bicarbonate (1×), brine (1×), dried over sodium sulfate and concentrated to dryness at reduced pressure. The crude residue was subjected to flash chromatography (silica gel, gradient elution from hexane to EtOAc) to afford 1.70 g (73%) of 1,1-dimethylethyl {3-[4-(propyloxy)-2-(trifluoromethyl)phenyl]-5-isoxazolyl}acetate as a light yellow oil. ¹H NMR (400 MHz, DMSO-d₆) δ ppm 7.58 (d, J=8.3 Hz, 1H) 7.31-7.39 (m, 2H) 6.56 (s, 1H) 4.08 (t, J=6.5 Hz, 2H) 3.98 (s, 2H) 1.77 (sxt, J=7.1 Hz, 2H) 1.43 (s, 9H) 1.00 (t, J=7.4 Hz, 3H). LCMS m/z 386 (M+1).

### Step B

### 1,1-Dimethylethyl bromo{3-[4-(propyloxy)-2-(trifluoromethyl)phenyl]-5-isoxazolyl}acetate

This compound was prepared in 35% yield by NBS bromination of 1,1-dimethylethyl {3-[4-(propyloxy)-2-(trifluoromethyl)phenyl]-5-isoxazolyl}acetate according to the method described herein in Example 2 for the synthesis of ethyl bromo{3-[4-(propyloxy)-2-(trifluoromethyl)phenyl]-5-isoxazolyl}acetate. ¹H NMR (400 MHz, CHLOROFORM-d) δ ppm 7.57 (d, J=8.6 Hz, 1H) 7.28 (d, J=2.6 Hz, 1H) 7.09 (dd, J=8.5, 2.6 Hz, 1H) 6.70 (s, 1H) 5.40 (s, 1H) 3.99 (t, J=6.5 Hz, 2H) 1.84 (sxt, J=7.1 Hz, 2H) 1.50 (s, 9H) 1.05 (t, J=7.4 Hz, 3H).

### Step C

### 1,1-Dimethylethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

Compound 6 was prepared in 47% yield from 1,1-dimethylethyl bromo{3-[4-(propyloxy)-2-(trifluoromethyl)phenyl]-5-isoxazolyl}acetate and 2-(2,3-difluorophenyl)-5H-imidazo[4,5-d]pyridazine according to the method described herein in Example 2 for the synthesis of ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate. ¹H NMR (400 MHz, DMSO-d₆) δ ppm 10.08 (d, J=1.0 Hz, 1H) 9.56 (d, J=1.0 Hz, 1H) 8.12-8.26 (m, 1H) 7.53-7.67 (m, 3H) 7.33-7.42 (m, 3H) 7.06 (s, 1H) 4.09 (t, J=6.5 Hz, 2H) 1.77 (sxt, J=7.1 Hz, 2H) 1.45 (s, 9H) 1.00 (t, J=7.4 Hz, 3H). LCMS m/z 616 (M+1).

### Example 5

### 1-Methylethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 4

A solution of 60 mg (0.11 mmol) of methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 4 mL of isopropanol was treated with 20 mg (0.15 mmol) of potassium carbonate. The resulting mixture was subjected to microwave heating at 100° C. for 20 minutes. After cooling to RT the mixture was treated with 0.5 mL of glacial AcOH and diluted with EtOAc. The resulting solution was washed with water (2×), brine (1×), dried over sodium sulfate and concentrated to dryness at reduced pressure. The crude residue was subjected to reverse phase HPLC purification as described herein for the preparation of ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate to afford 39 mg (62%) of 1-methylethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a white foam. ¹H NMR (400 MHz, CHLOROFORM-d) δ ppm 9.33 (s, 1H) 9.30 (s, 1H) 8.17-8.23 (m, 1H) 7.59 (d, J=8.6 Hz, 1H) 7.17-7.34 (m, 3H) 7.12 (dd, J=8.6, 2.4 Hz, 1H) 6.87 (s, 1H) 6.75 (s, 1H) 5.18-5.33 (m, 1H) 4.00 (t, J=6.5 Hz, 2H) 1.85 (sxt, J=7.1 Hz, 2H) 1.34-1.27 (m, 6H) 1.06 (t, J=7.4 Hz, 3H). LCMS m/z 602 (M+1).

### Example 6

### 3-Hydroxypropyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 5

A solution of 1.00 g (1.74 mmol) of methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 15 mL of 1:1 DME/1,3-dihydroxypropane was treated with 0.720 g (5.23 mmol) of K₂CO₃ and the resulting orange solution stirred at RT. After 5 hours LCMS indicated complete reaction. The mixture was treated with 3 mL of glacial AcOH and diluted with EtOAc. The resulting solution was washed with water (3×), saturated brine (1×), dried over sodium sulfate and concentrated to dryness at reduced pressure. The crude material was subjected to flash chromatography (silica gel, gradient elution from DCM to 9:1 DCM/MeOH) to afford 0.850 g (79%) of 3-hydroxypropyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a light yellow foam. ¹H NMR (400 MHz, CHLOROFORM-d) δ ppm 9.36 (d, J=1.0 Hz, 1H) 9.27 (d, J=1.0 Hz, 1H) 8.12-8.23 (m, 1H) 7.57 (d, J=8.6 Hz, 1H) 7.15-7.34 (m, 3H) 7.12 (dd, J=8.6, 2.5 Hz, 1H) 6.90 (s, 1H) 6.81 (s, 1H) 4.41-4.61 (m, 2H) 4.00 (t, J=6.5 Hz, 2H) 3.67 (t, J=5.9 Hz, 2H) 1.75-1.99 (m, 4H) 1.05 (t, J=7.4 Hz, 3H). LCMS m/z 618 (M+1).

### Example 7

### Butyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 7

A solution of 60 mg (0.11 mmol) of methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 5 mL of nBuOH was treated with 45 mg (0.33 mmol) of K₂CO₃ and the resulting orange solution stirred at RT. After 3 hours LCMS indicated complete conversion of the starting material to the desired product. The mixture was treated with 1 mL of AcOH and diluted with EtOAc. The resulting solution was washed with water (3×), brine (1×), dried over sodium sulfate and concentrated to dryness at reduced pressure. The crude material was subjected to flash chromatography (silica gel, gradient elution from DCM to 9:1 DCM/MeOH) to afford 43 mg (66%) of butyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a white foam. ¹H NMR (400 MHz, CHLOROFORM-d) δ ppm 9.31 (d, J=0.8 Hz, 1H) 9.28 (d, J=0.9 Hz, 1H) 8.16-8.23 (m, 1H) 7.58 (d, J=8.5 Hz, 1H) 7.16-7.33 (m, 3H) 7.12 (dd, J=8.6, 2.4 Hz, 1H) 6.87 (s, 1H) 6.77 (s, 1H) 4.24-4.44 (m, 2H) 4.00 (t, J=6.5 Hz, 2H) 1.76-1.91 (m, 2H) 1.57-1.71 (m, 2H) 1.22-1.38 (m, 2H) 1.06 (t, J=7.4 Hz, 3H) 0.89 (t, J=7.4 Hz, 3H). LCMS m/z 616 (M+1).

### Example 8

### 2-[(2-Hydroxyethyl)oxy]ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 8

Compound 8 was prepared in 76% yield from methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate and di(ethylene glycol) according to the method described in Example 6 for the synthesis of 3-hydroxypropyl [2-(2,3-difluorophenyl)-5H-imidazo[4,5-d]pyridazin-5-yl]{3-[4-(propyloxy)-2-(trifluoromethyl)phenyl]-5-isoxazolyl}acetate. ¹H NMR (400 MHz, DMSO-d₆) δ ppm 10.10 (d, J=0.9 Hz, 1H) 9.55 (d, J=0.9 Hz, 1H) 8.12-8.25 (m, 1H) 7.72 (s, 1H) 7.53-7.70 (m, 2H) 7.27-7.46 (m, 3H) 7.13 (s, 1H) 4.55 (t, J=5.2 Hz, 1H) 4.32-4.50 (m, 2H) 4.09 (t, J=6.5 Hz, 2H) 3.62 (t, J=4.5 Hz, 2H) 3.26-3.45 (m, 4H) 1.77 (sxt, J=7.1 Hz, 2H) 1.00 (t, J=7.4 Hz, 3H). LCMS m/z 648 (M+1).

### Example 9

### 2-{[2-(Methyloxy)ethyl]oxy}ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 9

A stirred solution of 0.100 g (0.174 mmol) of methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 8 mL of 1:1 di(ethylene glycol) methyl ether/DME was treated with 50 mg (0.35 mmol) of K₂CO₃ and the resulting orange solution stirred at RT. After 24 hours TLC (silica gel, 95:5 DCM/MeOH) indicated complete conversion of the starting material to a slightly lower R_{f} component. The mixture was treated with 1 mL of AcOH and diluted with EtOAc. The resulting solution was washed with water (5×), brine (1×), dried over sodium sulfate and concentrated to dryness at reduced pressure. The crude material was subjected to flash chromatography (silica gel, gradient elution from DCM to 9:1 MeOH/DCM) followed by reverse phase HPLC purification as described herein for the preparation of ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate to afford 67 mg (58%) of 2-{[2-(methyloxy)ethyl]oxy}ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a white foam. ¹H NMR (400 MHz, CHLOROFORM-d) δ ppm 9.31 (d, J=1.0 Hz, 1H) 9.28 (d, J=1.0 Hz, 1H) 8.14-8.23 (m, 1H) 7.57 (d, J=8.6 Hz, 1H) 7.17-7.33 (m, 3H) 7.12 (dd, J=8.6, 2.5 Hz, 1H) 6.95 (s, 1H) 6.84 (s, 1H) 4.51-4.60 (m, 1H) 4.41-4.50 (m, 1H) 4.00 (t, J=6.5 Hz, 2H) 3.71 (td, J=4.6, 1.0 Hz, 2H) 3.52-3.59 (m, 2H) 3.41-3.48 (m, 2H) 3.29 (s, 3H) 1.77-1.93 (m, 2H) 1.06 (t, J=7.4 Hz, 3H). LCMS m/z 662 (M+1).

### Example 10

### 2-({2-[(2-Hydroxyethyl)oxy]ethyl}oxy)ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 11

A stirred solution of 0.100 g (0.174 mmol) of methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 8 mL of 1:1 tri(ethylene glycol)/ DME was treated with 72 mg (0.52 mmol) of K₂CO₃ and the resulting orange solution stirred at RT. After 24 hours LCMS indicated complete reaction. The mixture was treated with 1 mL of AcOH and diluted with EtOAc. The resulting solution was washed with water (5×), brine (1×), dried over sodium sulfate and concentrated to dryness at reduced pressure. The crude material was subjected to flash chromatography (silica gel, gradient elution from DCM to 9:1 MeOH/DCM) to afford 55 mg (46%) of 2-({2-[(2-hydroxyethyl)oxy]ethyl}oxy)ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a white foam. ¹H NMR (400 MHz, CHLOROFORM-d) δ ppm 9.50 (s, 1H) 9.27 (d, J=0.9 Hz, 1H) 8.11-8.21 (m, 1H) 7.56 (d, J=8.6 Hz, 1H) 7.15-7.32 (m, 3H) 7.10 (dd, J=8.6, 2.5 Hz, 1H) 6.97 (s, 1H) 6.94 (s, 1H) 4.44-4.54 (m, 2H) 3.98 (t, J=6.5 Hz, 2H) 3.69-3.74 (m, 2H) 3.66 (td, J=4.5, 2.1 Hz, 2H) 3.50-3.62 (m, 6H) 1.83 (sxt, J=7.0 Hz, 2H) 1.04 (t, J=7.4 Hz, 3H). LCMS m/z 692 (M+1).

### Example 11

### 3-(Phosphonooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate mono sodium salt

### Compound 14

### Step A

### 3-(Phosphonooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

To a stirred solution of 0.100 g (0.162 mmol) of 3-hydroxypropyl [2-(2,3-difluorophenyl)-5H-imidazo[4,5-c]pyridazin-5-yl]{3-[4-(propyloxy)-2-(trifluoromethyl)phenyl]-5-isoxazolyl}acetate in 6 mL of anhydrous THF was added 151 μL (1.62 mmol) of POCl₃ and the resulting solution stirred at RT. After 2 hours LCMS indicated complete reaction. The solution was cooled in an ice water bath and treated with 1 mL of water by dropwise addition. The solution was then allowed to warm to RT. After 20 minutes the solution was concentrated nearly to dryness by rotary evaporation. The residue was dissolved in 2 mL of MeCN and the solution subjected to reverse phase HPLC purification (C18, gradient from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in DCM. The resulting solution was washed once with water, dried over sodium sulfate, and concentrated to dryness at reduced pressure to give 79 mg (70%) of 3-(phosphonooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a white solid. ¹H NMR (400 MHz, DMSO-d₆) δ ppm 10.07 (d, J=0.8 Hz, 1H) 9.50 (d, J=0.9 Hz, 1H) 8.14 (dd, J=7.8, 6.3 Hz, 1H) 7.46-7.72 (m, 3H) 7.26-7.41 (m, 3H) 7.07 (s, 1H) 4.31 (t, J=5.1 Hz, 2H) 4.04 (t, J=6.5 Hz, 2H) 3.77 (q, J=6.2 Hz 2H) 1.85 (qt, J=6.3 Hz, 2H) 1.72 (sxt, J=6.8 Hz, 2H) 0.95 (t, J=7.4 Hz, 3H). LCMS m/z 698 (M+1).

### Step B

### 3-(Phosphonooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate mono sodium salt

A solution of 67 mg (0.096 mmol) of 3-(phosphonooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 3 mL of MeCN was treated with 3 mL of water which afforded a cloudy solution. To this mixture was added 8.1 mg (0.096 mmol) of NaHCO₃ dissolved in 1 mL of water. This gave a slightly cloudy solution that was filtered and then concentrated to dryness at reduced pressure. The residue was dissolved in MeCN and concentrated to dryness twice and this was repeated using DCM. This afforded 56 mg (81%) of 3-(phosphonooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate mono sodium salt. ¹H NMR (400 MHz, DMSO-d₆) δ ppm 9.95-10.17 (br s, 1H) 9.36-9.44 (s, 1H) 8.08-8.20 (m, 1H) 7.43-7.60 (m, 3H) 7.18-7.36 (m, 4H) 4.08-4.22 (br s, 2H) 3.96-4.06 (m, 2H) 3.59-3.75 (m, 2H) 1.62-1.83 (m, 4H) 0.94 (t, J=7.2 Hz, 3H). LCMS m/z 698 (M+1).

### Example 12

### 3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoic acid sodium salt

### Compound 16

### Step A

### 3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoic acid

Periodic acid (0.244 g, 1.07 mmol) was added to 10 mL of anhydrous MeCN and the mixture stirred at RT for 15 minutes. The solid reagent slowly dissolved to afford a clear solution. A solution of 0.300 g (0.486 mmol) of 3-hydroxypropyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 3 mL of MeCN was added and the resulting solution was cooled in an ice water bath. The solution was then treated with 2.1 mg (9.7 μmol) of PCC. A light yellow precipitate was rapidly produced. The ice bath was removed and the reaction mixture was allowed to warm to RT with stirring. After 2 hours TLC (silica gel, 8:2 DCM/MeOH) indicated approximately 50% conversion of the starting material to a lower R_{f} component. The mixture was treated with additional portions of periodic acid (0.244 g, 1.07 mmol) and PCC (2.1 mg, 9.72 μmol) and stirring at RT continued. After another 1.5 hours TLC indicated complete reaction. The mixture was concentrated nearly to dryness by rotary evaporation. The residue was suspended in 75 mL of 9:1 CHCl₃/iPrOH and stirred rapidly with addition of 75 mL of 10% aqueous sodium bisulfite. After stirring for several minutes the phases were separated. The organic solution was washed with water (1×), brine (1×), dried over sodium sulfate and concentrated to dryness at reduced pressure. The crude material was subjected to reverse phase HPLC purification (C18, gradient from 9:1 water/0.1% TFA: MeCN/0.1% TFA to 100% MeCN/0.1°)/0 TFA over 10 minutes). Fractions containing pure product were combined and concentrated to dryness at reduced pressure. The residue was dissolved in DCM. The resulting solution was washed with water (2×), dried over sodium sulfate, and concentrated to dryness at reduced pressure to afford 0.233 g (76%) of 3-[([2-(2,3-difluorophenyl)-5H-imidazo[4,5-d]pyridazin-5-yl]{3-[4-(propyloxy)-2-(trifluoromethyl)phenyl]-5-isoxazolyl}acetyl)oxy]propanoic acid as a light tan foam. ¹H NMR (400 MHz, DMSO-d₆) δ ppm 12.38 (s, 1H) 10.05 (d, J=0.9 Hz, 1H) 9.48 (d, J=0.9 Hz, 1H) 8.06-8.22 (m, 1H) 7.47-7.70 (m, 3H) 7.24-7.40 (m, 3H) 7.04 (s, 1H) 4.29-4.53 (m, 2H) 4.04 (t, J=6.5 Hz, 2H) 2.56 (t, J=6.1 Hz, 2H) 1.72 (sxt, J=7.0 Hz, 2H) 0.95 (t, J=7.4 Hz, 3H). LCMS m/z 632 (M+1).

### Step B

### 3[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoic acid sodium salt

A solution of 0.100 g (0.158 mmol) of 3-[([2-(2,3-difluorophenyl)-5H-imidazo[4,5-d]pyridazin-5-yl]{3-[4-(propyloxy)-2-(trifluoromethyl)phenyl]-5-isoxazolyl}acetyl)oxy]propanoic acid in 3 mL of MeCN was treated with 3 mL of water which afforded a cloudy solution. To this mixture was added 13.3 mg (0.158 mmol) of NaHCO₃ dissolved in 1 mL of water. This gave a light yellow solution containing a small amount of insoluble solids. The solution was filtered and concentrated to dryness at reduced pressure. The residue was dissolved in MeCN and concentrated to dryness twice and this was repeated using DCM. This afforded 94 mg (91%) of 3-[([2-(2,3-difluorophenyl)-5H-imidazo[4,5-c]pyridazin-5-yl]{3-[4-(propyloxy)-2-(trifluoromethyl)phenyl]-5-isoxazolyl}acetyl)oxy]propanoic acid sodium salt as a yellow-orange powder. ¹H NMR (400 MHz, DEUTERIUM OXIDE) δ ppm 9.48 (s, 1H) 8.79 (s, 1H) 7.38-7.47 (m, 1H) 6.70-6.89 (m, 3H) 6.58 (s, 1H) 6.22-6.36 (m, 2H) 4.30-4.46 (m, 2H) 3.08 (br s, 2H) 2.38 (t, J=6.6 Hz, 2H) 0.89-1.05 (m, 2H) 0.21 (t, J=6.8 Hz, 3H). LCMS m/z 632 (M+1).

### Example 13

### 2-[2-(2,3-Difluorophenyl)-5H-imidazo[4,5-d]pyridazin-5-yl]-N-methyl-2-{3-[4-(propyloxy)-2-(trifluoromethyl)phenyl]-5-isoxazolyl}acetamide

### Compound 20

A solution of 0.100 g (0.170 mmol) of methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 8 mL of 2M MeNH₂/MeOH was stirred at RT. After 1.5 hours LCMS indicated complete conversion of the starting material to the desired amide. The solution was concentrated to dryness at reduced pressure. The residue was dissolved in DCM. The resulting solution was washed with 2% aqueous AcOH (1×), water (2×), dried over sodium sulfate, and concentrated to dryness at reduced pressure. The residue was subjected to reverse phase HPLC purification (C18, gradient from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in DCM. The solution was washed with water (2×), dried over sodium sulfate, and concentrated to dryness at reduced pressure to afford 74 mg (74%) of 2-[2-(2,3-difluorophenyl)-5H-imidazo[4,5-d]pyridazin-5-yl]-N-methyl-2-{3-[4-(propyloxy)-2-(trifluoromethyl)phenyl]-5-isoxazolyl}acetamide as a white solid. ¹H NMR (400 MHz, DMSO-d₆) δ ppm 9.89 (d, J=0.9 Hz, 1H) 9.47 (d, J=1.0 Hz, 1H) 8.46-8.52 (m, 1H) 8.09-8.16 (m, 1H) 7.64 (d, J=8.1 Hz, 1H) 7.46-7.59 (m, 1H) 7.25-7.40 (m, 3H) 7.16 (s, 1H) 6.99 (s, 1H) 4.04 (t, J=6.5 Hz, 2H) 2.68 (d, J=4.6 Hz, 3H) 1.64-1.80 (m, 2H) 0.95 (t, J=7.4 Hz, 3H). LCMS m/z 573 (M+1).

### Example 14

### 2-{[2-({2-[(2-Hydroxyethyl)oxy]ethyl}oxy)ethyl]oxy}ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 12

To a stirred solution of 0.100 g (0.174 mmol) of methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 6 mL of 1:1 tetra(ethylene glycol)/DME was added 72 mg (0.52 mmol) of potassium carbonate and the resulting orange solution was stirred at RT. After 3 days, LCMS indicated complete reaction. The mixture was treated with 1 mL of glacial AcOH and diluted with EtOAc. The resulting solution was washed with water (4×), dried over sodium sulfate, and concentrated to dryness at reduced pressure. The residue was subjected to reverse phase HPLC purification (C18, gradient from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in EtOAc. The solution was washed with water (2×), dried over sodium sulfate, and concentrated to dryness at reduced pressure to afford 56 mg (44%) of 2-{[2-({2-[(2-hydroxyethyl)oxy]ethyl}oxy)ethyl]oxy}ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a tacky, white foam. ¹H NMR (400 MHz, CHLOROFORM-d) δ ppm 9.54 (d, J=0.6 Hz, 1H) 9.27 (d, J=1.0 Hz, 1H) 8.16 (td, J=6.1, 1.5 Hz, 1H) 7.56 (d, J=8.5 Hz, 1H) 7.14-7.32 (m, 3H) 7.10 (dd, J=8.5, 2.5 Hz, 1H) 7.00 (s, 1H) 6.95 (s, 1H) 4.48-4.56 (m, 1H) 4.39-4.47 (m, 1H) 3.98 (t, J=6.5 Hz, 2H) 3.47-3.78 (m, 14H) 1.83 (sxt, J=7.0 Hz, 2H) 1.04 (t, J=7.3 Hz, 3H). LCMS m/z 736 (M+1).

### Example 15

### 2-Methylpropyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 169

A solution of 0.300 g (0.523 mmol) of methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 5 mL of 2-methyl-1-propanol was treated with 0.217 g (1.57 mmol) of potassium carbonate and the resulting mixture was stirred at RT. After 24 hours, LCMS indicated complete conversion of starting material to the desired product. The mixture was treated with 1 mL of AcOH and diluted with EtOAc. The solution was washed with water (3×), saturated brine (1×), dried over sodium sulfate and concentrated to dryness at reduced pressure. The crude material was subjected to reverse phase HPLC purification (C18, gradient from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in EtOAc. The resulting solution was washed with water (2×), dried over sodium sulfate, and concentrated to dryness at reduced pressure to afford 0.241 g (75%) of 2-methylpropyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a white foam. ¹H NMR (400 MHz, CHLOROFORM-d) δ ppm 9.66 (s, 1H) 9.36 (d, J=0.8 Hz, 1H) 8.12-8.22 (m, 1H) 7.56 (d, J=8.5 Hz, 1H) 7.26-7.36 (m, 2H) 7.18-7.26 (m, 1H) 7.10 (dd, J=8.6, 2.5 Hz, 1H) 6.94 (s, 1H) 6.90 (s, 1H) 4.12-4.20 (m, 1H) 4.03-4.10 (m, 1H) 3.98 (t, J=6.5 Hz, 2H) 1.89-2.01 (m, 1H) 1.83 (d, J=7.3 Hz, 2H) 1.04 (t, J=7.4 Hz, 3H) 0.86 (d, J=6.6 Hz, 6H). LCMS m/z 616 (M+1).

### Example 16

### Phenylmethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

### Compound 171

To a stirred solution of 0.350 g (0.610 mmol) of methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 6 mL of 1:1 DME/benzyl alcohol was added 0.253 g (1.83 mmol) of potassium carbonate and the resulting mixture stirred at RT. After 18 hours, LCMS indicated nearly complete conversion to the desired compound. The mixture was treated with 1 mL of AcOH and diluted with EtOAc. The cloudy solution was washed with water (3×), brine (1×), dried over sodium sulfate and concentrated to dryness at reduced pressure. The benzyl alcohol was removed by short path distillation. The residue was subjected to reverse phase HPLC purification (C18, gradient from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in DCM. The resulting solution was washed with water (2×), dried over sodium sulfate, and concentrated to dryness at reduced pressure to afford 215 mg of phenylmethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a white foam. ¹H NMR (400 MHz, CHLOROFORM-d) δ ppm 9.60 (s, 1H) 9.35 (d, J=0.8 Hz, 1H) 8.23 (ddd, J=7.9, 4.4, 1.7 Hz, 1H) 7.56 (d, J=8.6 Hz, 1H) 7.21-7.39 (m, 8H) 7.12 (dd, J=8.6, 2.5 Hz, 1H) 6.99 (s, 1H) 6.82 (s, 1H) 5.28-5.43 (m, 2H) 4.01 (t, J=6.5 Hz, 2H) 1.86 (sxt, J=7.0 Hz, 2H) 1.07 (t, J=7.4 Hz, 3H). LCMS m/z 650 (M+1).

### Example 17

### 3-(Sulfooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate sodium salt

### Compound 15

### Step A

### 3-(Sulfooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate

A solution of 0.300 g (0.486 mmol) of 3-hydroxypropyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 5 mL of dry DMF was treated with 0.464 g (2.91 mmol) of SO₃-pyridine complex and the resulting solution stirred at RT. After 2 hours, LCMS indicated complete conversion of the starting material to the desired intermediate. The solution was treated with 3 mL of saturated aqueous sodium bicarbonate, stirred for several minutes and then concentrated nearly to dryness by rotary evaporation. The residue was partitioned between 10% aqueous NaCl and EtOAc. The phases were separated and the aqueous solution extracted with two additional portions of EtOAc. The combined EtOAc solutions were dried over sodium sulfate and concentrated to dryness at reduced pressure. The residue was subjected to reverse phase HPLC purification (C18, gradient from 9:1 water/0.1% TFA: MeCN/0.1% TFA to 100% MeCN/0.1% TFA over 10 minutes). Fractions containing pure product were combined and concentrated to dryness at reduced pressure and the residue dissolved in EtOAc. The solution was washed with water (2×), dried over sodium sulfate, and concentrated to dryness to afford 0.270 g (80%) of 3-(sulfooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate as a light tan solid. ¹H NMR (400 MHz, CHLOROFORM-d) δ ppm 10.86 (br s, 1H) 9.50 (br s, 1H) 8.01-8.17 (m, 1H) 7.59 (s, 1H) 7.53 (d, J=8.6 Hz, 1H) 7.33-7.45 (m, 1H) 7.19-7.33 (m, 3H) 7.00-7.09 (m, 2H) 4.62 (br s, 1H) 4.35-4.45 (m, 1H) 4.07-4.24 (m, 2H) 3.96 (t, J=6.5 Hz, 2H) 1.93-2.12 (m, 2H) 1.77-1.88 (m, 2H) 1.04 (t, J=7.4 Hz, 3H). LCMS m/z 698 (M+1).

### Step B

### 3-(Sulfooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate sodium salt

A solution of 0.264 g (0.378 mmol) of 3-(sulfooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 5 mL of MeCN was treated with 5 mL of water to afford a cloudy suspension. To this was added 32 mg (0.378 mmol) of NaHCO₃ dissolved in 1 mL of water. This gave a clear solution that was filtered through a medium frit and concentrated to dryness at reduced pressure. The residue was dissolved in MeCN and concentrated to dryness. This was repeated with DCM to afford 0.260 g (96%) of 3-(sulfooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate sodium salt as a yellow powder. ¹H NMR (400 MHz, DEUTERIUM OXIDE) δ ppm 9.46 (s, 1H) 8.75 (s, 1H) 7.33-7.46 (m, 1H) 6.65-6.86 (m, 3H) 6.58 (s, 1H) 6.16-6.34 (m, 2H) 4.16-4.33 (m, 2H) 3.77-3.92 (m, 2H) 3.04 (br s, 2H) 1.72-1.91 (m, 2H) 0.82-1.02 (m, 2H) 0.18 (br s, 3H). LCMS m/z 698 (M+1).

### Example 18

### 4-(Dimethylamino)butyl [2-(2,3-difluorophenyl)-5H-imidazo[4,5-d]pyridazin-5-yl]{3-[4-(propyloxy)-2-(trifluoromethyl)phenyl]-5-isoxazolyl}acetate dihydrochloride

### Compound 173

A solution of 0.300 g (0.523 mmol) of methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate in 6 mL of 5:1 4-(dimethylamino)-1-butanol/DME was treated with 0.289 g (2.09 mmol) of potassium carbonate and the resulting orange solution stirred at RT. After 18 hours, the mixture was partitioned between EtOAc and 5% aqueous AcOH and the phases separated. The EtOAc solution was washed with water (4×), dried over sodium sulfate and concentrated to dryness at reduced pressure. The residue was subjected to reverse phase HPLC purification (C18, gradient from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in EtOAc. The solution was washed with 10% aqueous NaCl (3×), dried over sodium sulfate, and concentrated to dryness at reduced pressure. The residue was dissolved in 5 mL of DCM. The solution was treated with 0.12 mL of 4 N HCl/dioxane and concentrated to dryness at reduced pressure. The residue was redissolved in DCM and concentrated twice to afford 0.185 g (48%) of 4-(dimethylamino)butyl [2-(2,3-difluorophenyl)-5H-imidazo[4,5-d]pyridazin-5-yl]{3-[4-(propyloxy)-2-(trifluoromethyl)phenyl]-5-isoxazolyl}acetate dihydrochloride as a white foam. ¹H NMR (400 MHz, CHLOROFORM-d) δ ppm 11.65 (br s, 1H) 11.45 (s, 1H) 9.69 (s, 1H) 8.92 (s, 1H) 8.27 (t, J=6.8 Hz, 1H) 7.51 (d, J=8.5 Hz, 1H) 7.42 (q, J=7.8 Hz, 1H) 7.28-7.37 (m, 1H) 7.21-7.28 (m, 2H) 7.03-7.12 (m, 2H) 4.40 (t, J=5.3 Hz, 2H) 3.98 (t, J=6.5 Hz, 2H) 3.15 (br s, 2H) 2.88 (d, J=4.5 Hz, 6H) 2.02 (br s, 2H) 1.73-1.91 (m, 4H) 1.05 (t, J=7.3 Hz, 3H). LCMS m/z 659 (M+1).

### Example 19

### N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanine sodium salt

### Compound 175

### Step A

### N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanine

To a stirred solution of 50 mg (0.079 mmol) of 3-[([2-(2,3-difluorophenyl)-5H-imidazo[4,5-d]pyridazin-5-yl]{3-[4-(propyloxy)-2-(trifluoromethyl)phenyl]-5-isoxazolyl}acetyl)oxy]propanoic acid and 22 mg (0.119 mmol) of L-alanine-O-t-butyl ester hydrochloride in 5 mL of anhydrous MeCN was added 45 mg (0.119 mmol) of HATU followed by 42 μL (0.238 mmol) of DIEA. The resulting solution was stirred at RT. After 1.5 hours, LCMS indicated complete reaction. The solution was concentrated to dryness at reduced pressure and the residue dissolved in EtOAc. The solution was washed with brine (2×), dried over sodium sulfate and concentrated to dryness. The residue was dissolved in 2 mL of DCM and this solution treated with 4 mL of TFA. The resulting solution was stirred at RT. After 1 hour LCMS indicated complete conversion of the t-butyl ester intermediate to the desired carboxylic acid. The solution was concentrated to dryness at reduced pressure. The residue was redissolved in DCM and concentrated twice to remove residual TFA. The residue was subjected to reverse phase HPLC purification (C18, gradient from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in EtOAc. The resulting solution was washed once with water, dried over sodium sulfate, and concentrated to dryness at reduced pressure to give 47 mg (85%) of N-{3-[([2-(2,3-difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanine as a light yellow solid ¹H NMR (400 MHz, DMSO-d₆) δ ppm 12.48 (br s, 1H) 10.11 (d, J=2.6 Hz, 1H) 9.56 (s, 1H) 8.10-8.29 (m, 2H) 7.51-7.71 (m, 3H) 7.30-7.45 (m, 3H) 7.10 (d, J=6.7 Hz, 1H) 3.84-4.56 (m, 5H), 2.43-2.58 (m, 2H), 1.77 (sxt, J=7.0 Hz, 2H) 1.10 (dd, J=7.2, 5.1 Hz, 3H) 1.00 (t, J=7.4 Hz, 3H). LCMS m/z 703 (M+1).

### Step B

### N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanine sodium salt

A stirred solution of 42 mg (0.060 mmol) of N-{3-[([2-(2,3-difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanine in 4 mL of MeCN was treated with 4 mL of water to afford a cloudy solution. To this was added 5.0 mg (0.060 mmol) of sodium bicarbonate dissolved in 1 mL of water. This gave a transparent yellow solution that was concentrated to dryness at reduced pressure. The residue was dissolved in MeCN and concentrated to dryness twice. This was repeated using DCM to afford 43 mg (99%) of N-{3-[([2-(2,3-difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanine sodium salt as a yellow powder. ¹H NMR (400 MHz, DMSO-d₆) δ ppm 9.63 (br s, 1H) 9.36 (s, 1H) 8.17 (br s, 1H) 7.44-7.79 (m, 3H) 7.22-7.41 (m, 3H) 5.55-6.04 (m, 1H) 3.78-4.29 (m, 5H) 2.12-2.35 (m, 2H) 1.64-1.84 (m, 2H) 0.87-1.12 (m, 6H). LCMS m/z 703 (M+1).

### Example 20

### N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanyl-L-phenylalanine

### Compound 177

### Step A

### 1,1-Dimethylethyl N-{[(phenylmethyl)oxy]carbonyl}-L-alanyl-L-phenylalaninate

A stirred solution of 0.500 g (2.24 mmol) of CBz-Ala-OH and 0.577 g (2.24 mmol) of L-Phe-O-t-Bu hydrochloride in 15 mL of dry MeCN was treated with 0.978 mL (5.60 mmol) of DIEA followed by 0.937 g (2.46 mmol) of HATU. The resulting solution was stirred at RT. After 2 hours, LCMS indicated complete reaction. The solution was concentrated to dryness at reduced pressure and the residue dissolved in EtOAc. The resulting solution was washed with 10% aqueous citric acid (2×), saturated aqueous sodium bicarbonate (2×), dried over sodium sulfate and concentrated to dryness at reduced pressure. The residue was dissolved in a few mLs of EtOAc and the solution triturated with addition of hexane which led to the formation of a white crystalline solid. The suspension was stirred at RT for 1 hour. The solid was collected by filtration and dried in vacuo to afford 0.910 g (95%) of 1,1-dimethylethyl N-{[(phenylmethyl)oxy]carbonyl}-L-alanyl-L-phenylalaninate as a white solid. ¹H NMR (400 MHz, DMSO-d₆) δ ppm 8.13 (d, J=7.4 Hz, 1H) 7.14-7.43 (m, 11H) 5.00 (s, 2H) 4.32 (q, J=7.3 Hz, 1H) 4.07 (quin, J=7.5 Hz, 1H) 2.87-2.99 (m, 2H) 1.30 (s, 9H) 1.17 (d, J=7.1 Hz, 3H). LCMS m/z 427 (M+1).

### Step B

### 1,1-Dimethylethyl L-alanyl-L-phenylalaninate

A solution of 0.250 g (0.586 mmol) of 1,1-dimethylethyl N-{[(phenylmethyl)oxy]carbonyl}-L-alanyl-L-phenylalaninate in 20 mL of MeOH was subjected to hydrogenation at 50 psi in the presence of 25 mg of 10% Pd(C). After 1 hour the reaction vessel was purged with nitrogen, catalyst removed by filtration through celite and the filtrate concentrated to dryness at reduced pressure to afford 1,1-dimethylethyl L-alanyl-L-phenylalaninate in quantitative yield as a viscous oil. ¹H NMR (400 MHz, DMSO-d₆) δ ppm 8.07 (d, J=7.8 Hz, 1H) 7.24-7.32 (m, 2H) 7.15-7.24 (m, 3H) 4.38 (q, J=7.3 Hz, 1H) 3.23 (q, J=6.9 Hz, 1H) 2.87-3.03 (m, 2H) 1.75 (s, 2H) 1.33 (s, 9H) 1.06 (d, J=6.8 Hz, 3H). LCMS m/z 293 (M+1).

### Step C

### N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanyl-L-phenylalanine

To a stirred solution of 0.109 g (0.173 mmol) of 3-[([2-(2,3-difluorophenyl)-5H-imidazo[4,5-d]pyridazin-5-yl]{3-[4-(propyloxy)-2-(trifluoromethyl)phenyl]-5-isoxazolyl}acetyl)oxy]propanoic acid and 50 mg (0.173 mmol) of 1,1-dimethylethyl L-alanyl-L-phenylalaninate in 8 mL of anhydrous MeCN was added 98 mg (0.259 mmol) of HATU followed by 60 μL (0.35 mmol) of DIEA. The resulting solution was stirred at RT. After 40 minutes, analysis by LCMS indicated complete reaction. The solution was concentrated to dryness at reduced pressure. The residue was dissolved in DCM. The resulting solution was washed with brine (2×), dried over sodium sulfate and concentrated to dryness at reduced pressure. The residue was dissolved in 5 mL of DCM and the solution treated with 5 mL of TFA. After stirring the solution at RT for 3 hours LCMS indicated complete cleavage of the t-butyl ester. The solution was concentrated to dryness at reduced pressure. The residue was subjected to reverse phase HPLC purification (C18, gradient from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in DCM. The resulting solution was washed with water (2×), dried over sodium sulfate, and concentrated to dryness at reduced pressure to afford 84 mg (57%) of N-{3-[([2-(2,3-difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanyl-L-phenylalanine as a light yellow solid. ¹H NMR (400 MHz, DMSO-d₆) δ ppm 12.71 (br s, 1H) 10.08 (d, J=1.6 Hz, 1H) 9.52 (s, 1H) 7.95-8.24 (m, 3H) 7.52-7.70 (m, 3H) 7.29-7.46 (m, 3H) 7.03-7.29 (m, 6H) 4.32-4.54 (m, 3H) 4.24 (br. s., 1H) 4.08 (t, J=6.5 Hz, 2H) 2.97-3.09 (m, 1H) 2.77-2.96 (m, 1H) 2.33-2.59 (m, 2H) 1.57-1.86 (m, 2H) 0.83-1.09 (m, 6H). LCMS m/z 850 (M+1).

### Example 20

### Methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate

### Compound 178

### Step A

### 2-(2,3-difluorophenyl)-1H-imidazole

To a stirred solution of 2,3-difluorobenzaldehyde (1.0 kg, 7.037 mol) in 14 L of a 1:1 mixture of IPA/water, ammonium acetate (4.88 kg, 63.31 mol) was added at 25° C. to afford a white slurry. To the stirring slurry, 40% aqueous glyoxal was added over a 5 hour period. After 30 minutes HPLC indicated complete reaction. The golden slurry was filtered in vacuo to remove solids and the filter cake washed with IPA (2 L). The combined filtrate/wash was distilled under reduced pressure to ˜9.0 L and the concentrate temperature re-adjusted to 25° C. The concentrate was diluted with water (3.0 L) then extracted with dichloromethane (10 L). The organic layer was extracted with 1N HCl (10 L) and the stirring acidic aqueous phase neutralized to pH 7 with 50% sodium hydroxide. The resulting slurry was cooled to 5° C. where it was aged for 1 hour and then filtered. The filter cake was washed with water (2 L), concentrated to near dryness and further dried at elevated temperature at reduced pressure to give 1.097 kg (87%) of 2-(2,3-difluorophenyl)-1H-imidazole as a brown electrostatic solid. ¹H NMR (400 MHz, DMSO-d₆) δ ppm 12.44 (br. s., 1H) 7.70-7.82 (m, 1H) 7.30-7.42 (m, 1H) 7.15-7.28 (m, 2H) 7.06 (s, 1H), LCMS m/z 181 (M+1)

### Step B

### [2-(2,3-difluorphenyl)-1H-imidazole-4,5-yl]dimethanol

To a stirred slurry of 2-(2,3-difluorophenyl)-1H-imidazole (0.5 kg, 2.78 mol) in water (7.5 L) at 25° C., potassium hydroxide (0.312 kg, 5.551 mol) was added and the resultant mixture cooled to 25° C. and allowed to stir for 10 minutes. A vessel was purged with nitrogen and charged with 37% aqueous formaldehyde (3.685 kg, 41.67 mol), then 40% of the formaldehyde solution (by volume) was added to the imidazole mixture which was then heated to 50° C. and maintained for 1 hour. The remainder of the formaldehyde was added in three 20% portions at 50° C. followed by an additional 1 hour stir at temperature between each addition. After stirring for 2 hours following the last addition, HPLC indicated complete reaction. The reaction was cooled to 25° C. and stirred for 8 hours. The tan slurry was adjusted to pH 7.0 with phosphate buffer solution (2.5 L), stirred for an additional hour at 25° C. and filtered. The filter cake was washed with water (1.4 L), pulled to near dryness and further dried at elevated temperature at reduced pressure to give 0.531 kg (80%) of [2-(2,3-difluorphenyl)-1H-imidazole-4,5-yl]dimethanol as a tan solid. ¹H NMR (400 MHz, DMSO-d₆) δ ppm 12.29-12.20 (bs, 1H) 7.75-7.71 (m, 1H) 7.40-7.35 (m 1H) 7.25-7.21 (m, 1H) 4.8-4.65 (m, 2H) 4.6-4.45 (m, 4H), LCMS m/z 241 (M+1)

### Step C

### 2-(2,3-difluorphenyl)-1H-imidazole-4,5-dicarbaldehyde

To a stirred solution of [2-(2,3-difluorphenyl)-1H-imidazole-4,5-yl]dimethanol (0.6 kg, 2.50 mol) in methanol (9.0 L), manganese dioxide (6 kg, 69.01 mol) was added in four 25% portions at 25° C. followed by a 15 minute stir at temperature between each addition. After stirring for 2 hours at 25° C. following end of addition, HPLC indicated complete reaction. The reaction mixture was passed through a series of polishing filters in vacuo to remove excess manganese dioxide and the resultant filter cake washed with additional methanol (12 L). The methanolic filtrate/wash was solvent exchanged into 2-methyltetrahydrofuran distillation under reduced pressure and the resulting concentrate volume adjusted to 19 liters with additional 2-methyltetrahydrofuran. The solution was washed with 1N NaOH (3 L), concentrated in vacuo to 1.5 L and the stirring concentrate diluted with MTBE (4.5 L). The resulting slurry was aged at 25° C. for 1 hour and filtered. The filter cake was washed with MTBE (3L), concentrated to near dryness and further dried with elevated temperature at reduced pressure to give 447 g (76%) of 2-(2,3-difluorphenyl)-1H-imidazole-4,5-dicarbaldehyde as a golden solid. ¹H NMR (400 MHz, DMSO-d₆) δ ppm 10.21 (br. s., 1H) 10.06 (m, 2H) 7.92-7.89 (m, 1H) 7.28-7.25 (m, 1H) 7.16-7.12 (m, 1H), LCMS m/z 237 (M+H)

### Step D

### 4-bromo-2-(trifluoromethyl)benzaldehyde oxime

To a stirred solution of 5.5 L of MTBE (5 vol) was added 1.74 L of n-butyl lithium (2.5M in hexanes) (4.344 moles) and the solution was cooled to 0° C. A solution of 1.1 kg of 1,4-dibromo-2-(trifluoromethyl)benzene (3.619 moles) was prepared in 2.2 L of MTBE (2 vol). The 1,4-dibromo-2-(trifluoromethyl)benzene solution was added to the butyl lithium solution over a period of approximately 10 minutes keeping the reaction temperature below 25° C. The 1,4-dibromo-2-(trifluoromethyl)benzene holding vessel and pumping lines were washed with an additional 1.1 L MTBE (1 vol). The reaction mixture was stirred at 0° C. for 30 minutes. To the reaction mixture was added 0.31 L of N,N-Dimethylformamide (3.982 moles) and the mixture stirred for 30 minutes. Then 5.5 L water (5 vol) and 1.45 L 6M HCl (2.4 eq HCl) were added, the reaction mixture stirred at 20° C. for 30 minutes then separated and the aqueous phase discarded. The organic layer was washed twice with 2×5.5 L of water (5 vol) discarding the aqueous phase each time. The MTBE solution was concentrated under reduced pressure to minimum stir volume. Ethanol (5 vol) was charged to the reactor and concentrated under reduced pressure to minimum stir volume. Ethanol (6 vol) was then charged to the reactor. To this solution 0.256 L hydroxylamine (50% water) (4.34 moles) was added and the reaction stirred at 50° C. After condensation was complete 5.5 L water (5 vol) was added to the reactor and it was cooled to 20° C. Seed crystals were added to induce crystallization. 5.5 L of water (5-vol) was added to the reaction to complete crystallization. The solid was filtered and washed with 2.2 L of ethanol/water (1:1.7) (2 vol) and solid was dried in a vacuum oven at approximately 55° C. to afford 965.5 g (84%) of 4-bromo-2-(trifluoromethyl)benzaldehyde oxime as a white powder. ¹H NMR (400 MHz, DMSO-d₆) δ ppm 12.03 (s, 1H), 8.23 (q, J=2.33 Hz, 1H), 7.98 (s, 1H), 7.93 (d, J=1.23 Hz, 2H).

### Step E

### 2-{3-[4-bromo-2-(trifluoromethyl)phenyl]-5-isoxazolyl}ethanol

To a stirred solution of 0.915 kg 4-bromo-2-(trifluoromethyl)benzaldehyde oxime (3.41 moles) in 4.575 L DMF (5-vol) was added 0.480 kg N-chlorosuccinimide (3.58 moles) in small portions to regulate the exotherm. The reaction was stirred for 30 minutes and checked for completion. The reactor was then charged with 9.5 L MTBE (10 vol) and 4.575 L water (5 vol), and then the aqueous phase was discarded. The MTBE layer was washed 2×4.575 L water (5 vol). The reactor was charged with 0.354 L 3-butyn-1-ol (4.44 moles), then 0.523 L triethylamine (3.76 moles). After the exotherm subsided from TEA addition the reaction mixture was stirred at 50° C. for 3 hours. The reaction was checked for completion and cooled to 20-25° C. The reactor was charged with 9.5 L water (10 vol), stirred and separated and the aqueous phase discarded. The MTBE layer was washed 2×4.575 L water (5 vol), and the aqueous phases discarded. The MTBE solution was concentrated on a rotary evaporator to afford 915.1 g (80%) 2-{3-[4-bromo-2-(trifluoromethyl)phenyl]-5-isoxazolyl}ethanol as a brown oil. ¹H NMR (400 MHz, DMSO-d₆) δ ppm 8.11 (m, 1H), 8.04 (dd, J=8.3, 2.0 Hz, 1H), 7.60 (d, J=8.22 Hz, 1H), 6.54, (s, 1H), 4.93 (t, J=5.0 Hz, 1H), 3.74 (q, J=6.2 Hz, 2H), 2.97 (t, J=6.5 Hz, 2H).

### Step F

### 2-{3-[4-cyclopropyl-2-(trifluoromethyl)phenyl]-5-isoxazolyl}ethanol

A previously prepared solution of 833 g of 2-{3-[4-bromo-2-(trifluoromethyl)phenyl]-5-isoxazolyl}ethanol (2.48 mol) was concentrated to an oil in a 20-L jacketed laboratory reactor. 639 g of cyclopropyl boronic acid (7.44 mol), 5.8 L of toluene, and 6.7 L of water were charged into the reactor, and stirred. The reaction vessel was placed under vacuum, and refilled with nitrogen twice, then 101 g of dichloro(1,1′-bis(diphenylphosphino)ferrocene)palladium (II) dichloromethane adduct (0.124 mol) was added and the mixture heated to 55° C. 1.23 L of diisopropylethylamine (7.44 mol) was added and the reaction was stirred for 24 h. The reactor was charged with 420 g of activated carbon (Darco G60), and the resulting slurry was stirred for 55 min, and cooled to 20° C. The slurry was passed through an in-line filter and the filter was rinsed with 2.7 Kg toluene. The organic layers were combined and 3.3 L of water and 600 g of 50% NaOH (7.5 mol) was added. The mixture was stirred 10 min, and the lower layer was drained and discarded. 3.3 L of water and 160 g of concentrated sulfuric acid (1.63 mol) was added. The mixture was stirred for 10 min, and the lower layer was drained and discarded. The organic layer was washed with 3.3 L of water, and the aqueous layer was discarded. The resulting solution was then concentrated in vacuo providing 2-{3-[4-cyclopropyl-2-(trifluoromethyl)phenyl]-5-isoxazolyl}ethanol as an oil that is carried directly into the next reaction assuming 100% yield. ¹H NMR (400 MHz, DMSO, D₆) δ ppm: 7.62-7.60 (d, 1H, J=1.2 Hz), 7.52-7.48 (d, 1H, J=8.0 Hz), 7.45-7.41 (dd, 1H, J=8.0, 1.2), 6.46 (s, 1H), 4.94-4.91 (t, 1H, J=5.2 Hz), 3.76-3.70 (dt, 2H, J=5.5, 6.4), 2.98-2.93 (t, 2H, J=6.4), 2.17-2.09 (m, 1H), 1.09-1.04 (m, 2H), 0.85-0.79 (m, 2H).

### Step G

### {3-[4-cyclopropyl-2-(trifluoromethyl)phenyl]-5-isoxazolyl}acetic acid

A 1-L reactor was charged with 61 g of periodic acid (0.269 mol) and 580 ml acetonitrile and stirred for 40 min at 20° C. A solution of 35.3 g of 2-{3-[4-cyclopropyl-2-(trifluoromethyl)phenyl]-5-isoxazolyl}ethanol (0.119 mol) dissolved in 200 ml of acetonitrile was added, and the mixture was cooled to ˜2° C. and 0.53 g of pyrdinium chlorochromate was added (0.00244 mol). Once the resulting exotherm had subsided the reaction mixture was warmed to 20° C. and held for ˜2 h. Then 360 ml of water was added and the mixture was stirred for ˜5 min, the layers were allowed to separate and the lower aqueous layer was drained and discarded. 360 mL of a saturated solution of Na₂SO₃ was added all at once. The reaction mixture briefly turned brown and then lightened as the periodate species quenched. The mixture was stirred about 5 min, the layers were separated, and the lower aqueous layer was drained and discarded. The resulting solution was concentrated in vacuo, until ˜750 ml of solvent was removed. The solution was then cooled to 20° C. and 360 mL of 1N NaOH (0.36 mol) in water and 360 ml of MTBE were added. After stirring ˜5 min the lower aqueous layer was collected, and the upper organic layer was discarded. The aqueous layer was returned to the reactor and concentrated, 20 mL of H₂SO₄ (0.36 mol) was then added. The acidified aqueous layer was then extracted twice with 180 mL of MTBE. The combined organic layers were combined and washed once with 360 ml of water, then concentrated to dryness in vacuo using a rotary evaporator, and dried in a 60-65° C. vacuum oven to provide 28.49 g (81% yield) of {3-[4-cyclopropyl-2-(trifluoromethyl)phenyl]-5-isoxazolyl}acetic acid as a dark orange solid. ¹H NMR (400 MHz, CDCl₃) δ ppm: 7.55-7.50 (d, 1H, J=8.0 Hz), 7.47-7.65 (d, 1H, J=1.4 Hz), 7.29, 7.25 (dd, 1H, J=8, 1.4), 6.49 (s, 1H), 3.96 (s, 2H), 2.03-1.94 (m, 1H), 1.12-1.05 (m, 2H), 0.81-0.75 (m, 2H).

### Step H

### Methyl {3-[4-cyclopropyl-2-(trifluoromethyl)phenyl]-5-isoxazolyl}acetate

A 2-L round bottomed flask was charged with 880 ml of methanol and 2.5 mL of acetyl chloride (0.035 mol). The resulting solution was stirred for ˜10 min. 110 g of {3-[4-cyclopropyl-2-(trifluoromethyl)phenyl]-5-isoxazolyl}acetic acid was added and the reaction mixture was heated to 65° C. for 1 h. The reaction solution was cooled slightly, concentrated to dryness in vacuo, and the resulting oil dried overnight in a 65° C. vacuum oven to provide 114 g (99% yield) of methyl {3-[4-cyclopropyl-2-(trifluoromethyl)phenyl]-5-isoxazolyl}acetate. ¹H NMR (400 MHz, CDCl₃) δ ppm: 7.53-7.49 (d, 1H, J=7.8 Hz), 7.46 (s, 1H), 7.29-7.24 (d, 1H, J=7.8 Hz), 6.46 (s, 1H), 3.89 (s, 1H), 3.77 (s, 3H), 2.03-1.94 (m, 1H), 1.11-1.04 (m, 2H), 0.80-0.75 (m, 2H).

### Step I

### Bis(1,1-dimethylethyl) 1-[1-{3-[4-cyclopropyl-2-(trifluoromethyl)phenyl]-5-isoxazolyl}-2-(methyloxy)-2-oxoethyl]-1,2-hydrazinedicarboxylate

To a solution of 566 g (1.74 mol) of methyl {3-[4-cyclopropyl-2-(trifluoromethyl)phenyl]-5-isoxazolyl}acetate in 5.1 L of DMF in a 20 L reactor was added 389 g (1.69 mol) of di-t-butyl azodicarboxylate and 64.3 g (870 mmol) of lithium carbonate at 0° C. After being stirred at 0° C. for 1 h, the mixture was gradually warmed to 22° C. over 16 h and stirred overnight. Upon completion of the reaction, the mixture was cooled to 0° C., diluted with 5.6 L of methyl t-butyl ether (MTBE), quenched with 110 mL 1.91 mol) of acetic acid and treated with 5.6 L of water. After being warmed to ambient temperature, the layers were separated. The aqueous layer was back extracted twice with 3.4 L and 2.8 L of MTBE, respectively. The combined organic layers were washed successively with 2×2.8 L of water and 2.8 L of saturated brine. Upon concentration to about 2.8 L at reduced pressure, the light brown solution was diluted with 2 L of heptane and seeded with a crystalline sample of the target product at ambient temperature. Crystallization started shortly after the seeding. After being treated with 100 mL of MTBE for better stirring, the mixture was stirred overnight. The mixture was filtered over a ceramic funnel. The reactor was rinsed with 250 mL of MTBE and 2×250 mL heptane, and the rinsing containing product crystals was added to the filtration. The filtering cake was dried at 55° C. to provide first crop of 608 g of bis(1,1-dimethylethyl) 1-[1-{3-[4-cyclopropyl-2-(trifluoromethyl)phenyl]-5-isoxazolyl}-2-(methyloxy)-2-oxoethyl]-1,2-hydrazinedicarboxylate as a crystalline yellow solid. The filtrate was concentrated to 524 g, diluted with 75 mL of MTBE and 250 mL of heptane. After being seeded with a crystalline sample of the product, the mixture was stirred at ambient temperature overnight, filtered, washed with 2×20 mL of 2:1 mixture of heptane and MTBE, dried at 55° C. to give 145 g of second crop of product as a crystalline solid. The total yield from the two crops was 753 g (80.3% based on input of the limiting reagent di-t-butyl azodicarboxylate). ¹H NMR (broad peaks due to mixture of tautomers, 400 MHz, CDCl₃) δ ppm 7.50 (m, 2H), 6.29-6.27 (m, 3H), 3.82 (s, 3H), 1.98 (m, 1H), 1.48, 1.38 (s, 18H), 1.08 (m, 2H), 0.78 (m, 2H). HRMS (ESI+) m/z calcd for O₂₆H₃₃F₃N₃O₇ (MH⁺) 556.2265, found 556.2268.

### Step J

### Methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate

To a 20 L reactor was added 738 g (1.33 mol) of bis(1,1-dimethylethyl) 1-[1-{3-[4-cyclopropyl-2-(trifluoromethyl)phenyl]-5-isoxazolyl}-2-(methyloxy)-2-oxoethyl]-1,2-hydrazinedicarboxylate, 392 g (1.66 mol) of 2-(2,3-difluorophenyl)-1H-imidazole-4,5-dicarbaldehyde and 7.4 L of EtOAc. The mixture was cooled to 0° C., followed by addition of 1.03 L (4.71 mol) of 33 wt. % HBr in HOAc over about 5 min. The reaction was warmed to ambient temperature over 1 h and stirred overnight. After being cooled to 0° C., the mixture was treated with 5.7 L of water. After being warmed to ambient temperature, the layers were separated. The aqueous layer was back extracted twice with 3.6 L and 2.2 L of EtOAc, respectively. The combined organic layers were washed twice with 4.4 L and 3.7 L of saturated aqueous NaHCO₃, respectively, followed by wash with 3.7 L of saturated brine. Upon concentration to about 2.8 L at reduced pressure, the light brown solution was transferred to a round bottom flask, diluted with 250 mL of MTBE and seeded with a crystalline sample of the target product at ambient temperature. Crystallization started shortly after the seeding. After being stirred overnight, the mixture was filtered over a ceramic funnel. The flask was rinsed with 120 mL of MTBE and 140 mL of heptane, and the rinsing containing product crystals was added to the filtration. The filtering cake was dried at 60° C. to provide first crop of 394 g of methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate as a crystalline yellow solid. The filtrate was concentrated to about 700 mL, diluted with 100 mL of MTBE and 80 mL of heptane. Crystallization, filtration and air-drying gave 360 g of product. Recrystallization was carried out on this material by substantially dissolving the air-dried material in 250 mL of EtOAc, followed by addition of 300 mL of MTBE and 250 mL of heptanes. The mixture was stirred at ambient temperature overnight, filtered, washed with 50 mL of MTBE and 50 mL of heptane, and dried at 55° C. to give 222 g of second crop of product as a crystalline solid. The total yield from the two crops was 616 g (83%). ¹H NMR (400 MHz, CDCl₃) δ ppm 9.28 (s, 1H), 9.27 (s, 1H), 8.19 (t, J=6 Hz, 1H), 7.54 (d, J=8 Hz, 1H), 7.49 (m, 1H), 7.31 (d, J=8 Hz, 1H), (7.21-7.28 (m, 2H), 6.88 (s, 1H), 6.78 (s, 1H), 3.95 (s, 3H), 2.01 (m, 1H), 1.25 (m, 1H), 1.12 (m, 2H), 0.80 (m, 2H). HRMS (ESI+) m/z calcd for C₂₇H₁₉F₅N₅O₃ (MH+) 556.1402, found 556.1401.

### Example 21

### 2-[(2-hydroxyethyl)oxy]ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate

### Compound 179

To a stirred solution of 0.450 kg methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate (0.785 moles—made according Example 20 herein) in 4.5 L diethyleneglycol (10 vol) was added 1.09 L triethylamine (7.85 moles) and stirred at 40° C. for 2 hours. The reaction was checked for completion and then treated with 4.5 L ethyl acetate (10 vol), then 0.453 L acetic acid (7.925 moles). The reaction was washed with 4.5 L 20% NaCl (w/v, aq.) (10 vol) then 10% NaCl (w/v, aq.) (10 vo), then 2×4.5 L water (10 vol). The EtOAc solution was concentrated to approximately 2.25 L (5 vol) under reduced pressure with jacket temperature at 40° C. Seed crystals and heptanes (6 vol) were added to solution to induce crystallization. The mixture was then cooled to 20° C. and 3 vol heptanes was added. The solids were filtered and the cake washed with (1:1) EtOAc/Heptane (2 vol). The isolated solid was dried in a vacuum oven at 80° C. to afford 362 g (73%) of 24(2-hydroxyethyl)oxy]ethyl{3-[4-cyclopropyl-2-(trifluoromethyl)phenyl]-5-isoxazolyl}[2-(2,3-difluorophenyl)-5H-imidazo[4,5-d]pyridazin-5-yl]acetate as a tan solid. ¹H NMR (400 MHz, DMSO-d₆) δ ppm 10.10 (d, J=0.8 Hz, 1H), 9.55 (d, J=1.0 Hz, 1H), 8.19 (dd, J=7.9, 6.3 Hz, 1H), 7.73 (s, 1H), 7.64 (d, J=1.4 Hz, 1H), 7.58 (m, 2H), 7.46 (m, 1H), 7.39 (m, 1H), 7.14 (s, 1H), 4.56 (t, J=5.2 Hz, 1H), 4.41 (m, 2H), 3.62 (t, J=4.6 Hz, 2H), 3.38 (m, 2H), 2.15 (m, 1H), 1.08 (m, 2H), 0.84 (m, 2H).

### Example 22

### 2-[(2-hydroxyethyl)oxy]ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate

### Compound 180

To a stirred solution of 35 g methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate (0.061 moles—made according Example 20 herein), 293 g 1,1,1-tris(hydroxymethyl)ethane (2.44 moles), and 350 mL DMF (10 vol) was added 127 mL triethylamine (0.916 moles) and stirred at 30° C. for 5 hours. The reaction was checked for completion and cooled to 20 C. To this was added 700 mL EtOAc (20 vol) then 53 mL acetic acid (0.922 moles). The 700 mL brine (20 vol) was added and the mixture stirred, separated, and the aqueous phase was discarded. The EtOAc layer was washed 3×700 mL 10% NaCl (w/v, aq.) (10 vol). and then the organics concentrated on a rotary evaporator to approximately 1 volume. This was then diluted with 350 mL EtOAc (5 vol) and filtered to remove a salts. 70 mL heptane (2 vol) was added and crystallization occurred in approximately 5 minutes. An additional 105 mL heptane (3 vol) was then added and the mixture cooled to 0° C. The solid was filtered, and washed with (1:1) EtOAc/Heptane (2 vol). This was dried in a vacuum oven at 65° C. to afford 26.81 g (70%) 3-hydroxy-2-(hydroxymethyl)-2-methylpropyl {3-[4-cyclopropyl-2-(trifluoromethyl)phenyl]-5-isoxazolyl}[2-(2,3-difluorophenyl)-5H-imidazo[4,5-d]pyridazin-5-yl]acetate as a white solid. ¹H NMR (400 MHz, DMSO-d₆) δ ppm 10.11 (d, J=0.8 Hz, 1H), 9.57 (s, 1H), 8.21 (s, 1H), 7.71 (s, 1H), 7.65 (s, 1H), 7.58 (m, 2H), 7.46 (m, 1H), 7.38 (m, 1H), 7.12 (s, 1H), 4.52 (m, 2H), 4.12 (q, J=10.6 Hz, 2H), 3.10 (m, 4H), 2.15 (m, 1H), 1.08 (dd, J=8.4, 2.3 Hz, 2H), 0.84 (m, 2H), 0.65 (s, 3H).

### Example 23

### Ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate

### Compound 181

Prepared in 89% yield from methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate (made according Example 20 herein) and ethanol according to the procedure described in Example 22 herein for the synthesis of 2-[(2-hydroxyethyl)oxy]ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate. ¹H NMR (400 MHz, CHLOROFORM-d) δ ppm 9.35 (d, J=0.7 Hz, 1H) 9.30 (d, J=1.0 Hz, 1H) 8.15-8.25 (m, 1H) 7.56 (d, J=8.0 Hz, 1H) 7.50 (d, J=1.3 Hz, 1H) 7.18-7.37 (m, 3H) 6.90 (s, 1H) 6.81 (s, 1H) 4.30-4.53 (m, 2H) 1.95-2.08 (m, 1H) 1.34 (t, J=7.1 Hz, 3H) 1.06-1.18 (m, 2H) 0.75-0.86 (m, 2H). LCMS m/z 570 (M+1).

### Example 24

### 3-Hydroxypropyl {3-[4-cyclopropyl-2-(trifluoromethyl)phenyl]-5-isoxazolyl}[2-(2,3-difluorophenyl)-5H-imidazo[4,5-d]pyridazin-5-yl]acetate

### Compound 182

Prepared in 80% yield from methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate (made according Example 20 herein) and 1,3-propanediol according to the procedure described in Example 22 herein for the synthesis of 2-[(2-hydroxyethyl)oxy]ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate. ¹H NMR (400 MHz, CHLOROFORM-d) δ ppm 9.41 (d, J=0.7 Hz, 1H) 9.29 (d, J=0.9 Hz, 1H) 8.13-8.26 (m, 1H) 7.45-7.61 (m, 2H) 7.15-7.37 (m, 3H) 6.93 (s, 1H) 6.86 (s, 1H) 4.41-4.62 (m, 2H) 3.69 (t, J=5.9 Hz, 2H) 1.83-2.22 (m, 4H) 1.05-1.18 (m, 2H) 0.73-0.87 (m, 2H). LCMS m/z 600 (M+1).

### Example 25

### Methyl {3-[4-cyclobutyl-2-(trifluoromethyl)phenyl]-5-isoxazolyl}[2-(2,3-difluorophenyl)-5H-imidazo[4,5-d]pyridazin-5-yl]acetate

### Compound 183

### Step A

### Bis(1,1-dimethylethyl) 141-{3-[4-cyclobutyl-2-(trifluoromethyl)phenyl]-5-isoxazolyl}-2-(methyloxy)-2-oxoethyl]-1,2-hydrazinedicarboxylate

Prepared in 50% yield from methyl {3-[4-cyclobutyl-2-(trifluoromethyl)phenyl]-5-isoxazolyl}acetate according to the procedure described in Steps A-I of Example 20 herein for the synthesis of bis(1,1-dimethylethyl) 1-O-{3-[4-cyclopropyl-2-(trifluoromethyl)phenyl]-5-isoxazolyl}-2-(methyloxy)-2-oxoethyl]-1,2-hydrazinedicarboxylate. ¹H NMR (400 MHz, CHLOROFORM-d) δ ppm 7.38-7.69 (m, 3H) 5.90-6.74 (m, 3H) 3.84 (br. s., 3H) 3.55-3.73 (m, 1H) 2.32-2.51 (m, 2H) 2.00-2.28 (m, 3H) 1.83-1.98 (m, 1H) 1.15-1.65 (m, 18H). LCMS m/z 570 (M+1).

### Step B

### Methyl {3-[4-cyclobutyl-2-(trifluoromethyl)phenyl]-5-isoxazolyl}[2-(2,3-difluorophenyl)-5H-imidazo[4,5-d]pyridazin-5-yl]acetate

Prepared in 65% yield from bis(1,1-dimethylethyl) 1-[1-{3-[4-cyclobutyl-2-(trifluoromethyl)phenyl]-5-isoxazolyl}-2-(methyloxy)-2-oxoethyl]-1,2-hydrazinedicarboxylate and 2-(2,3-difluorophenyl)-1H-imidazole-4,5-dicarbaldehyde according to the procedure described in Example 20 herein for the synthesis of methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate. ¹H NMR (400 MHz, CHLOROFORM-d) δ ppm 9.39 (s, 1H) 9.31 (d, J=1.0 Hz, 1H) 8.21 (m, J=7.7, 6.1, 1.6, 1.6 Hz, 1H) 7.57-7.67 (m, 2H) 7.45-7.54 (m, 1H) 7.16-7.36 (m, 2H) 6.92 (s, 1H) 6.86 (s, 1H) 3.96 (s, 3H) 3.59-3.72 (m, 1H) 2.35-2.51 (m, 2H) 2.03-2.28 (m, 3H) 1.84-1.98 (m, 1H). LCMS m/z 570 (M+1).

### Example 26

### Propyl {3-[4-cyclopropyl-2-(trifluoromethyl)phenyl]-5-isoxazolyl}[2-(2,3-difluorophenyl)-5H-imidazo[4,5-d]pyridazin-5-yl]acetate

### Compound 184

Prepared in 82% yield from methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate (made according Example 20 herein) and n-propanol according to the procedure described in Example 22 herein for the synthesis of 2-[(2-hydroxyethyl)oxy]ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate. ¹H NMR (400 MHz, CHLOROFORM-d) δ ppm 9.35 (s, 1H) 9.30 (d, J=0.9 Hz, 1H) 8.17-8.25 (m, 1H) 7.54 (d, J=8.0 Hz, 1H) 7.50 (d, J=1.2 Hz, 1H) 7.17-7.35 (m, 3H) 6.90 (s, 1H) 6.82 (s, 1H) 4.23-4.40 (m, 2H) 1.96-2.07 (m, 1H) 1.70 (sxt, J=7.1 Hz, 2H) 1.06-1.16 (m, 2H) 0.90 (t, J=7.4 Hz, 3H) 0.77-0.85 (m, 2H). LCMS m/z 584 (M+1).

### Example 27

### 4-hydroxybutyl {3-[4-cyclopropyl-2-(trifluoromethyl)phenyl]-5-isoxazolyl}[2-(2,3-difluorophenyl)-5H-imidazo[4,5-d]pyridazin-5-yl]acetate

### Compound 185

Prepared in 64% yield from methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate (made according Example 20 herein) and 1,4-butanediol according to the procedure described in Example 22 herein for the synthesis of 2-[(2-hydroxyethyl)oxy]ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate. ¹H NMR (400 MHz, CHLOROFORM-d) δ ppm 9.41 (d, J=0.8 Hz, 1H) 9.29 (d, J=0.8 Hz, 1H) 8.13-8.26 (m, 1H) 7.45-7.61 (m, 2H) 7.15-7.37 (m, 3H) 6.93 (s, 1H) 6.86 (s, 1H) 4.41-4.60 (m, 2H) 3.69 (m, 2H) 2.05 (m, 1H) 1.80 (m, 2H) 1.58 (m, 2H), 1.13 (m, 2H), 0.82 (m, 2H). LCMS m/z 614 (M+1).

### Example 28

### 3-({bis[(1,1-dimethylethyl)oxy]phosphoryl}oxy)propyl {3-[4-cyclopropyl-2-(trifluoromethyl)phenyl]-5-isoxazolyl}[2-(2,3-difluorophenyl)-5H-imidazo[4,5-d]pyridazin-5-yl]acetate

### Compound 186

### Step A

To a solution of 3-hydroxypropyl {3-[4-cyclopropyl-2-(trifluoromethyl)phenyl]-5-isoxazolyl}[2-(2,3-difluorophenyl)-5H-imidazo[4,5-d]pyridazin-5-yl]acetate (3 g, 5.00 mmol) in Tetrahydrofuran (20 mL) at 0° C. was added tetrazole in MeCN (16.68 mL, 7.51 mmol, 0.5M) then di-t-butyl diethylamino phosphoramidite (1.622 g, 6.51 mmol) and the mixture stirred at 0 C for 16 hrs. Then at 0° C., H₂O₂ (30% aq., 5 mL) was added and the solids went into solution and the color lightened. After 30 minutes the reaction was complete. The mixture was poured into 1:1 EtOAc: water (200 mL) and the organics separated, dried (brine, Na2SO4), concentrated and purified on silica (DCM-MeCN eluting at 40% MeCN) to give the product phosphate trimester which was used directly in the subsequent Step B reaction.

### 3-(phosphonooxy)propyl {3-[4-cyclopropyl-2-(trifluoromethyl)phenyl]-5-isoxazolyl}[2-(2,3-difluorophenyl)-5H-imidazo[4,5-d]pyridazin-5-yl]acetate

### Step B

A solution of 3-({bis[(1,1-dimethylethyl)oxy]phosphoryl}oxy)propyl {3-[4-cyclopropyl-2-(trifluoromethyl)phenyl]-5-isoxazolyl}[2-(2,3-difluorophenyl)-5H-imidazo[4,5-d]pyridazin-5-yl]acetate (2.3 g, 2.91 mmol) in dichloromethane (30 mL) was treated with TFA (1.119 mL, 14.53 mmol) and the mixture stirred at RT for 1 hour. The solution was washed with water (3×50 mL) and then dried (brine, Na₂SO₄) and then concentrated to an oil. This was crystallized from EtOAc/MeCN: Et₂O giving the desired product (1.25 g, 61%). ¹H NMR (400 MHz, DMSO-d6) δ ppm 11.05 (br s, 1 h) 10.10 (s, 1H), 9.55 (s, 1H) 8.19 (m, 1H) 7.52-7.71 (m, 4H) 7.32-7.49 (m, 2H) 7.11 (s, 1H) 4.35 (m, 2H) 3.80 (m, 2H) 3.35 (br s, 1H) 2.15 (m, 1H) 1.87 (m, 2H) 1.08 (m, 2H) 0.82 (m, 2H). LCMS m/z 680 (M+1).

### Example 29

### Methyl [2-(2-fluorophenyl)-5H-imidazo[4,5-c]pyridin-5-yl]{3-[4-(propyloxy)-2-(trifluoromethyl)phenyl]-5-isoxazolyl}acetate

### Compound 187

To a stirred solution of 0.253 g (1.18 mmol) of 2-(2-fluorophenyl)-5H-imidazo[4,5-c]pyridine (J. Med. Chem. 1985, 28, 717-727) in 5 mL of DMF was added 0.491 g (3.55 mmol) of potassium carbonate. To the resulting mixture was added a solution of 0.500 g (1.18 mmol) of methyl bromo{3-[4-(propyloxy)-2-(trifluoromethyl)phenyl]-5-isoxazolyl}acetate in 2 mL of DMF over a 1 minute period. The mixture was stirred at RT. After 1 hour, LCMS indicated partial conversion of the 2-(2-fluorophenyl)-5H-imidazo[4,5-c]pyridine starting material to the desired product. The reaction mixture was treated with a second 0.500 g portion of methyl bromo{3-[4-(propyloxy)-2-(trifluoromethyl)phenyl]-5-isoxazolyl}acetate in 2 mL of DMF. After another 1 hour the mixture was treated with 1 mL of AcOH and diluted with EtOAc. The solution was washed with water (3×), brine (1×), dried over sodium sulfate and concentrated to dryness at reduced pressure. The residue was subjected to flash chromatography (silica gel, gradient from DCM to 9:1 DCM/MeOH) followed by reverse phase HPLC purification (C18, gradient from 9:1 water/0.1% TFA: MeCN/0.1% TFA to 100% MeCN/0.1% TFA over 15 minutes). Fractions containing pure product were combined and concentrated to dryness at reduced pressure. The residue was dissolved in EtOAc. The solution was washed with 10% aqueous sodium bicarbonate (3×), brine (1×), dried over sodium sulfate and concentrated to dryness at reduced pressure to afford 74 mg (11%) of methyl [2-(2-fluorophenyl)-5H-imidazo[4,5-c]pyridin-5-yl]{3-[4-(propyloxy)-2-(trifluoromethyl)phenyl]-5-isoxazolyl}acetate as an orange foam. ¹H NMR (400 MHz, DMSO-d₆) δ ppm 9.13 (d, J=1.5 Hz, 1H) 8.33 (td, J=7.7, 1.6 Hz, 1H) 8.22 (dd, J=7.0, 1.6 Hz, 1H) 7.89 (d, J=6.9 Hz, 1H) 7.68 (d, J=8.5 Hz, 1H) 7.58 (s, 1H) 7.45-7.55 (m, 1H) 7.27-7.44 (m, 4H) 7.26 (s, 1H) 4.09 (t, J=6.5 Hz, 2H) 3.88 (s, 3H) 1.77 (sxt, J=7.0 Hz, 2H) 0.99 (t, J=7.4 Hz, 3H). LCMS m/z 555 (M+1).

### Example 30

### Methyl [2-(2,3-difluorophenyl)-5H-imidazo[4,5-d]pyridazin-5-yl]{6-[4-(propyloxy)-2-(trifluoromethyl)phenyl]-3-pyridinyl}acetate

### Compound 39

### Step A

### Methyl (6-chloro-3-pyridinyl)acetate

To a stirred portion of 50 mL of MeOH was slowly added 7 mL of acetyl chloride. After 15 minutes 5.00 g (29.1 mmol) of 2-chloropyridine-5-acetic acid was added and the resulting solution stirred at RT. After 2 hours the solution was concentrated to dryness at reduced pressure. The residue was partitioned between EtOAc and 10% aqueous sodium carbonate and the phases separated. The EtOAc solution was washed with 10% aqueous NaCl (2×), saturated brine (1×), dried over sodium sulfate and concentrated to dryness at reduced pressure to afford methyl (6-chloro-3-pyridinyl)acetate as a light yellow oil in quantitative yield. ¹H NMR (400 MHz, DMSO-d₆) δ ppm 8.32 (d, J=2.3 Hz, 1H) 7.78 (dd, J=8.2, 2.4 Hz, 1H) 7.49 (d, J=8.2 Hz, 1H) 3.79 (s, 2H) 3.64 (s, 3H). LCMS m/z 186 (M+1).

### Step B

### Methyl {6-[4-(propyloxy)-2-(trifluoromethyl)phenyl]-3-pyridinyl}acetate

A solution of 1.00 g (5.39 mmol) of methyl (6-chloro-3-pyridinyl)acetate, 1.40 g (5.66 mmol) of [4-(propyloxy)-2-(trifluoromethyl)phenyl]boronic acid, and 1.22 g (8.08 mmol) of CsF in 25 mL of anhydrous DME was deoxygenated by bubbling a stream of nitrogen through for 5 minutes. The solution was treated with 0.187 g (0.162 mmol) of Pd(Ph₃P)₄ and heated to reflux with stirring under nitrogen. After 18 hours the mixture was cooled to RT and diluted with EtOAc. The resulting mixture was washed with 10% aqueous NaCl (1×), saturated aqueous brine (1×), dried over sodium sulfate and concentrated to dryness at reduced pressure. The crude material was subjected to flash chromatography (silica gel, gradient elution from hexane to EtOAc) to 1.48 g (78%) of methyl {6-[4-(propyloxy)-2-(trifluoromethyl)phenyl]-3-pyridinyl}acetate as a viscous yellow oil. ¹H NMR (400 MHz, DMSO-d₆) δ ppm 8.47 (d, J=1.9 Hz, 1H) 7.73 (dd, J=8.1, 2.2 Hz, 1H) 7.33-7.49 (m, 2H) 7.18-7.32 (m, 2H) 4.02 (t, J=6.5 Hz, 2H) 3.77 (s, 2H) 3.62 (s, 3H) 1.73 (sxt, J=7.0 Hz, 2H) 0.96 (t, J=7.4 Hz, 3H). LCMS m/z 354 (M+1).

### Step C

### Methyl bromo{6-[4-(propyloxy)-2-(trifluoromethyl)phenyl]-3-pyridinyl}acetate

A mixture of 0.250 g (0.708 mmol) of methyl {6-[4-(propyloxy)-2-(trifluoromethyl)phenyl]-3-pyridinyl}acetate, 0.139 g (0.778 mmol) of NBS, and 12 mg (0.071 mmol) of AIBN in 20 mL of CCl₄ was heated to reflux with stirring. After 5 hours TLC indicated partial conversion to a new, higher R_{f} component. The solution was treated with an additional 50 mg (0.28 mmol) of NBS and stirred at reflux for another 16 hours. The solution was cooled to RT and concentrated to dryness at reduced pressure. The residue was subjected to flash chromatography (silica gel, gradient elution from hexane to EtOAc) to afford 0.109 g (36%) yield of methyl bromo{6-[4-(propyloxy)-2-(trifluoromethyl)phenyl]-3-pyridinyl}acetate as a light yellow oil. ¹H NMR (400 MHz, CHLOROFORM-d) δ ppm 8.69 (d, J=2.2 Hz, 1H) 8.01 (dd, J=8.2, 2.3 Hz, 1H) 7.43 (d, J=8.2 Hz, 1H) 7.40 (d, J=8.5 Hz, 1H) 7.22-7.26 (m, 1H) 7.09 (dd, J=8.6, 2.5 Hz, 1H) 5.38 (s, 1H) 3.98 (t, J=6.5 Hz, 2H) 3.82 (s, 3H) 1.83 (sxt, J=7.1 Hz, 2H) 1.04 (t, J=7.4 Hz, 3H).

### Step D

### Methyl [2-(2,3-difluorophenyl)-5H-imidazo[4,5-d]pyridazin-5-yl]{6-[4-(propyloxy)-2-(trifluoromethyl)phenyl]-3-pyridinyl}acetate

A stirred mixture of 59 mg (0.25 mmol) of 2-(2,3-difluorophenyl)-5H-imidazo[4,5-d]pyridazine and 0.105 g (0.76 mmol) of potassium carbonate in 6 mL of DMF was cooled in an ice water bath. A solution of 0.11 g (0.25 mmol) of methyl bromo{6-[4-(propyloxy)-2-(trifluoromethyl)phenyl]-3-pyridinyl}acetate in 2 mL of DMF was added by dropwise addition and the resulting mixture allowed to warm to RT. After 1 hour the mixture was diluted with EtOAc, washed with half saturated brine (2×), saturated brine (1×), dried over sodium sulfate and concentrated to dryness at reduced pressure. The crude material was subjected to flash chromatography (silica gel, gradient elution from DCM to 9:1 DCM/MeOH) to afford 0.100 g (68%) of methyl [2-(2,3-difluorophenyl)-5H-imidazo[4,5-d]pyridazin-5-yl]{6-[4-(propyloxy)-2-(trifluoromethyl)phenyl]-3-pyridinyl}acetate as a light yellow foam. ¹H NMR (400 MHz, DMSO-d₆) δ ppm 10.14 (d, J=0.9 Hz, 1H) 9.53 (d, J=1.0 Hz, 1H) 8.88 (d, J=2.1 Hz, 1H) 8.10-8.21 (m, 2H) 7.46-7.64 (m, 3H) 7.28-7.41 (m, 4H) 4.07 (t, J=6.5 Hz, 2H) 3.82 (s, 3H) 1.77 (sxt, J=7.0 Hz, 2H) 1.00 (t, J=7.4 Hz, 3H). LCMS m/z 584 (M+1).

### Example 31

### Ethyl [2-(2,3-difluorophenyl)-5H-imidazo[4,5-d]pyridazin-5-yl]{6-[4-(propyloxy)-2-(trifluoromethyl)phenyl]-3-pyridinyl}acetate

### Compound 40

A solution of 60 mg (0.10 mmol) of methyl [2-(2,3-difluorophenyl)-5H-imidazo[4,5-d]pyridazin-5-yl]{6-[4-(propyloxy)-2-(trifluoromethyl)phenyl]-3-pyridinyl}acetate in 6 mL of absolute EtOH was treated with 30 mg (0.21 mmol) of K₂CO₃ and stirred at RT. After 1 hour LCMS indicated complete conversion to the ethyl ester. The mixture was treated with 1 mL of AcOH and diluted with EtOAc. The resulting solution was washed with 10% aqueous NaCl (1×), saturated aqueous NaCl (1×), dried over sodium sulfate and concentrated to dryness at reduced pressure. The residue was subjected to reverse phase HPLC purification (C18, gradient elution from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in EtOAc. The solution was washed with water (1×), brine (1×), dried over sodium sulfate, and concentrated to dryness at reduced pressure to afford 18 mg (29%) of ethyl [2-(2,3-difluorophenyl)-5H-imidazo[4,5-d]pyridazin-5-yl]{6-[4-(propyloxy)-2-(trifluoromethyl)phenyl]-3-pyridinyl}acetate as a white foam. ¹H NMR (400 MHz, CHLOROFORM-d) δ ppm 9.28 (d, J=0.9 Hz, 1H) 9.20 (s, 1H) 8.80 (d, J=2.1 Hz, 1H) 8.12-8.21 (m, 1H) 7.90 (dd, J=8.3, 2.4 Hz, 1H) 7.56 (d, J=8.2 Hz, 1H) 7.43 (d, J=8.5 Hz, 1H) 7.15-7.31 (m, 3H) 7.12 (dd, J=8.5, 2.5 Hz, 1H) 6.63 (s, 1H) 4.32-4.44 (m, 2H) 4.00 (t, J=6.5 Hz, 2H) 1.84 (sxt, J=7.1 Hz, 2H) 1.31 (t, J=7.1 Hz, 3H) 1.05 (t, J=7.4 Hz, 3H). LCMS m/z 598 (M+1).

### Example 32

### Propyl [2-(2,3-difluorophenyl)-5H-imidazo[4,5-d]pyridazin-5-yl]{6-[4-(propyloxy)-2-(trifluoromethyl)phenyl]-3-pyridinyl}acetate

### Compound 41

A solution of 60 mg (0.10 mmol) of methyl [2-(2,3-difluorophenyl)-5H-imidazo[4,5-d]pyridazin-5-yl]{6-[4-(propyloxy)-2-(trifluoromethyl)phenyl]-3-pyridinyl}acetate in 6 mL of n-propanol was treated with 28 mg (0.21 mmol) of K₂CO₃ and the resulting orange solution stirred at RT. After 1 hour LCMS indicated complete conversion of the starting material to the desired product. The mixture was treated with 1 mL of AcOH and diluted with EtOAc. The resulting solution was washed with water (3×), brine (1×), dried over sodium sulfate and concentrated to dryness at reduced pressure. The crude material was subjected to flash chromatography (silica gel, gradient elution from DCM to 9:1 DCM/MeOH) to afford 42 mg (67%) of propyl [2-(2,3-difluorophenyl)-5H-imidazo[4,5-d]pyridazin-5-yl]{6-[4-(propyloxy)-2-(trifluoromethyl)phenyl]-3-pyridinyl}acetate as a light yellow foam. ¹H NMR (400 MHz, DMSO-d₆) δ ppm 10.16 (d, J=0.8 Hz, 1H) 9.55 (d, J=0.9 Hz, 1H) 8.88 (d, J=2.1 Hz, 1H) 8.10-8.22 (m, 2H) 7.54-7.64 (m, 2H) 7.46-7.53 (m, 1H) 7.28-7.42 (m, 4H) 4.15-4.28 (m, 2H) 4.07 (t, J=6.5 Hz, 2H) 1.77 (sxt, J=7.0 Hz, 2H) 1.56 (sxt, J=7.0 Hz, 2H) 1.00 (t, J=7.4 Hz, 3H) 0.76 (t, J=7.4 Hz, 3H). LCMS m/z 612 (M+1).

### Example 33

### 1-Methylethyl [2-(2,3-difluorophenyl)-5H-imidazo[4,5-d]pyridazin-5-yl]{6-[4-(propyloxy)-2-(trifluoromethyl)phenyl]-3-pyridinyl}acetate

### Compound 42

A solution of 60 mg (0.10 mmol) of methyl [2-(2,3-difluorophenyl)-5H-imidazo[4,5-d]pyridazin-5-yl]{6-[4-(propyloxy)-2-(trifluoromethyl)phenyl]-3-pyridinyl}acetate in 4 mL of isopropanol was treated with 30 mg of K₂CO₃. The mixture was subjected to microwave heating at 100° C. for 10 minutes. After stirring overnight at RT, the mixture was treated with 1 mL of AcOH and diluted with EtOAc. The solution was washed with water (3×), brine (1×), dried over sodium sulfate and concentrated to dryness at reduced pressure. The crude material was subjected to flash chromatography (silica gel, gradient elution from DCM to 9:1 DCM/MeOH) to afford 29 mg (46%) of 1-methylethyl [2-(2,3-difluorophenyl)-5H-imidazo[4,5-d]pyridazin-5-yl]{6-[4-(propyloxy)-2-(trifluoromethyl)phenyl]-3-pyridinyl}acetate as a light yellow foam. ¹H NMR (400 MHz, CHLOROFORM-d) δ ppm 9.38 (s, 1H) 9.33 (s, 1H) 8.82 (d, J=2.1 Hz, 1H) 8.13-8.22 (m, 1H) 7.92 (dd, J=8.3, 2.4 Hz, 1H) 7.57 (d, J=8.2 Hz, 1H) 7.44 (d, J=8.5 Hz, 1H) 7.17-7.35 (m, 3H) 7.13 (dd, J=8.5, 2.4 Hz, 1H) 6.65 (s, 1H) 5.14-5.30 (m, 1H) 4.00 (t, J=6.5 Hz, 2H) 1.84 (sxt, J=7.1 Hz, 2H) 1.31 (d, J=6.3 Hz, 3H) 1.27 (d, J=6.3 Hz, 3H) 1.06 (t, J=7.4 Hz, 3H). LCMS m/z 612 (M+1).

### Example 34

### Methyl [2-(2,3-difluorophenyl)-5H-imidazo[4,5-d]pyridazin-5-yl]{2-[4-(propyloxy)-2-(trifluoromethyl)phenyl]-5-pyrimidinyl}acetate

### Compound 58

### Step A

### Methyl 2-[4-(propyloxy)-2-(trifluoromethyl)phenyl]-5-pyrimidinecarboxylate

A 20 mL microwave vial was charged with 0.500 g (2.90 mmol) of methyl-2-chloropyrimidine-5-carboxylic acid, 0.934 g (3.77 mmol) of [4-(propyloxy)-2-(trifluoromethyl)phenyl]boronic acid and 1.23 g (5.79 mmol) of K₃PO₄ followed by 9 mL of 8:1 dioxane/water. The mixture was deoxygenated by bubbling nitrogen through for 5 minutes, treated with 0.10 g (0.087 mmol) of Pd(Ph₃P)₄ and the vessel sealed. The mixture was subjected to microwave heating at 120° C. for 20 minutes. This same procedure was repeated twice. The crude mixtures from all three reactions were combined and partitioned between EtOAc and water. The phases were separated and the aqueous phase extracted with EtOAc (3×). The combined EtOAc solutions were washed with water (1×), saturated brine (1×), dried over sodium sulfate and concentrated to dryness at reduced pressure. The crude residue was purified by flash chromatography (silica gel, gradient elution from hexane to EtOAc) to afford 1.70 g (58%) of methyl 2-[4-(propyloxy)-2-(trifluoromethyl)phenyl]-5-pyrimidinecarboxylate as a white crystalline solid. ¹H NMR (400 MHz, DMSO-d₆) δ ppm 9.27 (s, 2H) 7.79 (d, J=8.6 Hz, 1H) 7.22-7.40 (m, 2H) 4.06 (t, J=6.5 Hz, 2H) 3.89 (s, 3H) 1.73 (sxt, J=7.0 Hz, 2H) 0.96 (t, J=7.4 Hz, 3H). LCMS m/z 341 (M+1).

### Step B

### 2-[4-(Propyloxy)-2-(trifluoromethyl)phenyl]-5-pyrimidinecarboxylic acid

A stirred solution of 1.00 g (2.94 mmol) of methyl 2-[4-(propyloxy)-2-(trifluoromethyl)phenyl]-5-pyrimidinecarboxylate in 30 mL of 2:1 THF/water was treated with 0.250 g (5.88 mmol) of LiOH monohydrate and the resulting solution stirred at RT. After 1 hour the solution was treated with 1 mL of glacial AcOH and concentrated by rotary evaporation to a volume of approximately 5 mL at which point a white solid had precipitated. The suspension was diluted with water and extracted with EtOAc (3×). The combined EtOAc extracts were washed with water (2×), dried over sodium sulfate and concentrated to dryness at reduced pressure to afford 0.92 g (96%) of 2-[4-(propyloxy)-2-(trifluoromethyl)phenyl]-5-pyrimidinecarboxylic acid as a white solid. ¹H NMR (400 MHz, DMSO-d₆) δ ppm 13.86 (br s, 1H) 9.29 (s, 2H) 7.83 (d, J=8.4 Hz, 1H) 7.28-7.46 (m, 2H) 4.10 (t, J=6.5 Hz, 2H) 1.78 (sxt, J=7.1 Hz, 2H) 1.01 (t, J=7.4 Hz, 3H). LCMS m/z 327 (M+1).

### Step C

### α-Diazo-1-{2-[4-(propyloxy)-2-(trifluoromethyl)phenyl]-5-pyrimidinyl}ethanone

A suspension of 0.25 g (0.766 mmol) of 2-[4-(propyloxy)-2-(trifluoromethyl)phenyl]-5-pyrimidinecarboxylic acid in 10 mL of thionyl chloride was heated to reflux with stirring. After 2 hours the solution was concentrated to dryness at reduced pressure. The residue was dissolved in DCM and concentrated twice to afford the acid chloride intermediate as a white solid. This material was dissolved in 30 mL of ethyl ether and the solution cooled in an ice water bath. To this solution was added a solution of diazomethane (prepared as described below) dropwise via addition funnel over a period of 10 minutes. The resulting solution was allowed to warm to RT. After 1.5 hours the solution was treated with excess silica gel and stirred for 15 minutes to destroy the unreacted diazomethane. The silica gel was removed by filtration and the filtrate concentrated to dryness at reduced pressure. The residue was subjected to flash chromatography (silica gel, gradient elution from hexane to EtOAc) to afford 0.213 g (79%) of α-diazo-1-{2-[4-(propyloxy)-2-(trifluoromethyl)phenyl]-5-pyrimidinyl}ethanone as a light yellow oil that slowly crystallized on sitting. ¹H NMR (400 MHz, CHLOROFORM-d) δ ppm 9.12 (s, 2H) 7.80 (d, J=8.7 Hz, 1H) 7.31 (d, J=2.4 Hz, 1H) 7.13 (dd, J=8.6, 2.5 Hz, 1H) 5.95 (s, 1H) 4.01 (t, J=6.5 Hz, 2H) 1.85 (sxt, J=7.1 Hz, 2H) 1.06 (t, J=7.4 Hz 3H). LCMS m/z 350 (M+1).

The diazomethane solution was prepared as follows. A solution of 12.9 g (230 mmol) of KOH in 30 mL of water was mixed with 30 mL of DCM and the stirred mixture cooled in an ice water bath. To the solution was added 1.58 g (7.66 mmol) of N-methyl-N-nitrosourea (50% by wt.) split into three portions over 5 minutes. After stirring for 30 minutes the yellow mixture was poured into a separatory funnel and the diazomethane/DCM solution drained into a flask containing 10 g of KOH pellets. After sitting over the KOH for 15 minutes while immersed in an ice water bath, the diazomethane solution was poured into an addition funnel and used as described above.

### Step D

### Methyl {2-[4-(propyloxy)-2-(trifluoromethyl)phenyl]-5-pyrimidinyl}acetate

To a refluxing solution of 0.210 g (0.598 mmol) of α-diazo-1-{2-[4-(propyloxy)-2-(trifluoromethyl)phenyl]-5-pyrimidinyl}ethanone in 10 mL of MeOH was added a solution of 40 mg (0.175 mmol) of silver benzoate and 0.25 mL (1.79 mmol) of triethylamine in 2 mL of MeOH by dropwise addition over a 5 minute period. The resulting dark red-brown solution was stirred at reflux for 1 hour and then allowed to cool to RT. The mixture was filtered through celite to remove solids and the filtrate concentrated to dryness at reduced pressure. The residue was dissolved in EtOAc. The solution was washed with saturated aqueous sodium bicarbonate (1×), dried over sodium sulfate, and concentrated to dryness at reduced pressure. The crude material was subjected to flash chromatography (silica gel, gradient elution from hexane to EtOAc) to afford 0.148 g (70%) of methyl {2-[4-(propyloxy)-2-(trifluoromethyl)phenyl]-5-pyrimidinyl}acetate as a light yellow oil. ¹H NMR (400 MHz, CHLOROFORM-d) δ ppm 8.73 (s, 2H) 7.68 (d, J=8.6 Hz, 1H) 7.27 (d, J=2.4 Hz, 1H) 7.09 (dd, J=8.6, 2.5 Hz, 1H) 3.98 (t, J=6.5 Hz, 2H) 3.74 (s, 3H) 3.66 (s, 2H) 1.82 (sxt, J=7.1 Hz, 2H) 1.03 (t, J=7.4 Hz 3H). LCMS m/z 355 (M+1).

### Step E

### Methyl bromo{2-[4-(propyloxy)-2-(trifluoromethyl)phenyl]-5-pyrimidinyl}acetate

A mixture of 0.145 g (0.409 mmol) of methyl {2-[4-(propyloxy)-2-(trifluoromethyl)phenyl]-5-pyrimidinyl}acetate, 0.146 g (0.818 mmol) of NBS, and 6.7 mg (0.041 mmol) of AIBN in 10 mL of CCl₄ was heated to reflux with stirring. After 3 hours the mixture was cooled to RT and concentrated to dryness at reduced pressure. The residue subjected to flash chromatography (silica gel, gradient elution from hexane to EtOAc) to afford 67 mg (38%) of methyl bromo{2-[4-(propyloxy)-2-(trifluoromethyl)phenyl]-5-pyrimidinyl}acetate as a colorless viscous oil. ¹H NMR (400 MHz, CHLOROFORM-d) δ ppm 8.95 (s, 2H) 7.74 (d, J=8.6 Hz, 1H) 7.28 (d, J=2.5 Hz, 1H) 7.10 (dd, J=8.6, 2.5 Hz, 1H) 5.32 (s, 1H) 3.99 (t, J=6.5 Hz, 2H) 3.84 (s, 3H) 1.75-1.89 (m, 2H) 1.04 (t, J=7.4 Hz 3H).

### Step F

### Methyl [2-(2,3-difluorophenyl)-5H-imidazo[4,5-d]pyridazin-5-yl]{2-[4-(propyloxy)-2-(trifluoromethyl)phenyl]-5-pyrimidinyl}acetate

A mixture of 38 mg (0.16 mmol) of 2-(2,3-difluorophenyl)-5H-imidazo[4,5-d]pyridazine in 5 mL of DMF was briefly warmed to dissolve the starting material. The resulting solution was treated with 62 mg (0.44 mmol) of K₂CO₃ and cooled in an ice water bath. A solution of 64 mg (0.15 mmol) of methyl bromo{2-[4-(propyloxy)-2-(trifluoromethyl)phenyl]-5-pyrimidinyl}acetate in 2 mL of DMF was added by dropwise addition. The mixture was allowed to warm to RT. After 2 hours the mixture was treated with 1 mL of glacial AcOH, diluted with EtOAc, washed with half saturated brine (2×), saturated brine (1×), dried over sodium sulfate and concentrated to dryness at reduced pressure. The crude residue was subjected to reverse phase HPLC purification (C18, gradient elution from 9:1 water/0.1% TFA: MeCN/0.1% TFA to 100% MeCN/0.1% TFA over 10 minutes). Fractions containing pure product were combined and concentrated to dryness at reduced pressure. The residue was dissolved in EtOAc. The solution was washed with saturated aqueous sodium bicarbonate (1×), brine (1×), dried over sodium sulfate and concentrated to dryness at reduced pressure to afford 42 mg (49%) of methyl [2-(2,3-difluorophenyl)-5H-imidazo[4,5-d]pyridazin-5-yl]{2-[4-(propyloxy)-2-(trifluoromethyl)phenyl]-5-pyrimidinyl}acetate as a light yellow foam. ¹H NMR (400 MHz, CHLOROFORM-d) δ ppm 9.63 (br s, 1H) 9.31 (s, 1H) 9.06 (s, 2H) 8.15 (dd, J=7.8, 6.1 Hz, 1H) 7.78 (d, J=8.6 Hz, 1H) 7.16-7.33 (m, 3H) 7.11 (dd, J=8.6, 2.5 Hz, 1H) 6.76 (s, 1H) 4.00 (t, J=6.5 Hz, 2H) 3.91 (s, 3H) 1.83 (sxt, J=7.1 Hz, 2H) 1.04 (t, J=7.4 Hz 3H). LCMS m/z 585 (M+1).

### Administration and Pharmaceutical Composition

The compounds, or pharmaceutically acceptable salts or solvates, described herein generally possess antiviral activity, including Flaviviridae family viruses, such as hepatitis C virus. The compounds may inhibit viral replication by inhibiting the enzymes involved in replication, including RNA dependent RNA polymerase. They may also inhibit other enzymes utilized in the activity or proliferation of Flaviviridae viruses.

In general, the compounds, or pharmaceutically acceptable salts or solvates, described herein will be administered in a therapeutically effective amount by any of the accepted modes of administration for agents that serve similar utilities. The actual amount of the compound, or pharmaceutically acceptable salt or solvate, described herein, i.e., the active ingredient, will depend upon numerous factors such as the severity of the disease to be treated, the age and relative health of the subject, the potency of the compound used, the route and form of administration, and other factors. The drug can be administered more than once a day, such as once or twice a day.

Therapeutically effective amounts of compounds, or pharmaceutically acceptable salts or solvates, described herein may range from approximately 0.01 to 50 mg per kilogram body weight of the recipient per day; such as about 0.01-25 mg/kg/day, for example, from about 0.1 to 10 mg/kg/day. Thus, in some embodiments, for administration to a 70 kg person, the dosage range would be about 7-70 mg per day.

This invention is not limited to any particular composition or pharmaceutical carrier, as such may vary. In general, compounds, or pharmaceutically acceptable salts or solvates, described herein will be administered as pharmaceutical compositions by any one of the following routes: oral, systemic (e.g., transdermal, intranasal or by suppository), or parenteral (e.g., intramuscular, intravenous or subcutaneous) administration. In some embodiments, for example, the compounds are water-soluble and administered orally via an aqueous solution. The manner of oral administration may be accomplished with a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another manner for administering compounds of described herein is inhalation.

The choice of formulation depends on various factors such as the mode of drug administration and bioavailability of the drug substance. For delivery via inhalation the compound can be formulated as liquid solution (e.g., aqueous solution), suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

If desired, bioavailability of the drug substance may be further increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound, or pharmaceutically acceptable salt or solvate, described herein in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and so forth. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

Compressed gases may be used to disperse a compound, or pharmaceutically acceptable salt or solvate, described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound, or pharmaceutically acceptable salt or solvate, described herein based on the total formulation, with the balance being one or more suitable pharmaceutical excipients. In some embodiments, the compound is present at a level of about 1-80 wt %. Representative pharmaceutical formulations are described in the Formulation Examples section below.

Also provided is a pharmaceutical composition comprising a therapeutically effective amount of a compound, or pharmaceutically acceptable salt or solvate, described herein in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O, Saunders and S. A. Raybuck, “Inosine Monophosphate Dehydrogenase Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine(Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods described herein contain a compound, or pharmaceutically acceptable salt or solvate, described herein and interferon. In some embodiments, the interferon is selected from interferon alpha 2B, pegylated interferon alpha, consensus interferon, interferon alpha 2A, and lymphoblastiod interferon tau.

In other embodiments, the compositions and methods described herein contain a compound, or pharmaceutically acceptable salt or solvate, described herein and a compound having anti-HCV activity is selected from interleukin 2, interleukin 6, interleukin 12, a compound that enhances the development of a type 1 helper T cell response, interfering RNA, anti-sense RNA, Imiquimod, ribavirin, an inosine 5′-monophospate dehydrogenase inhibitor, amantadine, and rimantadine.

In some embodiments, the compound having anti-HCV activity is Ribavirin, levovirin, viramidine, thymosin alpha-1, an inhibitor of NS3 serine protease, and inhibitor of inosine monophosphate dehydrogenase, interferon-alpha, or pegylated interferon-alpha alone or in combination with Ribavirin or viramidine.

In some embodiments, the compound having anti-HCV activity is said agent active against HCV is interferon-alpha or pegylated interferon-alpha alone or in combination with Ribavirin or viramidine.

In some embodiments, the present invention provides the use of a compound according to any of the Formulas or compounds described herein in the manufacture of a medicament for use in the treatment of a viral infection in a human.

In some embodiments, the present invention provides a pharmaceutical composition comprising a pharmaceutically acceptable carrier and a therapeutically effective amount of a compound according to any of the Formulas or compounds described herein or a pharmaceutically acceptable salt or solvate thereof.

In some embodiments, the present invention provides a pharmaceutical composition comprising a pharmaceutically acceptable carrier and a therapeutically effective amount of a compound having the structure:

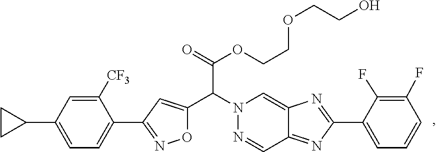

In some embodiments, the present invention provides a method for treating a viral infection in a patient said viral infection mediated at least in part by a virus in the Flaviviridae family of viruses which method comprises administering to the patient a therapeutically effective amount of a compound according to any of the Formulas or compounds described herein, or a pharmaceutically acceptable salt or solvate thereof.

In some embodiments, the present invention provides a method for treating a viral infection in a patient said viral infection mediated at least in part by a virus in the Flaviviridae family of viruses which method comprises administering to the patient a therapeutically effective amount of a compound according to any of the Formulas or compounds described herein, or a pharmaceutically acceptable salt or solvate thereof, wherein the compound is administered to the patient in combination with a therapeutically effective amount of one or more additional agent(s) active against the hepatitis C virus.

In some embodiments, the additional agent(s) active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

In some embodiments, the additional agent active against the hepatitis C virus is interferon.

In some embodiments, the additional agent active against the hepatitis C virus is ribavirin.

In some embodiments, the additional agents active against the hepatitis C virus is interferon in combination with ribavirin.

### Biological Examples

### Anti-Hepatitis C Activity

Compounds can exhibit anti-hepatitis C activity by inhibiting viral and host cell targets required in the replication cycle. A number of assays have been published to assess these activities. A general method that assesses the gross increase of HCV virus in culture is disclosed in U.S. Pat. No. 5,738,985 to Miles, et al. In vitro assays have been reported in Ferrari, et al., J. of Vir., 73:1649-1654, 1999; Ishii, et al., Hepatology, 29:1227-1235, 1999; Lohmann et al., J. of Bio. Chem., 274:10807-10815, 1999; and Yamashita, et al., J. of Bio. Chem., 273:15479-15486, 1998.

### Replicon Assay

Two cell lines were used for screening of compounds for inhibiting HCV RNA replication (genoytype 1a and 1b). Genotype la replicon cells, are a Huh-7 derived cell line bearing the genotype 1a H77NS3-5B bicistronic subgenomic replicon. See, Blight, et al., J. Virol. (2003) 77(5): 3181-3190.

The genotype 1a replicon contains several adaptive mutations (NS4B Q31H, NS5A K68R, NS5A S232I), the luciferase gene and encodes for neomycin resistance. The genotype 1b replicon, also referred to as the ET replicon, is stably transfected with RNA transcripts harboring a I₃₈₉luc-ubi-neo/NS3-3′/ET replicon with firefly luciferase-ubiquitin-neomycin phosphotransferase fusion protein and EMCV-IRES driven NS3-5B polyprotein containing the cell culture adaptive mutations (E1202G; T12801; K1846T) See, Kreiger, et al., Journal of Virology 75:4614-4624 (2001). Both cell lines were grown in DMEM, supplemented with 10% fetal calf serum, 2 mM Glutamine, Penicillin (100 IU/mL)/Streptomycin (100 μg/mL), 1×nonessential amino acids, and 250 μg/mL G418 (“Geneticin”). They were all available through Life Technologies (Bethesda, Md.). The cells were plated at 0.5×10⁴ cells/well in 384 well plates containing compounds. The final concentration of compounds ranged between 0.1 nM to 50 μM and the final DMSO concentration of 0.5%.

Luciferase activity was measured 48 hours later by adding a Steady glo (Promega, Madison, Wis.). Percent inhibition of replication data was plotted relative to no compound control. Under the same condition, cytotoxicity of the compounds was determined using cell titer glo (Promega, Madison, Wis.). EC₅₀ values were determined from a 10 point dose response curve using 2-4 fold serial dilutions for each compound, which spans a concentration range of at least a 1000 fold. Replicon EC₅₀ values, the concentration of compound required to inhibit 50% of the assay response, were calculated by curve fitting data to the Hill equation, using a non-linear least-squares curve-fitting program. The software tested the data for quality and rejected compounds with high and low activity before fitting the equation below.

y=a+[(b−a)/(1+(10^{x}/10^{c})^{d})]
where y=response, a=minimum response (i.e. no inhibition), b=maximum response, x=compound concentration, c=EC₅₀, and d=Hill coefficient. If the data fit did not meet quality control criteria, six secondary models with different levels of data constraint were used. Analysis was performed using an XC50 module and BioAssay Enterprise (Cambridge Soft).

As shown in Table 2 and Table 3 below, the compounds tested were found to exhibit EC₅₀ values of about 10,000 nM or less. In some embodiments, the compounds will exhibit EC₅₀ values of about 1500 nM or less, in some embodiments about 1000 nM or less, in some embodiments about 500 nM or less, in some embodiments about 100 nM or less, in some embodiments, about 40 nM or less, and in some embodiments, about 10 nM or less.

TABLE 2

| | HCV Genotype | HCV Genotype |
|---|---|---|
| Compound | 1A | 1B |
| Number | Replicon | Replicon |
| (From Table 1) | EC₅₀ (nM) | EC₅₀ (nM) |
| 1 | 17 | 3 |
| 2 | 12 | 4 |
| 3 | 20 | 5 |
| 4 | 30 | 10 |
| 5 | 24 | 6 |
| 6 | 65 | 16 |
| 7 | 11 | 5 |
| 8 | 7 | 3 |
| 9 | 2 | 4 |
| 11 | 21 | 3 |
| 12 | 17 | 1 |
| 14 | 42 | 3 |
| 15 | 14 | 2 |
| 16 | 32 | 3 |
| 173 | 20 | 3 |
| 20 | 920 | 65 |
| 39 | 9440 | 800 |
| 40 | — | 440 |
| 41 | 900 | 2700 |
| 42 | 980 | 510 |
| 58 | 2600 | 270 |

TABLE 3

| | HCV Genotype | HCV Genotype | |
|---|---|---|---|
| Compound | 1A | 1B | HCV Genotype |
| Number | Replicon | Replicon | 1B |
| (From Table 1) | EC₅₀ (nM) | EC₅₀ (nM) | CC₅₀ (μM) |
| 178 | 50 nM | 10 nM | 25 μM |
| 179 | 79 nM | 13 nM | 50 μM |
| 180 | 79 nM | 8 nM | — |
| 182 | 63 nM | 13 nM | >50 μM |
| 184 | 79 nM | 13 nM | 25 μM |
| 186 | 79 nM | 10 nM | 50 μM |

### Formulation Examples

The following are representative pharmaceutical formulations containing a compound of Formula (I), or a pharmaceutically acceptable salt or solvate.

### Formulation Example 1

### Tablet Formulation

The following ingredients may be mixed intimately and pressed into single scored tablets.

| | Quantity per |
|---|---|
| Ingredient | tablet, mg |
| compound | 400 |
| cornstarch | 50 |
| croscarmellose sodium | 25 |
| lactose | 120 |
| magnesium stearate | 5 |

### Formulation Example 2

### Capsule Formulation

The following ingredients may be mixed intimately and loaded into a hard-shell gelatin capsule.

| | Quantity per |
|---|---|
| Ingredient | capsule, mg |
| compound | 200 |
| lactose, spray-dried | 148 |
| magnesium stearate | 2 |

### Formulation Example 3

### Suspension Formulation

The following ingredients may be mixed to form a suspension for oral administration.

| Ingredient | Amount | |
|---|---|---|
| compound | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. (quantity |
| | sufficient to I00 mL |

### Formulation Example 4

### Injectable Formulation

The following ingredients may be mixed to form an injectable formulation.

| Ingredient | Amount |
|---|---|
| compound | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g may be prepared by mixing the compound with Witepsol® H-15 (triglycerides of saturated vegetable fatty acid; Riches-Nelson, Inc., New York), and it may have the following composition:

| Ingredient | Amount |
|---|---|
| compound | 500 mg |
| Witepsol ® H-15 | balance |

What is claimed is:

1. A compound that is Formula (I): 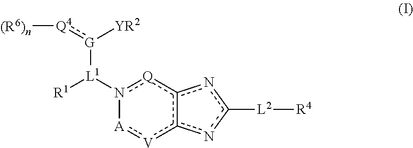
or a pharmaceutically acceptable salt or solvate thereof, wherein:
the dotted lines represent an optional double bond, provided that no two double bonds are adjacent to one another, and that the dotted lines represent at least 3 double bonds;
G is C when the bond between G and Q⁴ is a double bond and G is C-Q¹ when the bond between G and Q⁴ is a single bond;
A, Q, and V are independently selected from N and CR³;
L¹ is independently C₃₋₆ cycloalkylene or C₁₋₅ alkylene, where one or two CH₂ groups of said C₁₋₅ alkylene are optionally replaced with NR^{a}, S, (C═O), or O and optionally two adjacent carbon atoms form a double bond;
L² is a bond or independently C₃₋₆ cycloalkylene or is C₁₋₅ alkylene where one or two CH₂ groups of said C₁₋₅ alkylene are optionally replaced with NR^{b}, S, (C═O), or O and optionally two adjacent carbon atoms form a double bond or triple bond, and wherein said C₁₋₅ alkylene is optionally substituted with one to three groups independently selected from halo, alkyl, and spirocycloalkyl;
Y is a bond, O, S, or NR^{c};
Q¹ is selected from hydrogen, halo, amino, acylamino, alkyl, substituted alkyl, alkenyl, substituted alkenyl, alkynyl, substituted alkynyl, carboxy, carboxy ester, azido, hydroxy, alkoxy, substituted alkoxy, acyl, acyloxy, cyano, thiol, alkylthio, substituted alkylthio, and substituted sulfonyl;
Q⁴ is O, S, or NR⁷;
R¹ is selected from aryl, substituted aryl, heteroaryl, substituted heteroaryl, cycloalkyl, substituted cycloalkyl, heterocyclyl, and substituted heterocyclyl;
R² is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl, and a pharmaceutically acceptable counterion;
R³ is selected from hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl;
R⁴ is independently selected from aryl, substituted aryl, heteroaryl, substituted heteroaryl, heterocyclyl, substituted heterocyclyl, cycloalkyl, and substituted cycloalkyl;
R⁶ is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl, and a pharmaceutically acceptable counterion;
R⁷ is selected from hydrogen, halo, aminocarbonyl, imino, amidino, aminocarbonylamino, amidinocarbonylamino, carboxy, carboxy ester, hydroxy, alkoxy, substituted alkoxy, acyl, acyloxy, cyano, thiol, alkylthio, substituted alkylthio, sulfonyl, and substituted sulfonyl;
R^{a}, R^{b}, and R^{c} are independently selected from hydrogen, alkyl, and substituted alkyl; and
n is from 0 to 1, provided that n is 0 when the bond between G and Q⁴ is a double bond.

2. The compound of claim 1, wherein A is N.

3. The compound of claim 1, wherein Q and V are CR³, where R³ is independently selected from hydrogen and a lower alkyl.

4. The compound of claim 1, wherein V is N, and A and Q are CR³, where R³ is independently selected from hydrogen and a lower alkyl.

5. The compound of claim 1, wherein V, Q, and A are CR³, where R³ is independently selected from hydrogen and a lower alkyl.

6. The compound of claim 1, wherein R³ is hydrogen.

7. The compound of claim 1, wherein the bond between G and Q⁴ is a double bond.

8. The compound of claim 1, wherein Q⁴ is O.

9. The compound of claim 1, wherein Q⁴ is NR⁷.

10. The compound of claim 1, wherein the bond between G and Q⁴ is a single bond.

11. The compound of claim 1, wherein Q¹ is hydrogen.

12. The compound of claim 1, wherein n is 1.

13. The compound of claim 1, wherein n is 0.

14. The compound of claim 1, wherein R⁶ is hydrogen, alkyl, or substituted alkyl.

15. The compound of claim 1, wherein A is CR³ and R³ is hydrogen.

16. The compound of claim 1, wherein L² is a bond.

17. The compound of claim 1, wherein L¹ is CH₂.

18. The compound of claim 1, wherein Y is a bond, NH, or O.

19. The compound of a claim 1, wherein Y is a bond.

20. The compound of any of claim 1, wherein Y is NH.

21. The compound of any of claim 1 , wherein Y is O.

22. The compound of claim 1, wherein R⁶ is hydrogen, alkyl, or substituted alkyl.

23. The compound of claim 1, wherein R¹ is: 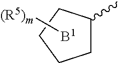
wherein,
ring B¹ is a 5-membered aromatic ring wherein 1 to 3 ring carbon atoms are optionally replaced by nitrogen or oxygen, wherein each nitrogen is optionally oxidized, and wherein ring B¹ may be optionally fused to a 5- or 6-membered aryl, substituted aryl, heteroaryl, substituted heteroaryl, heterocycle or substituted heterocycle to form a 8- or 9-membered bicyclic ring;
R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, substituted sulfonyl, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl; and
m is from 0 to 4.

24. The compound of claim 23, wherein the ring B¹ is selected from: 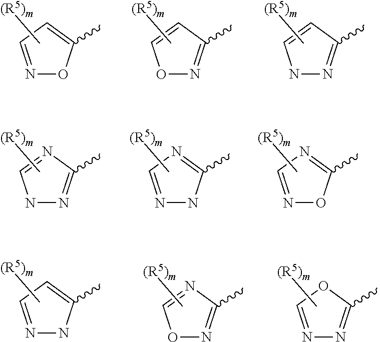
wherein,
m is from 0 to 4; and
R⁵ is independently selected from halo, haloalkyl, haloalkoxy, aryl, substituted aryl, heteroaryl, substituted heteroaryl, heterocyclyl, substituted heterocyclyl, cycloalkyl, and substituted cycloalkyl.

25. The compound of claim 24, wherein ring B¹ is selected from the group consisting of:

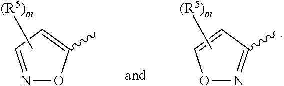

26. The compound of claim 25, wherein ring B is:

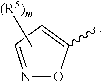

27. The compound of claim 1, wherein R¹ is: 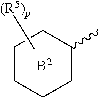
wherein,
ring B² is a 6-membered aromatic ring wherein 1 to 3 ring carbon atoms are optionally replaced by nitrogen or oxygen, wherein each nitrogen is optionally oxidized, and wherein ring B² may be optionally fused to a 5- or 6-membered aryl, substituted aryl, heteroaryl, substituted heteroaryl, heterocycle or substituted heterocycle to form a 9- or 10-membered bicyclic ring;
R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, substituted sulfonyl, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl; and
p is from 0 to 5.

28. The compound of claim 27, wherein the ring B² is selected from: 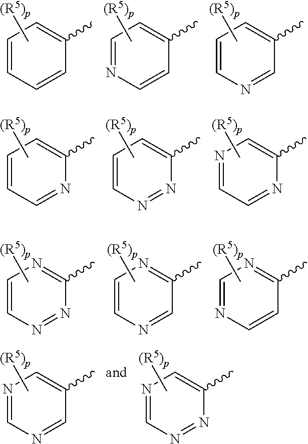
wherein,
p is 1, 2, 3, or 4; and
R⁵ is independently selected from halo, haloalkyl, haloalkoxy, aryl, substituted aryl, heteroaryl, substituted heteroaryl, heterocyclyl, substituted heterocyclyl, cycloalkyl, and substituted cycloalkyl.

29. The compound of claim 27, wherein the ring B² is selected from: 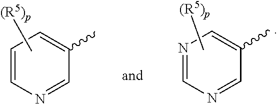
wherein,
m is from 0 to 5; and
R⁵ is independently selected from halo, haloalkyl, haloalkoxy, aryl, substituted aryl, heteroaryl, substituted heteroaryl, heterocyclyl, substituted heterocyclyl, cycloalkyl, and substituted cycloalkyl.

30. The compound of claim 1, wherein R² is selected from the group consisting of hydrogen, C₁₋₆ alkyl, aryl, -A¹, -A¹-(X¹)_{w}R⁸R⁹, -A¹-R¹⁰, -A¹-R¹¹, -A¹-N(R⁹)_{z}, -A¹-NHA²-R¹¹, -A¹-NHA²—NHA³R¹¹, and -A¹-R⁸R⁹; wherein:
A¹, A², A³ and A⁴ an are each independently selected from C₁₋₆ alkylene, wherein one to four independent CH₂ groups of each of said A¹, A², A³, and A⁴ are optionally substituted with one to two R¹² groups;
each X¹ is independently selected from the group consisting of —(R⁸-A¹), —(R⁸-A²), —(R⁸-A³), and —(R⁸-A⁴);
R⁸ is O;
each R⁹ is independently selected from the group consisting of hydrogen and C₁₋₆ alkyl;
R¹⁰ is selected from the group consisting of phosphate, phosphonate, and sulfate;
R¹¹ is carboxyl;
each R¹² is independently selected from the group consisting of C₁₋₆ alkyl, oxo, aryl, arylalkyl, and hydroxyl;
w is an integer from 1 to 3; and
z is an integer from 2 to 3.

31. The compound of claim 30, wherein R² is selected from the group consisting of hydrogen, C₁₋₆ alkyl, phenyl, -A¹, -A¹-(X¹)_{w}R⁸R⁹, -A¹-R¹⁰, A¹-R¹¹, -A¹-N(R⁹)_{z}, -A¹-NHA²-R¹¹, -A¹-NHA²-NHA³R¹¹, and -A¹-R⁸R⁹; wherein:
A¹, A², A³ and A⁴ an are each independently selected from C₁₋₆ alkylene, wherein one to four independent CH₂ groups of each of said A¹, A², A³, and A⁴ are optionally substituted with one to two R¹² groups;
each X¹ is independently selected from the group consisting of —(R⁸-A¹), —(R⁸-A²), —(R⁸-A³), and —(R⁸-A⁴);
R⁸ is O;
each R⁹ is independently selected from the group consisting of hydrogen and C₁₋₆ alkyl;
R¹⁰ is selected from the group consisting of phosphate, phosphonate, and sulfate;
R¹¹ is carboxyl;
each R¹² is independently selected from the group consisting of C₁₋₆ alkyl, oxo, aryl, arylalkyl, and hydroxyl;
w is an integer from 1 to 3; and
z is an integer from 2 to 3.

32. The compound of claim 1, wherein R² is selected from the group consisting of alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, and substituted sulfonyl.

33. The compound of claim 1, wherein R² is C₁₋₆ alkyl, and wherein said C₁₋₆ alkyl is substituted with 1 to 5 substituents independently selected from the group consisting of hydroxyl, alkoxy, substituted alkoxy, oxy, carboxyl, phosphate, sulfate, amino, substituted amino, quaternary amino, acylamino, aminocarbonyl, and aminocarbonylamino, or a combination thereof.

34. The compound of claim 1, wherein R² is C₁₋₆ alkyl, and wherein said C₁₋₆ alkyl is substituted with 1 to 5 substituents independently selected from the group consisting of hydroxyl, alkoxy, substituted alkoxy, oxy, carboxyl, phosphate, sulfate, amino, substituted amino, quaternary amino, acylamino, aminocarbonyl, and aminocarbonylamino.

35. The compound of claim 1, wherein R² is C₁-C₆ alkyl.

36. The compound of claim 1, wherein R² is aryl or substituted aryl.

37. The compound of claim 1, wherein R² is hydroxylalkoxyalkyl.

38. The compound of claim 37, wherein R² is hydroxylethoxyethyl.

39. The compound of claim 38, wherein R² is:

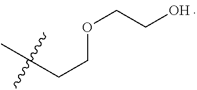

40. The compound of claim 1, wherein R² together with a pharmaceutically acceptable counterion forms a salt.

41. The compound of claim 40, wherein the pharmaceutically acceptable counterion is sodium.

42. The compound of claim 40, wherein the pharmaceutically acceptable counterion is selected from the group consisting of chlorine, bromine and flourine.

43. The compound of claim 1, wherein R⁵ is phenyl or heteroaryl, each of which is substituted with at least one group selected from the group consisting of alkyl, haloalkyl, cycloalkyl, and optionally substituted alkoxy.

44. The compound of claim 43, wherein R⁵ is selected from the group consisting of:

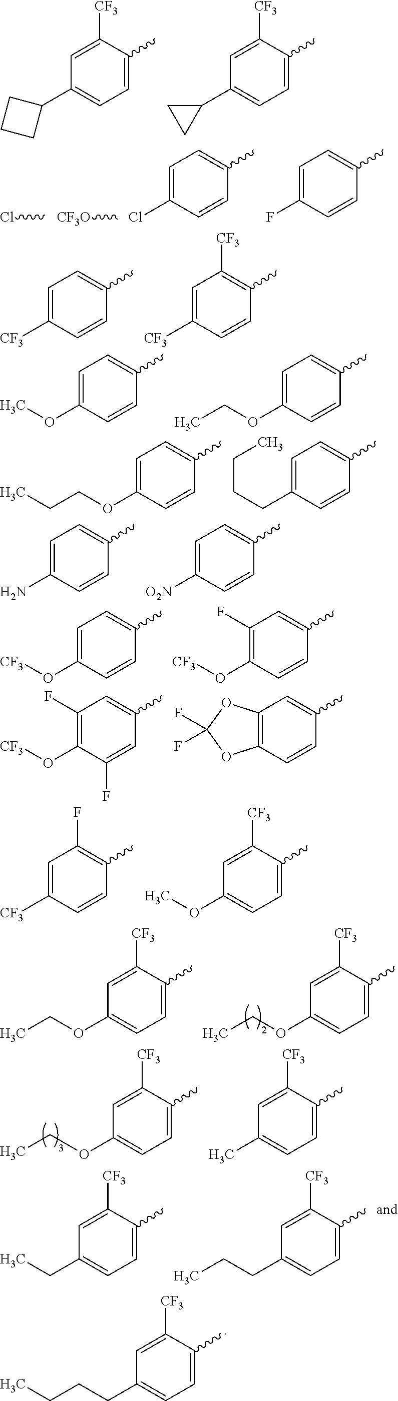

45. The compound of claim 44, wherein R⁵ is selected from:

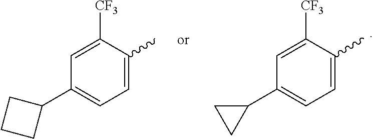

46. The compound of claim 45, wherein R⁵ is

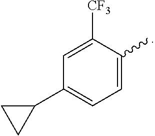

47. The compound of claim 23, wherein m is 1.

48. The compound of claim 27, wherein p is 1.

49. The compound of claim 1, wherein R⁴ is selected from the group consisting of aryl, substituted aryl, heteroaryl, and substituted heteroaryl.

50. The compound of claim 49, wherein R⁴ is aryl or substituted aryl.

51. The compound of claim 49, wherein R⁴ is phenyl or substituted phenyl.

52. The compound of claim 49, wherein R⁴ is aryl.

53. The compound of claim 49, wherein R⁴ is phenyl.

54. The compound of claim 1, wherein R⁴ is substituted with at least one halo group.

55. The compound of claim 54, wherein R⁴ is substituted with one to two fluoro groups.

56. The compound of claim 55, wherein R⁴ is fluorophenyl.

57. The compound of claim 55, wherein R⁴ is difluorophenyl.

58. The compound of claim 55, wherein R⁴ is 2-fluorophenyl.

59. The compound of claim 55, wherein R⁴ is 2,3-difluorophenyl.

60. The compound of claim 1 having the formula (IX) 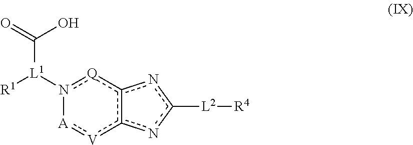
or a pharmaceutically acceptable salt thereof.

61. The compound of claim 1, having the formula (XI) 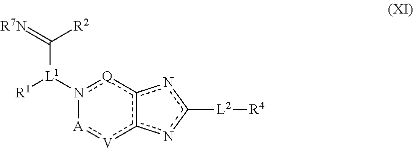
or a pharmaceutically acceptable salt thereof.

62. The compound of claim 1, having the formula (XII) 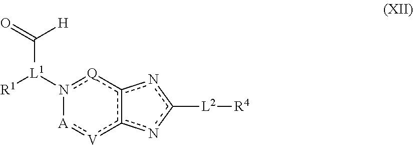
or a pharmaceutically acceptable salt thereof.

63. The compound of claim 1, having the formula (XIII) 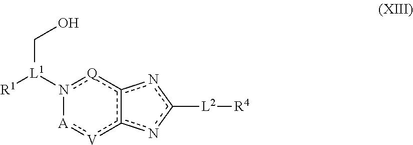
or a pharmaceutically acceptable salt thereof.

64. A compound that is Formula (II): 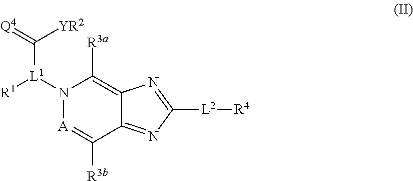
or a pharmaceutically acceptable salt or solvate thereof, wherein,
A is N or CR³;
L¹ is independently C₃₋₆ cycloalkylene or C₁₋₅ alkylene, where one or two CH₂ groups of said C₁₋₅ alkylene are optionally replaced with NR^{a}, S, (C═O), or O and optionally two adjacent carbon atoms form a double bond;
L² is a bond or independently C₃₋₆ cycloalkylene or is C₁₋₅ alkylene where one or two CH₂ groups of said C₁₋₅ alkylene are optionally replaced with NR^{b}, S, (C═O), or O and optionally two adjacent carbon atoms form a double bond or triple bond, and wherein said C₁₋₅ alkylene is optionally substituted with one to three groups independently selected from halo, alkyl, and spirocycloalkyl;
Y is a bond, O, S, or NR^{c};
Q⁴ is O, S, or NR⁷;
R¹ is selected from aryl, substituted aryl, heteroaryl, substituted heteroaryl, cycloalkyl, substituted cycloalkyl, heterocyclyl, and substituted heterocyclyl;
R² is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl, and a pharmaceutically acceptable counterion;
R³ is selected from hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl;
R^{3a} and R^{3b} are independently R³;
R⁴ is independently selected from aryl, substituted aryl, heteroaryl, substituted heteroaryl, heterocyclyl, substituted heterocyclyl, cycloalkyl, and substituted cycloalkyl;
R⁶ is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl, and a pharmaceutically acceptable counterion;
R⁷ is selected from hydrogen, halo, aminocarbonyl, imino, amidino, aminocarbonylamino, amidinocarbonylamino, carboxy, carboxy ester, hydroxy, alkoxy, substituted alkoxy, acyl, acyloxy, cyano, thiol, alkylthio, substituted alkylthio, sulfonyl, and substituted sulfonyl; and
R^{a}, R^{b}, and R^{c} are independently selected from hydrogen, alkyl, and substituted alkyl.

65. The compound of claim 64, wherein R^{3a} and R^{3b} are hydrogen.

66. The compound of claim 65, having the Formula (XIII): 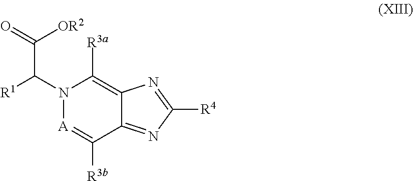
or a pharmaceutically acceptable salt thereof.

67. A compound that is Formula (I): 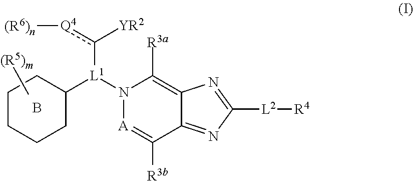
or a pharmaceutically acceptable salt or solvate thereof, wherein: [symbol]
represents a single or double bond;
ring B is a 6-membered aromatic ring wherein 1 to 3 ring carbon atoms are optionally replaced by nitrogen or oxygen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;
L¹ is independently C₃₋₆ cycloalkylene or C₁₋₅ alkylene, where one or two CH₂ groups of said C₁₋₅ alkylene are optionally replaced with NR^{a}, S, (C═O), or O and optionally two adjacent carbon atoms form a double bond;
L² is a bond or independently C₃₋₆ cycloalkylene or is C₁₋₅ alkylene where one or two CH₂ groups of said C₁₋₅ alkylene are optionally replaced with NR^{b}, S, (C═O), or O and optionally two adjacent carbon atoms form a double bond or triple bond, and wherein said C₁₋₅ alkylene is optionally substituted with one to three groups independently selected from halo, alkyl, and spirocycloalkyl;
Y is a bond, O, S, or NR^{c};
Q⁴ is O, S, or NR⁷;
R² is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl, and a pharmaceutically acceptable counterion;
R^{3a} and R^{3b} are independently selected from hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl;
R⁴ is independently selected from aryl, substituted aryl, heteroaryl, substituted heteroaryl, heterocyclyl, substituted heterocyclyl, cycloalkyl, and substituted cycloalkyl;
R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, substituted sulfonyl, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R⁶ is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl, and a pharmaceutically acceptable counterion;
R⁷ is selected from hydrogen, halo, aminocarbonyl, imino, amidino, aminocarbonylamino, amidinocarbonylamino, carboxy, carboxy ester, hydroxy, alkoxy, substituted alkoxy, acyl, acyloxy, cyano, thiol, alkylthio, substituted alkylthio, sulfonyl, and substituted sulfonyl;
R^{a}, R^{b}, and R^{c} are independently selected from hydrogen, alkyl, and substituted alkyl; and
m is from 0 to 5;
n is from 0 to 1, provided that n is 0 when [symbol] represents a double bond.

68. The compound of claim 67, wherein L² is a bond.

69. The compound of claim 67, wherein L¹ is CH₂.

70. The compound of claim 67, wherein Y is a bond.

71. The compound of claim 67, wherein Y is NH.

72. The compound of claim 67, wherein Y is O.

73. A compound that is Formula (II): 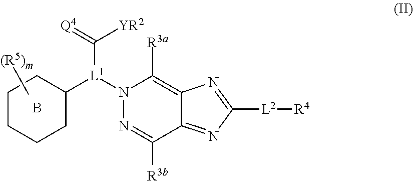
or a pharmaceutically acceptable salt thereof, wherein,
ring B is a 6-membered aromatic ring wherein 1 to 3 ring carbon atoms are optionally replaced by nitrogen or oxygen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;
L¹ is independently C₃₋₆ cycloalkylene or C₁₋₅ alkylene, where one or two CH₂ groups of said C₁₋₅ alkylene are optionally replaced with NR^{a}, S, (C═O), or O and optionally two adjacent carbon atoms form a double bond;
L² is a bond or independently C₃₋₆ cycloalkylene or is C₁₋₅ alkylene where one or two CH₂ groups of said C₁₋₅ alkylene are optionally replaced with NR^{b}, S, (C═O), or O and optionally two adjacent carbon atoms form a double bond or triple bond, and wherein said C₁₋₅ alkylene is optionally substituted with one to three groups independently selected from halo, alkyl, and spirocycloalkyl;
Y is a bond, O, or NR^{c};
Q⁴ is O or NR⁷;
R² is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, and substituted sulfonyl;
R^{3a} and R^{3b} are independently selected from hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl;
R⁴ is independently selected from aryl, substituted aryl, heteroaryl, substituted heteroaryl, heterocyclyl, substituted heterocyclyl, cycloalkyl, and substituted cycloalkyl;
R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, substituted sulfonyl, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R⁷ is selected from hydrogen, halo, aminocarbonyl, imino, amidino, aminocarbonylamino, amidinocarbonylamino, carboxy, carboxy ester, hydroxy, alkoxy, substituted alkoxy, acyl, acyloxy, cyano, thiol, alkylthio, substituted alkylthio, sulfonyl, and substituted sulfonyl;
R^{a}, R^{b}, and R^{c} are independently selected from hydrogen, alkyl, and substituted alkyl; and
m is 1, 2, 3, or 4.

74. The compound of claim 73 having the formula (VII) 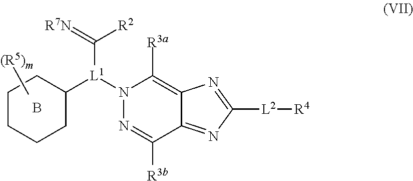
or a pharmaceutically acceptable salt thereof.

75. The compound of claim 73 having the formula (VIII) 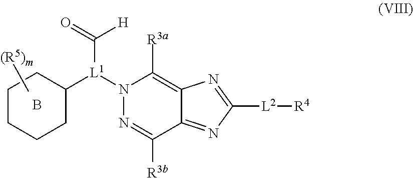
or a pharmaceutically acceptable salt thereof.

76. The compound of claim 73 having the formula (IX) 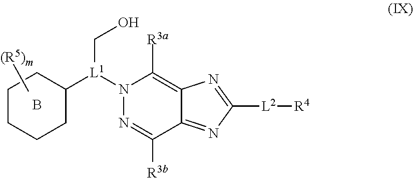
or a pharmaceutically acceptable salt or solvate thereof.

77. A compound that is Formula (III): 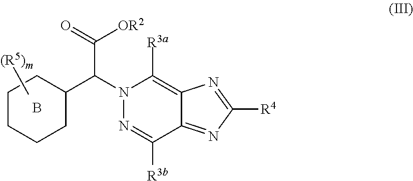
or a pharmaceutically acceptable salt thereof, wherein,
ring B is a 6-membered aromatic ring wherein 1 to 3 ring carbon atoms are optionally replaced by nitrogen or oxygen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;
R² is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, and substituted sulfonyl;
R^{3a} and R^{3b} are independently selected from hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl;
R⁴ is independently selected from aryl, substituted aryl, heteroaryl, substituted heteroaryl, heterocyclyl, substituted heterocyclyl, cycloalkyl, and substituted cycloalkyl;
R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, substituted sulfonyl, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl; and
m is 1, 2, 3, or 4.

78. The compound of claim 77, wherein R^{3a} and R^{3b} are hydrogen.

79. The compound of claim 77 having the formula (V) 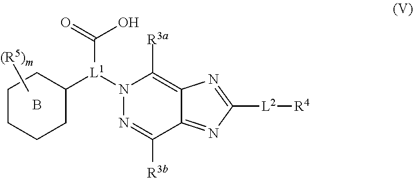
or a pharmaceutically acceptable salt thereof.

80. A compound that is Formula (I): 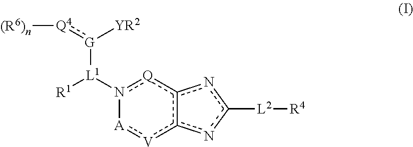
or a pharmaceutically acceptable salt or solvate thereof, wherein:
the dotted lines represent an optional double bond, provided that no two double bonds are adjacent to one another, and that the dotted lines represent at least 3 double bonds;
G is C when the bond between G and Q⁴ is a double bond and G is C-Q¹ when the bond between G and Q⁴ is a single bond;
A, Q, and V are independently selected from N and CR³;
L¹ is independently C₃₋₆ cycloalkylene or C₁₋₅ alkylene, where one or two CH₂ groups of said C₁₋₅ alkylene are optionally replaced with NR^{a}, S, (C═O), or O and optionally two adjacent carbon atoms form a double bond;
L² is a bond or independently C₃₋₆ cycloalkylene or is C₁₋₅ alkylene where one or two CH₂ groups of said C₁₋₅ alkylene are optionally replaced with NR^{b}, S, (C═O), or O and optionally two adjacent carbon atoms form a double bond or triple bond, and wherein said C₁₋₅ alkylene is optionally substituted with one to three groups independently selected from halo, alkyl, and spirocycloalkyl;
Y is a bond, O, S, or NR^{c};
Q¹ is selected from hydrogen, halo, amino, acylamino, alkyl, substituted alkyl, alkenyl, substituted alkenyl, alkynyl, substituted alkynyl, carboxy, carboxy ester, azido, hydroxy, alkoxy, substituted alkoxy, acyl, acyloxy, cyano, thiol, alkylthio, substituted alkylthio, and substituted sulfonyl;
Q⁴ is O, S, or NR⁷;
R¹ is selected from aryl, substituted aryl, heteroaryl, substituted heteroaryl, cycloalkyl, substituted cycloalkyl, heterocyclyl, and substituted heterocyclyl;
R² is selected from the group consisting of hydrogen, C₁₋₆ alkyl, aryl, -A¹, -A¹-(X¹)_{w}-R⁸R⁹, -A¹-R¹⁰, -A¹R¹¹, -A¹-N(R⁹)_{z}, -A¹-NHA²-R¹¹, -A¹-NHA²-NHA³R¹¹, and -A¹-R⁸R⁹; wherein:
A¹, A², A³, and A⁴ are each independently selected from C₁₋₆ alkylene, wherein one to four independent CH₂ groups of each of said A¹, A², A³, and A⁴ are optionally substituted with one to two R¹² groups;
each X¹ is independently selected from the group consisting of —(R⁸-A¹), —(R⁸-A²), —(R⁸-A³), and —(R⁸-A⁴);
R³ is selected from hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl;
R⁴ is independently selected from aryl, substituted aryl, heteroaryl, substituted heteroaryl, heterocyclyl, substituted heterocyclyl, cycloalkyl, and substituted cycloalkyl;
R⁶ is selected from hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, phosphate, phosphonate, phosphinate, phosphorodiamidate, phosphoroamidate monoester, phosphoroamidate diester, cyclic phosphoroamidate, cyclic phosphorodiamidate, phosphonamidate, sulfate, sulfonate, sulfonyl, substituted sulfonyl, and a pharmaceutically acceptable counterion;
R⁷ is selected from hydrogen, halo, aminocarbonyl, imino, amidino, aminocarbonylamino, amidinocarbonylamino, carboxy, carboxy ester, hydroxy, alkoxy, substituted alkoxy, acyl, acyloxy, cyano, thiol, alkylthio, substituted alkylthio, sulfonyl, and substituted sulfonyl;
R⁸ is O;
each R⁹ is independently selected from the group consisting of hydrogen and C₁₋₆ alkyl;
R¹⁰ is selected from the group consisting of phosphate, phosphonate, and sulfate;
R¹¹ is carboxyl;
each R¹² is independently selected from the group consisting of C₁₋₆ alkyl, oxo, aryl, arylalkyl, and hydroxyl;
R^{a}, R^{b}, and R^{c} are independently selected from hydrogen, alkyl, and substituted alkyl;
n is from 0 to 1, provided that n is 0 when the bond between G and Q⁴ is a double bond;
w is an integer from 1 to 3; and
z is an integer from 2 to 3.

81. A compound selected from the group consisting of:
Methyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
Ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
Propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
1-Methylethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
3-Hydroxypropyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
1,1-Dimethylethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
Butyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
2-[(2-Hydroxyethyl)oxy]ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
2-{[2-(Methyloxy)ethyl]oxy}ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
Hexyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
2-({2-[(2-Hydroxyethyl)oxy]ethyl}oxy)ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
2-{[2-({2-[(2-Hydroxyethyl)oxy]ethyl}oxy)ethyl]oxy}ethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
2,3-Dihydroxypropyl 2-[2-(2,3-difluorophenyl)-5H-imidazo[4,5-d]pyridazin-5-yl]-2-[3-[4-propyloxy-2-(trifluoromethyl)phenyl]isoxazol-5-yl]acetate,
3-(Phosphonooxy)propyl[2-(2,3-difluorophenyl)-5H-imidazo[4,5-d]pyridazin-5-yl]{3-[4-(propyloxy)-2-(trifluoromethyl)phenyl]-5-isoxazolyl}acetate mono sodium salt,
3-(Phosphonooxy)propyl[2-(2,3-difluorophenyl)-5H-imidazo[4,5-d]pyridazin-5-yl]{3-[4-(propyloxy)-2-(trifluoromethyl)phenyl]-5-isoxazolyl}acetate,
3-(Sulfooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate sodium salt,
3-(Sulfooxy)propyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoic acid sodium salt,
3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoic acid,
3-[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]oxypropyl-trimethyl-ammonium chloride,
N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanine,
N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanyl-L-phenylalanine,
2-[2-(2,3-Difluorophenyl)-5H-imidazo[4,5-d]pyridazin-5-yl]-N-methyl-2-{3-[4-(propyloxy)-2-(trifluoromethyl)phenyl]-5-isoxazolyl}acetamide,
2-[2-(2,3-Difluorophenyl)-5H-imidazo[4,5-d]pyridazin-5-yl]-N-ethyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-propyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-isopropyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-(3-hydroxypropyl)-2-[3-[4-propyloxy-2-trifluoromethyl)phenyl]isoxazol-5-yl]acetamide,
2-[2-(2,3-difluorophenyl)-5H-imidazo[4,5-d]pyridazin-5-yl]-N-tert-butyl-2-[3-[4-propyloxy-2-(trifluoromethyl)phenyl]isoxazol-5-yl]acetamide,
2-[2-(2,3-difluorophenyl)-5H-imidazo[4,5-d]pyridazin-5-yl]-N-butyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-[2-(2-hydroxyethoxy)ethyl]-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-[2-(2-methoxyethoxy)ethyl]-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-hexyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-[2-[2-(2-hydroxyethoxy)ethoxy]ethyl]-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-[2-[2-[2-(2-hydroxyethoxy)ethoxy]ethoxy]ethyl]-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-(2,3-dihydroxypropyl)-2-[3-[4-propyloxy-2-(trifluoromethyl)phenyl]isoxazol-5-yl]acetamide,
N-3-(phosphonooxy)propyl-2-[2-(2,3-difluorophenyl)-5H-imidazo[4,5-d]pyridazin-5-yl]-2-[3-[4-propyloxy-2-(trifluoromethyl)phenyl]isoxazol-5-yl]acetamide mono sodium salt,
N-3-(phosphonooxy)propyl-2-[2-(2,3-difluorophenyl)-5H-imidazo[4,5-d]pyridazin-5-yl]-2-[3-[4-propyloxy-2-(trifluoromethyl)phenyl]isoxazol-5-yl]acetamide,
N-3-(Sulfonyloxy)propyl-2-[2-(2,3-difluorophenyl)-5H-imidazo[4,5-d]pyridazin-5-yl]-2-[3-[4-propyloxy-2-(trifluoromethyl)phenyl]isoxazol-5-yl]acetamide mono sodium salt,
N-3-(Sulfonyloxy)propyl-2-[2-(2,3-difluorophenyl)-5H-imidazo[4,5-d]pyridazin-5-yl]-2-[3-[4-propyloxy-2-(trifluoromethyl)phenyl]isoxazol-5-yl]acetamide,
3-[[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]amino]propanoic acid sodium salt,
3-[[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]amino]propanoic acid,
3-[[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]amino]propyl-trimethyl-ammonium chloride,
2-[3-[[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]amino]propanoylamino]propanoic acid,
2-[2-[3-[[2-[2-(2,3-Difluorophenyl)-5H-imidazo[4,5-d]pyridazin-5-yl]-2-[3-[4-propyloxy-2-(trifluoromethyl)phenyl]isoxazol-5-yl]acetyl]amino]propanoylamino]propanoylamino]propanoic acid,
Methyl [2-(2,3-difluorophenyl)-5H-imidazo[4,5-d]pyridazin-5-yl]{6-[4-(propyloxy)-2-(trifluoromethyl)phenyl]-3-pyridinyl}acetate,
Ethyl [2-(2,3-difluorophenyl)-5H-imidazo[4,5-d]pyridazin-5-yl]{6-[4-(propyloxy)-2-(trifluoromethyl)phenyl]-3-pyridinyl}acetate,
Propyl [2-(2,3-difluorophenyl)-5H-imidazo[4,5-d]pyridazin-5-yl]{6-[4-(propyloxy)-2-(trifluoromethyl)phenyl]-3-pyridinyl}acetate,
1-Methylethyl [2-(2,3-difluorophenyl)-5H-imidazo[4,5-d]pyridazin-5-yl]{6-[4-(propyloxy)-2-(trifluoromethyl)phenyl]-3-pyridinyl}acetate,
3-Hydroxypropyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
Tert-butyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
Butyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
2-(2-Hydroxyethoxy)ethyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
2-(2-Methoxyethoxy)ethyl 2-[2-(2,3-difluorophenyl)-5H-imidazo[4,5-d]pyridazin-5-yl]-2-[6-[4-propoxy-2-(trifluoromethyl)phenyl]-3-pyridinyl]acetate,
Hexyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
2-[2-(2-Hydroxyethoxy)ethoxy]ethyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
2-[2-[2-(2-Hydroxyethoxy)ethoxy]ethoxy]ethyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
2,3-Dihydroxypropyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
3-(Phosphonooxy)propyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate mono sodium salt,
3-(Phosphonooxy)propyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
3-(Sulfonyloxy)propyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate mono sodium salt,
3-(Sulfonyloxy)propyl 2-[2-(2,3-difluorophenyl)-5H-imidazo[4,5-d]pyridazin-5-yl]-2-[6-[4-propyloxy-2-(trifluoromethyl)phenyl]-3-pyridinyl]acetate,
3-[2-[2-(2,3-Difluorophenyl)-5H-imidazo[4,5-d]pyridazin-5-yl]-2-[6-[4-propyloxy-2-(trifluoromethyl)phenyl]-3-pyridinyl]acetyl]oxypropanoic acid sodium salt,
3-[2-[2-(2,3-Difluorophenyl)-5H-imidazo[4,5-d]pyridazin-5-yl]-2-[6-[4-propyloxy-2-(trifluoromethyl)phenyl]-3-pyridinyl]acetyl]oxypropanoic acid,
3-[2-[2-(2,3-Difluorophenyl)-5H-imidazo[4,5-d]pyridazin-5-yl]-2-[6-[4-propyloxy-2-(trifluoromethyl)phenyl]-3-pyridinyl]acetyl]oxypropyl-dimethyl-ammonium chloride,
2-[3-[2-[2-(2,3-Difluorophenyl)-5H-imidazo[4,5-d]pyridazin-5-yl]-2-[6-[4-propyloxy-2-(trifluoromethyl)phenyl]-3-pyridinyl]acetyl]oxypropanoylamino]propanoic acid,
2-[2-[3-[2-[2-(2,3-Difluorophenyl)-5H-imidazo[4,5-d]pyridazin-5-yl]-2-[6-[4-propyloxy-2-(trifluoromethyl)phenyl]-3-pyridinyl]acetyl]oxypropanoylamino]propanoylamino]propanoic acid,
Methyl [2-(2,3-difluorophenyl)-5H-imidazo[4,5-d]pyridazin-5-yl]{2-[4-(propyloxy)-2-(trifluoromethyl)phenyl]-5-pyrimidinyl}acetate,
Ethyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
Propyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
Isopropyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
3-Hydroxypropyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
Tert-butyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
Butyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
2-(2-Hydroxyethoxy)ethyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
2-(2-Methoxyethoxy)ethyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
Hexyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
2-[2-(2-Hydroxyethoxy)ethoxy]ethyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
2-[2-[2-(2-Hydroxyethoxy)ethoxy]ethoxy]ethyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
2,3-Dihydroxypropyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
3-(Phosphonooxy)propyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate mono sodium salt,
3-(Phosphonooxy)propyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
3-(Sulfonyloxy)propyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate mono sodium salt,
3-(Sulfonyloxy)propyl 2-[2-(2,3-difluorophenyl)-5H-imidazo[4,5-d]pyridazin-5-yl]-2-[2-[4-propyloxy-2-(trifluoromethyl)phenyl]-5-pyrimidinyl]acetate,
3-[2-[2-(2,3-Difluorophenyl)-5H-imidazo[4,5-d]pyridazin-5-yl]-2-[2-[4-propyloxy-2-(trifluoromethyl)phenyl]-5-pyrimidinyl]acetyl]oxypropanoic acid sodium salt,
3-[2-[2-(2,3-Difluorophenyl)-5H-imidazo[4,5-d]pyridazin-5-yl]-2-[2-[4-propyloxy-2-(trifluoromethyl)phenyl]-5-pyrimidinyl]acetyl]oxypropanoic acid,
3-[2-[2-(2,3-Difluorophenyl)-5H-imidazo[4,5-d]pyridazin-5-yl]-2-[2-[4-propyloxy-2-(trifluoromethyl)phenyl]-5-pyrimidinyl]acetyl]oxypropyl-trimethyl-ammonium chloride,
2-[3-[2-[2-(2,3-Difluorophenyl)-5H-imidazo[4,5-d]pyridazin-5-yl]-2-[2-[4-propyloxy-2-(trifluoromethyl)phenyl]-5-pyrimidinyl]acetyl]oxypropanoylamino]propanoic acid,
2-[2-[3-[2-[2-(2,3-Difluorophenyl)-5H-imidazo[4,5-d]pyridazin-5-yl]-2-[2-[4-propyloxy-2-(trifluoromethyl)phenyl]-5-pyrimidinyl]acetyl]oxypropanoylamino]propanoylamino]propanoic acid,
Methyl 2-[6-[2,4-bis(trifluoromethyl)phenyl]pyridazin-3-yl]-2-[2-(2-fluorophenyl)-5H-imidazo[4,5-c]pyridin-5-yl]acetate,
Ethyl 2-[6-[2,4-bis(trifluoromethyl)phenyl]pyridazin-3-yl]-2-[2-(2-fluorophenyl)-5H-imidazo[4,5-c]pyridin-5-yl]acetate,
Propyl 2-[6-[2,4-bis(trifluoromethyl)phenyl]pyridazin-3-yl]-2-[2-(2-fluorophenyl)-5H-imidazo[4,5-c]pyridin-5-yl]acetate,
Isopropyl 2-[6-[2,4-bis(trifluoromethyl)phenyl]pyridazin-3-yl]-2-[2-(2-fluorophenyl)-5H-imidazo[4,5-c]pyridin-5-yl]acetate,
3-hydroxypropyl 2-[6-[2,4-bis(trifluoromethyl)phenyl]pyridazin-3-yl]-2-[2-(2-fluorophenyl)-5H-imidazo[4,5-c]pyridin-5-yl]acetate,
Tert-butyl 2-[6-[2,4-bis(trifluoromethyl)phenyl]pyridazin-3-yl]-2-[2-(2-fluorophenyl)-5H-imidazo[4,5-c]pyridin-5-yl]acetate,
Butyl 2-[6-[2,4-bis(trifluoromethyl)phenyl]pyridazin-3-yl]-2-[2-(2-fluorophenyl)-5H-imidazo[4,5-c]pyridin-5-yl]acetate,
2-(2-Hydroxyethoxy)ethyl 2-[6-[2,4-bis(trifluoromethyl)phenyl]pyridazin-3-yl]-2-[2-(2-fluorophenyl)-5H-imidazo[4,5-c]pyridin-5-yl]acetate,
2-(2-Methyloxyethoxy)ethyl 2-[6-[2,4-bis(trifluoromethyl)phenyl]pyridazin-3-yl]-2-[2-(2-fluorophenyl)-5H-imidazo[4,5-c]pyridin-5-yl]acetate,
Hexyl 2-[6-[2,4-bis(trifluoromethyl)phenyl]pyridazin-3-yl]-2-[2-(2-fluorophenyl)-5H-imidazo[4,5-c]pyridin-5-yl]acetate,
2-[2-(2-Hydroxyethoxy)ethoxy]ethyl 2-[6-[2,4-bis(trifluoromethyl)phenyl]pyridazin-3-yl]-2-[2-(2-fluorophenyl)-5H-imidazo[4,5-c]pyridin-5-yl]acetate,
2-[2-[2-(2-Hydroxyethoxy)ethoxy]ethoxy]ethyl 2-[6-[2,4-bis(trifluoromethyl)phenyl]pyridazin-3-yl]-2-[2-(2-fluorophenyl)-5H-imidazo[4,5-c]pyridin-5-yl]acetate,
2,3-Dihydroxypropyl 2-[6-[2,4-bis(trifluoromethyl)phenyl]pyridazin-3-yl]-2-[2-[(2-fluorophenyl)methyl]-5H-imidazo[4,5-c]pyridin-5-yl]acetate,
3-(Phosphonooxy)propyl 2-[6-[2,4-bis(trifluoromethyl)phenyl]pyridazin-3-yl]-2-[2-(2-fluorophenyl)-5H-imidazo[4,5-c]pyridin-5-yl]acetate mono sodium salt,
3-(Phosphonooxy)propyl 2-[6-[2,4-bis(trifluoromethyl)phenyl]pyridazin-3-yl]-2-[2-(2-fluorophenyl)-5H-imidazo[4,5-c]pyridin-5-yl]acetate,
3-(Sulfonyloxy)propyl 2-[6-[2,4-bis(trifluoromethyl)phenyl]pyridazin-3-yl]-2-[2-(2-fluorophenyl)-5H-imidazo[4,5-c]pyridin-5-yl]acetate mono sodium salt,
3-(Sulfonyloxy)propyl 2-[6-[2,4-bis(trifluoromethyl)phenyl]pyridazin-3-yl]-2-[2-(2-fluorophenyl)-5H-imidazo[4,5-c]pyridin-5-yl]acetate,
3-[2-[6-[2,4-Bis(trifluoromethyl)phenyl]pyridazin-3-yl]-2-[2-(2-fluorophenyl)-5H-imidazo[4,5-c]pyridin-5-yl]acetyl]oxypropanoate sodium salt,
3-[2-[6-[2,4-Bis(trifluoromethyl)phenyl]pyridazin-3-yl]-2-[2-(2-fluorophenyl)-5H-imidazo[4,5-c]pyridin-5-yl]acetyl]oxypropanoic acid,
3-[2-[6-[2,4-Bis(trifluoromethyl)phenyl]pyridazin-3-yl]-2-[2-(2-fluorophenyl)-5H-imidazo[4,5-c]pyridin-5-yl]acetyl]oxypropyl-trimethyl-ammonium chloride,
2-[3-[2-[6-[2,4-Bis(trifluoromethyl)phenyl]pyridazin-3-yl]-2-[2-(2-fluorophenyl)-5H-imidazo[4,5-c]pyridin-5-yl]acetyl]oxypropanoylamino]propanoic acid,
2-[2-[3-[2-[6-[2,4-Bis(trifluoromethyl)phenyl]pyridazin-3-yl]-2-[2-(2-fluorophenyl)-5H-imidazo[4,5-c]pyridin-5-yl]acetyl]oxypropanoylamino]propanoylamino]propanoic acid,
Methyl 2-[6-[2,4-bis(trifluoromethyl)phenyl]pyridazin-3-yl]-2-[8-(2-fluorophenyl)-1H-purin-1-yl]acetate,
Ethyl 2-[6-[2,4-bis(trifluoromethyl)phenyl]pyridazin-3-yl]-2-[8-(2-fluorophenyl)-1H-purin-1-yl]acetate,
Propyl 2-[6-[2,4-bis(trifluoromethyl)phenyl]pyridazin-3-yl]-2-[8-(2-fluorophenyl)-1H-purin-1-yl]acetate,
Isopropyl 2-[6-[2,4-bis(trifluoromethyl)phenyl]pyridazin-3-yl]-2-[8-(2-fluorophenyl)-1H-purin-1-yl]acetate,
3-Hydroxypropyl 2-[6-[2,4-bis(trifluoromethyl)phenyl]pyridazin-3-yl]-2-[8-(2-fluorophenyl)-1H-purin-1-yl]acetate,
Tert-butyl 2-[6-[2,4-bis(trifluoromethyl)phenyl]pyridazin-3-yl]-2-[8-(2-fluorophenyl)-1H-purin-1-yl]acetate,
Butyl 2-[6-[2,4-bis(trifluoromethyl)phenyl]pyridazin-3-yl]-2-[8-(2-fluorophenyl)-1H-purin-1-yl]acetate,
2-(2-Hydroxyethoxy)ethyl 2-[6-[2,4-bis(trifluoromethyl)phenyl]pyridazin-3-yl]-2-[8-(2-fluorophenyl)-1H-purin-1-yl]acetate,
2-(2-Methyloxyethoxy)ethyl 2-[6-[2,4-bis(trifluoromethyl)phenyl]pyridazin-3-yl]-2-[8-(2-fluorophenyl)-1H-purin-1-yl]acetate,
Hexyl 2-[6-[2,4-bis(trifluoromethyl)phenyl]pyridazin-3-yl]-2-[8-(2-fluorophenyl)-1H-purin-1-yl]acetate,
Methyl 2-[2-(2-fluorophenyl)-5H-imidazo[4,5-d]pyridazin-5-yl]-2-[3-[4-propyloxy-2-(trifluoromethyl)phenyl]isoxazol-5-yl]acetate,
Methyl 2-[2-(2,3-difluorophenyl)-5H-imidazo[4,5-d]pyridazin-5-yl]-2-[3-[2,4-bis(trifluoromethyl)phenyl]isoxazol-5-yl]acetate,
Methyl 2-[2-(2-fluorophenyl)-5H-imidazo[4,5-d]pyridazin-5-yl]-2-[3-[2,4-bis(trifluoromethyl)phenyl]isoxazol-5-yl]acetic acid,
Ethyl 2-[2-(2-fluorophenyl)-5H-imidazo[4,5-d]pyridazin-5-yl]-2-[3-[4-propyloxy-2-(trifluoromethyl)phenyl]isoxazol-5-yl]acetate,
Ethyl 2-[2-(2,3-difluorophenyl)-5H-imidazo[4,5-d]pyridazin-5-yl]-2-[3-[2,4-bis(trifluoromethyl)phenyl]isoxazol-5-yl]acetate,
Ethyl 2-[2-(2-fluorophenyl)-5H-imidazo[4,5-d]pyridazin-5-yl]-2-[3-[2,4-bis(trifluoromethyl)phenyl]isoxazol-5-yl]acetate,
Propyl 2-[2-(2-fluorophenyl)-5H-imidazo[4,5-d]pyridazin-5-yl]-2-[3-[4-propyloxy-2-(trifluoromethyl)phenyl]isoxazol-5-yl]acetate,
Propyl 2-[2-(2,3-difluorophenyl)-5H-imidazo[4,5-d]pyridazin-5-yl]-2-[3-[2,4-bis(trifluoromethyl)phenyl]isoxazol-5-yl]acetate,
Propyl 2-[2-(2-fluorophenyl)-5H-imidazo[4,5-d]pyridazin-5-yl]-2-[3-[2,4-bis(trifluoromethyl)phenyl]isoxazol-5-yl]acetate,
2-[2-(2-Fluorophenyl)-5H-imidazo[4,5-d]pyridazin-5-yl]-N-methyl-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-methyl-2-[3-[2,4-bis(trifluoromethyl)phenyl]isoxazol-5-yl]acetamide,
2-[2-(2-Fluorophenyl)-5H-imidazo[4,5-d]pyridazin-5-yl]-N-methyl-2-[3-[2,4-bis(trifluoromethyl)phenyl]isoxazol-5-yl]acetamide,
N-ethyl-2-[2-(2-Fluorophenyl)-5H-imidazo[4,5-d]pyridazin-5-yl]-2-[3-[4-propyloxy-2-(trifluoromethyl)phenyl]isoxazol-5-yl]acetamide,
2-[2-(2,3-Difluorophenyl)-5H-imidazo[4,5-d]pyridazin-5-yl]-N-ethyl-2-[3-[2,4-bis(trifluoromethyl)phenyl]isoxazol-5-yl]acetamide,
N-ethyl-2-[2-(2-fluorophenyl)-5H-imidazo[4,5-d]pyridazin-5-yl]-2-[3-[2,4-bis(trifluoromethyl)phenyl]isoxazol-5-yl]acetamide,
2-[2-(2-Fluorophenyl)-5H-imidazo[4,5-d]pyridazin-5-yl]-2-[3-[4-propyloxy-2-(trifluoromethyl)phenyl]isoxazol-5-yl]-N-propyl-acetamide,
2-[2-(2,3-Difluorophenyl)-5H-imidazo[4,5-d]pyridazin-5-yl]-2-[3-[2,4-bis(trifluoromethyl)phenyl]isoxazol-5-yl]-N-propyl-acetamide,
2-[2-(2-Fluorophenyl)-5H-imidazo[4,5-d]pyridazin-5-yl]-2-[3-[2,4-bis(trifluoromethyl)phenyl]isoxazol-5-yl]-N-propyl-acetamide,
Methyl 2-[2-(2-fluorophenyl)-5H-imidazo[4,5-d]pyridazin-5-yl]-2-[6-[4-propyloxy-2-(trifluoromethyl)phenyl]-3-pyridinyl]acetate,
Methyl 2-[2-(2,3-difluorophenyl)-5H-imidazo[4,5-d]pyridazin-5-yl]-2-[6-[2,4-bis(trifluoromethyl)phenyl]-3-pyridinyl]acetate,
Methyl 2-[2-(2-fluorophenyl)-5H-imidazo[4,5-d]pyridazin-5-yl]-2-[6-[2,4-bis(trifluoromethyl)phenyl]-3-pyridinyl]acetate,
Ethyl 2-[2-(2-fluorophenyl)-5H-imidazo[4,5-d]pyridazin-5-yl]-2-[6-[4-propyloxy-2-(trifluoromethyl)phenyl]-3-pyridinyl]acetate,
Ethyl 2-[2-(2,3-difluorophenyl)-5H-imidazo[4,5-d]pyridazin-5-yl]-2-[6-[2,4-bis(trifluoromethyl)phenyl]-3-pyridinyl]acetate,
Ethyl 2-[2-(2-fluorophenyl)-5H-imidazo[4,5-d]pyridazin-5-yl]-2-[6-[2,4-bis(trifluoromethyl)phenyl]-3-pyridinyl]acetate,
Propyl 2-[2-(2-fluorophenyl)-5H-imidazo[4,5-d]pyridazin-5-yl]-2-[6-[4-propyloxy-2-(trifluoromethyl)phenyl]-3-pyridinyl]acetate,
Propyl 2-[2-(2,3-difluorophenyl)-5H-imidazo[4,5-d]pyridazin-5-yl]-2-[6-[2,4-bis(trifluoromethyl)phenyl]-3-pyridinyl]acetate,
Propyl 2-[2-(2-fluorophenyl)-5H-imidazo[4,5-d]pyridazin-5-yl]-2-[6-[2,4-bis(trifluoromethyl)phenyl]-3-pyridinyl]acetate,
Methyl 2-[2-(2-fluorophenyl)-5H-imidazo[4,5-d]pyridazin-5-yl]-2-[2-[4-propyloxy-2-(trifluoromethyl)phenyl]-5-pyrimidinyl]acetate,
Methyl 2-[2-(2,3-difluorophenyl)-5H-imidazo[4,5-d]pyridazin-5-yl]-2-[2-[2,4-bis(trifluoromethyl)phenyl]-5-pyrimidinyl]acetate,
Methyl 2-[2-(2-fluorophenyl)-5H-imidazo[4,5-d]pyridazin-5-yl]-2-[2-[2,4-bis(trifluoromethyl)phenyl]-5-pyrimidinyl]acetate,
Ethyl 2-[2-(2-fluorophenyl)-5H-imidazo[4,5-d]pyridazin-5-yl]-2-[2-[4-propyloxy-2-(trifluoromethyl)phenyl]-5-pyrimidinyl]acetate,
Ethyl 2-[2-(2,3-difluorophenyl)-5H-imidazo[4,5-d]pyridazin-5-yl]-2-[2-[2,4-bis(trifluoromethyl)phenyl]-5-pyrimidinyl]acetate,
Ethyl 2-[2-(2-fluorophenyl)-5H-imidazo[4,5-d]pyridazin-5-yl]-2-[2-[2,4-bis(trifluoromethyl)phenyl]-5-pyrimidinyl]acetate,
Propyl 2-[2-(2-fluorophenyl)-5H-imidazo[4,5-d]pyridazin-5-yl]-2-[2-[4-propyloxy-2-(trifluoromethyl)phenyl]-5-pyrimidinyl]acetate,
Propyl 2-[2-(2,3-difluorophenyl)-5H-imidazo[4,5-d]pyridazin-5-yl]-2-[2-[2,4-bis(trifluoromethyl)phenyl]-5-pyrimidinyl]acetate,
Propyl 2-[2-(2-fluorophenyl)-5H-imidazo[4,5-d]pyridazin-5-yl]-2-[2-[2,4-bis(trifluoromethyl)phenyl]-5-pyrimidinyl]acetate,
Methyl 2-[2-(2,3-difluorophenyl)-5H-imidazo[4,5-c]pyridin-5-yl]-2-[6-[2,4-bis(trifluoromethyl)phenyl]pyridazin-3-yl]acetate,
Methyl 2-[2-(2-fluorophenyl)-5H-imidazo[4,5-c]pyridin-5-yl]-2-[6-[4-propyloxy-2-(trifluoromethyl)phenyl]pyridazin-3-yl]acetate,
Methyl 2-[2-(2,3-difluorophenyl)-5H-imidazo[4,5-c]pyridin-5-yl]-2-[6-[4-propyloxy-2-(trifluoromethyl)phenyl]pyridazin-3-yl]acetate,
Ethyl 2-[2-(2,3-difluorophenyl)-5H-imidazo[4,5-c]pyridin-5-yl]-2-[6-[2,4-bis(trifluoromethyl)phenyl]pyridazin-3-yl]acetate,
Ethyl 2-[2-(2-fluorophenyl)-5H-imidazo[4,5-c]pyridin-5-yl]-2-[6-[4-propyloxy-2-(trifluoromethyl)phenyl]pyridazin-3-yl]acetate,
Ethyl 2-[2-(2,3-difluorophenyl)-5H-imidazo[4,5-c]pyridin-5-yl]-2-[6-[4-propyloxy-2-(trifluoromethyl)phenyl]pyridazin-3-yl]acetate,
Propyl 2-[2-(2,3-difluorophenyl)-5H-imidazo[4,5-c]pyridin-5-yl]-2-[6-[2,4-bis(trifluoromethyl)phenyl]pyridazin-3-yl]acetate,
Propyl 2-[2-(2-fluorophenyl)-5H-imidazo[4,5-c]pyridin-5-yl]-2-[6-[4-propyloxy-2-(trifluoromethyl)phenyl]pyridazin-3-yl]acetate,
Propyl 2-[2-(2,3-difluorophenyl)-5H-imidazo[4,5-c]pyridin-5-yl]-2-[6-[4-propyloxy-2-(trifluoromethyl)phenyl]pyridazin-3-yl]acetate,
Methyl 2-[8-(2,3-difluorophenyl)-1H-purin-1-yl]-2-[6-[2,4-bis(trifluoromethyl)phenyl]pyridazin-3-yl]acetate,
Methyl 2-[8-(2-fluorophenyl)-1H-purin-1-yl]-2-[6-[4-propyloxy-2-(trifluoromethyl)phenyl]pyridazin-3-yl]acetate,
Methyl 2-[8-(2,3-difluorophenyl)-1H-purin-1-yl]-2-[6-[4-propyloxy-2-(trifluoromethyl)phenyl]pyridazin-3-yl]acetate,
Ethyl 2-[8-(2,3-difluorophenyl)-1H-purin-1-yl]-2-[6-[2,4-bis(trifluoromethyl)phenyl]pyridazin-3-yl]acetate,
Ethyl 2-[8-(2-fluorophenyl)-1H-purin-1-yl]-2-[6-[4-propyloxy-2-(trifluoromethyl)phenyl]pyridazin-3-yl]acetate,
Ethyl 2-[8-(2,3-difluorophenyl)-1H-purin-1-yl]-2-[6-[4-propyloxy-2-(trifluoromethyl)phenyl]pyridazin-3-yl]acetate,
Propyl 2-[8-(2,3-difluorophenyl)-1H-purin-1-yl]-2-[6-[2,4-bis(trifluoromethyl)phenyl]pyridazin-3-yl]acetate,
Propyl 2-[8-(2-fluorophenyl)-1H-purin-1-yl]-2-[6-[4-propyloxy-2-(trifluoromethyl)phenyl]pyridazin-3-yl]acetate,
Propyl 2-[8-[(2,3-difluorophenyl)methyl]-1H-purin-1-yl]-2-[6-[4-propyloxy-2-(trifluoromethyl)phenyl]pyridazin-3-yl]acetate,
2-Methylpropyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
Phenylmethyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
4-(Dimethylamino)butyl [2-(2,3-difluorophenyl)-5H-imidazo[4,5-d]pyridazin-5-yl]{3-[4-(propyloxy)-2-(trifluoromethyl)phenyl]-5-isoxazolyl}acetate dihydrochloride,
4-(Dimethylamino)butyl [2-(2,3-difluorophenyl)-5H-imidazo[4,5-d]pyridazin-5-yl]{3-[4-(propyloxy)-2-(trifluoromethyl)phenyl]-5-isoxazolyl}acetate,
N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanine sodium salt,
N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanine,
N-{3-[([2-(2,3-Difluorophenyl)-5H-imidazo[4,5-d]pyridazin-5-yl]{3-[4-(propyloxy)-2-(trifluoromethyl)phenyl]-5-isoxazolyl}acetyl)oxy]propanoyl}-L-alanyl-L-phenylalanine,
Methyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
2-[(2-hydroxyethyl)oxy]ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
3-hydroxy-2-(hydroxymethyl)-2-methylpropyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
Ethyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
3-Hydroxypropyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
Methyl {3-[4-cyclobutyl-2-(trifluoromethyl)phenyl]-5-isoxazolyl}[2-(2,3-difluorophenyl)-5H-imidazo[4,5-d]pyridazin-5-yl]acetate,
Propyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
4-hydroxybutyl {3-[4-cyclopropyl-2-(trifluoromethyl)phenyl]-5-isoxazolyl}[2-(2,3-difluorophenyl)-5H-imidazo[4,5-d]pyridazin-5-yl]acetate,
3-({bis[(1,1-dimethylethyl)oxy]phosphoryl}oxy)propyl {3-[4-cyclopropyl-2-(trifluoromethyl)phenyl]-5-isoxazolyl}[2-(2,3-difluorophenyl)-5H-imidazo[4,5-d]pyridazin-5-yl]acetate, and
Methyl [2-(2-fluorophenyl)-5H-imidazo[4,5-c]pyridin-5-yl]{3-[4-(propyloxy)-2-(trifluoromethyl)phenyl]-5-isoxazolyl}acetate,
and pharmaceutically acceptable salts and solvates thereof.

82. A pharmaceutical composition comprising a pharmaceutically acceptable carrier and a therapeutically effective amount of a compound of claim 1 or a pharmaceutically acceptable salt thereof.

83. A method for treating a viral infection in a patient said viral infection mediated at least in part by a virus in the Flaviviridae family of viruses which method comprises administering to the patient the compound of claim 1, or a pharmaceutically acceptable salt thereof.

84. The method of claim 83, wherein the viral infection is a hepatitis C viral infection.

85. The method of claim 83, wherein the compound is administered to the patient in combination with a therapeutically effective amount of one or more additional agents active against the hepatitis C virus.

86. The method of claim 85, wherein the additional agent active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

87. The method of claim 85, wherein said additional agent active against the hepatitis C virus is interferon.

88. The method of claim 85, wherein said additional agent active against the hepatitis C virus is ribavirin.

89. The method of claim 85, wherein said additional agent active against the hepatitis C virus is interferon in combination with ribavirin.
